(19) United States
(12) Patent Application Publication
(10) Pub. No.: US 2024/0336976 A1
(43) Pub. Date: Oct. 10, 2024

(54) GENE PANEL PREDICTIVE OF THE RESPONSE OF TRIPLE NEGATIVE BREAST CANCER TO NEOADJUVANT CHEMOTHERAPY FOR PRECISION MEDICINE

(71) Applicant: Qatar Foundation for Education, Science and Community Development, Doha (QA)

(72) Inventor: Nehad Alajez, Doha (QA)

(21) Appl. No.: 18/627,942

(22) Filed: Apr. 5, 2024

Related U.S. Application Data
(60) Provisional application No. 63/457,622, filed on Apr. 6, 2023.

(51) Int. Cl. C12Q 1/6886

(52) U.S. Cl. CPC C12Q 1/6886; C12Q 2600/106; C12Q 2600/118; C12Q 2600/158

(57) ABSTRACT

Gene signatures predictive of pathological complete response and residual disease in triple negative breast cancer patients have been identified. These discoveries have clinical implications in patient stratification and precision medicine.

### CROSS REFERENCE TO RELATED APPLICATIONS

This application claims the benefit of U.S. Provisional Application No. 63/457,622 filed Apr. 6, 2023, which is incorporated herein by reference in its entirety.

### FIELD

The present disclosure relates to methods for predicting responses to therapies for breast cancer.

### BACKGROUND

Triple negative breast cancer (TNBC) represents 15%-20% of all breast cancers and is characterized by the lack of hormone receptors (HR), including estrogen receptor (ER) and progesterone receptor (PR) expression, in addition to the lack of epidermal growth factor receptor-2 (HER-2) amplification. Despite the lower incidence of TNBC compared to other molecular subtypes, TNBC presents with an advanced tumor stage and patients have worse overall survival (OS) compared to other types of breast cancer. Neoadjuvant chemotherapy (NAC) followed by surgery is the standard-of-care clinical practice for TNBC with larger tumors.

Longitudinal patterns of TNBC response to NAC are oftentimes used to assess treatment response and likelihood for OS. Pathological complete response (pCR) is associated with better prognosis, while residual disease (RD) predicts worse clinical outcomes.

There is a need for timely identification of patients who will not benefit from NAC so as to avoid exposure of this patient group to toxic drugs and unnecessary delays in surgical intervention.

### SUMMARY

Provided herein are gene signatures predictive of pCR and RD in TNBC. Also provided are gene signatures predictive of disease free survival (DFS), distant metastasis free survival (DMFS) or overall survival (OS) in basal breast cancer patients.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, as will occur to one having ordinary skill in the art and having the benefit of this disclosure.

FIG. 1. Venn diagram depicting the overlap among genes predictive of pCR from the GSE164458_A, GSE164458_B, GSE164458_C, and GSE106977 discovery cohorts.

FIG. 2. Protein-protein interaction (PPI) network analysis of the 56 common genes predictive of pCR.

FIG. 3. Receiver Operating Curve (ROC) analysis for the combined pCR score (mean expression) in the GSE164458_A, GSE164458_B, GSE164458_C, and GSE106977 discovery cohorts and RJNA688066 and GSE192341 validation cohorts. AUC and p values as reindicted for each plot.

FIG. 4. Venn diagram depicting the overlap among genes predictive of RD from the GSE164458_A, GSE164458_B, GSE164458_C, and GSE106977 discovery cohorts.

FIG. 5. ROC analysis for the combined RD score (mean expression) in the GSE164458_A, GSE164458_B, GSE164458_C, and GSE106977 discovery cohorts and RJNA688066 and GSE192341 validation cohorts. AUC and p values as reindicted for each plot

FIG. 6. ROC analysis for the combined pCR/RD score (mean expression) in the GSE164458_A, GSE164458_B, GSE164458_C, and GSE106977, RJNA688066 and GSE192341 cohorts. AUC and p values as reindicted for each plot.

FIG. 7A. Ability of the identified 56 gene panel predictive of pCR to predict disease free survival (DFS).

FIG. 7B. Ability of the identified 56 gene panel predictive of pCR to predict distant metastasis-free survival (DMFS).

FIG. 7C. Ability of the identified 56 gene panel predictive of pCR to predict overall survival (OS) in a large cohort of basal breast cancer.

### DETAILED DESCRIPTION

Triple negative breast cancer (TNBC) patients exhibiting pathological complete response (pCR) have better clinical outcomes compared to those with residual disease (RD). Therefore, robust biomarkers that can predict pCR may help with triage and resource prioritization in patients with TNBC. In this investigation, we identified gene panels predictive of pCR and RD in TNBC using four discovery and two additional validation cohorts (total TNBC=741). Our data identified 56 genes predictive of pCR with high specificity and sensitivity across all six cohorts using receiver operating characteristic curve (ROC) area under curve (AUC) analysis and asymptotic p-value <0.05. Additionally, we identified 31 gene signatures predictive of RD, which performed very well in four of the six cohorts. The pCR-derived signature also predicted better relapse-free survival (RFS), overall survival (OS), and distant-metastasis-free survival (DMFS) in an independent cohort of basal breast cancer.

While single cell analysis of tumor heterogeneity and clonal evolution has been studied and has been linked to variable responses to NAC in TNBC, currently there are no predictive signatures for RD and pCR in the context of NAC in clinical practice.

We previously reported the presence of immune gene expression as the hallmark of pCR in TNBC employing single cell analysis. In the current study, we utilized four different cohorts and identified the gene signatures predicative of pCR and RD. The identified gene panes were tested in the same four cohorts and were validated in additional two cohorts using receiver operating characteristic (ROC) analysis. The identified pCR gene panel also predicted better disease-free survival (DFS) in an additional cohort of basal breast cancer. Taken together, we have identified gene panel predictive of pCR and RD, which can also predict clinical outcomes in patients with TNBC.

### Definitions

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Numerical values relating to measurements are subject to measurement errors that place limits on their accuracy. For this reason, all numerical values provided herein, unless otherwise indicated, are to be understood as being modified by the term “about.” Accordingly, the term about refers to an error of +10%. Otherwise, the last decimal place of a numerical value provided herein indicates its degree of accuracy. Where no other error margins are given, the maximum margin is ascertained by applying the rounding-off convention to the last decimal place or last significant digit when a decimal is not present in the given numerical value.

The term “area under the curve (AUC)” refers to an index that summarizes the “overall” location of the entire ROC curve. The AUC can be interpreted as the probability that a randomly chosen diseased subject is rated or ranked as more likely to be diseased than a randomly chosen non-diseased subject. The AUC refers to the average value of sensitivity (true positive rate) against the false positive rate (1-specificity). An AUC=1 means that the diagnostic test is perfect in the differentiation between the diseased and non-diseased. An AUC=0.5 means that the curve is located on the diagonal line in ROC space and there is no predictive power.

“Neoadjuvant chemotherapy” is chemotherapy that a patient with cancer receives before the primary course of treatment. In some embodiments, neoadjuvant therapy may shrink a cancerous tumor prior to other treatments, such as, e.g., surgery.

In some embodiments, provided is a method for predicting pathological complete response (pCR) in triple negative cancer patients (TNBC) based on measuring transcript per million expression values of genes in a gene panel, wherein the gene panel comprises one or more of the following genes: LMNB1, TFDP1, SMC4, NASP, RAD54L, NUP210, SEMA4D, BRIP1, GBP5, STIL, FERMT3, LAMP3, SLAMF8, SNX20, HIST1H2BO, SLC25A19, IL32, KIF18A, LAP3, PLEKHO1, TUBGCP3, CORO1A, ZBED2, TCN2, MSL2, CHAF1A, UNC13D, SKA2, IFI30, ZNF672, CTLA4, MAGOH, CYTH1, LCP2, TRA2B, CD300A, IRF8, IKZF1, BIRC3, FAM78A, CXCL10, HCST, POLA1, KCNAB2, PDCD1LG2, NCAPH2, RRM2, FOXM1, C5orf56, RAD51AP1, ANKRD10, CD53, ILF3, CARS2, CD52, and DAPK2 (Gene Panel 1).

In some embodiments, provided is a method for predicting residual disease (RD) in triple negative cancer patients (TNBC) based on measuring transcript per million expression values of genes in a gene panel, wherein the gene panel comprises one or more of the following genes: AKR1C2, ALS2CL, BAMBI, CAB39L, CAV2, CBR3.AS1, CCDC149, CD46, CHST1, CLDN12, EPB41L5, FBXL16, FITM2, FSIP2, FSTL3, HSF4, INHBB, LONP2, MATN3, NQO1, PLLP, PTGR2, PTK6, SERINC3, SLC2A10, SNX21, TACC2, THSD4, TMEM150A, TP53INP2, and UNC5CL (Gene Panel 2).

In some embodiments, provided is a method for predicting disease free survival (DFS), distant metastasis free survival (DMFS) or overall survival (OS) in basal breast cancer patients based on measuring transcript per million expression values of genes in Gene Panel 1.

In some embodiments, provided is a method for predicting disease free survival (DFS), distant metastasis free survival (DMFS) or overall survival (OS) in basal breast cancer patients based on measuring transcript per million expression values of genes in Gene Panel 2.

In some embodiments, for the methods herein, for Gene Panel 1, the area under the curve (AUC) calculated using ROC curve analysis is at least about 0.6 for each gene.

In some embodiments, for the methods herein, for Gene Panel 2, the area under the curve (AUC) calculated using ROC curve analysis is at least about 0.6 for each gene.

In some embodiments, provided is a method for selecting patients suffering from triple negative breast cancer for neoadjuvant chemotherapy, the method comprising measuring transcript per million expression values of genes in Gene Panel 1 and selecting patients for neoadjuvant chemotherapy if the AUC for each gene in the gene panel is at least about 0.6.

In some embodiments, provided is a method for selecting patients suffering from triple negative breast cancer for neoadjuvant chemotherapy, the method comprising measuring transcript per million expression values of genes Gene Panel 2 and selecting patients for neoadjuvant chemotherapy if the AUC for each gene in the gene panel is at least about 0.6.

Provided is a kit comprising Gene Panel 1. Provided is a kit comprising Gene Panel 2. Provided is a kit comprising Gene Panel 1 and Gene Panel 2.

### EXAMPLES

### Methods

### Study Cohort

TABLE 1
Characteristic features of study population in the discovery and validation cohorts.

| | Cohort ID | | | | | |
|---|---|---|---|---|---|---|
| | GSE164458_A | GSE164458_B | GSE164458_C | GSE106977 | PRJNA688066 | GSE192341 |
| BC subtype | 237 | 122 | 123 | 119 | 90 | 50 |
| TNBC (n) | | | | | | |
| NAC | PTX + CARB + | PTX + CARB+ | PTX | ANT and/or | Taxane- | Taxane- |
| treatment | VELI | | | Taxanes/+/−CARB | based | based |
| Response | | | | | | |
| to NAC | | | | | | |
| pCR | 127 | 69 | 40 | 46 | 38 | 21 |
| RD | 110 | 53 | 83 | 73 | 52 | 29 |

BC: breast cancer; NAC: neoadjuvant chemotherapy; pCR: Pathological complete response; RD: Residual disease; PTX: PACLITAXEL; CARB: CARBOPLATIN; VELI: VELIPARIB; ANT: ANTHRACYCLINE:

### Data Retrieval and Bioinformatics

Expression data from the discovery cohorts were retrieved from the gene omnibus (GEO) datasets (GSE164458_A, GSE164458_B, GSE164458_C, GSE192341 and GSE106977. FASTQ files from the PRJNA688066 dataset were mapped and aligned to the hg38 reference genome using CLC genomics workbench 20.0 using default settings as we described before [Olsson, A., et al., Nature, 2016. 537 (7622): p. 698-702, Shaath, H., et al., Cell Death Discov, 2021. 7(1): p. 23]. Data from GSE192341 was used as an additional validation cohort. Log 2 transformed TPM (transcript per million) expression values were used for the analysis.

### Discriminant Analyses

To determine the ability of predicted variables to discriminate between pCR and RD, discriminant analyses were performed using ROC curve analysis to determine the AUC value using IBM SPSS statistics version 26. Asymptotic p-value <0.05 were considered significant.

### Survival Analysis

Gene signature predictive of pCR were subjected to Kaplan-Meier survival analysis (mean expression) using KMplotter database (kmplot.com) in basal breast cancer disease cohort (RFS: n=442, OS: n=296, DMFS: n=283) as described before [10].

### Results

### Identification of Gene Panel Predictive of pCR and RD in TNBC

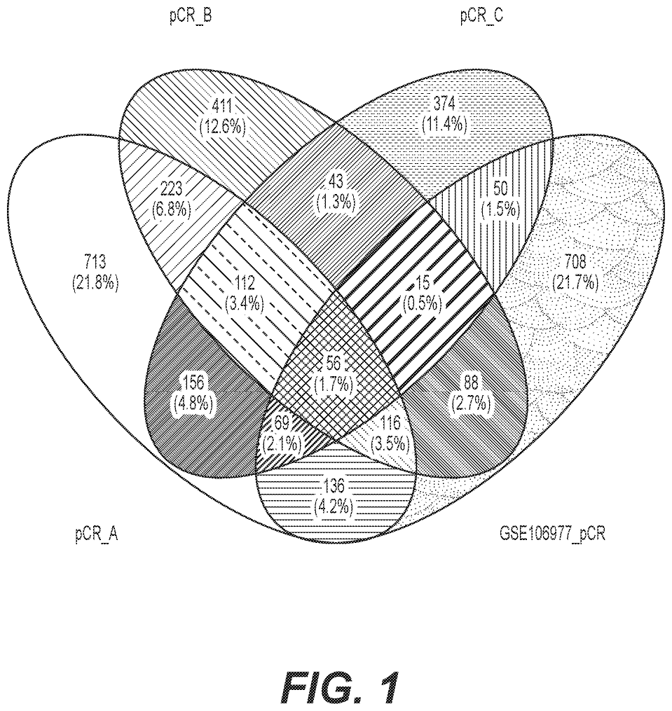

To identify the gene set which can discriminate patients exhibiting pCR from RD, the gene expression data from the first discovery cohort GSE164458_A (pCR=127, RD=110) were subjected to ROC analysis, which identified 1,583 genes predictive of pCR (Table S1) and 384 genes predictive of RD (Table S2), AUC<0.05. A similar analysis was conducted on GSE164458_B dataset (pCR=69, RD=53) and identified 1,064 genes predictive of pCR (Table S3) and 487 genes predictive of (Table S4). ROC analysis performed on the GSE164458_C (pCR=40, RD=83) identified 875 genes predictive of pCR and 318 genes predictive of RD (Table S6). ROC analysis performed on the GSE106977 dataset (pCR=46 and RD=73) identified 1243 genes associated with pCR (Table S7) and 736 genes associated with RD (Table S8). We subsequently looked for common predictive genes across the four cohorts and identified 56 genes predictive of pCR namely: LMNB1, TFDP1, SMC4, NASP, RAD54L, NUP210, SEMA4D, BRIP1, GBP5, STIL, FERMT3, LAMP3, SLAMF8, SNX20, HIST1H2BO, SLC25A19, IL32, KIF18A, LAP3, PLEKHO1, TUBGCP3, CORO1A, ZBED2, TCN2, MSL2, CHAF1A, UNC13D, SKA2, IFI30, ZNF672, CTLA4, MAGOH, CYTH1, LCP2, TRA2B, CD300A, IRF8, IKZF1, BIRC3, FAM78A, CXCL10, HCST, POLA1, KCNAB2, PDCD1LG2, NCAPH2, RRM2, FOXM1, C5orf56, RAD51AP1, ANKRD10, CD53, ILF3, CARS2, CD52, and DAPK2 (FIG. 1).

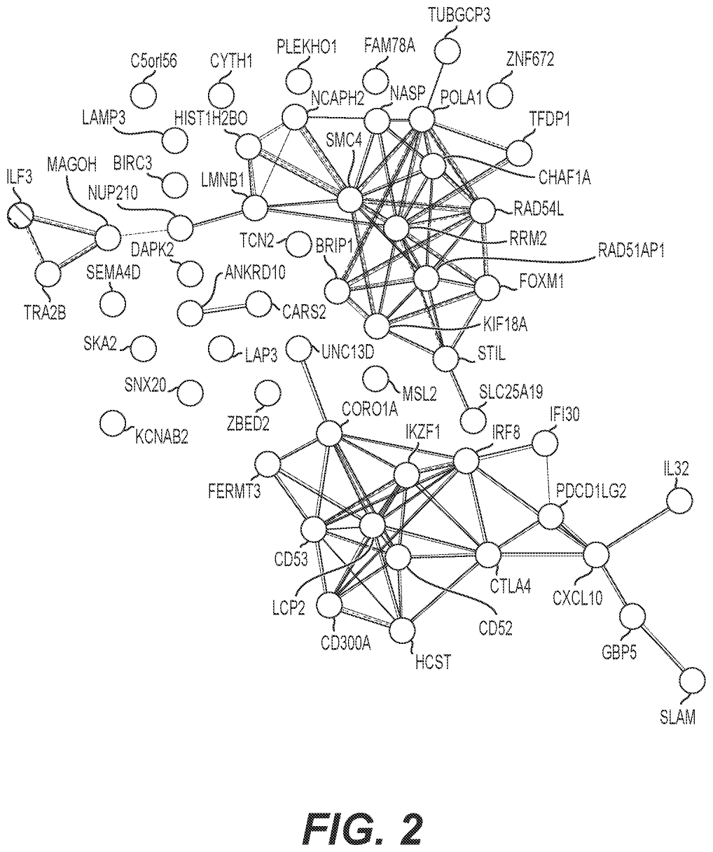

PPI network analysis on gene predictive of pCR highlighted enrichment in functional categories indicative of mitotic and immune cell functions (FIG. 2), PPI enrichment p-value <1.0×10⁻¹⁶.

### Assessing the Discriminative Potential of Combined pCR Scores

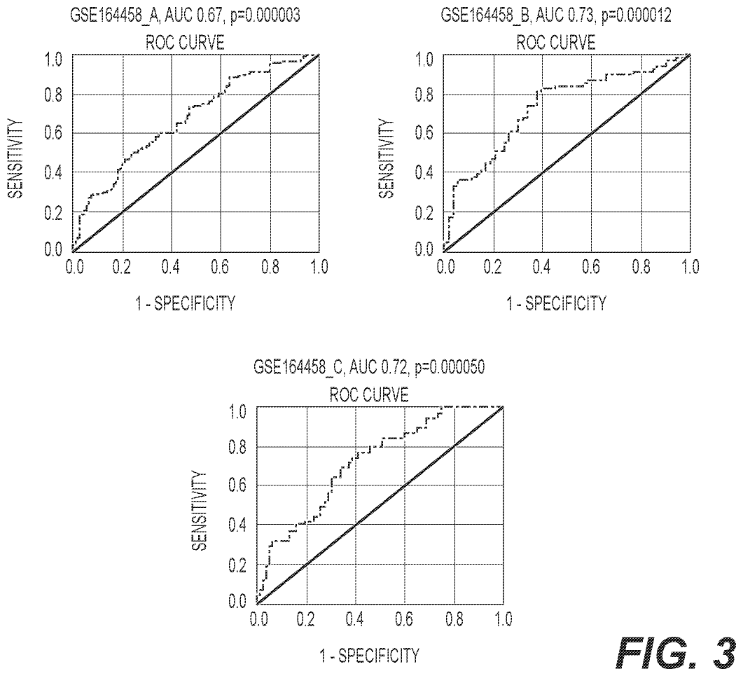

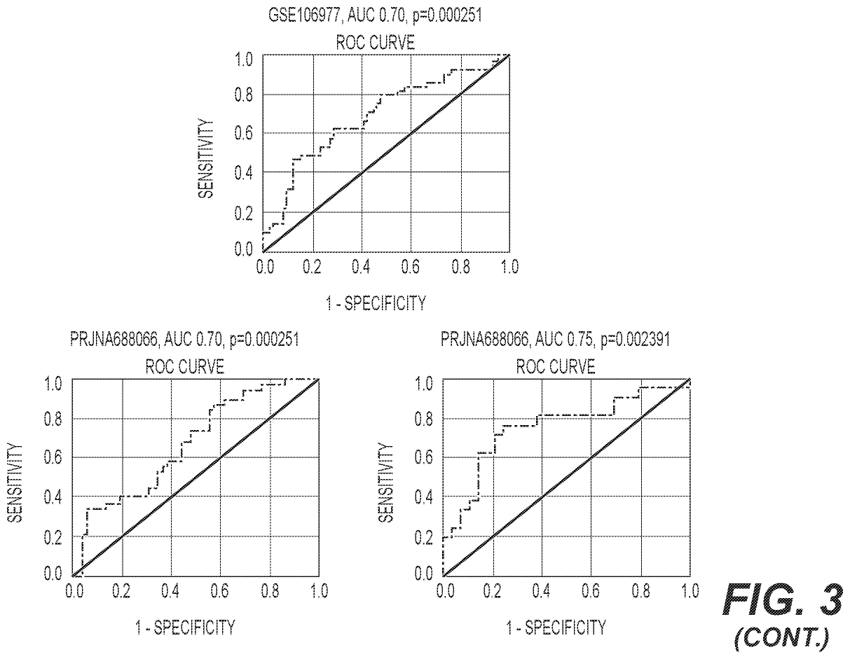

We subsequently assessed the ability of the combined pCR, RD, and pCR.RD scores to discriminate pCR from RD. The combined pCR score was able to discriminate pCR and RD with high accuracy for the discovery (GSE164458_A, AUC=0.67, p=0.000003; GSE164458_B, AUC=0.73, p=0.000012; GSE164458_C, AUC=0.72, p=0.000050; and GSE106977, AUC=0.70, p=0.000251, FIG. 3). We subsequently validated the performance on the 56-PCR gene panel in two additional cohorts which also revealed significant discriminative power (PRJNA688066, AUC=0.70, p=0.000251; PRJNA688066, AUC=0.75, p=0.002391, FIG. 3). Taken together, our data revealed the ability of the identified pCR gene signature to discriminate pCR and RD with high specificity and sensitivity.

### Assessing the Discriminative Potential of Combined RD Scores

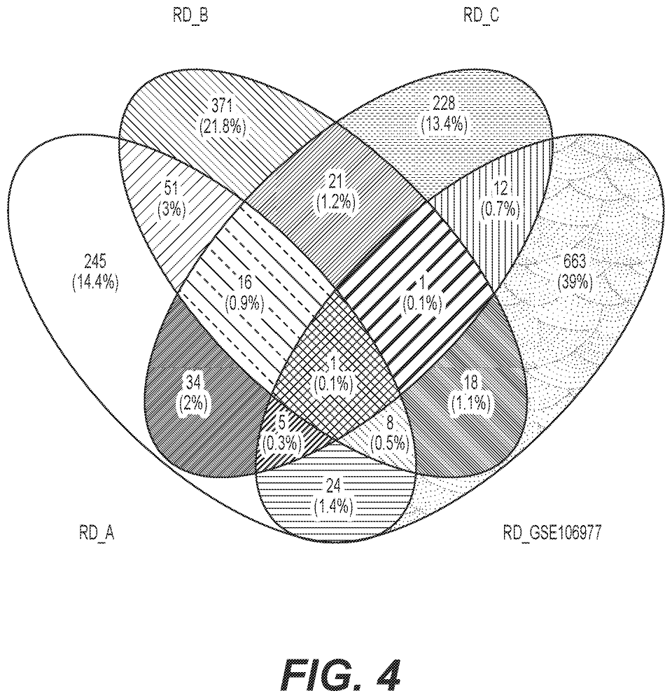

When crossing the gene panels predictive of RD, we observed only one gene (CLDN12) to be common among all four discovery cohorts (FIG. 4). Therefore, we took a less conservative approach and considered any gene that was predictive of RD in any three out of four discovery cohorts. This led to the identification of thirty-one genes predictive of RD namely: AKR1C2, ALS2CL, BAMBI, CAB39L, CAV2, CBR3.AS1, CCDC149, CD46, CHST1, CLDN12, EPB41L5, FBXL16, FITM2, FSIP2, FSTL3, HSF4, INHBB, LONP2, MATN3, NQO1, PLLP, PTGR2, PTK6, SERINC3, SLC2A10, SNX21, TACC2, THSD4, TMEM150A, TP53INP2, and UNC5CL, FIG. 4.

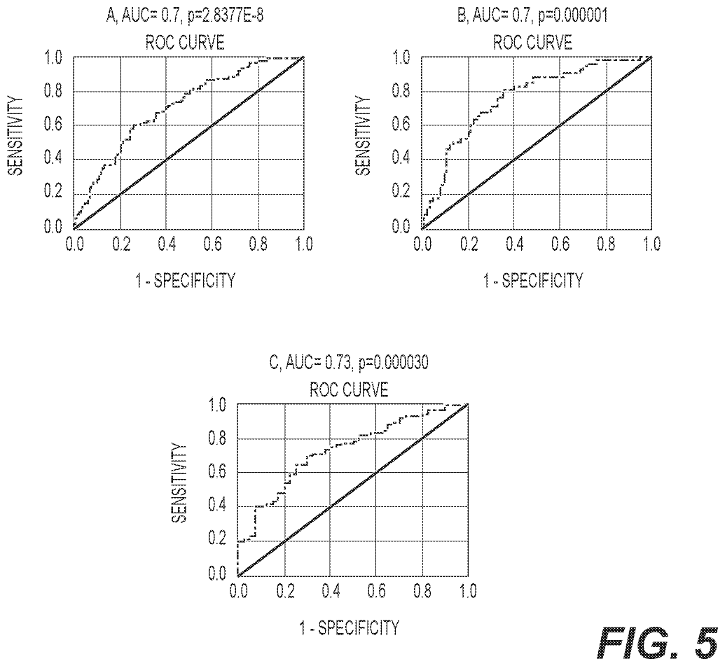

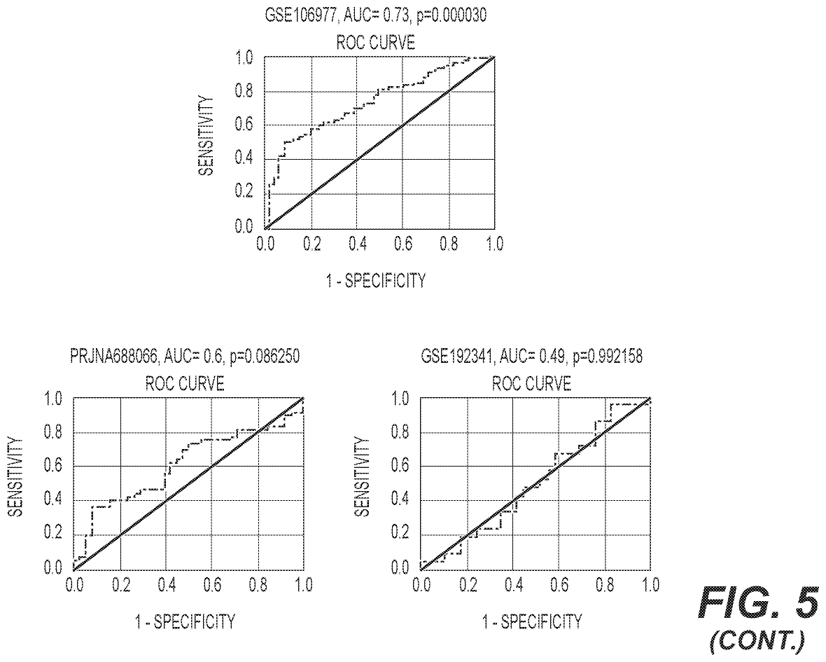

The ability of the 31 RD gene panel to discriminate RD from pCR was assessed in the discovery (GSE164458_A, AUC=0.7, p=2.8×10⁻⁸; GSE164458_B, AUC=0.7, p=0.000001; GSE164458_C, AUC=0.73, p=0.000030; and GSE106977, AUC=0.73, p=0.000030, FIG. 5). We subsequently validated the performance of the 31-RD gene panel in two additional cohorts (PRJNA688066, AUC=0.6, p=0.086250; GSE192341, AUC=0.49, p=0.992158, which revealed lack of validation when compared to the pCR gene panel, FIG. 5). These data highlighted that genes predictive of pCR are more consistent across different cohorts compared to genes predictive of RD.

### Assessing the Discriminative Potential of Combined pCR and RD Scores

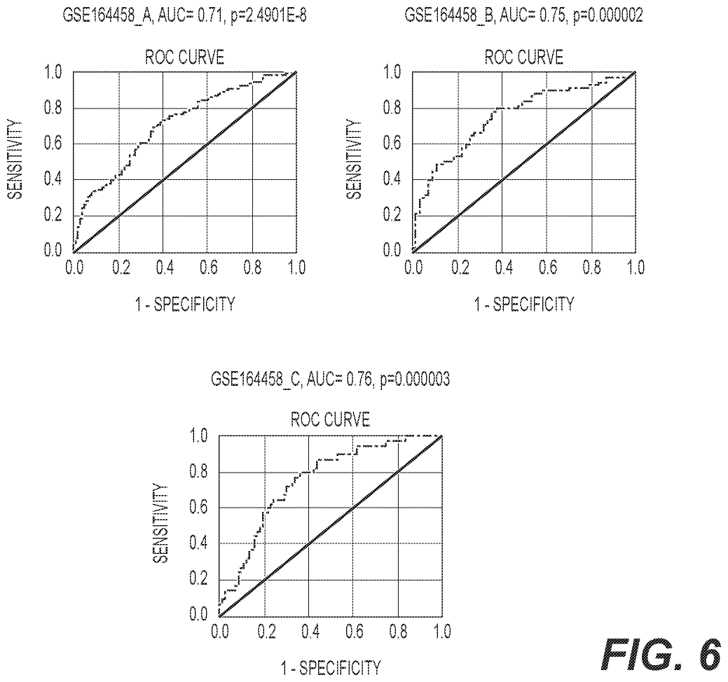

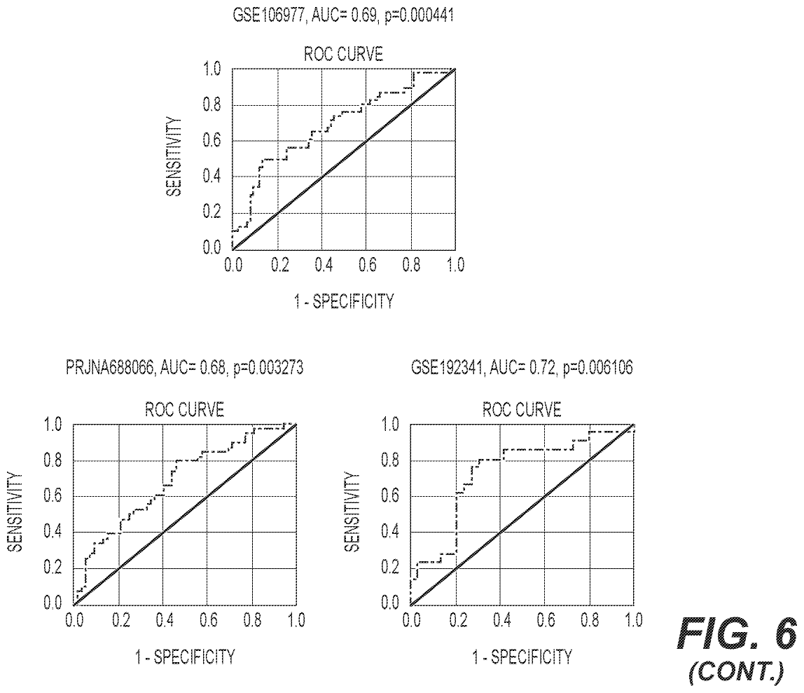

We subsequently assessed the combination of the ratio (pCR/RD) scores in discriminating TNBC patients with pCR and RD. Using this strategy, we observed significant discriminative potential of this combined score in discriminating pCR and RD in the six TNBC cohorts (GSE164458_A, AUC=0.71, p=2.4×10⁻⁸; GSE164458_B, AUC=0.75, p=0.000002; GSE164458_C, AUC=0.76, p=0.000003; GSE106977, AUC=0.69, p=0.0004, PRJNA688066, AUC=0.68, p=0.003; and GSE192341, AUC=0.72, p=0.006, FIG. 6).

Ability of pCR Gene Signature to Predict Clinical Outcome in Patients with Basal Breast Cancer

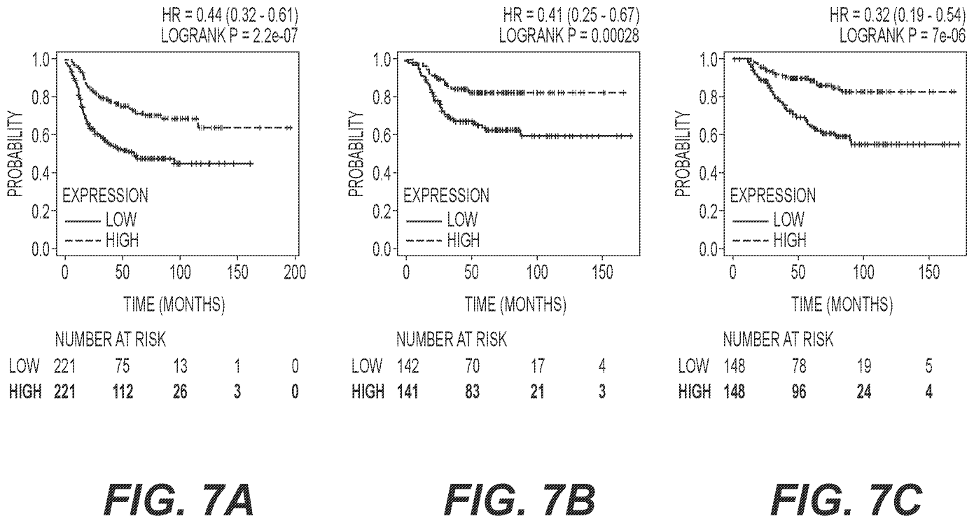

To assess the ability of identified 56 gene panel predictive of pCR in predicting patients' survival, we performed Kaplan Myer survival analysis and revealed significant predictive power of the identified pCR gene panel in predicting DFS (HR=0.44 (0.32-0.61) log-rank P=2.2×10⁻⁰⁷), Distant metastasis free survival (DMFS, HR=0.41 (0.25-0.67) log-rank P=0.00028) and overall survival (OS, HR=0.32 (0.19-0.54) log=rank P=7.0×10⁻⁰⁶), FIG. 7.

We identified gene signatures predicative of pCR and RD in TNBC with potential clinical implications in patient stratification and precision medicine.

These results can be developed into an assay (for example using the Nanostring platform) to test triple negative breast cancer patients prior to receiving treatment and to decide if they should receive neoadjuvant chemotherapy or an alternative treatment plan.

TABLE 1
Genes predictive of pCR from the GSE164458_A cohort.
Area under the curve

| | | | | Asymptotic 95% | |
|---|---|---|---|---|---|
| | | | | Confidence | |
| Test | | | | Interval | |
| Result | | Std. | Asymptotic | Lower | Upper |
| Variable(s) | Area | Error^{a} | Sig.^{b} | Bound | Bound |
| LMNB1 | 0.669 | 0.035 | 0.000 | 0.601 | 0.737 |
| IL4I1 | 0.668 | 0.035 | 0.000 | 0.599 | 0.737 |
| NUP160 | 0.667 | 0.035 | 0.000 | 0.599 | 0.736 |
| HELLS | 0.659 | 0.036 | 0.000 | 0.589 | 0.729 |
| MCM2 | 0.656 | 0.035 | 0.000 | 0.587 | 0.726 |
| MCM3 | 0.655 | 0.035 | 0.000 | 0.586 | 0.724 |
| ADAM8 | 0.655 | 0.036 | 0.000 | 0.585 | 0.725 |
| NFKB1 | 0.654 | 0.036 | 0.000 | 0.584 | 0.724 |
| TYMS | 0.654 | 0.036 | 0.000 | 0.584 | 0.724 |
| MCM6 | 0.654 | 0.036 | 0.000 | 0.583 | 0.724 |
| CNOT9 | 0.653 | 0.035 | 0.000 | 0.584 | 0.723 |
| PCNA | 0.652 | 0.035 | 0.000 | 0.583 | 0.722 |
| SYK | 0.651 | 0.036 | 0.000 | 0.582 | 0.721 |
| ZBED6CL | 0.651 | 0.036 | 0.000 | 0.582 | 0.721 |
| CCNA2 | 0.650 | 0.036 | 0.000 | 0.580 | 0.720 |
| DEK | 0.650 | 0.035 | 0.000 | 0.580 | 0.719 |
| MOV10 | 0.650 | 0.036 | 0.000 | 0.579 | 0.720 |
| GMNN | 0.649 | 0.036 | 0.000 | 0.579 | 0.718 |
| LAG3 | 0.648 | 0.036 | 0.000 | 0.577 | 0.718 |
| ORC1 | 0.647 | 0.036 | 0.000 | 0.577 | 0.718 |
| KLHDC7B | 0.647 | 0.036 | 0.000 | 0.576 | 0.718 |
| CGAS | 0.647 | 0.036 | 0.000 | 0.577 | 0.717 |
| CDV3 | 0.647 | 0.036 | 0.000 | 0.577 | 0.717 |
| DNMT1 | 0.645 | 0.036 | 0.000 | 0.574 | 0.715 |
| IKBKE | 0.644 | 0.036 | 0.000 | 0.574 | 0.715 |
| CDC45 | 0.644 | 0.036 | 0.000 | 0.574 | 0.714 |
| DCAF7 | 0.644 | 0.036 | 0.000 | 0.573 | 0.715 |
| MCM5 | 0.644 | 0.036 | 0.000 | 0.573 | 0.714 |
| TNFRSF9 | 0.643 | 0.036 | 0.000 | 0.573 | 0.713 |
| MFHAS1 | 0.642 | 0.036 | 0.000 | 0.572 | 0.712 |
| HMGB2 | 0.642 | 0.036 | 0.000 | 0.572 | 0.712 |
| KIF11 | 0.641 | 0.036 | 0.000 | 0.570 | 0.712 |
| PHF19 | 0.641 | 0.036 | 0.000 | 0.570 | 0.711 |
| SRSF1 | 0.640 | 0.036 | 0.000 | 0.569 | 0.710 |
| USP1 | 0.639 | 0.036 | 0.000 | 0.568 | 0.710 |
| MIR613 | 0.639 | 0.036 | 0.000 | 0.568 | 0.709 |
| MIR7854 | 0.639 | 0.036 | 0.000 | 0.568 | 0.710 |
| DNAJC9 | 0.639 | 0.036 | 0.000 | 0.568 | 0.709 |
| E2F8 | 0.638 | 0.036 | 0.000 | 0.568 | 0.709 |
| HK3 | 0.638 | 0.036 | 0.000 | 0.567 | 0.710 |
| ITGAM | 0.638 | 0.036 | 0.000 | 0.568 | 0.709 |
| TFDP1 | 0.638 | 0.036 | 0.000 | 0.566 | 0.709 |
| DONSON | 0.638 | 0.036 | 0.000 | 0.566 | 0.709 |
| CELF1 | 0.637 | 0.036 | 0.000 | 0.567 | 0.708 |
| PLK4 | 0.637 | 0.036 | 0.000 | 0.567 | 0.708 |
| SMC4 | 0.637 | 0.036 | 0.000 | 0.567 | 0.707 |
| NUAK2 | 0.637 | 0.036 | 0.000 | 0.566 | 0.707 |
| SH3BP1 | 0.636 | 0.036 | 0.000 | 0.565 | 0.708 |
| AFAP1.AS1 | 0.636 | 0.036 | 0.000 | 0.566 | 0.707 |
| MYO7A | 0.636 | 0.036 | 0.000 | 0.566 | 0.706 |
| CLSPN | 0.636 | 0.036 | 0.000 | 0.565 | 0.707 |
| CNTLN | 0.636 | 0.036 | 0.000 | 0.565 | 0.706 |
| NFKB2 | 0.635 | 0.036 | 0.000 | 0.565 | 0.706 |
| HASPIN | 0.635 | 0.036 | 0.000 | 0.565 | 0.706 |
| ZNF367 | 0.635 | 0.036 | 0.000 | 0.564 | 0.706 |
| EXOSC9 | 0.634 | 0.036 | 0.000 | 0.563 | 0.705 |
| IL2RA | 0.634 | 0.036 | 0.000 | 0.563 | 0.704 |
| FBXO18 | 0.634 | 0.036 | 0.000 | 0.562 | 0.705 |
| CEP135 | 0.633 | 0.036 | 0.000 | 0.562 | 0.704 |
| KIF4A | 0.633 | 0.036 | 0.000 | 0.562 | 0.703 |
| CENPE | 0.632 | 0.036 | 0.000 | 0.561 | 0.704 |
| HMCES | 0.632 | 0.036 | 0.000 | 0.561 | 0.703 |
| EZH2 | 0.632 | 0.036 | 0.000 | 0.561 | 0.703 |
| EGFL6 | 0.632 | 0.036 | 0.000 | 0.560 | 0.703 |
| NFKBIE | 0.631 | 0.036 | 0.000 | 0.560 | 0.702 |
| CKAP5 | 0.631 | 0.036 | 0.001 | 0.560 | 0.702 |
| TPM3 | 0.631 | 0.036 | 0.001 | 0.560 | 0.702 |
| NECAP2 | 0.631 | 0.036 | 0.001 | 0.560 | 0.702 |
| NASP | 0.630 | 0.036 | 0.001 | 0.559 | 0.701 |
| ETV7 | 0.630 | 0.037 | 0.001 | 0.558 | 0.702 |
| FGR | 0.630 | 0.037 | 0.001 | 0.558 | 0.702 |
| RBM17 | 0.630 | 0.036 | 0.001 | 0.559 | 0.700 |
| HNRNPM | 0.630 | 0.036 | 0.001 | 0.558 | 0.701 |
| RAD54L | 0.629 | 0.036 | 0.001 | 0.559 | 0.700 |
| CCP110 | 0.629 | 0.037 | 0.001 | 0.557 | 0.702 |
| CEP55 | 0.629 | 0.037 | 0.001 | 0.558 | 0.701 |
| FEN1 | 0.629 | 0.036 | 0.001 | 0.558 | 0.700 |
| YPEL4 | 0.629 | 0.036 | 0.001 | 0.558 | 0.700 |
| CD247 | 0.629 | 0.036 | 0.001 | 0.558 | 0.700 |
| NUP210 | 0.629 | 0.036 | 0.001 | 0.557 | 0.700 |
| POLD3 | 0.628 | 0.037 | 0.001 | 0.557 | 0.700 |
| MSN | 0.628 | 0.036 | 0.001 | 0.556 | 0.699 |
| FCHO1 | 0.628 | 0.037 | 0.001 | 0.555 | 0.701 |
| SEMA4D | 0.628 | 0.036 | 0.001 | 0.556 | 0.699 |
| EMILIN2 | 0.628 | 0.037 | 0.001 | 0.555 | 0.700 |
| OIP5 | 0.627 | 0.037 | 0.001 | 0.556 | 0.699 |
| CNTRL | 0.627 | 0.037 | 0.001 | 0.555 | 0.699 |
| NUF2 | 0.627 | 0.036 | 0.001 | 0.556 | 0.698 |
| CD7 | 0.627 | 0.037 | 0.001 | 0.555 | 0.699 |
| HCK | 0.626 | 0.036 | 0.001 | 0.555 | 0.698 |
| FANCA | 0.626 | 0.037 | 0.001 | 0.554 | 0.698 |
| MTMR4 | 0.626 | 0.036 | 0.001 | 0.555 | 0.697 |
| CHAF1B | 0.626 | 0.037 | 0.001 | 0.554 | 0.698 |
| TNFAIP3 | 0.626 | 0.036 | 0.001 | 0.555 | 0.697 |
| RFFL | 0.626 | 0.036 | 0.001 | 0.555 | 0.697 |
| BRIP1 | 0.626 | 0.036 | 0.001 | 0.555 | 0.697 |
| ZC4H2 | 0.626 | 0.037 | 0.001 | 0.554 | 0.697 |
| FASLG | 0.626 | 0.036 | 0.001 | 0.555 | 0.696 |
| MSH2 | 0.625 | 0.036 | 0.001 | 0.554 | 0.696 |
| PRF1 | 0.625 | 0.036 | 0.001 | 0.554 | 0.696 |
| RELT | 0.625 | 0.036 | 0.001 | 0.554 | 0.696 |
| NCAPG2 | 0.625 | 0.037 | 0.001 | 0.553 | 0.697 |
| ACAT2 | 0.625 | 0.037 | 0.001 | 0.553 | 0.697 |
| ALYREF | 0.625 | 0.036 | 0.001 | 0.554 | 0.696 |
| CTSW | 0.625 | 0.036 | 0.001 | 0.554 | 0.696 |
| SPOCK2 | 0.625 | 0.036 | 0.001 | 0.553 | 0.696 |
| AKAP5 | 0.625 | 0.036 | 0.001 | 0.553 | 0.696 |
| RASSF2 | 0.624 | 0.036 | 0.001 | 0.553 | 0.696 |
| ZBTB14 | 0.624 | 0.037 | 0.001 | 0.552 | 0.696 |
| CDK2 | 0.624 | 0.036 | 0.001 | 0.553 | 0.695 |
| HIST1H1B | 0.624 | 0.037 | 0.001 | 0.552 | 0.696 |
| E2F3 | 0.624 | 0.036 | 0.001 | 0.553 | 0.695 |
| WDR76 | 0.624 | 0.036 | 0.001 | 0.553 | 0.695 |
| RBL1 | 0.624 | 0.037 | 0.001 | 0.552 | 0.696 |
| POLA2 | 0.624 | 0.036 | 0.001 | 0.553 | 0.695 |
| GBP5 | 0.624 | 0.036 | 0.001 | 0.552 | 0.695 |
| PLEK | 0.623 | 0.036 | 0.001 | 0.553 | 0.694 |
| GZMB | 0.623 | 0.036 | 0.001 | 0.553 | 0.694 |
| MSH6 | 0.623 | 0.036 | 0.001 | 0.552 | 0.694 |
| ICAM1 | 0.623 | 0.036 | 0.001 | 0.552 | 0.695 |
| GRB2 | 0.623 | 0.036 | 0.001 | 0.552 | 0.694 |
| COPA | 0.623 | 0.036 | 0.001 | 0.552 | 0.694 |
| WDHD1 | 0.623 | 0.037 | 0.001 | 0.551 | 0.695 |
| IL2RB | 0.623 | 0.036 | 0.001 | 0.552 | 0.694 |
| DENND4B | 0.623 | 0.036 | 0.001 | 0.551 | 0.694 |
| PSMC3 | 0.623 | 0.036 | 0.001 | 0.552 | 0.694 |
| RMI1 | 0.623 | 0.036 | 0.001 | 0.551 | 0.694 |
| DCLRE1B | 0.623 | 0.036 | 0.001 | 0.551 | 0.694 |
| LINC01547 | 0.623 | 0.037 | 0.001 | 0.551 | 0.695 |
| LAS1L | 0.622 | 0.036 | 0.001 | 0.551 | 0.694 |
| TMPO | 0.622 | 0.037 | 0.001 | 0.551 | 0.694 |
| RELB | 0.622 | 0.036 | 0.001 | 0.551 | 0.694 |
| COTL1 | 0.622 | 0.036 | 0.001 | 0.551 | 0.693 |
| LPIN2 | 0.622 | 0.036 | 0.001 | 0.551 | 0.693 |
| U62317.2 | 0.622 | 0.036 | 0.001 | 0.551 | 0.694 |
| HSD3B7 | 0.622 | 0.037 | 0.001 | 0.550 | 0.694 |
| MYO1G | 0.622 | 0.036 | 0.001 | 0.550 | 0.693 |
| POLE | 0.622 | 0.037 | 0.001 | 0.550 | 0.693 |
| LYZ | 0.621 | 0.036 | 0.001 | 0.550 | 0.693 |
| KIF2A | 0.621 | 0.037 | 0.001 | 0.550 | 0.693 |
| CEP97 | 0.621 | 0.037 | 0.001 | 0.549 | 0.693 |
| USF1 | 0.621 | 0.036 | 0.001 | 0.550 | 0.692 |
| SH2D2A | 0.621 | 0.036 | 0.001 | 0.549 | 0.692 |
| EML4 | 0.620 | 0.036 | 0.001 | 0.549 | 0.692 |
| DGKZ | 0.620 | 0.036 | 0.001 | 0.550 | 0.691 |
| KNTC1 | 0.620 | 0.037 | 0.001 | 0.549 | 0.692 |
| APOBEC3G | 0.620 | 0.036 | 0.001 | 0.549 | 0.691 |
| CDCA2 | 0.620 | 0.036 | 0.001 | 0.549 | 0.691 |
| FANCE | 0.620 | 0.036 | 0.001 | 0.549 | 0.691 |
| MORN3 | 0.620 | 0.036 | 0.001 | 0.548 | 0.691 |
| IL15RA | 0.620 | 0.036 | 0.001 | 0.549 | 0.691 |
| STIL | 0.620 | 0.036 | 0.001 | 0.548 | 0.691 |
| DDX5 | 0.620 | 0.037 | 0.001 | 0.547 | 0.692 |
| UHRF1 | 0.620 | 0.037 | 0.001 | 0.548 | 0.691 |
| ANKRD22 | 0.620 | 0.036 | 0.002 | 0.548 | 0.691 |
| FERMT3 | 0.619 | 0.037 | 0.002 | 0.547 | 0.692 |
| SCNM1 | 0.619 | 0.037 | 0.002 | 0.547 | 0.692 |
| PASK | 0.619 | 0.036 | 0.002 | 0.548 | 0.690 |
| BID | 0.619 | 0.037 | 0.002 | 0.548 | 0.691 |
| ITGB2 | 0.619 | 0.037 | 0.002 | 0.547 | 0.691 |
| RPA1 | 0.619 | 0.036 | 0.002 | 0.548 | 0.690 |
| PSTPIP1 | 0.619 | 0.037 | 0.002 | 0.547 | 0.690 |
| ACP2 | 0.619 | 0.037 | 0.002 | 0.547 | 0.691 |
| FN3KRP | 0.619 | 0.037 | 0.002 | 0.546 | 0.691 |
| MCM10 | 0.619 | 0.037 | 0.002 | 0.547 | 0.690 |
| SMG5 | 0.619 | 0.036 | 0.002 | 0.547 | 0.690 |
| RNASEH1 | 0.619 | 0.037 | 0.002 | 0.547 | 0.690 |
| MDK | 0.619 | 0.036 | 0.002 | 0.547 | 0.690 |
| PMF1 | 0.619 | 0.037 | 0.002 | 0.547 | 0.690 |
| STAT1 | 0.618 | 0.036 | 0.002 | 0.547 | 0.690 |
| ZDHHC18 | 0.618 | 0.036 | 0.002 | 0.547 | 0.690 |
| ZNF687 | 0.618 | 0.037 | 0.002 | 0.547 | 0.690 |
| RFX5 | 0.618 | 0.036 | 0.002 | 0.547 | 0.690 |
| TRIM14 | 0.618 | 0.037 | 0.002 | 0.546 | 0.690 |
| EMID1 | 0.618 | 0.036 | 0.002 | 0.547 | 0.689 |
| GTF2E2 | 0.618 | 0.036 | 0.002 | 0.547 | 0.689 |
| SCO2 | 0.618 | 0.037 | 0.002 | 0.546 | 0.690 |
| PSTPIP2 | 0.618 | 0.037 | 0.002 | 0.546 | 0.690 |
| TRAF1 | 0.618 | 0.037 | 0.002 | 0.546 | 0.689 |
| NKG7 | 0.617 | 0.036 | 0.002 | 0.546 | 0.689 |
| SEL1L3 | 0.617 | 0.036 | 0.002 | 0.546 | 0.689 |
| IL12RB1 | 0.617 | 0.036 | 0.002 | 0.546 | 0.689 |
| LGI2 | 0.617 | 0.036 | 0.002 | 0.546 | 0.689 |
| GPRIN1 | 0.617 | 0.037 | 0.002 | 0.546 | 0.689 |
| PARP1 | 0.617 | 0.037 | 0.002 | 0.546 | 0.689 |
| SLC25A44 | 0.617 | 0.036 | 0.002 | 0.545 | 0.688 |
| LAMP3 | 0.617 | 0.037 | 0.002 | 0.545 | 0.688 |
| SLAMF8 | 0.617 | 0.036 | 0.002 | 0.545 | 0.688 |
| SNX20 | 0.617 | 0.036 | 0.002 | 0.545 | 0.688 |
| NXT1 | 0.617 | 0.037 | 0.002 | 0.545 | 0.688 |
| ADTRP | 0.617 | 0.036 | 0.002 | 0.545 | 0.688 |
| TACO1 | 0.617 | 0.036 | 0.002 | 0.545 | 0.688 |
| TRAF2 | 0.617 | 0.037 | 0.002 | 0.545 | 0.688 |
| RCN1 | 0.616 | 0.036 | 0.002 | 0.545 | 0.688 |
| USP37 | 0.616 | 0.037 | 0.002 | 0.545 | 0.688 |
| TNFAIP2 | 0.616 | 0.037 | 0.002 | 0.545 | 0.688 |
| ILF2 | 0.616 | 0.036 | 0.002 | 0.545 | 0.688 |
| NUP85 | 0.616 | 0.037 | 0.002 | 0.544 | 0.689 |
| LGALS9 | 0.616 | 0.037 | 0.002 | 0.544 | 0.689 |
| PIK3CD | 0.616 | 0.037 | 0.002 | 0.545 | 0.688 |
| SOWAHD | 0.616 | 0.036 | 0.002 | 0.545 | 0.687 |
| SLA2 | 0.616 | 0.036 | 0.002 | 0.544 | 0.688 |
| GBP1 | 0.616 | 0.037 | 0.002 | 0.544 | 0.688 |
| DHTKD1 | 0.616 | 0.037 | 0.002 | 0.544 | 0.688 |
| CCR5 | 0.616 | 0.036 | 0.002 | 0.545 | 0.687 |
| NDUFS2 | 0.616 | 0.037 | 0.002 | 0.544 | 0.687 |
| CSF1 | 0.616 | 0.037 | 0.002 | 0.543 | 0.689 |
| PTPN7 | 0.616 | 0.036 | 0.002 | 0.544 | 0.687 |
| SIGLEC7 | 0.615 | 0.037 | 0.002 | 0.543 | 0.687 |
| COLGALT1 | 0.615 | 0.037 | 0.002 | 0.543 | 0.687 |
| FAM171A1 | 0.615 | 0.037 | 0.002 | 0.543 | 0.687 |
| DOK2 | 0.615 | 0.037 | 0.002 | 0.543 | 0.687 |
| DTX3L | 0.615 | 0.037 | 0.002 | 0.542 | 0.688 |
| DTNBP1 | 0.615 | 0.037 | 0.002 | 0.543 | 0.687 |
| CLEC12A | 0.615 | 0.036 | 0.002 | 0.544 | 0.686 |
| EFHD2 | 0.615 | 0.037 | 0.002 | 0.542 | 0.687 |
| NDC80 | 0.615 | 0.037 | 0.002 | 0.543 | 0.686 |
| PRPF3 | 0.615 | 0.036 | 0.002 | 0.543 | 0.686 |
| ING1 | 0.615 | 0.037 | 0.002 | 0.543 | 0.687 |
| ACTRT3 | 0.615 | 0.037 | 0.002 | 0.543 | 0.687 |
| ELF4 | 0.615 | 0.036 | 0.002 | 0.543 | 0.686 |
| LRRC61 | 0.614 | 0.036 | 0.002 | 0.543 | 0.686 |
| MICAL1 | 0.614 | 0.037 | 0.002 | 0.543 | 0.686 |
| HIST1H2AL | 0.614 | 0.037 | 0.002 | 0.542 | 0.687 |
| FGD6 | 0.614 | 0.036 | 0.002 | 0.543 | 0.686 |
| FUBP1 | 0.614 | 0.037 | 0.002 | 0.542 | 0.686 |
| NSD2 | 0.614 | 0.036 | 0.002 | 0.543 | 0.686 |
| TYMP | 0.614 | 0.037 | 0.002 | 0.542 | 0.686 |
| HIC2 | 0.614 | 0.037 | 0.002 | 0.542 | 0.686 |
| SIRPG | 0.614 | 0.037 | 0.002 | 0.542 | 0.686 |
| NUP188 | 0.614 | 0.037 | 0.002 | 0.542 | 0.686 |
| GFI1 | 0.614 | 0.036 | 0.002 | 0.542 | 0.685 |
| WARS | 0.614 | 0.037 | 0.003 | 0.542 | 0.686 |
| PRR11 | 0.614 | 0.037 | 0.003 | 0.542 | 0.686 |
| MCM4 | 0.614 | 0.037 | 0.003 | 0.542 | 0.686 |
| RANBP1 | 0.614 | 0.037 | 0.003 | 0.542 | 0.685 |
| ECT2 | 0.614 | 0.037 | 0.003 | 0.542 | 0.685 |
| GINS1 | 0.614 | 0.036 | 0.003 | 0.542 | 0.685 |
| ADAR | 0.614 | 0.037 | 0.003 | 0.541 | 0.686 |
| NRAS | 0.613 | 0.037 | 0.003 | 0.541 | 0.686 |
| AL357060.2 | 0.613 | 0.037 | 0.003 | 0.542 | 0.685 |
| NINL | 0.613 | 0.037 | 0.003 | 0.541 | 0.685 |
| CHTOP | 0.613 | 0.036 | 0.003 | 0.542 | 0.685 |
| CDKAL1 | 0.613 | 0.036 | 0.003 | 0.542 | 0.685 |
| LMNB2 | 0.613 | 0.037 | 0.003 | 0.541 | 0.686 |
| ITGB2.AS1 | 0.613 | 0.037 | 0.003 | 0.541 | 0.685 |
| NCL | 0.613 | 0.037 | 0.003 | 0.541 | 0.685 |
| KPNB1 | 0.613 | 0.037 | 0.003 | 0.540 | 0.686 |
| RRM1 | 0.613 | 0.036 | 0.003 | 0.542 | 0.684 |
| NCAPG | 0.613 | 0.036 | 0.003 | 0.542 | 0.684 |
| RBM15 | 0.613 | 0.036 | 0.003 | 0.541 | 0.684 |
| S100A9 | 0.613 | 0.037 | 0.003 | 0.540 | 0.686 |
| SMYD4 | 0.613 | 0.037 | 0.003 | 0.541 | 0.685 |
| SGPP2 | 0.613 | 0.037 | 0.003 | 0.540 | 0.685 |
| RBM10 | 0.613 | 0.037 | 0.003 | 0.541 | 0.685 |
| HIST1H2BO | 0.613 | 0.037 | 0.003 | 0.540 | 0.685 |
| CSF2RB | 0.612 | 0.036 | 0.003 | 0.541 | 0.684 |
| SIGLEC10 | 0.612 | 0.037 | 0.003 | 0.541 | 0.684 |
| CD2 | 0.612 | 0.036 | 0.003 | 0.541 | 0.684 |
| SLC25A19 | 0.612 | 0.037 | 0.003 | 0.540 | 0.685 |
| MIAT | 0.612 | 0.037 | 0.003 | 0.541 | 0.684 |
| CD3G | 0.612 | 0.037 | 0.003 | 0.540 | 0.684 |
| TMEM176B | 0.612 | 0.037 | 0.003 | 0.540 | 0.684 |
| NUP54 | 0.612 | 0.037 | 0.003 | 0.539 | 0.685 |
| DHX36 | 0.612 | 0.037 | 0.003 | 0.539 | 0.685 |
| KTI12 | 0.612 | 0.037 | 0.003 | 0.539 | 0.685 |
| SV2A | 0.612 | 0.037 | 0.003 | 0.540 | 0.684 |
| GTSE1 | 0.612 | 0.037 | 0.003 | 0.540 | 0.684 |
| CD40 | 0.612 | 0.037 | 0.003 | 0.540 | 0.684 |
| SMURF2 | 0.612 | 0.037 | 0.003 | 0.540 | 0.684 |
| TTI2 | 0.612 | 0.037 | 0.003 | 0.539 | 0.684 |
| IL32 | 0.612 | 0.037 | 0.003 | 0.540 | 0.683 |
| C21orf58 | 0.612 | 0.036 | 0.003 | 0.540 | 0.683 |
| ITGB7 | 0.611 | 0.037 | 0.003 | 0.539 | 0.684 |
| LIG1 | 0.611 | 0.037 | 0.003 | 0.540 | 0.683 |
| LRRK1 | 0.611 | 0.037 | 0.003 | 0.540 | 0.683 |
| KIF18A | 0.611 | 0.037 | 0.003 | 0.539 | 0.683 |
| ACOT7 | 0.611 | 0.037 | 0.003 | 0.538 | 0.684 |
| STX11 | 0.611 | 0.036 | 0.003 | 0.540 | 0.682 |
| BIRC5 | 0.611 | 0.037 | 0.003 | 0.539 | 0.683 |
| LAPTM5 | 0.611 | 0.037 | 0.003 | 0.539 | 0.683 |
| HIST1H2AB | 0.611 | 0.037 | 0.003 | 0.538 | 0.683 |
| CORO1C | 0.611 | 0.037 | 0.003 | 0.539 | 0.683 |
| DLGAP5 | 0.611 | 0.037 | 0.003 | 0.538 | 0.683 |
| MTHFD1 | 0.611 | 0.037 | 0.003 | 0.539 | 0.683 |
| HIST1H3J | 0.611 | 0.037 | 0.003 | 0.539 | 0.683 |
| MPZ | 0.611 | 0.037 | 0.003 | 0.539 | 0.683 |
| PFKP | 0.611 | 0.037 | 0.003 | 0.539 | 0.683 |
| DDX42 | 0.611 | 0.037 | 0.003 | 0.539 | 0.683 |
| DDX23 | 0.611 | 0.037 | 0.003 | 0.539 | 0.683 |
| SGO2 | 0.611 | 0.037 | 0.003 | 0.539 | 0.682 |
| MAP3K14 | 0.611 | 0.036 | 0.003 | 0.539 | 0.682 |
| HELB | 0.611 | 0.037 | 0.003 | 0.539 | 0.683 |
| WNT10A | 0.611 | 0.037 | 0.003 | 0.539 | 0.683 |
| VPS72 | 0.611 | 0.037 | 0.003 | 0.538 | 0.683 |
| DRAM1 | 0.611 | 0.037 | 0.003 | 0.539 | 0.682 |
| CDT1 | 0.611 | 0.037 | 0.003 | 0.538 | 0.683 |
| MIR4632 | 0.611 | 0.037 | 0.003 | 0.538 | 0.683 |
| MTMR1 | 0.610 | 0.037 | 0.003 | 0.539 | 0.682 |
| MELK | 0.610 | 0.037 | 0.003 | 0.538 | 0.682 |
| KIF18B | 0.610 | 0.037 | 0.003 | 0.538 | 0.683 |
| HNRNPF | 0.610 | 0.036 | 0.003 | 0.539 | 0.682 |
| CCNF | 0.610 | 0.037 | 0.003 | 0.538 | 0.682 |
| SOCS3 | 0.610 | 0.037 | 0.003 | 0.538 | 0.683 |
| C17orf97 | 0.610 | 0.036 | 0.003 | 0.539 | 0.682 |
| OSBPL11 | 0.610 | 0.036 | 0.003 | 0.539 | 0.682 |
| DAPP1 | 0.610 | 0.037 | 0.004 | 0.538 | 0.682 |
| DEDD | 0.610 | 0.037 | 0.004 | 0.538 | 0.682 |
| HJURP | 0.610 | 0.037 | 0.004 | 0.538 | 0.682 |
| HIST1H3B | 0.610 | 0.037 | 0.004 | 0.538 | 0.682 |
| KLHL22 | 0.610 | 0.037 | 0.004 | 0.538 | 0.682 |
| ECEL1 | 0.610 | 0.037 | 0.004 | 0.538 | 0.682 |
| ITK | 0.610 | 0.037 | 0.004 | 0.538 | 0.682 |
| MIR3142HG | 0.610 | 0.037 | 0.004 | 0.538 | 0.682 |
| MIR6894 | 0.610 | 0.037 | 0.004 | 0.538 | 0.682 |
| IFNAR2 | 0.610 | 0.037 | 0.004 | 0.538 | 0.682 |
| HIST1H1D | 0.610 | 0.037 | 0.004 | 0.537 | 0.683 |
| ARHGAP30 | 0.610 | 0.037 | 0.004 | 0.537 | 0.682 |
| CENPM | 0.610 | 0.037 | 0.004 | 0.536 | 0.683 |
| TOPBP1 | 0.610 | 0.037 | 0.004 | 0.538 | 0.681 |
| MKI67 | 0.609 | 0.037 | 0.004 | 0.537 | 0.681 |
| UBASH3A | 0.609 | 0.037 | 0.004 | 0.537 | 0.682 |
| NUP62 | 0.609 | 0.037 | 0.004 | 0.537 | 0.682 |
| E2F1 | 0.609 | 0.037 | 0.004 | 0.537 | 0.681 |
| APOBEC3D | 0.609 | 0.037 | 0.004 | 0.537 | 0.681 |
| SPAG5 | 0.609 | 0.037 | 0.004 | 0.537 | 0.681 |
| TNFAIP8L2 | 0.609 | 0.037 | 0.004 | 0.537 | 0.681 |
| MGAT3 | 0.609 | 0.037 | 0.004 | 0.537 | 0.681 |
| LRRC45 | 0.609 | 0.037 | 0.004 | 0.537 | 0.681 |
| CXCL13 | 0.609 | 0.037 | 0.004 | 0.537 | 0.681 |
| EIPR1 | 0.609 | 0.037 | 0.004 | 0.538 | 0.681 |
| CEP131 | 0.609 | 0.037 | 0.004 | 0.536 | 0.682 |
| ATP6V0A2 | 0.609 | 0.037 | 0.004 | 0.537 | 0.682 |
| UBE2L6 | 0.609 | 0.037 | 0.004 | 0.536 | 0.682 |
| LAP3 | 0.609 | 0.037 | 0.004 | 0.537 | 0.681 |
| NFATC2IP | 0.609 | 0.037 | 0.004 | 0.537 | 0.681 |
| BBS7 | 0.609 | 0.037 | 0.004 | 0.537 | 0.681 |
| HIST1H3C | 0.609 | 0.037 | 0.004 | 0.537 | 0.681 |
| TAF3 | 0.609 | 0.037 | 0.004 | 0.537 | 0.681 |
| GZMA | 0.609 | 0.037 | 0.004 | 0.537 | 0.680 |
| RAB42 | 0.609 | 0.037 | 0.004 | 0.537 | 0.681 |
| CYP27A1 | 0.609 | 0.037 | 0.004 | 0.536 | 0.681 |
| DOK3 | 0.609 | 0.037 | 0.004 | 0.536 | 0.681 |
| ZAP70 | 0.609 | 0.037 | 0.004 | 0.537 | 0.681 |
| CHD4 | 0.609 | 0.037 | 0.004 | 0.537 | 0.680 |
| CCDC137 | 0.609 | 0.037 | 0.004 | 0.537 | 0.680 |
| LCP1 | 0.609 | 0.037 | 0.004 | 0.537 | 0.680 |
| HNRNPD | 0.609 | 0.037 | 0.004 | 0.536 | 0.681 |
| TMEM176A | 0.609 | 0.037 | 0.004 | 0.537 | 0.680 |
| ANKLE2 | 0.609 | 0.037 | 0.004 | 0.536 | 0.681 |
| HAUS1 | 0.608 | 0.037 | 0.004 | 0.537 | 0.680 |
| ICOS | 0.608 | 0.037 | 0.004 | 0.537 | 0.680 |
| TACC3 | 0.608 | 0.037 | 0.004 | 0.537 | 0.680 |
| PRIM1 | 0.608 | 0.037 | 0.004 | 0.536 | 0.680 |
| SNX27 | 0.608 | 0.037 | 0.004 | 0.536 | 0.681 |
| PIM2 | 0.608 | 0.037 | 0.004 | 0.536 | 0.680 |
| RFWD3 | 0.608 | 0.037 | 0.004 | 0.536 | 0.681 |
| PLEKHO1 | 0.608 | 0.036 | 0.004 | 0.536 | 0.680 |
| GBP1P1 | 0.608 | 0.037 | 0.004 | 0.536 | 0.680 |
| HIST1H2BB | 0.608 | 0.037 | 0.004 | 0.535 | 0.680 |
| PRPF38A | 0.608 | 0.037 | 0.004 | 0.536 | 0.680 |
| NR1H3 | 0.608 | 0.037 | 0.004 | 0.536 | 0.680 |
| MIR3145 | 0.608 | 0.037 | 0.004 | 0.536 | 0.680 |
| TBX21 | 0.608 | 0.036 | 0.004 | 0.536 | 0.679 |
| CC2D1B | 0.608 | 0.037 | 0.004 | 0.535 | 0.680 |
| IL23A | 0.608 | 0.037 | 0.004 | 0.535 | 0.680 |
| NCF2 | 0.608 | 0.037 | 0.004 | 0.536 | 0.679 |
| EXO1 | 0.608 | 0.037 | 0.004 | 0.536 | 0.679 |
| FUT7 | 0.608 | 0.037 | 0.004 | 0.536 | 0.679 |
| TUBGCP3 | 0.608 | 0.037 | 0.004 | 0.534 | 0.681 |
| JPT1 | 0.607 | 0.037 | 0.004 | 0.535 | 0.679 |
| ST6GALNAC5 | 0.607 | 0.037 | 0.004 | 0.535 | 0.680 |
| FAS | 0.607 | 0.036 | 0.004 | 0.536 | 0.679 |
| ZGRF1 | 0.607 | 0.037 | 0.004 | 0.534 | 0.680 |
| APOBR | 0.607 | 0.037 | 0.004 | 0.535 | 0.680 |
| SASH3 | 0.607 | 0.037 | 0.004 | 0.534 | 0.680 |
| RFC1 | 0.607 | 0.037 | 0.005 | 0.535 | 0.679 |
| GBP2 | 0.607 | 0.037 | 0.005 | 0.535 | 0.679 |
| SLC7A7 | 0.607 | 0.037 | 0.005 | 0.535 | 0.679 |
| AKAP7 | 0.607 | 0.037 | 0.005 | 0.535 | 0.679 |
| CORO1A | 0.607 | 0.037 | 0.005 | 0.534 | 0.679 |
| KIF2C | 0.607 | 0.037 | 0.005 | 0.535 | 0.679 |
| PTPN22 | 0.607 | 0.037 | 0.005 | 0.535 | 0.679 |
| CD96 | 0.607 | 0.037 | 0.005 | 0.535 | 0.679 |
| PIF1 | 0.607 | 0.037 | 0.005 | 0.535 | 0.678 |
| RHBDF2 | 0.607 | 0.037 | 0.005 | 0.534 | 0.679 |
| VAV1 | 0.607 | 0.037 | 0.005 | 0.534 | 0.679 |
| NFAM1 | 0.607 | 0.037 | 0.005 | 0.534 | 0.679 |
| CD226 | 0.607 | 0.037 | 0.005 | 0.535 | 0.679 |
| FAM208B | 0.607 | 0.037 | 0.005 | 0.534 | 0.679 |
| CD3D | 0.607 | 0.037 | 0.005 | 0.534 | 0.679 |
| OSM | 0.606 | 0.037 | 0.005 | 0.534 | 0.679 |
| LIN9 | 0.606 | 0.037 | 0.005 | 0.535 | 0.678 |
| POLD1 | 0.606 | 0.037 | 0.005 | 0.534 | 0.678 |
| LIMD2 | 0.606 | 0.037 | 0.005 | 0.534 | 0.679 |
| BHLHA15 | 0.606 | 0.037 | 0.005 | 0.534 | 0.678 |
| BTN2A2 | 0.606 | 0.037 | 0.005 | 0.534 | 0.678 |
| HIST1H3F | 0.606 | 0.037 | 0.005 | 0.534 | 0.678 |
| PYCARD | 0.606 | 0.037 | 0.005 | 0.534 | 0.678 |
| MIR3161 | 0.606 | 0.037 | 0.005 | 0.534 | 0.678 |
| CSTF2 | 0.606 | 0.037 | 0.005 | 0.534 | 0.678 |
| MMS22L | 0.606 | 0.038 | 0.005 | 0.532 | 0.679 |
| CDKN2A | 0.606 | 0.037 | 0.005 | 0.533 | 0.678 |
| AKNA | 0.606 | 0.037 | 0.005 | 0.533 | 0.678 |
| ANKRD16 | 0.606 | 0.037 | 0.005 | 0.534 | 0.678 |
| ACTL6A | 0.606 | 0.037 | 0.005 | 0.534 | 0.677 |
| TOP2A | 0.606 | 0.037 | 0.005 | 0.533 | 0.678 |
| TNFRSF1B | 0.605 | 0.037 | 0.005 | 0.533 | 0.678 |
| CIITA | 0.605 | 0.037 | 0.005 | 0.534 | 0.677 |
| AGPS | 0.605 | 0.037 | 0.005 | 0.533 | 0.678 |
| ZBED2 | 0.605 | 0.037 | 0.005 | 0.533 | 0.677 |
| RNF213 | 0.605 | 0.037 | 0.005 | 0.533 | 0.677 |
| ZNF267 | 0.605 | 0.037 | 0.005 | 0.534 | 0.677 |
| SRGN | 0.605 | 0.037 | 0.005 | 0.533 | 0.677 |
| MVB12B | 0.605 | 0.037 | 0.005 | 0.533 | 0.677 |
| SMC1A | 0.605 | 0.037 | 0.005 | 0.532 | 0.678 |
| CDC25C | 0.605 | 0.037 | 0.005 | 0.533 | 0.677 |
| KIF20A | 0.605 | 0.037 | 0.005 | 0.533 | 0.677 |
| TUBA1B | 0.605 | 0.037 | 0.005 | 0.532 | 0.678 |
| DGCR8 | 0.605 | 0.037 | 0.005 | 0.533 | 0.677 |
| TCN2 | 0.605 | 0.037 | 0.005 | 0.533 | 0.677 |
| MFSD14C | 0.605 | 0.037 | 0.005 | 0.532 | 0.677 |
| PLA2G7 | 0.605 | 0.037 | 0.005 | 0.533 | 0.677 |
| S100A8 | 0.605 | 0.037 | 0.005 | 0.532 | 0.678 |
| HIST1H1E | 0.605 | 0.037 | 0.005 | 0.532 | 0.677 |
| PBK | 0.605 | 0.037 | 0.005 | 0.533 | 0.676 |
| LAT2 | 0.605 | 0.037 | 0.005 | 0.532 | 0.677 |
| MEX3A | 0.605 | 0.037 | 0.006 | 0.532 | 0.677 |
| RALY | 0.605 | 0.037 | 0.006 | 0.533 | 0.676 |
| ARNTL2 | 0.605 | 0.037 | 0.006 | 0.532 | 0.677 |
| SLC38A5 | 0.605 | 0.037 | 0.006 | 0.533 | 0.676 |
| ZWINT | 0.605 | 0.037 | 0.006 | 0.532 | 0.677 |
| CRKL | 0.605 | 0.037 | 0.006 | 0.532 | 0.677 |
| HIST2H2AB | 0.604 | 0.037 | 0.006 | 0.532 | 0.677 |
| CAPRIN1 | 0.604 | 0.037 | 0.006 | 0.532 | 0.677 |
| LCK | 0.604 | 0.037 | 0.006 | 0.532 | 0.676 |
| SMC3 | 0.604 | 0.037 | 0.006 | 0.532 | 0.676 |
| SENP1 | 0.604 | 0.037 | 0.006 | 0.531 | 0.677 |
| DEPDC1 | 0.604 | 0.037 | 0.006 | 0.532 | 0.676 |
| TIGIT | 0.604 | 0.037 | 0.006 | 0.532 | 0.676 |
| IRF1 | 0.604 | 0.037 | 0.006 | 0.532 | 0.676 |
| MTFR2 | 0.604 | 0.037 | 0.006 | 0.532 | 0.676 |
| ANP32E | 0.604 | 0.037 | 0.006 | 0.532 | 0.676 |
| ESCO2 | 0.604 | 0.037 | 0.006 | 0.532 | 0.676 |
| RHEBL1 | 0.604 | 0.037 | 0.006 | 0.532 | 0.676 |
| ICAM2 | 0.604 | 0.037 | 0.006 | 0.532 | 0.676 |
| TTLL4 | 0.604 | 0.037 | 0.006 | 0.532 | 0.676 |
| SPIN4 | 0.604 | 0.037 | 0.006 | 0.531 | 0.676 |
| TSPAN14 | 0.604 | 0.037 | 0.006 | 0.531 | 0.676 |
| KPNA2 | 0.604 | 0.037 | 0.006 | 0.532 | 0.675 |
| CALHM6 | 0.604 | 0.037 | 0.006 | 0.532 | 0.675 |
| GGA2 | 0.604 | 0.037 | 0.006 | 0.532 | 0.675 |
| DEF6 | 0.604 | 0.037 | 0.006 | 0.532 | 0.676 |
| TP73 | 0.604 | 0.037 | 0.006 | 0.531 | 0.676 |
| TIPIN | 0.604 | 0.037 | 0.006 | 0.532 | 0.675 |
| PLK1 | 0.604 | 0.037 | 0.006 | 0.532 | 0.675 |
| IL2RG | 0.603 | 0.037 | 0.006 | 0.531 | 0.675 |
| ADGRG5 | 0.603 | 0.037 | 0.006 | 0.531 | 0.676 |
| TAGAP | 0.603 | 0.037 | 0.006 | 0.531 | 0.675 |
| MAP4K1 | 0.603 | 0.037 | 0.006 | 0.531 | 0.675 |
| PRPF4 | 0.603 | 0.037 | 0.006 | 0.531 | 0.675 |
| IL12RB2 | 0.603 | 0.037 | 0.006 | 0.531 | 0.676 |
| SRP68 | 0.603 | 0.037 | 0.006 | 0.531 | 0.675 |
| ZDHHC13 | 0.603 | 0.038 | 0.006 | 0.529 | 0.677 |
| AL354751.3 | 0.603 | 0.037 | 0.006 | 0.531 | 0.675 |
| KIF22 | 0.603 | 0.037 | 0.006 | 0.530 | 0.676 |
| CDC25A | 0.603 | 0.037 | 0.006 | 0.531 | 0.675 |
| MSL2 | 0.603 | 0.037 | 0.006 | 0.530 | 0.676 |
| TPX2 | 0.603 | 0.037 | 0.007 | 0.530 | 0.675 |
| EPSTI1 | 0.603 | 0.037 | 0.007 | 0.530 | 0.675 |
| SMC2 | 0.602 | 0.037 | 0.007 | 0.530 | 0.675 |
| SLBP | 0.602 | 0.037 | 0.007 | 0.531 | 0.674 |
| CD3E | 0.602 | 0.037 | 0.007 | 0.530 | 0.675 |
| HDGF | 0.602 | 0.037 | 0.007 | 0.530 | 0.674 |
| IQGAP3 | 0.602 | 0.037 | 0.007 | 0.530 | 0.674 |
| HNRNPL | 0.602 | 0.037 | 0.007 | 0.530 | 0.674 |
| GNLY | 0.602 | 0.037 | 0.007 | 0.530 | 0.674 |
| TSPOAP1.AS1 | 0.602 | 0.037 | 0.007 | 0.529 | 0.675 |
| PSMB10 | 0.602 | 0.037 | 0.007 | 0.530 | 0.674 |
| HCLS1 | 0.602 | 0.037 | 0.007 | 0.529 | 0.675 |
| CTSC | 0.602 | 0.037 | 0.007 | 0.530 | 0.674 |
| SLAMF1 | 0.602 | 0.037 | 0.007 | 0.530 | 0.674 |
| PIK3R6 | 0.602 | 0.037 | 0.007 | 0.530 | 0.674 |
| MCMBP | 0.602 | 0.037 | 0.007 | 0.530 | 0.674 |
| CEP192 | 0.602 | 0.037 | 0.007 | 0.530 | 0.674 |
| DSN1 | 0.602 | 0.037 | 0.007 | 0.530 | 0.674 |
| FUS | 0.602 | 0.037 | 0.007 | 0.529 | 0.675 |
| AC013264.1 | 0.602 | 0.037 | 0.007 | 0.530 | 0.674 |
| VCAM1 | 0.602 | 0.037 | 0.007 | 0.529 | 0.674 |
| NFKBIB | 0.602 | 0.037 | 0.007 | 0.529 | 0.674 |
| RIBC2 | 0.602 | 0.037 | 0.007 | 0.529 | 0.674 |
| CYBA | 0.602 | 0.037 | 0.007 | 0.530 | 0.674 |
| MTA2 | 0.602 | 0.037 | 0.007 | 0.530 | 0.674 |
| SFXN5 | 0.602 | 0.037 | 0.007 | 0.529 | 0.674 |
| SUV39H1 | 0.602 | 0.037 | 0.007 | 0.529 | 0.675 |
| FNDC3B | 0.602 | 0.037 | 0.007 | 0.529 | 0.674 |
| TOR3A | 0.602 | 0.037 | 0.007 | 0.530 | 0.674 |
| MOXD1 | 0.602 | 0.037 | 0.007 | 0.529 | 0.674 |
| BMS1 | 0.602 | 0.037 | 0.007 | 0.529 | 0.674 |
| TBCD | 0.602 | 0.037 | 0.007 | 0.529 | 0.674 |
| NLN | 0.602 | 0.037 | 0.007 | 0.529 | 0.674 |
| DHX38 | 0.602 | 0.037 | 0.007 | 0.530 | 0.673 |
| RIOX1 | 0.602 | 0.037 | 0.007 | 0.530 | 0.673 |
| RASGRP1 | 0.601 | 0.037 | 0.007 | 0.530 | 0.673 |
| DBF4B | 0.601 | 0.037 | 0.007 | 0.529 | 0.674 |
| NCSTN | 0.601 | 0.037 | 0.007 | 0.529 | 0.673 |
| ARFGAP2 | 0.601 | 0.037 | 0.007 | 0.529 | 0.673 |
| SLA | 0.601 | 0.037 | 0.007 | 0.529 | 0.673 |
| GLOD4 | 0.601 | 0.037 | 0.007 | 0.529 | 0.674 |
| FDPS | 0.601 | 0.037 | 0.007 | 0.529 | 0.674 |
| FBF1 | 0.601 | 0.037 | 0.007 | 0.528 | 0.675 |
| CD274 | 0.601 | 0.037 | 0.007 | 0.529 | 0.673 |
| SLC15A3 | 0.601 | 0.037 | 0.007 | 0.529 | 0.673 |
| RUSC1.AS1 | 0.601 | 0.037 | 0.007 | 0.529 | 0.674 |
| ARRB2 | 0.601 | 0.037 | 0.007 | 0.528 | 0.674 |
| GTPBP4 | 0.601 | 0.037 | 0.007 | 0.529 | 0.673 |
| PHTF1 | 0.601 | 0.037 | 0.007 | 0.529 | 0.673 |
| DSCR3 | 0.601 | 0.037 | 0.007 | 0.529 | 0.673 |
| S1PR4 | 0.601 | 0.037 | 0.007 | 0.528 | 0.674 |
| RASSF4 | 0.601 | 0.037 | 0.007 | 0.529 | 0.673 |
| HIST1H2AI | 0.601 | 0.037 | 0.007 | 0.529 | 0.673 |
| GPR171 | 0.601 | 0.037 | 0.007 | 0.528 | 0.674 |
| FTSJ3 | 0.601 | 0.037 | 0.007 | 0.529 | 0.673 |
| CHAF1A | 0.601 | 0.037 | 0.007 | 0.528 | 0.674 |
| NOP9 | 0.601 | 0.037 | 0.007 | 0.528 | 0.673 |
| TRIM28 | 0.601 | 0.037 | 0.007 | 0.529 | 0.673 |
| PJA1 | 0.601 | 0.037 | 0.007 | 0.529 | 0.673 |
| TXNDC5 | 0.601 | 0.037 | 0.007 | 0.529 | 0.673 |
| PCM1 | 0.601 | 0.037 | 0.007 | 0.529 | 0.673 |
| FAM161A | 0.601 | 0.037 | 0.008 | 0.528 | 0.673 |
| ITGAE | 0.601 | 0.037 | 0.008 | 0.529 | 0.672 |
| RFC4 | 0.601 | 0.037 | 0.008 | 0.528 | 0.673 |
| SIT1 | 0.601 | 0.037 | 0.008 | 0.528 | 0.673 |
| UBE2Q1.AS1 | 0.600 | 0.037 | 0.008 | 0.528 | 0.673 |
| JAML | 0.600 | 0.037 | 0.008 | 0.528 | 0.673 |
| ARHGEF19 | 0.600 | 0.037 | 0.008 | 0.528 | 0.673 |
| BTLA | 0.600 | 0.037 | 0.008 | 0.527 | 0.674 |
| VASH2 | 0.600 | 0.037 | 0.008 | 0.528 | 0.673 |
| NCF4 | 0.600 | 0.037 | 0.008 | 0.528 | 0.672 |
| TOMM40L | 0.600 | 0.037 | 0.008 | 0.528 | 0.672 |
| UNC13D | 0.600 | 0.037 | 0.008 | 0.527 | 0.673 |
| ZBP1 | 0.600 | 0.037 | 0.008 | 0.527 | 0.673 |
| SETDB1 | 0.600 | 0.037 | 0.008 | 0.527 | 0.673 |
| CEP250 | 0.600 | 0.037 | 0.008 | 0.528 | 0.672 |
| HUWE1 | 0.600 | 0.037 | 0.008 | 0.527 | 0.673 |
| GZMM | 0.600 | 0.037 | 0.008 | 0.528 | 0.672 |
| SKA2 | 0.600 | 0.037 | 0.008 | 0.527 | 0.673 |
| RNF4 | 0.600 | 0.037 | 0.008 | 0.527 | 0.672 |
| SNORD88A | 0.600 | 0.037 | 0.008 | 0.528 | 0.672 |
| PRKCA | 0.600 | 0.037 | 0.008 | 0.528 | 0.672 |
| PGAM1 | 0.600 | 0.037 | 0.008 | 0.527 | 0.672 |
| CNOT10 | 0.600 | 0.038 | 0.008 | 0.526 | 0.673 |
| SMCHD1 | 0.600 | 0.037 | 0.008 | 0.528 | 0.672 |
| ZNF831 | 0.600 | 0.037 | 0.008 | 0.527 | 0.672 |
| LBR | 0.600 | 0.037 | 0.008 | 0.527 | 0.672 |
| SDK1 | 0.599 | 0.037 | 0.008 | 0.527 | 0.672 |
| IKZF3 | 0.599 | 0.037 | 0.008 | 0.527 | 0.672 |
| ERI1 | 0.599 | 0.037 | 0.008 | 0.527 | 0.671 |
| JAK3 | 0.599 | 0.037 | 0.008 | 0.527 | 0.672 |
| RUVBL1 | 0.599 | 0.037 | 0.008 | 0.527 | 0.672 |
| YEATS2 | 0.599 | 0.037 | 0.008 | 0.526 | 0.672 |
| HPF1 | 0.599 | 0.037 | 0.008 | 0.527 | 0.671 |
| ASB13 | 0.599 | 0.037 | 0.009 | 0.527 | 0.672 |
| FAM111B | 0.599 | 0.037 | 0.009 | 0.527 | 0.671 |
| CRELD2 | 0.599 | 0.037 | 0.009 | 0.527 | 0.671 |
| IFI30 | 0.599 | 0.037 | 0.009 | 0.527 | 0.671 |
| DHFR | 0.599 | 0.037 | 0.009 | 0.527 | 0.671 |
| KIF15 | 0.599 | 0.037 | 0.009 | 0.526 | 0.671 |
| IL21R | 0.599 | 0.037 | 0.009 | 0.527 | 0.671 |
| MCUB | 0.599 | 0.038 | 0.009 | 0.525 | 0.672 |
| SERBP1 | 0.599 | 0.037 | 0.009 | 0.526 | 0.671 |
| PLEKHO2 | 0.599 | 0.037 | 0.009 | 0.526 | 0.672 |
| RNF122 | 0.599 | 0.037 | 0.009 | 0.526 | 0.671 |
| ZNF142 | 0.598 | 0.037 | 0.009 | 0.526 | 0.671 |
| KIAA1841 | 0.598 | 0.037 | 0.009 | 0.526 | 0.671 |
| GRAP2 | 0.598 | 0.037 | 0.009 | 0.526 | 0.671 |
| CBX5 | 0.598 | 0.037 | 0.009 | 0.525 | 0.672 |
| CDCA7 | 0.598 | 0.037 | 0.009 | 0.526 | 0.671 |
| VRK2 | 0.598 | 0.037 | 0.009 | 0.526 | 0.671 |
| PDE6D | 0.598 | 0.037 | 0.009 | 0.526 | 0.671 |
| AC021028.1 | 0.598 | 0.037 | 0.009 | 0.526 | 0.671 |
| APOL3 | 0.598 | 0.037 | 0.009 | 0.526 | 0.670 |
| STOX2 | 0.598 | 0.037 | 0.009 | 0.525 | 0.671 |
| TCIRG1 | 0.598 | 0.037 | 0.009 | 0.525 | 0.671 |
| LAX1 | 0.598 | 0.037 | 0.009 | 0.526 | 0.671 |
| CTSZ | 0.598 | 0.037 | 0.009 | 0.525 | 0.671 |
| HTATSF1 | 0.598 | 0.037 | 0.009 | 0.525 | 0.671 |
| TESPA1 | 0.598 | 0.037 | 0.009 | 0.525 | 0.670 |
| C17orf62 | 0.598 | 0.037 | 0.009 | 0.525 | 0.671 |
| SCLT1 | 0.598 | 0.037 | 0.009 | 0.526 | 0.670 |
| CARD11 | 0.598 | 0.037 | 0.009 | 0.525 | 0.671 |
| C1QA | 0.598 | 0.037 | 0.009 | 0.526 | 0.670 |
| IFI16 | 0.598 | 0.037 | 0.009 | 0.525 | 0.671 |
| PGS1 | 0.598 | 0.037 | 0.009 | 0.524 | 0.671 |
| ATP11A | 0.598 | 0.038 | 0.010 | 0.524 | 0.671 |
| CENPK | 0.598 | 0.037 | 0.010 | 0.525 | 0.671 |
| RSPO3 | 0.598 | 0.037 | 0.010 | 0.525 | 0.670 |
| DNA2 | 0.598 | 0.037 | 0.010 | 0.525 | 0.670 |
| SLC25A25 | 0.598 | 0.037 | 0.010 | 0.525 | 0.670 |
| ELAVL2 | 0.598 | 0.037 | 0.010 | 0.525 | 0.670 |
| GSTO1 | 0.598 | 0.037 | 0.010 | 0.525 | 0.670 |
| ANKFY1 | 0.597 | 0.037 | 0.010 | 0.524 | 0.671 |
| X1.Sep | 0.597 | 0.037 | 0.010 | 0.525 | 0.670 |
| HCFC1 | 0.597 | 0.037 | 0.010 | 0.525 | 0.670 |
| SNRPD1 | 0.597 | 0.037 | 0.010 | 0.526 | 0.669 |
| TXNL4B | 0.597 | 0.037 | 0.010 | 0.525 | 0.670 |
| HAUS5 | 0.597 | 0.037 | 0.010 | 0.525 | 0.670 |
| ZNF672 | 0.597 | 0.037 | 0.010 | 0.525 | 0.670 |
| OGFRP1 | 0.597 | 0.037 | 0.010 | 0.525 | 0.670 |
| IMPA2 | 0.597 | 0.037 | 0.010 | 0.525 | 0.669 |
| CIT | 0.597 | 0.037 | 0.010 | 0.525 | 0.670 |
| MIR4261 | 0.597 | 0.037 | 0.010 | 0.525 | 0.670 |
| SLC2A6 | 0.597 | 0.037 | 0.010 | 0.525 | 0.669 |
| PSMB4 | 0.597 | 0.037 | 0.010 | 0.525 | 0.669 |
| POU5F1P4 | 0.597 | 0.037 | 0.010 | 0.525 | 0.669 |
| LRRCC1 | 0.597 | 0.037 | 0.010 | 0.525 | 0.669 |
| BCL2A1 | 0.597 | 0.037 | 0.010 | 0.524 | 0.669 |
| PYHIN1 | 0.597 | 0.037 | 0.010 | 0.524 | 0.670 |
| ZC3H12A | 0.597 | 0.037 | 0.010 | 0.524 | 0.669 |
| FBXO5 | 0.597 | 0.037 | 0.010 | 0.524 | 0.669 |
| LRRC42 | 0.597 | 0.037 | 0.010 | 0.525 | 0.669 |
| PEA15 | 0.597 | 0.037 | 0.010 | 0.524 | 0.669 |
| CD74 | 0.597 | 0.037 | 0.010 | 0.524 | 0.669 |
| TSPAN32 | 0.597 | 0.037 | 0.010 | 0.524 | 0.669 |
| PIMREG | 0.596 | 0.037 | 0.010 | 0.524 | 0.669 |
| SIPA1L1 | 0.596 | 0.037 | 0.010 | 0.525 | 0.668 |
| CD86 | 0.596 | 0.037 | 0.010 | 0.524 | 0.669 |
| IL27RA | 0.596 | 0.037 | 0.010 | 0.525 | 0.668 |
| CCNJ | 0.596 | 0.037 | 0.010 | 0.523 | 0.669 |
| C1RL.AS1 | 0.596 | 0.037 | 0.011 | 0.524 | 0.668 |
| ITGAL | 0.596 | 0.037 | 0.011 | 0.524 | 0.669 |
| CTLA4 | 0.596 | 0.037 | 0.011 | 0.524 | 0.668 |
| PRKCQ | 0.596 | 0.037 | 0.011 | 0.524 | 0.669 |
| PSIP1 | 0.596 | 0.037 | 0.011 | 0.524 | 0.669 |
| HIST1H2BN | 0.596 | 0.037 | 0.011 | 0.524 | 0.669 |
| GDI2 | 0.596 | 0.037 | 0.011 | 0.524 | 0.668 |
| CENPW | 0.596 | 0.037 | 0.011 | 0.523 | 0.669 |
| MAGOH | 0.596 | 0.037 | 0.011 | 0.524 | 0.668 |
| HNRNPK | 0.596 | 0.037 | 0.011 | 0.524 | 0.668 |
| SLAMF7 | 0.596 | 0.037 | 0.011 | 0.524 | 0.668 |
| DOK1 | 0.596 | 0.037 | 0.011 | 0.523 | 0.669 |
| KDM1B | 0.596 | 0.037 | 0.011 | 0.524 | 0.668 |
| PIK3AP1 | 0.596 | 0.037 | 0.011 | 0.524 | 0.668 |
| HIST1H3H | 0.596 | 0.037 | 0.011 | 0.523 | 0.669 |
| SLAMF6 | 0.596 | 0.037 | 0.011 | 0.523 | 0.669 |
| DDB2 | 0.596 | 0.037 | 0.011 | 0.523 | 0.669 |
| CCNB1 | 0.596 | 0.037 | 0.011 | 0.524 | 0.668 |
| PPIF | 0.596 | 0.037 | 0.011 | 0.523 | 0.669 |
| KIF1A | 0.596 | 0.037 | 0.011 | 0.523 | 0.669 |
| APOBEC3B | 0.596 | 0.037 | 0.011 | 0.524 | 0.668 |
| USP21 | 0.596 | 0.037 | 0.011 | 0.524 | 0.668 |
| CYTH1 | 0.596 | 0.037 | 0.011 | 0.523 | 0.669 |
| VASH1 | 0.596 | 0.037 | 0.011 | 0.523 | 0.669 |
| CD38 | 0.596 | 0.037 | 0.011 | 0.524 | 0.668 |
| ANKRD33B | 0.596 | 0.037 | 0.011 | 0.524 | 0.668 |
| LCP2 | 0.596 | 0.037 | 0.011 | 0.523 | 0.668 |
| ZNF43 | 0.596 | 0.037 | 0.011 | 0.523 | 0.668 |
| GRK6 | 0.596 | 0.037 | 0.011 | 0.523 | 0.668 |
| PTMA | 0.596 | 0.037 | 0.011 | 0.524 | 0.668 |
| TRA2B | 0.596 | 0.037 | 0.011 | 0.523 | 0.668 |
| TTF2 | 0.596 | 0.037 | 0.011 | 0.524 | 0.668 |
| DENND3 | 0.596 | 0.037 | 0.011 | 0.523 | 0.669 |
| CTSB | 0.596 | 0.037 | 0.011 | 0.523 | 0.668 |
| SUSD1 | 0.596 | 0.037 | 0.011 | 0.524 | 0.668 |
| GADD45A | 0.596 | 0.037 | 0.011 | 0.522 | 0.669 |
| UCK2 | 0.596 | 0.037 | 0.011 | 0.522 | 0.669 |
| PINX1 | 0.596 | 0.037 | 0.011 | 0.524 | 0.668 |
| LRRC8C | 0.596 | 0.037 | 0.011 | 0.523 | 0.668 |
| RUFY4 | 0.596 | 0.037 | 0.011 | 0.523 | 0.668 |
| QSER1 | 0.596 | 0.037 | 0.011 | 0.522 | 0.669 |
| TOMM40 | 0.596 | 0.037 | 0.011 | 0.524 | 0.667 |
| CTSS | 0.596 | 0.037 | 0.011 | 0.524 | 0.668 |
| ANP32B | 0.595 | 0.037 | 0.011 | 0.523 | 0.668 |
| CD300A | 0.595 | 0.037 | 0.011 | 0.523 | 0.668 |
| PIK3CD.AS1 | 0.595 | 0.037 | 0.011 | 0.523 | 0.668 |
| MBNL1.AS1 | 0.595 | 0.037 | 0.011 | 0.523 | 0.668 |
| JAK1 | 0.595 | 0.037 | 0.011 | 0.523 | 0.668 |
| KHDRBS1 | 0.595 | 0.037 | 0.011 | 0.522 | 0.668 |
| WDR34 | 0.595 | 0.037 | 0.011 | 0.523 | 0.668 |
| GINS2 | 0.595 | 0.037 | 0.011 | 0.522 | 0.669 |
| HS3ST3B1 | 0.595 | 0.037 | 0.011 | 0.523 | 0.668 |
| LEPROTL1 | 0.595 | 0.037 | 0.011 | 0.523 | 0.667 |
| TNFSF14 | 0.595 | 0.038 | 0.012 | 0.521 | 0.669 |
| CIP2A | 0.595 | 0.037 | 0.012 | 0.523 | 0.668 |
| MRPL9 | 0.595 | 0.037 | 0.012 | 0.523 | 0.668 |
| TTC39C | 0.595 | 0.037 | 0.012 | 0.523 | 0.667 |
| TELO2 | 0.595 | 0.037 | 0.012 | 0.522 | 0.668 |
| TOP2B | 0.595 | 0.037 | 0.012 | 0.522 | 0.668 |
| FMNL1 | 0.595 | 0.037 | 0.012 | 0.523 | 0.668 |
| LYN | 0.595 | 0.037 | 0.012 | 0.523 | 0.667 |
| NONO | 0.595 | 0.037 | 0.012 | 0.522 | 0.668 |
| KLHL5 | 0.595 | 0.037 | 0.012 | 0.523 | 0.667 |
| SLC17A9 | 0.595 | 0.037 | 0.012 | 0.522 | 0.668 |
| SOAT1 | 0.595 | 0.037 | 0.012 | 0.523 | 0.667 |
| PRKX | 0.595 | 0.038 | 0.012 | 0.521 | 0.668 |
| SEPHS1 | 0.595 | 0.037 | 0.012 | 0.523 | 0.667 |
| LRRC25 | 0.595 | 0.037 | 0.012 | 0.522 | 0.668 |
| HDAC4 | 0.595 | 0.037 | 0.012 | 0.522 | 0.667 |
| APOBEC3F | 0.595 | 0.037 | 0.012 | 0.522 | 0.668 |
| MMP12 | 0.595 | 0.037 | 0.012 | 0.522 | 0.667 |
| MGAT3.AS1 | 0.595 | 0.037 | 0.012 | 0.522 | 0.667 |
| IRF8 | 0.595 | 0.037 | 0.012 | 0.522 | 0.667 |
| CXCR3 | 0.595 | 0.037 | 0.012 | 0.523 | 0.667 |
| LSP1 | 0.595 | 0.037 | 0.012 | 0.522 | 0.667 |
| GAR1 | 0.595 | 0.037 | 0.012 | 0.522 | 0.667 |
| ERCC6L | 0.595 | 0.037 | 0.012 | 0.523 | 0.667 |
| IKZF1 | 0.595 | 0.037 | 0.012 | 0.522 | 0.667 |
| RNF19B | 0.595 | 0.037 | 0.012 | 0.522 | 0.667 |
| PLSCR1 | 0.594 | 0.037 | 0.012 | 0.521 | 0.668 |
| CST7 | 0.594 | 0.037 | 0.012 | 0.522 | 0.667 |
| BIRC3 | 0.594 | 0.037 | 0.012 | 0.521 | 0.668 |
| RRP1B | 0.594 | 0.037 | 0.012 | 0.521 | 0.667 |
| BUB1 | 0.594 | 0.037 | 0.012 | 0.522 | 0.667 |
| NUSAP1 | 0.594 | 0.037 | 0.012 | 0.521 | 0.667 |
| C8G | 0.594 | 0.037 | 0.012 | 0.522 | 0.667 |
| CD40LG | 0.594 | 0.037 | 0.012 | 0.521 | 0.667 |
| WNK3 | 0.594 | 0.037 | 0.012 | 0.521 | 0.667 |
| EOMES | 0.594 | 0.037 | 0.012 | 0.522 | 0.667 |
| CAPG | 0.594 | 0.037 | 0.012 | 0.521 | 0.667 |
| DCLRE1C | 0.594 | 0.037 | 0.012 | 0.521 | 0.667 |
| SUPT16H | 0.594 | 0.037 | 0.012 | 0.522 | 0.666 |
| EGR2 | 0.594 | 0.037 | 0.012 | 0.522 | 0.667 |
| YES1 | 0.594 | 0.037 | 0.012 | 0.522 | 0.667 |
| FAM78A | 0.594 | 0.037 | 0.013 | 0.521 | 0.667 |
| VANGL2 | 0.594 | 0.037 | 0.013 | 0.522 | 0.666 |
| PTAFR | 0.594 | 0.037 | 0.013 | 0.521 | 0.667 |
| TBC1D1 | 0.594 | 0.037 | 0.013 | 0.522 | 0.666 |
| CYTH4 | 0.594 | 0.037 | 0.013 | 0.521 | 0.667 |
| GPR19 | 0.594 | 0.038 | 0.013 | 0.520 | 0.667 |
| RAC2 | 0.594 | 0.037 | 0.013 | 0.520 | 0.667 |
| ARHGEF11 | 0.594 | 0.037 | 0.013 | 0.521 | 0.667 |
| SRSF2 | 0.594 | 0.037 | 0.013 | 0.521 | 0.667 |
| CD4 | 0.594 | 0.037 | 0.013 | 0.521 | 0.666 |
| AC008105.3 | 0.594 | 0.037 | 0.013 | 0.521 | 0.666 |
| TNIP1 | 0.594 | 0.037 | 0.013 | 0.520 | 0.667 |
| FAM53B | 0.594 | 0.037 | 0.013 | 0.521 | 0.666 |
| HNRNPUL1 | 0.594 | 0.037 | 0.013 | 0.520 | 0.667 |
| C1S | 0.593 | 0.037 | 0.013 | 0.521 | 0.666 |
| CD163 | 0.593 | 0.038 | 0.013 | 0.520 | 0.667 |
| EHBP1L1 | 0.593 | 0.037 | 0.013 | 0.521 | 0.666 |
| GGA3 | 0.593 | 0.038 | 0.013 | 0.519 | 0.667 |
| GPR161 | 0.593 | 0.037 | 0.013 | 0.521 | 0.666 |
| TMEM14EP | 0.593 | 0.037 | 0.013 | 0.520 | 0.666 |
| NLRC5 | 0.593 | 0.037 | 0.013 | 0.521 | 0.666 |
| NEMP1 | 0.593 | 0.037 | 0.013 | 0.520 | 0.667 |
| HSPA14 | 0.593 | 0.037 | 0.013 | 0.521 | 0.666 |
| PTTG1 | 0.593 | 0.037 | 0.013 | 0.520 | 0.667 |
| GLE1 | 0.593 | 0.037 | 0.013 | 0.520 | 0.666 |
| SIGLEC9 | 0.593 | 0.037 | 0.013 | 0.520 | 0.666 |
| PAQR8 | 0.593 | 0.037 | 0.013 | 0.520 | 0.666 |
| PAK3 | 0.593 | 0.037 | 0.013 | 0.521 | 0.665 |
| RBP5 | 0.593 | 0.037 | 0.013 | 0.521 | 0.665 |
| C1RL | 0.593 | 0.037 | 0.014 | 0.520 | 0.666 |
| KIF20B | 0.593 | 0.037 | 0.014 | 0.521 | 0.665 |
| CCNE2 | 0.593 | 0.037 | 0.014 | 0.521 | 0.665 |
| SNRPB | 0.593 | 0.037 | 0.014 | 0.521 | 0.665 |
| NARF | 0.593 | 0.037 | 0.014 | 0.520 | 0.666 |
| CDC7 | 0.593 | 0.037 | 0.014 | 0.520 | 0.665 |
| PHF21A | 0.593 | 0.037 | 0.014 | 0.520 | 0.666 |
| RASAL3 | 0.593 | 0.037 | 0.014 | 0.520 | 0.666 |
| MRPL38 | 0.593 | 0.037 | 0.014 | 0.520 | 0.666 |
| FKBP15 | 0.593 | 0.037 | 0.014 | 0.520 | 0.666 |
| RTKN2 | 0.593 | 0.037 | 0.014 | 0.520 | 0.666 |
| SOCS1 | 0.593 | 0.037 | 0.014 | 0.521 | 0.665 |
| PCNT | 0.593 | 0.037 | 0.014 | 0.520 | 0.666 |
| SPTAN1 | 0.593 | 0.037 | 0.014 | 0.520 | 0.665 |
| PARP14 | 0.593 | 0.037 | 0.014 | 0.519 | 0.666 |
| BATF2 | 0.593 | 0.037 | 0.014 | 0.519 | 0.666 |
| MCPH1 | 0.593 | 0.037 | 0.014 | 0.520 | 0.665 |
| LYAR | 0.593 | 0.037 | 0.014 | 0.520 | 0.665 |
| MIR8055 | 0.593 | 0.037 | 0.014 | 0.520 | 0.665 |
| CHMP6 | 0.593 | 0.037 | 0.014 | 0.519 | 0.666 |
| SRSF4 | 0.593 | 0.037 | 0.014 | 0.520 | 0.665 |
| HIST1H1C | 0.592 | 0.037 | 0.014 | 0.519 | 0.666 |
| MAD2L2 | 0.592 | 0.037 | 0.014 | 0.519 | 0.666 |
| MYD88 | 0.592 | 0.037 | 0.014 | 0.519 | 0.665 |
| NUGGC | 0.592 | 0.037 | 0.014 | 0.520 | 0.665 |
| CTDP1 | 0.592 | 0.037 | 0.014 | 0.520 | 0.665 |
| GBP4 | 0.592 | 0.037 | 0.014 | 0.520 | 0.665 |
| SSRP1 | 0.592 | 0.037 | 0.014 | 0.520 | 0.665 |
| FAM104A | 0.592 | 0.037 | 0.014 | 0.520 | 0.665 |
| CXCL9 | 0.592 | 0.037 | 0.014 | 0.520 | 0.664 |
| CXCL10 | 0.592 | 0.037 | 0.014 | 0.520 | 0.665 |
| FANCG | 0.592 | 0.037 | 0.014 | 0.520 | 0.665 |
| HMMR | 0.592 | 0.037 | 0.014 | 0.520 | 0.665 |
| MLKL | 0.592 | 0.037 | 0.014 | 0.519 | 0.665 |
| C1QC | 0.592 | 0.037 | 0.014 | 0.520 | 0.665 |
| NFKBIA | 0.592 | 0.037 | 0.015 | 0.519 | 0.665 |
| USP10 | 0.592 | 0.037 | 0.015 | 0.519 | 0.665 |
| SLFN12L | 0.592 | 0.037 | 0.015 | 0.520 | 0.664 |
| ASF1B | 0.592 | 0.037 | 0.015 | 0.519 | 0.665 |
| RAD51 | 0.592 | 0.037 | 0.015 | 0.519 | 0.664 |
| IFNG | 0.592 | 0.037 | 0.015 | 0.520 | 0.664 |
| CISH | 0.592 | 0.037 | 0.015 | 0.519 | 0.664 |
| SP6 | 0.592 | 0.037 | 0.015 | 0.519 | 0.665 |
| CENPX | 0.592 | 0.037 | 0.015 | 0.519 | 0.665 |
| APOBEC3C | 0.592 | 0.037 | 0.015 | 0.519 | 0.664 |
| S1PR2 | 0.592 | 0.037 | 0.015 | 0.519 | 0.664 |
| BCAT1 | 0.592 | 0.037 | 0.015 | 0.519 | 0.665 |
| CWC27 | 0.592 | 0.037 | 0.015 | 0.519 | 0.665 |
| GABPB2 | 0.592 | 0.038 | 0.015 | 0.518 | 0.665 |
| RHOG | 0.592 | 0.037 | 0.015 | 0.519 | 0.664 |
| THEMIS2 | 0.592 | 0.038 | 0.015 | 0.518 | 0.665 |
| NMT2 | 0.592 | 0.037 | 0.015 | 0.519 | 0.664 |
| MTX1 | 0.592 | 0.037 | 0.015 | 0.519 | 0.664 |
| CMPK2 | 0.592 | 0.038 | 0.015 | 0.518 | 0.665 |
| STAG1 | 0.591 | 0.037 | 0.015 | 0.519 | 0.664 |
| P2RY10 | 0.591 | 0.037 | 0.015 | 0.518 | 0.665 |
| KIAA1211 | 0.591 | 0.037 | 0.015 | 0.519 | 0.664 |
| SH2D1A | 0.591 | 0.037 | 0.015 | 0.519 | 0.664 |
| SMCO4 | 0.591 | 0.037 | 0.015 | 0.518 | 0.664 |
| GIMAP6 | 0.591 | 0.037 | 0.015 | 0.519 | 0.664 |
| TRPV2 | 0.591 | 0.037 | 0.015 | 0.519 | 0.664 |
| TRAF3IP3 | 0.591 | 0.037 | 0.015 | 0.518 | 0.664 |
| FCRL5 | 0.591 | 0.037 | 0.015 | 0.519 | 0.664 |
| IL18BP | 0.591 | 0.037 | 0.016 | 0.518 | 0.664 |
| CCT3 | 0.591 | 0.037 | 0.016 | 0.519 | 0.663 |
| RBM23 | 0.591 | 0.037 | 0.016 | 0.518 | 0.665 |
| CYBB | 0.591 | 0.037 | 0.016 | 0.519 | 0.664 |
| TMEM51 | 0.591 | 0.038 | 0.016 | 0.517 | 0.665 |
| E2F7 | 0.591 | 0.037 | 0.016 | 0.518 | 0.664 |
| TMEM158 | 0.591 | 0.037 | 0.016 | 0.518 | 0.664 |
| DNAJB12 | 0.591 | 0.037 | 0.016 | 0.518 | 0.664 |
| GPR84 | 0.591 | 0.037 | 0.016 | 0.518 | 0.664 |
| UBTF | 0.591 | 0.037 | 0.016 | 0.519 | 0.663 |
| SFMBT2 | 0.591 | 0.037 | 0.016 | 0.519 | 0.663 |
| PRELID3A | 0.591 | 0.037 | 0.016 | 0.518 | 0.664 |
| HIST1H2AH | 0.591 | 0.037 | 0.016 | 0.518 | 0.663 |
| IFIH1 | 0.591 | 0.037 | 0.016 | 0.518 | 0.664 |
| BLM | 0.591 | 0.037 | 0.016 | 0.519 | 0.663 |
| ARSB | 0.591 | 0.037 | 0.016 | 0.517 | 0.664 |
| ACBD7 | 0.591 | 0.037 | 0.016 | 0.518 | 0.664 |
| HIRIP3 | 0.591 | 0.037 | 0.016 | 0.518 | 0.663 |
| CENPF | 0.591 | 0.037 | 0.016 | 0.518 | 0.664 |
| NMI | 0.591 | 0.037 | 0.016 | 0.517 | 0.664 |
| CISD2 | 0.591 | 0.037 | 0.016 | 0.518 | 0.663 |
| CRTC2 | 0.591 | 0.037 | 0.016 | 0.517 | 0.664 |
| DHX37 | 0.591 | 0.037 | 0.016 | 0.517 | 0.664 |
| SP140 | 0.590 | 0.037 | 0.016 | 0.517 | 0.664 |
| TIFA | 0.590 | 0.038 | 0.016 | 0.516 | 0.664 |
| TRIM16 | 0.590 | 0.037 | 0.016 | 0.518 | 0.663 |
| HNRNPA0 | 0.590 | 0.037 | 0.016 | 0.518 | 0.663 |
| CDCA5 | 0.590 | 0.037 | 0.017 | 0.518 | 0.662 |
| COL4A4 | 0.590 | 0.037 | 0.017 | 0.517 | 0.663 |
| ODF3B | 0.590 | 0.037 | 0.017 | 0.517 | 0.664 |
| C7orf49 | 0.590 | 0.037 | 0.017 | 0.517 | 0.663 |
| PIK3R5 | 0.590 | 0.037 | 0.017 | 0.518 | 0.663 |
| AURKB | 0.590 | 0.037 | 0.017 | 0.517 | 0.663 |
| C1R | 0.590 | 0.037 | 0.017 | 0.518 | 0.663 |
| GXYLT1 | 0.590 | 0.037 | 0.017 | 0.517 | 0.663 |
| CD14 | 0.590 | 0.037 | 0.017 | 0.517 | 0.664 |
| FLAD1 | 0.590 | 0.037 | 0.017 | 0.517 | 0.663 |
| ASCC3 | 0.590 | 0.037 | 0.017 | 0.517 | 0.663 |
| MIR6753 | 0.590 | 0.037 | 0.017 | 0.517 | 0.663 |
| CDC20 | 0.590 | 0.037 | 0.017 | 0.517 | 0.662 |
| WDR1 | 0.590 | 0.037 | 0.017 | 0.516 | 0.663 |
| RNF165 | 0.590 | 0.038 | 0.017 | 0.516 | 0.663 |
| BUB1B | 0.590 | 0.037 | 0.017 | 0.517 | 0.663 |
| CASP2 | 0.590 | 0.037 | 0.017 | 0.517 | 0.663 |
| DDX46 | 0.590 | 0.037 | 0.017 | 0.517 | 0.662 |
| IL17RA | 0.590 | 0.037 | 0.017 | 0.517 | 0.662 |
| GABBR2 | 0.590 | 0.037 | 0.017 | 0.517 | 0.662 |
| DOCK2 | 0.590 | 0.037 | 0.017 | 0.517 | 0.662 |
| L3MBTL3 | 0.590 | 0.037 | 0.017 | 0.517 | 0.662 |
| POU2AF1 | 0.590 | 0.037 | 0.017 | 0.517 | 0.663 |
| TRIM25 | 0.590 | 0.038 | 0.017 | 0.515 | 0.664 |
| MARCO | 0.590 | 0.037 | 0.017 | 0.517 | 0.662 |
| SAMSN1 | 0.590 | 0.037 | 0.017 | 0.517 | 0.662 |
| NCF1C | 0.590 | 0.037 | 0.017 | 0.517 | 0.662 |
| ARHGAP25 | 0.590 | 0.037 | 0.017 | 0.517 | 0.662 |
| C4orf46 | 0.590 | 0.037 | 0.017 | 0.517 | 0.662 |
| GMPS | 0.590 | 0.037 | 0.017 | 0.517 | 0.662 |
| HCST | 0.589 | 0.037 | 0.018 | 0.517 | 0.662 |
| FRMD3 | 0.589 | 0.037 | 0.018 | 0.517 | 0.662 |
| SLC7A11 | 0.589 | 0.037 | 0.018 | 0.516 | 0.663 |
| DTL | 0.589 | 0.037 | 0.018 | 0.517 | 0.662 |
| ZNF48 | 0.589 | 0.037 | 0.018 | 0.517 | 0.662 |
| SIGLEC1 | 0.589 | 0.037 | 0.018 | 0.516 | 0.663 |
| FCER1G | 0.589 | 0.037 | 0.018 | 0.517 | 0.662 |
| TRIM59 | 0.589 | 0.037 | 0.018 | 0.517 | 0.662 |
| CD6 | 0.589 | 0.037 | 0.018 | 0.517 | 0.662 |
| AKAP8 | 0.589 | 0.037 | 0.018 | 0.517 | 0.662 |
| CD300LF | 0.589 | 0.037 | 0.018 | 0.517 | 0.662 |
| ACSL5 | 0.589 | 0.037 | 0.018 | 0.517 | 0.662 |
| STAP1 | 0.589 | 0.038 | 0.018 | 0.516 | 0.663 |
| PTPN6 | 0.589 | 0.037 | 0.018 | 0.516 | 0.662 |
| MAPK14 | 0.589 | 0.037 | 0.018 | 0.517 | 0.661 |
| YWHAH | 0.589 | 0.037 | 0.018 | 0.517 | 0.662 |
| IL10RA | 0.589 | 0.037 | 0.018 | 0.516 | 0.662 |
| TLK2 | 0.589 | 0.037 | 0.018 | 0.516 | 0.662 |
| PLCL2 | 0.589 | 0.037 | 0.018 | 0.517 | 0.661 |
| ZMYND15 | 0.589 | 0.037 | 0.018 | 0.516 | 0.662 |
| CNBP | 0.589 | 0.037 | 0.018 | 0.516 | 0.662 |
| CDCA8 | 0.589 | 0.037 | 0.018 | 0.515 | 0.662 |
| RPS6KA4 | 0.589 | 0.037 | 0.018 | 0.516 | 0.661 |
| MSI1 | 0.589 | 0.037 | 0.018 | 0.516 | 0.661 |
| ZNF79 | 0.589 | 0.037 | 0.018 | 0.516 | 0.662 |
| GIMAP5 | 0.589 | 0.037 | 0.018 | 0.516 | 0.661 |
| SF3B4 | 0.589 | 0.037 | 0.018 | 0.516 | 0.662 |
| HDAC2 | 0.589 | 0.037 | 0.019 | 0.516 | 0.661 |
| CXCR6 | 0.589 | 0.037 | 0.019 | 0.516 | 0.661 |
| NCAPD2 | 0.589 | 0.037 | 0.019 | 0.516 | 0.662 |
| CNOT7 | 0.589 | 0.037 | 0.019 | 0.516 | 0.661 |
| RIPOR3 | 0.589 | 0.037 | 0.019 | 0.516 | 0.661 |
| COL4A2.AS1 | 0.589 | 0.037 | 0.019 | 0.516 | 0.661 |
| CCR2 | 0.589 | 0.037 | 0.019 | 0.516 | 0.661 |
| PSMD2 | 0.589 | 0.037 | 0.019 | 0.516 | 0.661 |
| SAAL1 | 0.589 | 0.037 | 0.019 | 0.516 | 0.661 |
| C5 | 0.589 | 0.037 | 0.019 | 0.515 | 0.662 |
| NFXL1 | 0.589 | 0.037 | 0.019 | 0.516 | 0.661 |
| XPO6 | 0.589 | 0.037 | 0.019 | 0.516 | 0.661 |
| RNF150 | 0.589 | 0.037 | 0.019 | 0.516 | 0.661 |
| MIR6731 | 0.589 | 0.037 | 0.019 | 0.516 | 0.662 |
| DENND2D | 0.589 | 0.037 | 0.019 | 0.516 | 0.662 |
| BAK1 | 0.588 | 0.037 | 0.019 | 0.515 | 0.661 |
| ETV3 | 0.588 | 0.037 | 0.019 | 0.515 | 0.662 |
| OTUD5 | 0.588 | 0.037 | 0.019 | 0.515 | 0.662 |
| PI4KB | 0.588 | 0.038 | 0.019 | 0.515 | 0.662 |
| MIR6851 | 0.588 | 0.037 | 0.019 | 0.516 | 0.661 |
| DYRK1A | 0.588 | 0.037 | 0.019 | 0.516 | 0.661 |
| COL22A1 | 0.588 | 0.037 | 0.019 | 0.515 | 0.661 |
| HPS3 | 0.588 | 0.037 | 0.019 | 0.516 | 0.661 |
| KIF23 | 0.588 | 0.037 | 0.019 | 0.515 | 0.661 |
| DHX9 | 0.588 | 0.037 | 0.019 | 0.516 | 0.661 |
| PATL1 | 0.588 | 0.037 | 0.019 | 0.515 | 0.662 |
| AC100861.1 | 0.588 | 0.037 | 0.019 | 0.515 | 0.661 |
| POLA1 | 0.588 | 0.037 | 0.019 | 0.515 | 0.661 |
| CARMIL2 | 0.588 | 0.037 | 0.019 | 0.515 | 0.661 |
| IL1RN | 0.588 | 0.038 | 0.019 | 0.514 | 0.662 |
| MIR4269 | 0.588 | 0.038 | 0.020 | 0.514 | 0.662 |
| IL10 | 0.588 | 0.037 | 0.020 | 0.515 | 0.661 |
| METTL4 | 0.588 | 0.037 | 0.020 | 0.516 | 0.660 |
| PTPRC | 0.588 | 0.037 | 0.020 | 0.515 | 0.661 |
| SLC2A3 | 0.588 | 0.037 | 0.020 | 0.515 | 0.661 |
| TRAT1 | 0.588 | 0.037 | 0.020 | 0.515 | 0.660 |
| PRMT9 | 0.588 | 0.037 | 0.020 | 0.515 | 0.661 |
| LSM4 | 0.588 | 0.037 | 0.020 | 0.515 | 0.660 |
| NAT10 | 0.588 | 0.037 | 0.020 | 0.515 | 0.661 |
| BLMH | 0.588 | 0.037 | 0.020 | 0.515 | 0.661 |
| HLA.DQA1 | 0.588 | 0.037 | 0.020 | 0.515 | 0.660 |
| CHEK1 | 0.588 | 0.037 | 0.020 | 0.515 | 0.661 |
| USP18 | 0.588 | 0.037 | 0.020 | 0.515 | 0.661 |
| ZC3H12D | 0.588 | 0.037 | 0.020 | 0.515 | 0.660 |
| FKBP11 | 0.588 | 0.037 | 0.020 | 0.515 | 0.661 |
| UBASH3B | 0.588 | 0.037 | 0.020 | 0.514 | 0.661 |
| DENND1A | 0.588 | 0.037 | 0.020 | 0.515 | 0.660 |
| ERLIN1 | 0.588 | 0.037 | 0.020 | 0.515 | 0.660 |
| ATP11C | 0.588 | 0.037 | 0.020 | 0.515 | 0.660 |
| CENPH | 0.588 | 0.037 | 0.020 | 0.515 | 0.661 |
| UBE2I | 0.588 | 0.037 | 0.020 | 0.515 | 0.660 |
| DHX8 | 0.588 | 0.037 | 0.020 | 0.514 | 0.661 |
| SELPLG | 0.588 | 0.037 | 0.020 | 0.515 | 0.660 |
| FBXO6 | 0.588 | 0.037 | 0.020 | 0.515 | 0.660 |
| FBXL19 | 0.587 | 0.037 | 0.020 | 0.515 | 0.660 |
| RAP2B | 0.587 | 0.037 | 0.020 | 0.515 | 0.660 |
| FNBP1 | 0.587 | 0.037 | 0.020 | 0.515 | 0.660 |
| FAAH2 | 0.587 | 0.037 | 0.020 | 0.515 | 0.660 |
| PI4K2A | 0.587 | 0.037 | 0.020 | 0.515 | 0.660 |
| GZMH | 0.587 | 0.037 | 0.020 | 0.515 | 0.660 |
| TNIP2 | 0.587 | 0.037 | 0.020 | 0.514 | 0.660 |
| PPP1R16B | 0.587 | 0.037 | 0.020 | 0.514 | 0.660 |
| IDO1 | 0.587 | 0.037 | 0.020 | 0.515 | 0.660 |
| GNL3L | 0.587 | 0.038 | 0.021 | 0.514 | 0.661 |
| RASGEF1B | 0.587 | 0.037 | 0.021 | 0.514 | 0.660 |
| ESPL1 | 0.587 | 0.037 | 0.021 | 0.514 | 0.660 |
| B4GALT3 | 0.587 | 0.037 | 0.021 | 0.514 | 0.660 |
| AC124319.2 | 0.587 | 0.037 | 0.021 | 0.515 | 0.659 |
| ANXA6 | 0.587 | 0.037 | 0.021 | 0.514 | 0.660 |
| RASGEF1A | 0.587 | 0.038 | 0.021 | 0.513 | 0.661 |
| GPATCH4 | 0.587 | 0.037 | 0.021 | 0.515 | 0.659 |
| CD8A | 0.587 | 0.037 | 0.021 | 0.515 | 0.659 |
| EPB41 | 0.587 | 0.037 | 0.021 | 0.515 | 0.659 |
| KNL1 | 0.587 | 0.037 | 0.021 | 0.514 | 0.660 |
| SDF2L1 | 0.587 | 0.037 | 0.021 | 0.514 | 0.660 |
| BARD1 | 0.587 | 0.037 | 0.021 | 0.514 | 0.660 |
| ARHGAP18 | 0.587 | 0.037 | 0.021 | 0.514 | 0.660 |
| SOD2 | 0.587 | 0.037 | 0.021 | 0.514 | 0.660 |
| HAPLN3 | 0.587 | 0.037 | 0.021 | 0.514 | 0.660 |
| PES1 | 0.587 | 0.037 | 0.021 | 0.514 | 0.659 |
| LRRC40 | 0.587 | 0.037 | 0.021 | 0.514 | 0.659 |
| MAD2L1 | 0.587 | 0.037 | 0.021 | 0.514 | 0.659 |
| DCAF8 | 0.587 | 0.037 | 0.021 | 0.514 | 0.659 |
| TMEM18 | 0.587 | 0.037 | 0.021 | 0.514 | 0.659 |
| THOC1 | 0.587 | 0.037 | 0.021 | 0.514 | 0.660 |
| KCNAB2 | 0.587 | 0.037 | 0.021 | 0.514 | 0.659 |
| NUBP1 | 0.587 | 0.037 | 0.021 | 0.514 | 0.659 |
| TNFRSF11B | 0.587 | 0.037 | 0.022 | 0.514 | 0.659 |
| CKS1B | 0.587 | 0.037 | 0.022 | 0.514 | 0.659 |
| TUBG1 | 0.587 | 0.037 | 0.022 | 0.514 | 0.659 |
| MOB3C | 0.587 | 0.037 | 0.022 | 0.514 | 0.659 |
| NEMP2 | 0.587 | 0.037 | 0.022 | 0.514 | 0.659 |
| ARHGAP19 | 0.587 | 0.038 | 0.022 | 0.513 | 0.660 |
| ARPC2 | 0.587 | 0.037 | 0.022 | 0.514 | 0.659 |
| INCENP | 0.586 | 0.037 | 0.022 | 0.514 | 0.659 |
| MX2 | 0.586 | 0.038 | 0.022 | 0.513 | 0.660 |
| SSX2IP | 0.586 | 0.037 | 0.022 | 0.513 | 0.660 |
| MYO1F | 0.586 | 0.037 | 0.022 | 0.514 | 0.659 |
| PFN1 | 0.586 | 0.037 | 0.022 | 0.513 | 0.660 |
| TMC8 | 0.586 | 0.037 | 0.022 | 0.513 | 0.660 |
| SCML4 | 0.586 | 0.037 | 0.022 | 0.513 | 0.659 |
| PLAUR | 0.586 | 0.037 | 0.022 | 0.513 | 0.660 |
| NEK2 | 0.586 | 0.037 | 0.022 | 0.513 | 0.659 |
| GTF3C5 | 0.586 | 0.037 | 0.022 | 0.513 | 0.660 |
| RBBP8 | 0.586 | 0.037 | 0.022 | 0.514 | 0.659 |
| FAM20A | 0.586 | 0.037 | 0.022 | 0.513 | 0.659 |
| FAF1 | 0.586 | 0.038 | 0.022 | 0.512 | 0.660 |
| GVINP1 | 0.586 | 0.037 | 0.022 | 0.513 | 0.659 |
| HSH2D | 0.586 | 0.037 | 0.022 | 0.513 | 0.659 |
| SEMA4A | 0.586 | 0.037 | 0.022 | 0.513 | 0.659 |
| CCL2 | 0.586 | 0.037 | 0.022 | 0.514 | 0.659 |
| TNNI2 | 0.586 | 0.037 | 0.022 | 0.513 | 0.659 |
| TOP3A | 0.586 | 0.038 | 0.022 | 0.513 | 0.660 |
| ID2 | 0.586 | 0.037 | 0.022 | 0.513 | 0.659 |
| IRF2 | 0.586 | 0.037 | 0.022 | 0.513 | 0.659 |
| IL17RB | 0.586 | 0.037 | 0.022 | 0.513 | 0.659 |
| HIST1H2BJ | 0.586 | 0.037 | 0.023 | 0.513 | 0.659 |
| MX1 | 0.586 | 0.038 | 0.023 | 0.512 | 0.660 |
| MZB1 | 0.586 | 0.037 | 0.023 | 0.513 | 0.659 |
| DIAPH1 | 0.586 | 0.037 | 0.023 | 0.513 | 0.659 |
| JAK2 | 0.586 | 0.037 | 0.023 | 0.513 | 0.659 |
| CHI3L2 | 0.586 | 0.038 | 0.023 | 0.512 | 0.660 |
| STRA6 | 0.586 | 0.037 | 0.023 | 0.513 | 0.659 |
| OGG1 | 0.586 | 0.037 | 0.023 | 0.512 | 0.659 |
| CHML | 0.586 | 0.038 | 0.023 | 0.512 | 0.660 |
| TCOF1 | 0.586 | 0.037 | 0.023 | 0.513 | 0.659 |
| IRF4 | 0.586 | 0.037 | 0.023 | 0.513 | 0.659 |
| OASL | 0.586 | 0.037 | 0.023 | 0.512 | 0.659 |
| MIR155HG | 0.586 | 0.037 | 0.023 | 0.513 | 0.659 |
| CD80 | 0.586 | 0.037 | 0.023 | 0.513 | 0.658 |
| GRAMD1B | 0.586 | 0.037 | 0.023 | 0.513 | 0.658 |
| C1QB | 0.586 | 0.037 | 0.023 | 0.513 | 0.658 |
| ARHGAP9 | 0.586 | 0.037 | 0.023 | 0.512 | 0.659 |
| RAD51D | 0.586 | 0.037 | 0.023 | 0.513 | 0.658 |
| PELP1 | 0.586 | 0.037 | 0.023 | 0.513 | 0.658 |
| MIS18A | 0.585 | 0.037 | 0.023 | 0.513 | 0.658 |
| TMEM131L | 0.585 | 0.037 | 0.023 | 0.512 | 0.658 |
| HIST1H2BH | 0.585 | 0.037 | 0.023 | 0.512 | 0.658 |
| SPI1 | 0.585 | 0.037 | 0.023 | 0.512 | 0.658 |
| KIF14 | 0.585 | 0.037 | 0.024 | 0.513 | 0.658 |
| CDC6 | 0.585 | 0.037 | 0.024 | 0.513 | 0.658 |
| COL4A2 | 0.585 | 0.037 | 0.024 | 0.512 | 0.658 |
| HIST1H2BF | 0.585 | 0.037 | 0.024 | 0.512 | 0.658 |
| DNAJB11 | 0.585 | 0.037 | 0.024 | 0.513 | 0.658 |
| CBLN3 | 0.585 | 0.037 | 0.024 | 0.513 | 0.658 |
| CCRL2 | 0.585 | 0.037 | 0.024 | 0.512 | 0.658 |
| PDCD1LG2 | 0.585 | 0.037 | 0.024 | 0.513 | 0.658 |
| ATAD2 | 0.585 | 0.037 | 0.024 | 0.512 | 0.659 |
| LRRC8B | 0.585 | 0.037 | 0.024 | 0.513 | 0.657 |
| FGD3 | 0.585 | 0.038 | 0.024 | 0.512 | 0.659 |
| DCUN1D2 | 0.585 | 0.037 | 0.024 | 0.512 | 0.658 |
| ANKRD44 | 0.585 | 0.037 | 0.024 | 0.512 | 0.658 |
| SEC24C | 0.585 | 0.037 | 0.024 | 0.512 | 0.658 |
| CYTIP | 0.585 | 0.037 | 0.024 | 0.512 | 0.658 |
| CD48 | 0.585 | 0.037 | 0.024 | 0.512 | 0.658 |
| EIF4G2 | 0.585 | 0.037 | 0.024 | 0.512 | 0.658 |
| LINC00996 | 0.585 | 0.037 | 0.024 | 0.512 | 0.658 |
| POGZ | 0.585 | 0.037 | 0.024 | 0.512 | 0.658 |
| IRF9 | 0.585 | 0.037 | 0.024 | 0.512 | 0.658 |
| TLR6 | 0.585 | 0.037 | 0.024 | 0.512 | 0.658 |
| SLC28A3 | 0.585 | 0.037 | 0.024 | 0.512 | 0.658 |
| MARCKS | 0.585 | 0.037 | 0.024 | 0.512 | 0.657 |
| GPR174 | 0.585 | 0.037 | 0.024 | 0.512 | 0.658 |
| BTN3A2 | 0.585 | 0.037 | 0.024 | 0.512 | 0.658 |
| TNFAIP8 | 0.585 | 0.037 | 0.024 | 0.512 | 0.658 |
| IGLL5 | 0.585 | 0.037 | 0.024 | 0.512 | 0.658 |
| PSMC3IP | 0.585 | 0.037 | 0.024 | 0.512 | 0.658 |
| ASPHD2 | 0.585 | 0.037 | 0.024 | 0.512 | 0.657 |
| NCAPH2 | 0.585 | 0.037 | 0.025 | 0.512 | 0.657 |
| PLBD1 | 0.585 | 0.037 | 0.025 | 0.512 | 0.657 |
| TLDC2 | 0.585 | 0.037 | 0.025 | 0.512 | 0.658 |
| AMER1 | 0.585 | 0.037 | 0.025 | 0.512 | 0.657 |
| PARL | 0.585 | 0.037 | 0.025 | 0.512 | 0.657 |
| PUS1 | 0.585 | 0.037 | 0.025 | 0.511 | 0.658 |
| RACGAP1 | 0.585 | 0.037 | 0.025 | 0.511 | 0.658 |
| PLA2G2D | 0.585 | 0.037 | 0.025 | 0.511 | 0.658 |
| ALPK1 | 0.584 | 0.038 | 0.025 | 0.510 | 0.659 |
| TDP1 | 0.584 | 0.037 | 0.025 | 0.512 | 0.657 |
| SLC16A3 | 0.584 | 0.037 | 0.025 | 0.512 | 0.657 |
| SUZ12 | 0.584 | 0.037 | 0.025 | 0.511 | 0.658 |
| HIST1H4D | 0.584 | 0.037 | 0.025 | 0.511 | 0.658 |
| LINC00426 | 0.584 | 0.037 | 0.025 | 0.512 | 0.657 |
| SIRT7 | 0.584 | 0.037 | 0.025 | 0.511 | 0.658 |
| MELTF | 0.584 | 0.038 | 0.025 | 0.511 | 0.658 |
| PRKDC | 0.584 | 0.037 | 0.025 | 0.512 | 0.657 |
| SLC25A28 | 0.584 | 0.037 | 0.025 | 0.512 | 0.657 |
| AMPD3 | 0.584 | 0.037 | 0.025 | 0.511 | 0.658 |
| PPIL1 | 0.584 | 0.037 | 0.025 | 0.512 | 0.657 |
| GNA13 | 0.584 | 0.037 | 0.025 | 0.511 | 0.657 |
| HIST1H3D | 0.584 | 0.037 | 0.025 | 0.511 | 0.657 |
| HIST1H1A | 0.584 | 0.037 | 0.025 | 0.511 | 0.657 |
| VAV2 | 0.584 | 0.037 | 0.026 | 0.512 | 0.657 |
| UBAP2L | 0.584 | 0.038 | 0.026 | 0.510 | 0.658 |
| EPB41L2 | 0.584 | 0.037 | 0.026 | 0.511 | 0.657 |
| PTPRO | 0.584 | 0.037 | 0.026 | 0.511 | 0.657 |
| RRM2 | 0.584 | 0.037 | 0.026 | 0.511 | 0.657 |
| SPN | 0.584 | 0.037 | 0.026 | 0.511 | 0.657 |
| FZD2 | 0.584 | 0.037 | 0.026 | 0.512 | 0.656 |
| GMIP | 0.584 | 0.037 | 0.026 | 0.510 | 0.657 |
| XRN2 | 0.584 | 0.037 | 0.026 | 0.512 | 0.656 |
| CASP3 | 0.584 | 0.037 | 0.026 | 0.511 | 0.656 |
| TGM2 | 0.584 | 0.037 | 0.026 | 0.511 | 0.657 |
| PNKD | 0.584 | 0.037 | 0.026 | 0.512 | 0.656 |
| SLC39A1 | 0.584 | 0.037 | 0.026 | 0.511 | 0.657 |
| MIR1827 | 0.584 | 0.037 | 0.026 | 0.511 | 0.657 |
| GLIPR2 | 0.584 | 0.037 | 0.026 | 0.510 | 0.657 |
| HIST2H2AC | 0.584 | 0.037 | 0.026 | 0.511 | 0.657 |
| UBB | 0.584 | 0.037 | 0.026 | 0.511 | 0.657 |
| ITPRIPL1 | 0.584 | 0.037 | 0.026 | 0.511 | 0.656 |
| CCNE1 | 0.584 | 0.038 | 0.026 | 0.510 | 0.658 |
| SAP30BP | 0.584 | 0.038 | 0.026 | 0.510 | 0.657 |
| PRUNE1 | 0.584 | 0.037 | 0.026 | 0.510 | 0.657 |
| HIST1H4B | 0.584 | 0.037 | 0.027 | 0.511 | 0.656 |
| GIMAP2 | 0.583 | 0.037 | 0.027 | 0.511 | 0.656 |
| ATAD5 | 0.583 | 0.038 | 0.027 | 0.509 | 0.658 |
| RCC2 | 0.583 | 0.037 | 0.027 | 0.510 | 0.657 |
| LRRC20 | 0.583 | 0.037 | 0.027 | 0.511 | 0.656 |
| PLEKHG4B | 0.583 | 0.038 | 0.027 | 0.510 | 0.657 |
| MIR302A | 0.583 | 0.037 | 0.027 | 0.511 | 0.656 |
| CHD1 | 0.583 | 0.038 | 0.027 | 0.510 | 0.657 |
| SPIB | 0.583 | 0.037 | 0.027 | 0.511 | 0.656 |
| SEC14L1 | 0.583 | 0.037 | 0.027 | 0.511 | 0.656 |
| NCKAP1L | 0.583 | 0.037 | 0.027 | 0.511 | 0.656 |
| CAPN15 | 0.583 | 0.037 | 0.027 | 0.510 | 0.656 |
| SP3 | 0.583 | 0.037 | 0.027 | 0.510 | 0.657 |
| ATAD3A | 0.583 | 0.037 | 0.027 | 0.510 | 0.656 |
| DDIT4 | 0.583 | 0.037 | 0.027 | 0.511 | 0.656 |
| APOL6 | 0.583 | 0.037 | 0.027 | 0.510 | 0.656 |
| NANP | 0.583 | 0.037 | 0.027 | 0.510 | 0.656 |
| CCNB2 | 0.583 | 0.037 | 0.027 | 0.510 | 0.656 |
| PLCH1 | 0.583 | 0.037 | 0.027 | 0.510 | 0.656 |
| CENPI | 0.583 | 0.037 | 0.027 | 0.511 | 0.656 |
| RAPGEF1 | 0.583 | 0.037 | 0.027 | 0.511 | 0.656 |
| HIST1H2BL | 0.583 | 0.037 | 0.027 | 0.510 | 0.656 |
| CH25H | 0.583 | 0.037 | 0.027 | 0.511 | 0.656 |
| LYSMD1 | 0.583 | 0.037 | 0.028 | 0.510 | 0.656 |
| FCGR1A | 0.583 | 0.037 | 0.028 | 0.511 | 0.655 |
| AXIN1 | 0.583 | 0.037 | 0.028 | 0.510 | 0.655 |
| HIRA | 0.583 | 0.037 | 0.028 | 0.510 | 0.656 |
| HAUS8 | 0.583 | 0.037 | 0.028 | 0.510 | 0.656 |
| COCH | 0.583 | 0.037 | 0.028 | 0.510 | 0.656 |
| NLRC3 | 0.583 | 0.037 | 0.028 | 0.510 | 0.656 |
| ARPC5L | 0.583 | 0.037 | 0.028 | 0.510 | 0.656 |
| TMEM150B | 0.583 | 0.037 | 0.028 | 0.510 | 0.656 |
| PRR5L | 0.583 | 0.038 | 0.028 | 0.509 | 0.656 |
| TRAF3 | 0.583 | 0.037 | 0.028 | 0.510 | 0.655 |
| PML | 0.583 | 0.037 | 0.028 | 0.509 | 0.656 |
| METTL16 | 0.583 | 0.037 | 0.028 | 0.510 | 0.655 |
| SLC16A1.AS1 | 0.583 | 0.037 | 0.028 | 0.510 | 0.656 |
| IFNG.AS1 | 0.583 | 0.037 | 0.028 | 0.510 | 0.655 |
| HLA.DQB1 | 0.583 | 0.037 | 0.028 | 0.510 | 0.655 |
| HPSE | 0.583 | 0.037 | 0.028 | 0.509 | 0.656 |
| FOXI1 | 0.583 | 0.037 | 0.028 | 0.510 | 0.655 |
| GZF1 | 0.583 | 0.037 | 0.028 | 0.510 | 0.656 |
| FKBP5 | 0.583 | 0.038 | 0.028 | 0.509 | 0.656 |
| SRGAP2 | 0.583 | 0.038 | 0.028 | 0.509 | 0.656 |
| TNS3 | 0.583 | 0.038 | 0.028 | 0.509 | 0.657 |
| BCS1L | 0.583 | 0.037 | 0.028 | 0.510 | 0.656 |
| PTGS1 | 0.583 | 0.038 | 0.028 | 0.509 | 0.656 |
| TUBB6 | 0.583 | 0.037 | 0.028 | 0.509 | 0.656 |
| TONSL | 0.583 | 0.037 | 0.028 | 0.510 | 0.655 |
| UBE2S | 0.583 | 0.037 | 0.028 | 0.510 | 0.656 |
| CHST11 | 0.582 | 0.037 | 0.029 | 0.510 | 0.655 |
| PSMD1 | 0.582 | 0.037 | 0.029 | 0.510 | 0.655 |
| BEND3 | 0.582 | 0.038 | 0.029 | 0.509 | 0.656 |
| FAM83D | 0.582 | 0.038 | 0.029 | 0.509 | 0.656 |
| SIMC1 | 0.582 | 0.037 | 0.029 | 0.510 | 0.655 |
| ROR2 | 0.582 | 0.037 | 0.029 | 0.509 | 0.655 |
| NR4A3 | 0.582 | 0.037 | 0.029 | 0.509 | 0.655 |
| CNP | 0.582 | 0.038 | 0.029 | 0.508 | 0.656 |
| UCHL1 | 0.582 | 0.037 | 0.029 | 0.509 | 0.655 |
| MCOLN2 | 0.582 | 0.037 | 0.029 | 0.509 | 0.655 |
| CLDN23 | 0.582 | 0.037 | 0.029 | 0.510 | 0.655 |
| RAB35 | 0.582 | 0.037 | 0.029 | 0.509 | 0.655 |
| DEPDC5 | 0.582 | 0.038 | 0.029 | 0.508 | 0.656 |
| PGD | 0.582 | 0.038 | 0.029 | 0.509 | 0.656 |
| HAX1 | 0.582 | 0.037 | 0.029 | 0.509 | 0.655 |
| IFT122 | 0.582 | 0.037 | 0.029 | 0.509 | 0.655 |
| ESS2 | 0.582 | 0.037 | 0.029 | 0.509 | 0.655 |
| NET1 | 0.582 | 0.037 | 0.029 | 0.509 | 0.655 |
| CDC123 | 0.582 | 0.037 | 0.029 | 0.509 | 0.655 |
| MIR1276 | 0.582 | 0.037 | 0.029 | 0.509 | 0.655 |
| ACP5 | 0.582 | 0.038 | 0.030 | 0.507 | 0.657 |
| PIP5K1A | 0.582 | 0.038 | 0.030 | 0.508 | 0.656 |
| WNT6 | 0.582 | 0.037 | 0.030 | 0.509 | 0.655 |
| STRADA | 0.582 | 0.037 | 0.030 | 0.509 | 0.655 |
| CDC42EP4 | 0.582 | 0.037 | 0.030 | 0.509 | 0.654 |
| UBE2Q1 | 0.582 | 0.037 | 0.030 | 0.509 | 0.655 |
| STMN1 | 0.582 | 0.037 | 0.030 | 0.509 | 0.655 |
| PTBP1 | 0.582 | 0.037 | 0.030 | 0.509 | 0.655 |
| FOXM1 | 0.582 | 0.037 | 0.030 | 0.509 | 0.655 |
| ARHGEF39 | 0.582 | 0.038 | 0.030 | 0.508 | 0.656 |
| YY1AP1 | 0.582 | 0.037 | 0.030 | 0.509 | 0.654 |
| C18orf8 | 0.582 | 0.037 | 0.030 | 0.509 | 0.654 |
| PTPN2 | 0.582 | 0.037 | 0.030 | 0.509 | 0.654 |
| HEPH | 0.582 | 0.037 | 0.030 | 0.509 | 0.654 |
| ORM1 | 0.582 | 0.038 | 0.030 | 0.508 | 0.656 |
| C5orf56 | 0.582 | 0.037 | 0.030 | 0.509 | 0.655 |
| CALML4 | 0.582 | 0.037 | 0.030 | 0.509 | 0.654 |
| CLEC10A | 0.582 | 0.037 | 0.030 | 0.509 | 0.655 |
| PDE4DIP | 0.582 | 0.037 | 0.030 | 0.509 | 0.654 |
| BTAF1 | 0.582 | 0.037 | 0.030 | 0.508 | 0.655 |
| ITGAX | 0.582 | 0.037 | 0.030 | 0.508 | 0.655 |
| HIST1H2AD | 0.582 | 0.037 | 0.030 | 0.509 | 0.654 |
| PAPSS1 | 0.582 | 0.038 | 0.030 | 0.508 | 0.655 |
| LY6D | 0.582 | 0.037 | 0.030 | 0.508 | 0.655 |
| HMGN4 | 0.581 | 0.038 | 0.031 | 0.508 | 0.655 |
| PPM1G | 0.581 | 0.037 | 0.031 | 0.508 | 0.655 |
| INPP4A | 0.581 | 0.037 | 0.031 | 0.508 | 0.655 |
| NUP153 | 0.581 | 0.037 | 0.031 | 0.509 | 0.654 |
| FLOT2 | 0.581 | 0.037 | 0.031 | 0.509 | 0.654 |
| TRMT11 | 0.581 | 0.037 | 0.031 | 0.508 | 0.655 |
| TVP23C | 0.581 | 0.037 | 0.031 | 0.508 | 0.654 |
| GZMK | 0.581 | 0.037 | 0.031 | 0.508 | 0.655 |
| CTR9 | 0.581 | 0.037 | 0.031 | 0.508 | 0.655 |
| DCK | 0.581 | 0.037 | 0.031 | 0.508 | 0.654 |
| CKAP2L | 0.581 | 0.038 | 0.031 | 0.508 | 0.655 |
| DUSP12 | 0.581 | 0.037 | 0.031 | 0.508 | 0.654 |
| CENPA | 0.581 | 0.037 | 0.031 | 0.508 | 0.654 |
| SARDH | 0.581 | 0.037 | 0.031 | 0.509 | 0.654 |
| LINC02004 | 0.581 | 0.037 | 0.031 | 0.508 | 0.654 |
| IGSF9 | 0.581 | 0.037 | 0.031 | 0.509 | 0.653 |
| ERAP1 | 0.581 | 0.037 | 0.031 | 0.508 | 0.654 |
| PIK3CG | 0.581 | 0.037 | 0.031 | 0.508 | 0.654 |
| MIR4479 | 0.581 | 0.037 | 0.031 | 0.508 | 0.654 |
| KLHDC9 | 0.581 | 0.037 | 0.031 | 0.508 | 0.654 |
| C16orf54 | 0.581 | 0.037 | 0.031 | 0.508 | 0.654 |
| TMEM200A | 0.581 | 0.037 | 0.031 | 0.509 | 0.654 |
| FOXP3 | 0.581 | 0.037 | 0.031 | 0.509 | 0.654 |
| CAND2 | 0.581 | 0.038 | 0.032 | 0.507 | 0.655 |
| NXN | 0.581 | 0.037 | 0.032 | 0.509 | 0.654 |
| HIST1H2AG | 0.581 | 0.037 | 0.032 | 0.508 | 0.654 |
| GPSM2 | 0.581 | 0.037 | 0.032 | 0.508 | 0.654 |
| KDELC1 | 0.581 | 0.037 | 0.032 | 0.508 | 0.654 |
| PCYT2 | 0.581 | 0.037 | 0.032 | 0.508 | 0.654 |
| HIST1H2BK | 0.581 | 0.037 | 0.032 | 0.508 | 0.654 |
| HSPA5 | 0.581 | 0.037 | 0.032 | 0.508 | 0.654 |
| FOXK2 | 0.581 | 0.037 | 0.032 | 0.508 | 0.654 |
| LACTB | 0.581 | 0.037 | 0.032 | 0.508 | 0.654 |
| H1FX | 0.581 | 0.037 | 0.032 | 0.508 | 0.654 |
| CFLAR | 0.581 | 0.038 | 0.032 | 0.507 | 0.654 |
| RAB8A | 0.581 | 0.037 | 0.032 | 0.508 | 0.654 |
| GYG1 | 0.581 | 0.037 | 0.032 | 0.508 | 0.653 |
| X9.Sep | 0.581 | 0.037 | 0.032 | 0.507 | 0.654 |
| EID3 | 0.581 | 0.037 | 0.032 | 0.508 | 0.654 |
| NEIL3 | 0.581 | 0.037 | 0.032 | 0.508 | 0.653 |
| CHDH | 0.581 | 0.037 | 0.032 | 0.508 | 0.654 |
| RPA2 | 0.581 | 0.037 | 0.032 | 0.508 | 0.654 |
| MCM7 | 0.581 | 0.037 | 0.032 | 0.508 | 0.653 |
| CXorf21 | 0.581 | 0.037 | 0.032 | 0.508 | 0.654 |
| HIST1H4A | 0.581 | 0.037 | 0.032 | 0.508 | 0.654 |
| GMEB1 | 0.581 | 0.037 | 0.032 | 0.508 | 0.653 |
| ING2 | 0.581 | 0.037 | 0.032 | 0.508 | 0.653 |
| PSME2 | 0.581 | 0.037 | 0.032 | 0.508 | 0.653 |
| SMARCA5 | 0.581 | 0.037 | 0.033 | 0.508 | 0.653 |
| MIR645 | 0.581 | 0.037 | 0.033 | 0.508 | 0.653 |
| AIM2 | 0.581 | 0.038 | 0.033 | 0.507 | 0.654 |
| ATF5 | 0.581 | 0.037 | 0.033 | 0.508 | 0.653 |
| C1orf112 | 0.580 | 0.037 | 0.033 | 0.508 | 0.653 |
| C11orf21 | 0.580 | 0.037 | 0.033 | 0.507 | 0.653 |
| RARRES1 | 0.580 | 0.037 | 0.033 | 0.508 | 0.653 |
| PTPRJ | 0.580 | 0.037 | 0.033 | 0.508 | 0.653 |
| AFG3L2 | 0.580 | 0.037 | 0.033 | 0.508 | 0.653 |
| UBA2 | 0.580 | 0.037 | 0.033 | 0.508 | 0.653 |
| OBP2B | 0.580 | 0.038 | 0.033 | 0.506 | 0.655 |
| MRPL28 | 0.580 | 0.037 | 0.033 | 0.507 | 0.653 |
| TTK | 0.580 | 0.037 | 0.033 | 0.507 | 0.654 |
| CRK | 0.580 | 0.037 | 0.033 | 0.508 | 0.653 |
| WDR62 | 0.580 | 0.037 | 0.033 | 0.508 | 0.653 |
| SELL | 0.580 | 0.038 | 0.033 | 0.507 | 0.654 |
| KBTBD4 | 0.580 | 0.037 | 0.033 | 0.507 | 0.653 |
| LRRC8C.DT | 0.580 | 0.037 | 0.033 | 0.507 | 0.653 |
| PAPLN | 0.580 | 0.037 | 0.033 | 0.507 | 0.654 |
| LTF | 0.580 | 0.037 | 0.033 | 0.507 | 0.653 |
| MAP3K3 | 0.580 | 0.037 | 0.033 | 0.507 | 0.653 |
| GUF1 | 0.580 | 0.037 | 0.034 | 0.507 | 0.653 |
| ADAMDEC1 | 0.580 | 0.037 | 0.034 | 0.507 | 0.653 |
| IFI35 | 0.580 | 0.038 | 0.034 | 0.506 | 0.654 |
| PALB2 | 0.580 | 0.037 | 0.034 | 0.507 | 0.653 |
| MIR4754 | 0.580 | 0.037 | 0.034 | 0.507 | 0.653 |
| FCGR3A | 0.580 | 0.037 | 0.034 | 0.507 | 0.653 |
| NAA15 | 0.580 | 0.037 | 0.034 | 0.507 | 0.653 |
| IFI44 | 0.580 | 0.037 | 0.034 | 0.506 | 0.653 |
| DEPDC1B | 0.580 | 0.037 | 0.034 | 0.507 | 0.653 |
| MBD4 | 0.580 | 0.037 | 0.034 | 0.507 | 0.652 |
| CD180 | 0.580 | 0.037 | 0.034 | 0.507 | 0.653 |
| CDKN2C | 0.580 | 0.037 | 0.034 | 0.507 | 0.653 |
| CCDC88B | 0.580 | 0.037 | 0.034 | 0.507 | 0.653 |
| EIF4E2 | 0.580 | 0.037 | 0.034 | 0.507 | 0.652 |
| SKA3 | 0.580 | 0.037 | 0.034 | 0.507 | 0.653 |
| SLC9A9.AS1 | 0.580 | 0.037 | 0.034 | 0.507 | 0.653 |
| LEMD2 | 0.580 | 0.037 | 0.034 | 0.507 | 0.652 |
| RAD51AP1 | 0.580 | 0.038 | 0.034 | 0.506 | 0.654 |
| CD79B | 0.580 | 0.038 | 0.034 | 0.506 | 0.653 |
| GPKOW | 0.580 | 0.038 | 0.034 | 0.505 | 0.654 |
| CTTNBP2NL | 0.580 | 0.037 | 0.034 | 0.507 | 0.653 |
| DNPH1 | 0.580 | 0.037 | 0.035 | 0.506 | 0.653 |
| STAT3 | 0.580 | 0.038 | 0.035 | 0.506 | 0.653 |
| GAB3 | 0.580 | 0.037 | 0.035 | 0.507 | 0.653 |
| STAMBPL1 | 0.580 | 0.037 | 0.035 | 0.507 | 0.652 |
| SMAD1 | 0.580 | 0.037 | 0.035 | 0.507 | 0.652 |
| LY6E | 0.579 | 0.037 | 0.035 | 0.507 | 0.652 |
| SOS1 | 0.579 | 0.037 | 0.035 | 0.507 | 0.652 |
| KDM2B | 0.579 | 0.037 | 0.035 | 0.507 | 0.652 |
| IGFALS | 0.579 | 0.037 | 0.035 | 0.506 | 0.653 |
| KCNA3 | 0.579 | 0.037 | 0.035 | 0.507 | 0.652 |
| HLA.DQB1.AS1 | 0.579 | 0.037 | 0.035 | 0.507 | 0.652 |
| SF3B1 | 0.579 | 0.038 | 0.035 | 0.505 | 0.654 |
| ROCK1 | 0.579 | 0.037 | 0.035 | 0.507 | 0.652 |
| HDHD5 | 0.579 | 0.037 | 0.035 | 0.507 | 0.652 |
| AC087392.1 | 0.579 | 0.037 | 0.035 | 0.507 | 0.652 |
| RRP1 | 0.579 | 0.037 | 0.035 | 0.506 | 0.653 |
| SNW1 | 0.579 | 0.037 | 0.035 | 0.506 | 0.652 |
| TBC1D2B | 0.579 | 0.037 | 0.035 | 0.506 | 0.652 |
| ABHD8 | 0.579 | 0.037 | 0.035 | 0.506 | 0.652 |
| FPR3 | 0.579 | 0.037 | 0.035 | 0.506 | 0.652 |
| VGLL1 | 0.579 | 0.037 | 0.036 | 0.506 | 0.652 |
| ANKRD10 | 0.579 | 0.037 | 0.036 | 0.506 | 0.653 |
| PRCC | 0.579 | 0.037 | 0.036 | 0.506 | 0.652 |
| PDCD10 | 0.579 | 0.037 | 0.036 | 0.506 | 0.652 |
| ISY1 | 0.579 | 0.037 | 0.036 | 0.506 | 0.652 |
| ISG20L2 | 0.579 | 0.037 | 0.036 | 0.506 | 0.652 |
| PSMD4 | 0.579 | 0.037 | 0.036 | 0.506 | 0.652 |
| FRMD4A | 0.579 | 0.037 | 0.036 | 0.506 | 0.652 |
| TBC1D17 | 0.579 | 0.037 | 0.036 | 0.506 | 0.652 |
| ADPGK | 0.579 | 0.037 | 0.036 | 0.506 | 0.651 |
| CCL8 | 0.579 | 0.037 | 0.036 | 0.506 | 0.652 |
| CAMK1 | 0.579 | 0.037 | 0.036 | 0.506 | 0.652 |
| TNFSF4 | 0.579 | 0.037 | 0.036 | 0.506 | 0.652 |
| TRIM38 | 0.579 | 0.037 | 0.036 | 0.506 | 0.652 |
| FYB1 | 0.579 | 0.037 | 0.037 | 0.506 | 0.651 |
| TTC7A | 0.579 | 0.037 | 0.037 | 0.506 | 0.652 |
| EXOSC2 | 0.579 | 0.038 | 0.037 | 0.505 | 0.652 |
| TIMELESS | 0.579 | 0.037 | 0.037 | 0.506 | 0.651 |
| GK | 0.579 | 0.038 | 0.037 | 0.505 | 0.652 |
| MZT2A | 0.579 | 0.038 | 0.037 | 0.504 | 0.653 |
| CD84 | 0.579 | 0.037 | 0.037 | 0.506 | 0.651 |
| PTTG2 | 0.579 | 0.037 | 0.037 | 0.506 | 0.651 |
| LONRF1 | 0.579 | 0.037 | 0.037 | 0.506 | 0.651 |
| POLQ | 0.579 | 0.037 | 0.037 | 0.506 | 0.651 |
| CD53 | 0.578 | 0.037 | 0.037 | 0.505 | 0.652 |
| SART1 | 0.578 | 0.037 | 0.037 | 0.505 | 0.652 |
| PARP12 | 0.578 | 0.037 | 0.037 | 0.505 | 0.652 |
| BATF | 0.578 | 0.037 | 0.037 | 0.505 | 0.652 |
| TMSB10 | 0.578 | 0.037 | 0.038 | 0.505 | 0.651 |
| LY9 | 0.578 | 0.037 | 0.038 | 0.505 | 0.652 |
| FANCL | 0.578 | 0.037 | 0.038 | 0.505 | 0.652 |
| MMP9 | 0.578 | 0.037 | 0.038 | 0.505 | 0.651 |
| UBE2C | 0.578 | 0.038 | 0.038 | 0.505 | 0.652 |
| UBALD2 | 0.578 | 0.037 | 0.038 | 0.505 | 0.651 |
| HDGFL2 | 0.578 | 0.038 | 0.038 | 0.504 | 0.653 |
| NCAPH | 0.578 | 0.037 | 0.038 | 0.505 | 0.651 |
| ACAP1 | 0.578 | 0.037 | 0.038 | 0.505 | 0.651 |
| CASP6 | 0.578 | 0.037 | 0.038 | 0.505 | 0.651 |
| C15orf48 | 0.578 | 0.038 | 0.038 | 0.505 | 0.652 |
| SEMA7A | 0.578 | 0.038 | 0.038 | 0.505 | 0.652 |
| CHAMP1 | 0.578 | 0.037 | 0.038 | 0.505 | 0.651 |
| HIST1H3I | 0.578 | 0.037 | 0.038 | 0.505 | 0.651 |
| TMEM107 | 0.578 | 0.038 | 0.038 | 0.503 | 0.653 |
| PLEKHA4 | 0.578 | 0.037 | 0.038 | 0.505 | 0.651 |
| TNFSF15 | 0.578 | 0.037 | 0.038 | 0.505 | 0.651 |
| RHOH | 0.578 | 0.037 | 0.038 | 0.505 | 0.651 |
| B3GNT5 | 0.578 | 0.037 | 0.038 | 0.505 | 0.651 |
| RAB40B | 0.578 | 0.037 | 0.038 | 0.505 | 0.652 |
| MIER1 | 0.578 | 0.037 | 0.038 | 0.506 | 0.650 |
| ZBTB24 | 0.578 | 0.037 | 0.038 | 0.505 | 0.651 |
| TBC1D10B | 0.578 | 0.038 | 0.038 | 0.504 | 0.652 |
| MRM3 | 0.578 | 0.037 | 0.038 | 0.506 | 0.651 |
| NDUFAF5 | 0.578 | 0.037 | 0.039 | 0.505 | 0.651 |
| SRSF3 | 0.578 | 0.037 | 0.039 | 0.505 | 0.651 |
| TSEN54 | 0.578 | 0.037 | 0.039 | 0.505 | 0.651 |
| CLSTN3 | 0.578 | 0.037 | 0.039 | 0.505 | 0.651 |
| ELP6 | 0.578 | 0.037 | 0.039 | 0.505 | 0.651 |
| RIPOR2 | 0.578 | 0.038 | 0.039 | 0.504 | 0.652 |
| TAPBPL | 0.578 | 0.037 | 0.039 | 0.505 | 0.651 |
| TMEM156 | 0.578 | 0.037 | 0.039 | 0.504 | 0.651 |
| REXO5 | 0.578 | 0.037 | 0.039 | 0.504 | 0.651 |
| TRIM16L | 0.578 | 0.037 | 0.039 | 0.505 | 0.651 |
| CCDC18 | 0.578 | 0.037 | 0.039 | 0.505 | 0.651 |
| SLFN11 | 0.578 | 0.037 | 0.039 | 0.505 | 0.650 |
| PLAC8 | 0.578 | 0.037 | 0.039 | 0.505 | 0.651 |
| TMEM201 | 0.578 | 0.038 | 0.039 | 0.504 | 0.652 |
| MED15 | 0.578 | 0.037 | 0.039 | 0.505 | 0.651 |
| AMBRA1 | 0.578 | 0.037 | 0.039 | 0.505 | 0.651 |
| KLHL25 | 0.578 | 0.038 | 0.039 | 0.504 | 0.651 |
| LARP4B | 0.578 | 0.037 | 0.039 | 0.505 | 0.651 |
| ABI3 | 0.578 | 0.037 | 0.039 | 0.505 | 0.651 |
| CMIP | 0.578 | 0.037 | 0.039 | 0.504 | 0.651 |
| TPP2 | 0.578 | 0.037 | 0.039 | 0.505 | 0.651 |
| HAVCR2 | 0.578 | 0.037 | 0.039 | 0.505 | 0.650 |
| ZYG11A | 0.578 | 0.037 | 0.039 | 0.505 | 0.651 |
| BTN2A1 | 0.578 | 0.038 | 0.039 | 0.504 | 0.651 |
| SPTY2D1 | 0.578 | 0.038 | 0.039 | 0.504 | 0.652 |
| CCR1 | 0.578 | 0.038 | 0.039 | 0.504 | 0.651 |
| MRC1 | 0.578 | 0.037 | 0.039 | 0.505 | 0.651 |
| SDC3 | 0.578 | 0.038 | 0.039 | 0.504 | 0.651 |
| TAF5 | 0.578 | 0.037 | 0.040 | 0.505 | 0.650 |
| ZMIZ2 | 0.578 | 0.037 | 0.040 | 0.505 | 0.650 |
| ADM2 | 0.577 | 0.037 | 0.040 | 0.505 | 0.650 |
| PARP9 | 0.577 | 0.038 | 0.040 | 0.503 | 0.652 |
| PRKCB | 0.577 | 0.038 | 0.040 | 0.504 | 0.651 |
| AOAH | 0.577 | 0.037 | 0.040 | 0.505 | 0.650 |
| SNORD20 | 0.577 | 0.037 | 0.040 | 0.504 | 0.650 |
| ILF3 | 0.577 | 0.037 | 0.040 | 0.504 | 0.651 |
| CMC2 | 0.577 | 0.038 | 0.040 | 0.502 | 0.652 |
| ZNF775 | 0.577 | 0.037 | 0.040 | 0.505 | 0.650 |
| TRMU | 0.577 | 0.037 | 0.040 | 0.504 | 0.650 |
| HIST1H2AE | 0.577 | 0.037 | 0.040 | 0.504 | 0.650 |
| SRPK1 | 0.577 | 0.037 | 0.040 | 0.504 | 0.651 |
| PTK2B | 0.577 | 0.037 | 0.040 | 0.504 | 0.650 |
| DIS3L | 0.577 | 0.037 | 0.040 | 0.505 | 0.650 |
| MDM1 | 0.577 | 0.038 | 0.040 | 0.503 | 0.651 |
| GART | 0.577 | 0.037 | 0.040 | 0.504 | 0.650 |
| ZC3H4 | 0.577 | 0.037 | 0.040 | 0.504 | 0.651 |
| ARHGAP45 | 0.577 | 0.038 | 0.040 | 0.504 | 0.651 |
| ASPM | 0.577 | 0.037 | 0.040 | 0.504 | 0.651 |
| CCAR1 | 0.577 | 0.037 | 0.040 | 0.504 | 0.651 |
| MAK16 | 0.577 | 0.037 | 0.041 | 0.504 | 0.650 |
| NUDT5 | 0.577 | 0.037 | 0.041 | 0.504 | 0.651 |
| SBK1 | 0.577 | 0.037 | 0.041 | 0.504 | 0.650 |
| DLST | 0.577 | 0.037 | 0.041 | 0.504 | 0.650 |
| AL121944.1 | 0.577 | 0.037 | 0.041 | 0.504 | 0.650 |
| C11orf84 | 0.577 | 0.037 | 0.041 | 0.504 | 0.650 |
| ELOVL6 | 0.577 | 0.038 | 0.041 | 0.503 | 0.651 |
| RBBP7 | 0.577 | 0.037 | 0.041 | 0.504 | 0.650 |
| CEP72 | 0.577 | 0.038 | 0.041 | 0.502 | 0.652 |
| WASHC2A | 0.577 | 0.037 | 0.041 | 0.504 | 0.650 |
| URM1 | 0.577 | 0.037 | 0.041 | 0.504 | 0.650 |
| NUP107 | 0.577 | 0.037 | 0.041 | 0.503 | 0.650 |
| HIVEP1 | 0.577 | 0.037 | 0.041 | 0.504 | 0.650 |
| FGD2 | 0.577 | 0.037 | 0.041 | 0.503 | 0.650 |
| CDK5RAP2 | 0.577 | 0.037 | 0.041 | 0.504 | 0.650 |
| RAB20 | 0.577 | 0.037 | 0.041 | 0.504 | 0.650 |
| TLR8 | 0.577 | 0.037 | 0.041 | 0.504 | 0.649 |
| BPTF | 0.577 | 0.037 | 0.042 | 0.504 | 0.650 |
| DOCK10 | 0.577 | 0.037 | 0.042 | 0.504 | 0.650 |
| FLVCR2 | 0.577 | 0.037 | 0.042 | 0.504 | 0.650 |
| SELE | 0.577 | 0.038 | 0.042 | 0.503 | 0.651 |
| MRPS21 | 0.577 | 0.037 | 0.042 | 0.504 | 0.650 |
| WIPF1 | 0.577 | 0.037 | 0.042 | 0.504 | 0.650 |
| H3F3B | 0.577 | 0.037 | 0.042 | 0.503 | 0.650 |
| UPF2 | 0.577 | 0.037 | 0.042 | 0.503 | 0.650 |
| PFDN2 | 0.577 | 0.037 | 0.042 | 0.504 | 0.650 |
| DPYD.AS1 | 0.577 | 0.037 | 0.042 | 0.503 | 0.650 |
| CARS2 | 0.577 | 0.038 | 0.042 | 0.503 | 0.650 |
| TRIM21 | 0.577 | 0.037 | 0.042 | 0.503 | 0.650 |
| CSF2RA | 0.577 | 0.037 | 0.042 | 0.504 | 0.649 |
| E2F2 | 0.576 | 0.038 | 0.042 | 0.503 | 0.650 |
| FCRL3 | 0.576 | 0.038 | 0.042 | 0.503 | 0.650 |
| GNG3 | 0.576 | 0.037 | 0.042 | 0.504 | 0.649 |
| MUTYH | 0.576 | 0.038 | 0.042 | 0.503 | 0.650 |
| MIR5094 | 0.576 | 0.037 | 0.043 | 0.503 | 0.650 |
| SMG6 | 0.576 | 0.037 | 0.043 | 0.503 | 0.650 |
| CXCL16 | 0.576 | 0.038 | 0.043 | 0.503 | 0.650 |
| CXCL5 | 0.576 | 0.037 | 0.043 | 0.503 | 0.649 |
| SRSF7 | 0.576 | 0.037 | 0.043 | 0.503 | 0.649 |
| DDX58 | 0.576 | 0.038 | 0.043 | 0.502 | 0.651 |
| JAKMIP1 | 0.576 | 0.037 | 0.043 | 0.504 | 0.649 |
| GAS2L3 | 0.576 | 0.038 | 0.043 | 0.502 | 0.651 |
| UNK | 0.576 | 0.038 | 0.043 | 0.502 | 0.650 |
| MADD | 0.576 | 0.037 | 0.043 | 0.504 | 0.649 |
| MIIP | 0.576 | 0.037 | 0.043 | 0.504 | 0.649 |
| ZC3H7B | 0.576 | 0.038 | 0.043 | 0.502 | 0.650 |
| IL7R | 0.576 | 0.037 | 0.043 | 0.503 | 0.649 |
| EWSR1 | 0.576 | 0.037 | 0.043 | 0.503 | 0.649 |
| ARAP1 | 0.576 | 0.037 | 0.043 | 0.503 | 0.649 |
| IL4R | 0.576 | 0.037 | 0.043 | 0.503 | 0.649 |
| RPTOR | 0.576 | 0.038 | 0.043 | 0.502 | 0.650 |
| RSRC1 | 0.576 | 0.037 | 0.043 | 0.503 | 0.649 |
| TCEANC2 | 0.576 | 0.038 | 0.044 | 0.502 | 0.650 |
| TAGLN2 | 0.576 | 0.037 | 0.044 | 0.503 | 0.649 |
| HIST1H2AJ | 0.576 | 0.037 | 0.044 | 0.503 | 0.649 |
| DTYMK | 0.576 | 0.037 | 0.044 | 0.503 | 0.649 |
| CPT1B | 0.576 | 0.037 | 0.044 | 0.503 | 0.649 |
| GIMAP1 | 0.576 | 0.037 | 0.044 | 0.503 | 0.649 |
| LGALS3BP | 0.576 | 0.037 | 0.044 | 0.503 | 0.649 |
| CD52 | 0.576 | 0.037 | 0.044 | 0.502 | 0.649 |
| ARNT2 | 0.576 | 0.038 | 0.044 | 0.502 | 0.650 |
| JMJD6 | 0.576 | 0.037 | 0.044 | 0.503 | 0.649 |
| FIG4 | 0.576 | 0.037 | 0.044 | 0.503 | 0.649 |
| CBX1 | 0.576 | 0.037 | 0.044 | 0.503 | 0.649 |
| WDR37 | 0.576 | 0.037 | 0.044 | 0.503 | 0.648 |
| MARK1 | 0.576 | 0.037 | 0.044 | 0.503 | 0.649 |
| PATZ1 | 0.576 | 0.037 | 0.044 | 0.502 | 0.649 |
| INTS7 | 0.576 | 0.037 | 0.044 | 0.503 | 0.649 |
| ACP4 | 0.576 | 0.038 | 0.045 | 0.502 | 0.649 |
| CCDC150 | 0.576 | 0.038 | 0.045 | 0.502 | 0.649 |
| P2RX1 | 0.576 | 0.037 | 0.045 | 0.502 | 0.649 |
| RNF168 | 0.576 | 0.037 | 0.045 | 0.503 | 0.649 |
| HACD1 | 0.576 | 0.037 | 0.045 | 0.502 | 0.649 |
| SERPINB9 | 0.576 | 0.037 | 0.045 | 0.503 | 0.649 |
| RPS19BP1 | 0.576 | 0.037 | 0.045 | 0.502 | 0.649 |
| FXR2 | 0.576 | 0.037 | 0.045 | 0.503 | 0.648 |
| MCUR1 | 0.576 | 0.037 | 0.045 | 0.503 | 0.648 |
| DZIP3 | 0.576 | 0.037 | 0.045 | 0.502 | 0.649 |
| SNORD13E | 0.576 | 0.037 | 0.045 | 0.503 | 0.649 |
| LDHB | 0.576 | 0.038 | 0.045 | 0.502 | 0.649 |
| BTN3A3 | 0.576 | 0.037 | 0.045 | 0.502 | 0.649 |
| SS18L2 | 0.575 | 0.038 | 0.045 | 0.502 | 0.649 |
| EFNB1 | 0.575 | 0.038 | 0.045 | 0.501 | 0.650 |
| ASH1L.AS1 | 0.575 | 0.038 | 0.045 | 0.502 | 0.649 |
| UHRF1BP1 | 0.575 | 0.037 | 0.045 | 0.503 | 0.648 |
| AQP9 | 0.575 | 0.038 | 0.045 | 0.502 | 0.649 |
| CDC42SE2 | 0.575 | 0.037 | 0.045 | 0.503 | 0.648 |
| UBE2T | 0.575 | 0.037 | 0.045 | 0.502 | 0.648 |
| DDX11 | 0.575 | 0.037 | 0.046 | 0.502 | 0.649 |
| CENPU | 0.575 | 0.037 | 0.046 | 0.503 | 0.648 |
| FAM19A3 | 0.575 | 0.037 | 0.046 | 0.502 | 0.649 |
| XAF1 | 0.575 | 0.038 | 0.046 | 0.502 | 0.649 |
| FANCI | 0.575 | 0.037 | 0.046 | 0.502 | 0.648 |
| TPGS2 | 0.575 | 0.037 | 0.046 | 0.502 | 0.648 |
| TEDC2 | 0.575 | 0.037 | 0.046 | 0.502 | 0.648 |
| TMPO.AS1 | 0.575 | 0.037 | 0.046 | 0.502 | 0.649 |
| ADAM19 | 0.575 | 0.037 | 0.046 | 0.502 | 0.649 |
| RNF166 | 0.575 | 0.037 | 0.046 | 0.502 | 0.648 |
| ARL14EP | 0.575 | 0.037 | 0.046 | 0.502 | 0.649 |
| DDIAS | 0.575 | 0.037 | 0.046 | 0.502 | 0.648 |
| SGO1 | 0.575 | 0.037 | 0.046 | 0.502 | 0.648 |
| GFM1 | 0.575 | 0.037 | 0.046 | 0.502 | 0.648 |
| RGS1 | 0.575 | 0.038 | 0.046 | 0.501 | 0.649 |
| APBB1IP | 0.575 | 0.037 | 0.046 | 0.502 | 0.648 |
| CDC14B | 0.575 | 0.037 | 0.046 | 0.502 | 0.648 |
| SF3A3 | 0.575 | 0.037 | 0.046 | 0.502 | 0.649 |
| AP4B1 | 0.575 | 0.037 | 0.046 | 0.502 | 0.648 |
| RGS16 | 0.575 | 0.038 | 0.047 | 0.501 | 0.649 |
| EXOC6 | 0.575 | 0.037 | 0.047 | 0.502 | 0.648 |
| NCK1 | 0.575 | 0.037 | 0.047 | 0.502 | 0.648 |
| RPN1 | 0.575 | 0.037 | 0.047 | 0.502 | 0.648 |
| PSMA1 | 0.575 | 0.037 | 0.047 | 0.501 | 0.648 |
| TATDN2 | 0.575 | 0.037 | 0.047 | 0.502 | 0.648 |
| API5 | 0.575 | 0.037 | 0.047 | 0.502 | 0.648 |
| C17orf53 | 0.575 | 0.037 | 0.047 | 0.501 | 0.648 |
| TMPRSS3 | 0.575 | 0.038 | 0.047 | 0.501 | 0.649 |
| PMAIP1 | 0.575 | 0.037 | 0.047 | 0.501 | 0.648 |
| CHRNB2 | 0.575 | 0.037 | 0.047 | 0.502 | 0.648 |
| AGPAT5 | 0.575 | 0.037 | 0.047 | 0.502 | 0.648 |
| TYROBP | 0.575 | 0.037 | 0.047 | 0.502 | 0.648 |
| UGT8 | 0.575 | 0.038 | 0.047 | 0.501 | 0.648 |
| PRAG1 | 0.575 | 0.037 | 0.047 | 0.502 | 0.647 |
| ZCCHC11 | 0.575 | 0.038 | 0.047 | 0.501 | 0.649 |
| CD5 | 0.575 | 0.037 | 0.047 | 0.502 | 0.648 |
| LARS2 | 0.575 | 0.037 | 0.047 | 0.501 | 0.648 |
| DAPK2 | 0.575 | 0.037 | 0.047 | 0.501 | 0.648 |
| CFLAR.AS1 | 0.575 | 0.038 | 0.048 | 0.501 | 0.649 |
| SPATA5 | 0.575 | 0.038 | 0.048 | 0.501 | 0.648 |
| WASHC2C | 0.575 | 0.038 | 0.048 | 0.501 | 0.648 |
| KARS | 0.575 | 0.037 | 0.048 | 0.502 | 0.647 |
| CARHSP1 | 0.575 | 0.037 | 0.048 | 0.502 | 0.647 |
| CEP128 | 0.575 | 0.037 | 0.048 | 0.501 | 0.648 |
| GNAI2 | 0.575 | 0.038 | 0.048 | 0.501 | 0.648 |
| IFI44L | 0.575 | 0.038 | 0.048 | 0.501 | 0.648 |
| ABHD18 | 0.575 | 0.037 | 0.048 | 0.501 | 0.648 |
| RFC5 | 0.575 | 0.038 | 0.048 | 0.501 | 0.648 |
| SAMHD1 | 0.575 | 0.037 | 0.048 | 0.501 | 0.648 |
| UBE2Z | 0.575 | 0.038 | 0.048 | 0.501 | 0.648 |
| OAS3 | 0.574 | 0.037 | 0.048 | 0.501 | 0.648 |
| MED12 | 0.574 | 0.037 | 0.048 | 0.502 | 0.647 |
| USP31 | 0.574 | 0.037 | 0.048 | 0.501 | 0.648 |
| TRIM45 | 0.574 | 0.037 | 0.048 | 0.501 | 0.647 |
| ARHGAP4 | 0.574 | 0.037 | 0.048 | 0.501 | 0.647 |
| TBC1D10C | 0.574 | 0.037 | 0.048 | 0.501 | 0.648 |
| AL590705.5 | 0.574 | 0.038 | 0.048 | 0.501 | 0.648 |
| SLC8A1 | 0.574 | 0.037 | 0.048 | 0.501 | 0.647 |
| AL109955.1 | 0.574 | 0.037 | 0.049 | 0.502 | 0.647 |
| SETD1A | 0.574 | 0.038 | 0.049 | 0.500 | 0.648 |
| PLGRKT | 0.574 | 0.037 | 0.049 | 0.501 | 0.648 |
| ITGB3BP | 0.574 | 0.037 | 0.049 | 0.501 | 0.648 |
| FCRL1 | 0.574 | 0.038 | 0.049 | 0.500 | 0.648 |
| H2AFZ | 0.574 | 0.037 | 0.049 | 0.501 | 0.647 |
| ORAI2 | 0.574 | 0.037 | 0.049 | 0.501 | 0.647 |
| TMEM104 | 0.574 | 0.037 | 0.049 | 0.501 | 0.647 |
| ALOX5AP | 0.574 | 0.037 | 0.049 | 0.501 | 0.647 |
| CBX2 | 0.574 | 0.037 | 0.049 | 0.501 | 0.647 |
| MRPS16 | 0.574 | 0.037 | 0.049 | 0.501 | 0.647 |
| SIGLEC14 | 0.574 | 0.037 | 0.049 | 0.501 | 0.647 |
| DPYD | 0.574 | 0.037 | 0.049 | 0.501 | 0.647 |
| HIST1H2BI | 0.574 | 0.038 | 0.049 | 0.500 | 0.648 |
| CLP1 | 0.574 | 0.037 | 0.049 | 0.501 | 0.647 |
| HOMER3 | 0.574 | 0.037 | 0.049 | 0.501 | 0.647 |
| SNRPA | 0.574 | 0.037 | 0.049 | 0.501 | 0.647 |
| FUT4 | 0.574 | 0.037 | 0.049 | 0.501 | 0.647 |
| DDX20 | 0.574 | 0.037 | 0.049 | 0.501 | 0.647 |

TABLE 2
Genes predictive of RD from the GSE164458_A cohort.
Area under the curve

| | | | | Asymptotic 95% | |
|---|---|---|---|---|---|
| | | | | Confidence | |
| Test | | | | Interval | |
| Result | | Std. | Asymptotic | Lower | Upper |
| Variable(s) | Area | Error^{a} | Sig.^{b} | Bound | Bound |
| CAB39L | 0.667 | 0.036 | 0.000 | 0.597 | 0.737 |
| FBXL16 | 0.665 | 0.036 | 0.000 | 0.595 | 0.735 |
| GPRC5C | 0.652 | 0.036 | 0.000 | 0.582 | 0.722 |
| RHOB | 0.649 | 0.036 | 0.000 | 0.579 | 0.718 |
| FAM234B | 0.642 | 0.036 | 0.000 | 0.571 | 0.713 |
| TMEM99 | 0.638 | 0.036 | 0.000 | 0.569 | 0.708 |
| DEPTOR | 0.638 | 0.036 | 0.000 | 0.566 | 0.709 |
| PTK6 | 0.636 | 0.036 | 0.000 | 0.565 | 0.707 |
| TMEM150A | 0.629 | 0.037 | 0.001 | 0.558 | 0.701 |
| TMEM106B | 0.629 | 0.036 | 0.001 | 0.557 | 0.700 |
| MIR4728 | 0.628 | 0.036 | 0.001 | 0.557 | 0.699 |
| RNF152 | 0.627 | 0.036 | 0.001 | 0.556 | 0.698 |
| CTSF | 0.627 | 0.037 | 0.001 | 0.555 | 0.698 |
| SUGT1 | 0.624 | 0.036 | 0.001 | 0.553 | 0.695 |
| PPFIA3 | 0.623 | 0.036 | 0.001 | 0.552 | 0.694 |
| SCGB2A1 | 0.623 | 0.037 | 0.001 | 0.551 | 0.694 |
| SC5D | 0.623 | 0.036 | 0.001 | 0.552 | 0.694 |
| FSIP2 | 0.622 | 0.036 | 0.001 | 0.551 | 0.693 |
| CCDC126 | 0.621 | 0.036 | 0.001 | 0.550 | 0.692 |
| EBPL | 0.621 | 0.037 | 0.001 | 0.549 | 0.692 |
| RNF170 | 0.619 | 0.037 | 0.002 | 0.547 | 0.692 |
| MPV17 | 0.618 | 0.037 | 0.002 | 0.546 | 0.690 |
| SERINC2 | 0.618 | 0.037 | 0.002 | 0.547 | 0.690 |
| ISPD | 0.616 | 0.037 | 0.002 | 0.544 | 0.689 |
| SUCLA2 | 0.616 | 0.037 | 0.002 | 0.544 | 0.688 |
| JHDM1D.AS1 | 0.615 | 0.036 | 0.002 | 0.544 | 0.687 |
| SNX13 | 0.615 | 0.037 | 0.002 | 0.542 | 0.687 |
| VPS36 | 0.614 | 0.036 | 0.002 | 0.543 | 0.685 |
| LMX1B | 0.614 | 0.036 | 0.003 | 0.543 | 0.685 |
| CRIP2 | 0.614 | 0.037 | 0.003 | 0.542 | 0.685 |
| SERINC3 | 0.613 | 0.037 | 0.003 | 0.541 | 0.684 |
| DALRD3 | 0.612 | 0.036 | 0.003 | 0.541 | 0.684 |
| PDZD2 | 0.612 | 0.036 | 0.003 | 0.541 | 0.683 |
| PRKAA1 | 0.612 | 0.037 | 0.003 | 0.539 | 0.684 |
| EPB41L5 | 0.611 | 0.037 | 0.003 | 0.540 | 0.683 |
| VWA8 | 0.611 | 0.037 | 0.003 | 0.538 | 0.683 |
| MIR591 | 0.610 | 0.037 | 0.003 | 0.538 | 0.682 |
| OSER1.AS1 | 0.610 | 0.037 | 0.003 | 0.538 | 0.682 |
| MT.CO3 | 0.610 | 0.037 | 0.003 | 0.538 | 0.682 |
| PEX11A | 0.609 | 0.036 | 0.004 | 0.538 | 0.681 |
| RNF103 | 0.609 | 0.037 | 0.004 | 0.537 | 0.681 |
| TMED3 | 0.609 | 0.037 | 0.004 | 0.536 | 0.682 |
| KBTBD7 | 0.608 | 0.037 | 0.004 | 0.536 | 0.681 |
| MEST | 0.608 | 0.037 | 0.004 | 0.536 | 0.680 |
| REXO2 | 0.608 | 0.037 | 0.004 | 0.535 | 0.680 |
| ZSCAN31 | 0.607 | 0.037 | 0.005 | 0.535 | 0.678 |
| LSMEM1 | 0.606 | 0.037 | 0.005 | 0.534 | 0.678 |
| ZNF273 | 0.606 | 0.037 | 0.005 | 0.534 | 0.678 |
| FBXL8 | 0.606 | 0.037 | 0.005 | 0.533 | 0.678 |
| PCDH1 | 0.606 | 0.037 | 0.005 | 0.533 | 0.678 |
| NEIL1 | 0.605 | 0.037 | 0.005 | 0.533 | 0.678 |
| ZNF280D | 0.605 | 0.037 | 0.005 | 0.533 | 0.677 |
| ATP13A4 | 0.605 | 0.037 | 0.005 | 0.533 | 0.677 |
| KIAA1324 | 0.605 | 0.037 | 0.006 | 0.532 | 0.677 |
| INHBB | 0.604 | 0.037 | 0.006 | 0.532 | 0.676 |
| MIR5692A1 | 0.602 | 0.037 | 0.007 | 0.530 | 0.675 |
| SIAE | 0.602 | 0.037 | 0.007 | 0.530 | 0.675 |
| KLF9 | 0.602 | 0.037 | 0.007 | 0.530 | 0.674 |
| MAN1B1.AS1 | 0.602 | 0.037 | 0.007 | 0.529 | 0.675 |
| TSC22D1 | 0.602 | 0.037 | 0.007 | 0.529 | 0.675 |
| YPEL5 | 0.602 | 0.037 | 0.007 | 0.530 | 0.674 |
| HIBADH | 0.602 | 0.037 | 0.007 | 0.529 | 0.674 |
| PHLDA1 | 0.601 | 0.037 | 0.007 | 0.529 | 0.674 |
| UFM1 | 0.601 | 0.037 | 0.007 | 0.529 | 0.673 |
| AC011472.1 | 0.601 | 0.037 | 0.007 | 0.529 | 0.673 |
| CD46 | 0.601 | 0.037 | 0.008 | 0.529 | 0.673 |
| C15orf65 | 0.600 | 0.037 | 0.008 | 0.528 | 0.673 |
| GAMT | 0.600 | 0.037 | 0.008 | 0.528 | 0.673 |
| COG3 | 0.600 | 0.037 | 0.008 | 0.527 | 0.673 |
| MIR579 | 0.600 | 0.037 | 0.008 | 0.528 | 0.672 |
| SLC5A4 | 0.600 | 0.037 | 0.008 | 0.528 | 0.672 |
| RNF11 | 0.600 | 0.037 | 0.008 | 0.527 | 0.672 |
| C1QTNF3 | 0.600 | 0.037 | 0.008 | 0.527 | 0.672 |
| SLC35F5 | 0.600 | 0.037 | 0.008 | 0.528 | 0.672 |
| MIR590 | 0.599 | 0.037 | 0.009 | 0.527 | 0.671 |
| HTR7P1 | 0.599 | 0.037 | 0.009 | 0.527 | 0.671 |
| PDGFD | 0.599 | 0.037 | 0.009 | 0.526 | 0.671 |
| GHR | 0.599 | 0.037 | 0.009 | 0.526 | 0.671 |
| VIPR1 | 0.599 | 0.037 | 0.009 | 0.526 | 0.671 |
| PLPPR2 | 0.598 | 0.037 | 0.009 | 0.526 | 0.671 |
| GPALPP1 | 0.598 | 0.037 | 0.009 | 0.526 | 0.670 |
| MMP15 | 0.598 | 0.037 | 0.009 | 0.525 | 0.671 |
| CBR3.AS1 | 0.598 | 0.037 | 0.009 | 0.526 | 0.670 |
| PPP1R3C | 0.598 | 0.037 | 0.009 | 0.526 | 0.670 |
| FOLR1 | 0.597 | 0.038 | 0.010 | 0.524 | 0.671 |
| FITM2 | 0.597 | 0.037 | 0.010 | 0.524 | 0.671 |
| STX1A | 0.597 | 0.037 | 0.010 | 0.525 | 0.670 |
| SPRED2 | 0.597 | 0.037 | 0.010 | 0.524 | 0.670 |
| KIAA1024 | 0.597 | 0.037 | 0.010 | 0.524 | 0.671 |
| TGFB2 | 0.597 | 0.038 | 0.010 | 0.523 | 0.671 |
| HOXC10 | 0.597 | 0.037 | 0.010 | 0.525 | 0.669 |
| HOMER2 | 0.597 | 0.037 | 0.010 | 0.525 | 0.669 |
| CAV2 | 0.597 | 0.037 | 0.010 | 0.524 | 0.669 |
| GABARAPL1 | 0.596 | 0.037 | 0.010 | 0.524 | 0.669 |
| TOM1L2 | 0.596 | 0.037 | 0.011 | 0.524 | 0.669 |
| SPRYD7 | 0.596 | 0.037 | 0.011 | 0.524 | 0.668 |
| RB1 | 0.596 | 0.037 | 0.011 | 0.523 | 0.669 |
| EEF1A2 | 0.596 | 0.037 | 0.011 | 0.523 | 0.669 |
| SCARNA6 | 0.596 | 0.037 | 0.011 | 0.523 | 0.668 |
| RCOR3 | 0.596 | 0.037 | 0.011 | 0.523 | 0.669 |
| THSD1 | 0.596 | 0.037 | 0.011 | 0.524 | 0.668 |
| CPLX1 | 0.595 | 0.037 | 0.011 | 0.524 | 0.667 |
| COPS2 | 0.595 | 0.037 | 0.011 | 0.522 | 0.669 |
| HSF4 | 0.595 | 0.037 | 0.011 | 0.523 | 0.668 |
| MLPH | 0.595 | 0.037 | 0.011 | 0.522 | 0.668 |
| SSFA2 | 0.595 | 0.037 | 0.011 | 0.523 | 0.668 |
| SOD3 | 0.595 | 0.038 | 0.012 | 0.521 | 0.670 |
| FMO4 | 0.595 | 0.037 | 0.012 | 0.523 | 0.668 |
| NIPAL2 | 0.595 | 0.037 | 0.012 | 0.522 | 0.668 |
| STXBP1 | 0.595 | 0.037 | 0.012 | 0.523 | 0.667 |
| BCAP29 | 0.595 | 0.037 | 0.012 | 0.522 | 0.668 |
| BLCAP | 0.595 | 0.037 | 0.012 | 0.522 | 0.668 |
| OSGIN1 | 0.595 | 0.037 | 0.012 | 0.523 | 0.667 |
| C1orf116 | 0.595 | 0.037 | 0.012 | 0.522 | 0.667 |
| MATN3 | 0.595 | 0.037 | 0.012 | 0.522 | 0.667 |
| MFAP3L | 0.595 | 0.037 | 0.012 | 0.522 | 0.667 |
| C14orf28 | 0.594 | 0.037 | 0.012 | 0.522 | 0.667 |
| ETFRF1 | 0.594 | 0.037 | 0.012 | 0.521 | 0.667 |
| SLC48A1 | 0.594 | 0.037 | 0.013 | 0.521 | 0.667 |
| LMBRD2 | 0.594 | 0.037 | 0.013 | 0.521 | 0.666 |
| LAMB2 | 0.594 | 0.037 | 0.013 | 0.521 | 0.666 |
| DHRS12 | 0.594 | 0.037 | 0.013 | 0.521 | 0.666 |
| RCBTB1 | 0.594 | 0.037 | 0.013 | 0.521 | 0.666 |
| PCDHB13 | 0.593 | 0.037 | 0.013 | 0.520 | 0.666 |
| LRIG3 | 0.593 | 0.037 | 0.014 | 0.521 | 0.665 |
| HSPB7 | 0.593 | 0.037 | 0.014 | 0.520 | 0.666 |
| SPOPL | 0.593 | 0.037 | 0.014 | 0.520 | 0.665 |
| SECISBP2L | 0.592 | 0.037 | 0.014 | 0.519 | 0.666 |
| TBC1D24 | 0.592 | 0.037 | 0.014 | 0.519 | 0.665 |
| LINC01376 | 0.592 | 0.037 | 0.014 | 0.520 | 0.665 |
| TBX3 | 0.592 | 0.037 | 0.014 | 0.520 | 0.664 |
| NEAT1 | 0.592 | 0.038 | 0.015 | 0.518 | 0.665 |
| RERG.AS1 | 0.592 | 0.037 | 0.015 | 0.519 | 0.664 |
| FAM174B | 0.592 | 0.037 | 0.015 | 0.519 | 0.665 |
| HOXC11 | 0.592 | 0.037 | 0.015 | 0.520 | 0.664 |
| TGFB2.AS1 | 0.591 | 0.037 | 0.015 | 0.518 | 0.665 |
| ANO1 | 0.591 | 0.037 | 0.015 | 0.519 | 0.664 |
| APOOL | 0.591 | 0.037 | 0.015 | 0.519 | 0.664 |
| TACC2 | 0.591 | 0.037 | 0.016 | 0.519 | 0.663 |
| ZG16B | 0.591 | 0.037 | 0.016 | 0.519 | 0.664 |
| METRN | 0.591 | 0.037 | 0.016 | 0.519 | 0.663 |
| BBS10 | 0.591 | 0.037 | 0.016 | 0.518 | 0.663 |
| SRPK3 | 0.590 | 0.037 | 0.016 | 0.518 | 0.663 |
| CLDN12 | 0.590 | 0.037 | 0.016 | 0.518 | 0.663 |
| ESRRG | 0.590 | 0.037 | 0.017 | 0.517 | 0.663 |
| CCDC152 | 0.590 | 0.037 | 0.017 | 0.518 | 0.663 |
| HOXC.AS1 | 0.590 | 0.037 | 0.017 | 0.518 | 0.662 |
| MT.ND3 | 0.590 | 0.037 | 0.017 | 0.517 | 0.663 |
| SCGB1D2 | 0.590 | 0.037 | 0.017 | 0.518 | 0.662 |
| MIRLET7A1 | 0.590 | 0.037 | 0.017 | 0.517 | 0.663 |
| TM7SF2 | 0.590 | 0.037 | 0.017 | 0.516 | 0.663 |
| TLE2 | 0.590 | 0.037 | 0.017 | 0.518 | 0.662 |
| VPS37D | 0.590 | 0.037 | 0.017 | 0.516 | 0.663 |
| SLC38A2 | 0.589 | 0.037 | 0.018 | 0.516 | 0.663 |
| HOTAIR | 0.589 | 0.037 | 0.018 | 0.517 | 0.661 |
| ALG11 | 0.589 | 0.037 | 0.018 | 0.516 | 0.662 |
| MED4 | 0.589 | 0.037 | 0.018 | 0.517 | 0.661 |
| MMAB | 0.589 | 0.037 | 0.018 | 0.516 | 0.662 |
| CD55 | 0.589 | 0.037 | 0.018 | 0.516 | 0.662 |
| PTGR2 | 0.589 | 0.037 | 0.018 | 0.516 | 0.661 |
| MIGA1 | 0.589 | 0.037 | 0.018 | 0.516 | 0.661 |
| MIR8066 | 0.589 | 0.037 | 0.018 | 0.516 | 0.661 |
| GULP1 | 0.589 | 0.037 | 0.019 | 0.516 | 0.661 |
| MIR125B1 | 0.588 | 0.037 | 0.019 | 0.516 | 0.661 |
| SPDYA | 0.588 | 0.037 | 0.019 | 0.515 | 0.661 |
| NDFIP2 | 0.588 | 0.037 | 0.019 | 0.515 | 0.661 |
| MIR4786 | 0.588 | 0.037 | 0.019 | 0.516 | 0.661 |
| PPP1R13B | 0.588 | 0.037 | 0.019 | 0.515 | 0.661 |
| KLHL13 | 0.588 | 0.037 | 0.019 | 0.516 | 0.661 |
| LINC01235 | 0.588 | 0.037 | 0.019 | 0.515 | 0.661 |
| THSD4 | 0.588 | 0.037 | 0.020 | 0.515 | 0.661 |
| MEGF6 | 0.588 | 0.037 | 0.020 | 0.515 | 0.661 |
| PDZD8 | 0.588 | 0.037 | 0.020 | 0.515 | 0.661 |
| ADCY6 | 0.588 | 0.037 | 0.020 | 0.515 | 0.660 |
| SOS2 | 0.588 | 0.037 | 0.020 | 0.515 | 0.660 |
| MIR6829 | 0.588 | 0.037 | 0.020 | 0.515 | 0.660 |
| PCYOX1 | 0.587 | 0.037 | 0.020 | 0.515 | 0.660 |
| TOMM7 | 0.587 | 0.037 | 0.020 | 0.515 | 0.660 |
| ZNF680 | 0.587 | 0.037 | 0.020 | 0.515 | 0.660 |
| AGT | 0.587 | 0.037 | 0.020 | 0.515 | 0.660 |
| PLLP | 0.587 | 0.037 | 0.020 | 0.515 | 0.660 |
| MIR30E | 0.587 | 0.037 | 0.020 | 0.515 | 0.660 |
| TMPRSS2 | 0.587 | 0.037 | 0.021 | 0.515 | 0.660 |
| HES1 | 0.587 | 0.037 | 0.021 | 0.515 | 0.660 |
| EFNA3 | 0.587 | 0.037 | 0.021 | 0.514 | 0.660 |
| SPDEF | 0.587 | 0.037 | 0.021 | 0.514 | 0.660 |
| MYEF2 | 0.587 | 0.038 | 0.021 | 0.513 | 0.661 |
| NQO1 | 0.587 | 0.037 | 0.021 | 0.514 | 0.660 |
| PIP | 0.587 | 0.037 | 0.021 | 0.514 | 0.660 |
| MAPRE3 | 0.587 | 0.037 | 0.022 | 0.513 | 0.660 |
| LIN7B | 0.587 | 0.038 | 0.022 | 0.513 | 0.660 |
| SNORD41 | 0.586 | 0.037 | 0.022 | 0.514 | 0.659 |
| IGFBP5 | 0.586 | 0.037 | 0.022 | 0.514 | 0.659 |
| ZKSCAN1 | 0.586 | 0.037 | 0.022 | 0.514 | 0.659 |
| CHST1 | 0.586 | 0.037 | 0.022 | 0.514 | 0.659 |
| CCL28 | 0.586 | 0.037 | 0.023 | 0.513 | 0.658 |
| REEP3 | 0.586 | 0.037 | 0.023 | 0.513 | 0.659 |
| SHC2 | 0.586 | 0.037 | 0.023 | 0.513 | 0.658 |
| TMEM182 | 0.586 | 0.037 | 0.023 | 0.513 | 0.658 |
| CMBL | 0.586 | 0.037 | 0.023 | 0.513 | 0.658 |
| PBOV1 | 0.586 | 0.037 | 0.023 | 0.513 | 0.659 |
| SLC2A10 | 0.586 | 0.037 | 0.023 | 0.513 | 0.659 |
| AMFR | 0.585 | 0.038 | 0.023 | 0.512 | 0.659 |
| TAS2R5 | 0.585 | 0.038 | 0.024 | 0.512 | 0.659 |
| FAM227B | 0.585 | 0.037 | 0.024 | 0.513 | 0.658 |
| RAB11FIP5 | 0.585 | 0.037 | 0.024 | 0.513 | 0.657 |
| LAPTM4A | 0.585 | 0.037 | 0.025 | 0.512 | 0.658 |
| WDFY3 | 0.584 | 0.037 | 0.025 | 0.512 | 0.657 |
| WDFY2 | 0.584 | 0.037 | 0.025 | 0.512 | 0.657 |
| REEP6 | 0.584 | 0.037 | 0.026 | 0.511 | 0.657 |
| CNTN1 | 0.584 | 0.037 | 0.026 | 0.511 | 0.657 |
| ZEB1.AS1 | 0.584 | 0.037 | 0.026 | 0.511 | 0.657 |
| SLC9A3R2 | 0.584 | 0.037 | 0.026 | 0.511 | 0.657 |
| RPL6 | 0.584 | 0.037 | 0.026 | 0.511 | 0.657 |
| SLC25A23 | 0.584 | 0.037 | 0.026 | 0.511 | 0.657 |
| CHD9 | 0.584 | 0.037 | 0.026 | 0.511 | 0.656 |
| MFSD5 | 0.584 | 0.037 | 0.026 | 0.510 | 0.657 |
| HOXC.AS2 | 0.584 | 0.037 | 0.026 | 0.511 | 0.656 |
| PDCD4 | 0.583 | 0.038 | 0.027 | 0.509 | 0.658 |
| SLPI | 0.583 | 0.037 | 0.027 | 0.510 | 0.657 |
| SCARNA13 | 0.583 | 0.037 | 0.027 | 0.510 | 0.656 |
| ICA1 | 0.583 | 0.038 | 0.027 | 0.509 | 0.657 |
| MAPK10 | 0.583 | 0.037 | 0.027 | 0.510 | 0.656 |
| INTS6 | 0.583 | 0.037 | 0.028 | 0.510 | 0.656 |
| SCGB2A2 | 0.583 | 0.037 | 0.028 | 0.510 | 0.656 |
| SMIM1 | 0.583 | 0.037 | 0.028 | 0.510 | 0.656 |
| FABP3 | 0.583 | 0.037 | 0.028 | 0.510 | 0.656 |
| SNORA12 | 0.583 | 0.037 | 0.028 | 0.510 | 0.656 |
| COG6 | 0.583 | 0.037 | 0.028 | 0.510 | 0.656 |
| PREPL | 0.583 | 0.037 | 0.028 | 0.509 | 0.656 |
| OTUD6B.AS1 | 0.583 | 0.037 | 0.028 | 0.510 | 0.655 |
| GNAI1 | 0.583 | 0.037 | 0.028 | 0.509 | 0.656 |
| RTN2 | 0.583 | 0.037 | 0.028 | 0.510 | 0.655 |
| ETFBKMT | 0.583 | 0.037 | 0.028 | 0.509 | 0.656 |
| LONP2 | 0.583 | 0.037 | 0.028 | 0.510 | 0.655 |
| SNX21 | 0.582 | 0.037 | 0.029 | 0.510 | 0.655 |
| PXMP4 | 0.582 | 0.038 | 0.029 | 0.509 | 0.656 |
| SHANK2.AS1 | 0.582 | 0.037 | 0.029 | 0.510 | 0.655 |
| LCA5 | 0.582 | 0.037 | 0.029 | 0.509 | 0.655 |
| PXDC1 | 0.582 | 0.037 | 0.029 | 0.509 | 0.655 |
| TTC28.AS1 | 0.582 | 0.037 | 0.029 | 0.509 | 0.655 |
| BCAM | 0.582 | 0.038 | 0.029 | 0.508 | 0.656 |
| CLDN8 | 0.582 | 0.037 | 0.030 | 0.509 | 0.655 |
| DYNC2LI1 | 0.582 | 0.037 | 0.030 | 0.509 | 0.654 |
| TMBIM6 | 0.582 | 0.038 | 0.030 | 0.508 | 0.655 |
| ASPG | 0.582 | 0.037 | 0.030 | 0.509 | 0.654 |
| ATRAID | 0.582 | 0.037 | 0.030 | 0.509 | 0.654 |
| TMEM135 | 0.582 | 0.037 | 0.030 | 0.509 | 0.654 |
| AL033397.2 | 0.582 | 0.037 | 0.030 | 0.508 | 0.655 |
| SBDS | 0.582 | 0.037 | 0.030 | 0.509 | 0.654 |
| BAMBI | 0.582 | 0.037 | 0.030 | 0.509 | 0.654 |
| SNORA11 | 0.581 | 0.038 | 0.031 | 0.508 | 0.655 |
| GATA2 | 0.581 | 0.037 | 0.031 | 0.508 | 0.655 |
| FSTL3 | 0.581 | 0.038 | 0.031 | 0.507 | 0.655 |
| ANG | 0.581 | 0.037 | 0.031 | 0.509 | 0.654 |
| FGF13 | 0.581 | 0.038 | 0.031 | 0.508 | 0.655 |
| ALS2CL | 0.581 | 0.037 | 0.031 | 0.508 | 0.654 |
| MPLKIP | 0.581 | 0.037 | 0.032 | 0.508 | 0.653 |
| SLC12A2 | 0.581 | 0.037 | 0.032 | 0.508 | 0.654 |
| NDST1 | 0.581 | 0.037 | 0.032 | 0.508 | 0.654 |
| FOXL1 | 0.581 | 0.037 | 0.032 | 0.508 | 0.654 |
| LZTFL1 | 0.581 | 0.037 | 0.032 | 0.508 | 0.653 |
| CA5B | 0.580 | 0.037 | 0.033 | 0.507 | 0.654 |
| PRORSD1P | 0.580 | 0.037 | 0.033 | 0.508 | 0.653 |
| TOGARAM1 | 0.580 | 0.037 | 0.033 | 0.507 | 0.654 |
| MALAT1 | 0.580 | 0.038 | 0.033 | 0.507 | 0.654 |
| NIPSNAP2 | 0.580 | 0.037 | 0.033 | 0.508 | 0.653 |
| PCDHGC5 | 0.580 | 0.037 | 0.033 | 0.507 | 0.653 |
| ADCY9 | 0.580 | 0.037 | 0.033 | 0.508 | 0.653 |
| TRPM4 | 0.580 | 0.037 | 0.033 | 0.507 | 0.653 |
| SYBU | 0.580 | 0.038 | 0.033 | 0.507 | 0.654 |
| NFIA | 0.580 | 0.037 | 0.033 | 0.507 | 0.653 |
| ZNF850 | 0.580 | 0.038 | 0.033 | 0.506 | 0.654 |
| MIR550A2 | 0.580 | 0.037 | 0.034 | 0.507 | 0.653 |
| ZNF562 | 0.580 | 0.037 | 0.034 | 0.507 | 0.653 |
| SCARNA18 | 0.580 | 0.037 | 0.034 | 0.507 | 0.653 |
| TPT1 | 0.580 | 0.037 | 0.034 | 0.507 | 0.653 |
| PRR15L | 0.580 | 0.037 | 0.034 | 0.506 | 0.653 |
| HOOK3 | 0.580 | 0.037 | 0.034 | 0.506 | 0.653 |
| TMEM25 | 0.580 | 0.038 | 0.035 | 0.506 | 0.653 |
| NPDC1 | 0.580 | 0.037 | 0.035 | 0.507 | 0.652 |
| UTP14C | 0.579 | 0.037 | 0.035 | 0.506 | 0.653 |
| CASC4 | 0.579 | 0.038 | 0.035 | 0.505 | 0.654 |
| GTPBP10 | 0.579 | 0.037 | 0.035 | 0.506 | 0.653 |
| X2.Mar | 0.579 | 0.038 | 0.035 | 0.506 | 0.653 |
| BSG | 0.579 | 0.037 | 0.036 | 0.506 | 0.652 |
| ARFGEF3 | 0.579 | 0.037 | 0.036 | 0.506 | 0.652 |
| ZNF138 | 0.579 | 0.037 | 0.036 | 0.506 | 0.652 |
| ADIPOR1 | 0.579 | 0.037 | 0.036 | 0.506 | 0.652 |
| TAX1BP1 | 0.579 | 0.037 | 0.036 | 0.506 | 0.652 |
| AC012085.2 | 0.579 | 0.037 | 0.036 | 0.505 | 0.652 |
| TPCN1 | 0.579 | 0.037 | 0.037 | 0.506 | 0.652 |
| CPEB2 | 0.579 | 0.037 | 0.037 | 0.505 | 0.652 |
| PPP1R3D | 0.579 | 0.037 | 0.037 | 0.506 | 0.652 |
| LRRC75B | 0.579 | 0.037 | 0.037 | 0.505 | 0.652 |
| SH3RF2 | 0.579 | 0.037 | 0.037 | 0.505 | 0.652 |
| PKP4.AS1 | 0.578 | 0.037 | 0.037 | 0.505 | 0.651 |
| ATG14 | 0.578 | 0.038 | 0.038 | 0.505 | 0.652 |
| SLC22A5 | 0.578 | 0.037 | 0.038 | 0.505 | 0.651 |
| CCDC149 | 0.578 | 0.037 | 0.038 | 0.506 | 0.651 |
| MT.CO1 | 0.578 | 0.038 | 0.038 | 0.505 | 0.652 |
| ZNF702P | 0.578 | 0.037 | 0.038 | 0.505 | 0.651 |
| PTPRS | 0.578 | 0.038 | 0.038 | 0.504 | 0.652 |
| ZNF569 | 0.578 | 0.037 | 0.038 | 0.506 | 0.651 |
| UBL3 | 0.578 | 0.037 | 0.039 | 0.505 | 0.651 |
| FAM46A | 0.578 | 0.037 | 0.039 | 0.505 | 0.651 |
| CAV1 | 0.578 | 0.037 | 0.039 | 0.505 | 0.651 |
| SYT7 | 0.578 | 0.037 | 0.039 | 0.505 | 0.651 |
| ACY1 | 0.578 | 0.037 | 0.039 | 0.505 | 0.650 |
| TSPAN13 | 0.578 | 0.038 | 0.039 | 0.504 | 0.651 |
| LINC01473 | 0.578 | 0.037 | 0.039 | 0.505 | 0.650 |
| PCMTD1 | 0.578 | 0.037 | 0.039 | 0.504 | 0.651 |
| AKR1C2 | 0.578 | 0.037 | 0.040 | 0.505 | 0.650 |
| TMEM267 | 0.578 | 0.037 | 0.040 | 0.505 | 0.650 |
| MIPOL1 | 0.578 | 0.037 | 0.040 | 0.504 | 0.651 |
| DNAJA4 | 0.578 | 0.037 | 0.040 | 0.505 | 0.650 |
| SNORA70G | 0.577 | 0.037 | 0.040 | 0.505 | 0.650 |
| TMEM87A | 0.577 | 0.037 | 0.040 | 0.505 | 0.650 |
| FAM222A | 0.577 | 0.038 | 0.040 | 0.503 | 0.651 |
| SYNGR1 | 0.577 | 0.037 | 0.040 | 0.504 | 0.651 |
| SPATA6 | 0.577 | 0.037 | 0.040 | 0.505 | 0.650 |
| FZD4 | 0.577 | 0.037 | 0.041 | 0.505 | 0.650 |
| ZBTB7C | 0.577 | 0.037 | 0.041 | 0.504 | 0.650 |
| TRIM13 | 0.577 | 0.037 | 0.041 | 0.504 | 0.650 |
| SYPL1 | 0.577 | 0.037 | 0.041 | 0.504 | 0.650 |
| KBTBD6 | 0.577 | 0.037 | 0.041 | 0.504 | 0.650 |
| GALK2 | 0.577 | 0.037 | 0.041 | 0.504 | 0.650 |
| GRB7 | 0.577 | 0.037 | 0.042 | 0.504 | 0.650 |
| TSC22D3 | 0.577 | 0.037 | 0.042 | 0.503 | 0.650 |
| GSTM3 | 0.577 | 0.038 | 0.042 | 0.502 | 0.651 |
| ZNF595 | 0.577 | 0.037 | 0.042 | 0.503 | 0.650 |
| ARRDC3 | 0.577 | 0.037 | 0.042 | 0.504 | 0.649 |
| SPATA7 | 0.577 | 0.037 | 0.042 | 0.504 | 0.650 |
| ZFYVE21 | 0.576 | 0.037 | 0.042 | 0.503 | 0.649 |
| EMP1 | 0.576 | 0.037 | 0.043 | 0.504 | 0.649 |
| HOXC9 | 0.576 | 0.037 | 0.043 | 0.504 | 0.649 |
| NCBP2.AS1 | 0.576 | 0.037 | 0.043 | 0.503 | 0.649 |
| FBXO27 | 0.576 | 0.037 | 0.043 | 0.503 | 0.649 |
| MED4.AS1 | 0.576 | 0.037 | 0.044 | 0.503 | 0.649 |
| TIMP3 | 0.576 | 0.037 | 0.044 | 0.503 | 0.649 |
| ZNF823 | 0.576 | 0.037 | 0.044 | 0.503 | 0.649 |
| AC103760.1 | 0.576 | 0.038 | 0.044 | 0.502 | 0.650 |
| ZNF704 | 0.576 | 0.037 | 0.044 | 0.503 | 0.649 |
| GRAMD1C | 0.576 | 0.037 | 0.044 | 0.503 | 0.648 |
| MIR604 | 0.576 | 0.037 | 0.044 | 0.503 | 0.648 |
| HSPB1 | 0.576 | 0.037 | 0.044 | 0.502 | 0.649 |
| PCDHGB8P | 0.576 | 0.037 | 0.044 | 0.503 | 0.649 |
| STEAP4 | 0.576 | 0.037 | 0.044 | 0.503 | 0.649 |
| PCDHGA10 | 0.576 | 0.037 | 0.044 | 0.503 | 0.649 |
| CRYL1 | 0.576 | 0.038 | 0.045 | 0.502 | 0.649 |
| DBI | 0.576 | 0.038 | 0.045 | 0.501 | 0.650 |
| UNC5CL | 0.576 | 0.037 | 0.045 | 0.502 | 0.649 |
| GGCX | 0.576 | 0.037 | 0.045 | 0.503 | 0.648 |
| AKR7A3 | 0.576 | 0.037 | 0.045 | 0.503 | 0.648 |
| PTCH2 | 0.575 | 0.037 | 0.045 | 0.502 | 0.649 |
| SIX2 | 0.575 | 0.037 | 0.045 | 0.502 | 0.649 |
| MIR149 | 0.575 | 0.037 | 0.045 | 0.502 | 0.649 |
| TRIM4 | 0.575 | 0.037 | 0.046 | 0.502 | 0.648 |
| DICER1.AS1 | 0.575 | 0.037 | 0.046 | 0.502 | 0.648 |
| AC005083.1 | 0.575 | 0.037 | 0.046 | 0.502 | 0.648 |
| MAP4K5 | 0.575 | 0.037 | 0.046 | 0.502 | 0.648 |
| C10orf113 | 0.575 | 0.037 | 0.046 | 0.502 | 0.648 |
| CPNE8 | 0.575 | 0.037 | 0.047 | 0.502 | 0.648 |
| PRRT3.AS1 | 0.575 | 0.037 | 0.047 | 0.502 | 0.648 |
| MAPKAPK5.AS1 | 0.575 | 0.038 | 0.047 | 0.500 | 0.650 |
| PAX8.AS1 | 0.575 | 0.038 | 0.047 | 0.501 | 0.649 |
| DPH6 | 0.575 | 0.038 | 0.047 | 0.501 | 0.649 |
| KLC4 | 0.575 | 0.038 | 0.048 | 0.501 | 0.648 |
| SH3BP4 | 0.575 | 0.037 | 0.048 | 0.501 | 0.648 |
| NCOA1 | 0.575 | 0.037 | 0.048 | 0.502 | 0.647 |
| NDRG3 | 0.575 | 0.037 | 0.048 | 0.502 | 0.648 |
| HEY1 | 0.575 | 0.037 | 0.048 | 0.501 | 0.648 |
| MRPS6 | 0.575 | 0.038 | 0.048 | 0.501 | 0.648 |
| ZNF597 | 0.575 | 0.037 | 0.048 | 0.502 | 0.647 |
| RMDN1 | 0.575 | 0.038 | 0.048 | 0.501 | 0.648 |
| CASC19 | 0.574 | 0.038 | 0.048 | 0.501 | 0.648 |
| EBAG9 | 0.574 | 0.038 | 0.048 | 0.501 | 0.648 |
| AC104986.2 | 0.574 | 0.038 | 0.049 | 0.501 | 0.648 |
| MIR128.1 | 0.574 | 0.037 | 0.049 | 0.502 | 0.647 |
| TRPT1 | 0.574 | 0.038 | 0.049 | 0.499 | 0.649 |
| NUDT9 | 0.574 | 0.038 | 0.049 | 0.500 | 0.648 |
| MNAT1 | 0.574 | 0.038 | 0.049 | 0.500 | 0.648 |
| CES4A | 0.574 | 0.037 | 0.049 | 0.501 | 0.648 |
| MEAF6 | 0.574 | 0.037 | 0.049 | 0.501 | 0.647 |

TABLE 3
Genes predictive of pCR from the GSE164458_B cohort.
Area under the curve

| | | | | Asymptotic 95% | |
|---|---|---|---|---|---|
| | | | | Confidence | |
| Test | | | | Interval | |
| Result | | Std. | Asymptotic | Lower | Upper |
| Variable(s) | Area | Error^{a} | Sig.^{b} | Bound | Bound |
| HS3ST3B1 | 0.714 | 0.048 | 0.000 | 0.620 | 0.807 |
| CENPW | 0.712 | 0.046 | 0.000 | 0.621 | 0.802 |
| CXCL13 | 0.701 | 0.049 | 0.000 | 0.605 | 0.797 |
| GZMB | 0.701 | 0.048 | 0.000 | 0.607 | 0.794 |
| XPO6 | 0.699 | 0.048 | 0.000 | 0.606 | 0.793 |
| ZNF543 | 0.698 | 0.048 | 0.000 | 0.604 | 0.791 |
| SOCS3 | 0.697 | 0.049 | 0.000 | 0.601 | 0.793 |
| CTLA4 | 0.695 | 0.049 | 0.000 | 0.598 | 0.792 |
| CCL13 | 0.690 | 0.050 | 0.000 | 0.592 | 0.787 |
| ME2 | 0.687 | 0.049 | 0.000 | 0.591 | 0.782 |
| PDCD1LG2 | 0.685 | 0.050 | 0.000 | 0.587 | 0.783 |
| BUB1B | 0.684 | 0.048 | 0.000 | 0.590 | 0.779 |
| SOCS1 | 0.683 | 0.048 | 0.001 | 0.590 | 0.777 |
| XPNPEP1 | 0.683 | 0.051 | 0.001 | 0.584 | 0.782 |
| PARL | 0.682 | 0.049 | 0.001 | 0.586 | 0.778 |
| C11orf84 | 0.682 | 0.049 | 0.001 | 0.586 | 0.777 |
| FBXO5 | 0.681 | 0.049 | 0.001 | 0.585 | 0.777 |
| PAK3 | 0.679 | 0.049 | 0.001 | 0.582 | 0.775 |
| TIGIT | 0.678 | 0.049 | 0.001 | 0.582 | 0.774 |
| C12orf4 | 0.678 | 0.049 | 0.001 | 0.582 | 0.774 |
| HASPIN | 0.678 | 0.049 | 0.001 | 0.582 | 0.773 |
| C19orf48 | 0.678 | 0.049 | 0.001 | 0.582 | 0.773 |
| ICOS | 0.677 | 0.049 | 0.001 | 0.581 | 0.773 |
| BRIP1 | 0.677 | 0.050 | 0.001 | 0.580 | 0.774 |
| ANKRD22 | 0.677 | 0.050 | 0.001 | 0.578 | 0.775 |
| WBP11 | 0.676 | 0.050 | 0.001 | 0.578 | 0.774 |
| CD274 | 0.675 | 0.050 | 0.001 | 0.577 | 0.774 |
| ARNTL2 | 0.675 | 0.049 | 0.001 | 0.579 | 0.772 |
| C17orf53 | 0.675 | 0.049 | 0.001 | 0.579 | 0.772 |
| IFNG | 0.675 | 0.050 | 0.001 | 0.578 | 0.773 |
| ATF7IP | 0.675 | 0.049 | 0.001 | 0.579 | 0.771 |
| VTI1A | 0.675 | 0.049 | 0.001 | 0.578 | 0.771 |
| KIAA1551 | 0.675 | 0.050 | 0.001 | 0.578 | 0.772 |
| GBP1 | 0.674 | 0.049 | 0.001 | 0.577 | 0.771 |
| GABPB1 | 0.673 | 0.050 | 0.001 | 0.575 | 0.771 |
| SCAF8 | 0.672 | 0.048 | 0.001 | 0.578 | 0.767 |
| ACAA2 | 0.671 | 0.049 | 0.001 | 0.575 | 0.767 |
| STAT1 | 0.671 | 0.049 | 0.001 | 0.575 | 0.767 |
| ZNF416 | 0.671 | 0.049 | 0.001 | 0.576 | 0.766 |
| SLC25A28 | 0.670 | 0.050 | 0.001 | 0.573 | 0.768 |
| UHRF1 | 0.670 | 0.050 | 0.001 | 0.573 | 0.767 |
| CCDC150 | 0.670 | 0.049 | 0.001 | 0.574 | 0.766 |
| LINC01806 | 0.669 | 0.050 | 0.001 | 0.571 | 0.767 |
| CALHM6 | 0.668 | 0.050 | 0.002 | 0.571 | 0.765 |
| TBP | 0.667 | 0.048 | 0.002 | 0.573 | 0.762 |
| GBP5 | 0.667 | 0.050 | 0.002 | 0.569 | 0.765 |
| PRMT1 | 0.666 | 0.049 | 0.002 | 0.570 | 0.762 |
| MBD2 | 0.666 | 0.050 | 0.002 | 0.569 | 0.763 |
| DYNLT1 | 0.666 | 0.049 | 0.002 | 0.570 | 0.761 |
| MSL3 | 0.664 | 0.049 | 0.002 | 0.568 | 0.761 |
| FEN1 | 0.664 | 0.051 | 0.002 | 0.564 | 0.764 |
| MYO1G | 0.663 | 0.050 | 0.002 | 0.565 | 0.761 |
| NAT10 | 0.663 | 0.049 | 0.002 | 0.567 | 0.759 |
| CXCL9 | 0.663 | 0.050 | 0.002 | 0.565 | 0.761 |
| ZBED2 | 0.663 | 0.050 | 0.002 | 0.565 | 0.761 |
| ZBED6CL | 0.663 | 0.049 | 0.002 | 0.567 | 0.758 |
| STRAP | 0.663 | 0.050 | 0.002 | 0.564 | 0.761 |
| TRAFD1 | 0.662 | 0.049 | 0.002 | 0.565 | 0.758 |
| DUSP2 | 0.662 | 0.050 | 0.002 | 0.565 | 0.759 |
| FOXK2 | 0.662 | 0.051 | 0.002 | 0.563 | 0.761 |
| CD3D | 0.662 | 0.050 | 0.002 | 0.565 | 0.759 |
| LGR5 | 0.662 | 0.049 | 0.002 | 0.565 | 0.758 |
| CCL8 | 0.661 | 0.050 | 0.002 | 0.563 | 0.760 |
| STAMBPL1 | 0.661 | 0.050 | 0.002 | 0.563 | 0.760 |
| ZNF618 | 0.660 | 0.050 | 0.002 | 0.563 | 0.758 |
| RAD51AP1 | 0.660 | 0.050 | 0.002 | 0.563 | 0.758 |
| XPO7 | 0.660 | 0.050 | 0.002 | 0.563 | 0.758 |
| IRF1 | 0.660 | 0.050 | 0.002 | 0.562 | 0.758 |
| PRF1 | 0.660 | 0.049 | 0.003 | 0.564 | 0.756 |
| ING1 | 0.660 | 0.050 | 0.003 | 0.562 | 0.758 |
| ADAM19 | 0.660 | 0.049 | 0.003 | 0.563 | 0.756 |
| SIRPG | 0.660 | 0.049 | 0.003 | 0.563 | 0.756 |
| MORN3 | 0.660 | 0.050 | 0.003 | 0.562 | 0.758 |
| LDHB | 0.659 | 0.051 | 0.003 | 0.560 | 0.758 |
| TRIM44 | 0.658 | 0.050 | 0.003 | 0.560 | 0.756 |
| LAG3 | 0.658 | 0.049 | 0.003 | 0.562 | 0.755 |
| GMPS | 0.658 | 0.050 | 0.003 | 0.560 | 0.757 |
| YEATS2 | 0.658 | 0.050 | 0.003 | 0.561 | 0.756 |
| EFTUD2 | 0.658 | 0.050 | 0.003 | 0.560 | 0.756 |
| FBXL14 | 0.658 | 0.050 | 0.003 | 0.559 | 0.756 |
| NUP210 | 0.658 | 0.051 | 0.003 | 0.559 | 0.757 |
| PLEKHO1 | 0.658 | 0.050 | 0.003 | 0.559 | 0.756 |
| ZNF614 | 0.658 | 0.049 | 0.003 | 0.561 | 0.754 |
| MIR6504 | 0.658 | 0.050 | 0.003 | 0.559 | 0.756 |
| PRR11 | 0.657 | 0.050 | 0.003 | 0.560 | 0.754 |
| RNF150 | 0.657 | 0.050 | 0.003 | 0.558 | 0.756 |
| IL18R1 | 0.657 | 0.050 | 0.003 | 0.558 | 0.755 |
| ZNF749 | 0.656 | 0.050 | 0.003 | 0.559 | 0.753 |
| ZNF267 | 0.656 | 0.050 | 0.003 | 0.558 | 0.754 |
| WDFY1 | 0.656 | 0.049 | 0.003 | 0.559 | 0.752 |
| SH3BP5 | 0.656 | 0.051 | 0.003 | 0.556 | 0.756 |
| LRRC18 | 0.656 | 0.050 | 0.003 | 0.557 | 0.754 |
| CD52 | 0.655 | 0.050 | 0.003 | 0.557 | 0.754 |
| KNL1 | 0.655 | 0.050 | 0.003 | 0.557 | 0.754 |
| SIPA1L1 | 0.655 | 0.051 | 0.003 | 0.555 | 0.756 |
| TNFSF15 | 0.655 | 0.049 | 0.003 | 0.559 | 0.751 |
| AIM2 | 0.655 | 0.050 | 0.003 | 0.557 | 0.753 |
| CCNB2 | 0.655 | 0.050 | 0.003 | 0.558 | 0.752 |
| ZBTB2 | 0.654 | 0.050 | 0.004 | 0.556 | 0.753 |
| CAPRIN1 | 0.654 | 0.050 | 0.004 | 0.557 | 0.751 |
| POP4 | 0.654 | 0.051 | 0.004 | 0.555 | 0.754 |
| IL21R | 0.654 | 0.050 | 0.004 | 0.556 | 0.752 |
| BCL2L11 | 0.654 | 0.052 | 0.004 | 0.552 | 0.755 |
| IL4I1 | 0.654 | 0.051 | 0.004 | 0.555 | 0.753 |
| DZIP1 | 0.654 | 0.050 | 0.004 | 0.555 | 0.753 |
| IL2RA | 0.654 | 0.052 | 0.004 | 0.552 | 0.756 |
| PLCH1 | 0.654 | 0.051 | 0.004 | 0.554 | 0.754 |
| TCF3 | 0.654 | 0.051 | 0.004 | 0.553 | 0.754 |
| FYN | 0.654 | 0.050 | 0.004 | 0.556 | 0.751 |
| PDHX | 0.654 | 0.050 | 0.004 | 0.555 | 0.752 |
| COL22A1 | 0.654 | 0.050 | 0.004 | 0.556 | 0.751 |
| GBP1P1 | 0.653 | 0.051 | 0.004 | 0.554 | 0.753 |
| GLDC | 0.653 | 0.050 | 0.004 | 0.554 | 0.752 |
| MALT1 | 0.653 | 0.050 | 0.004 | 0.554 | 0.752 |
| CASP7 | 0.653 | 0.050 | 0.004 | 0.554 | 0.751 |
| CORO1A | 0.652 | 0.050 | 0.004 | 0.555 | 0.750 |
| TPM3P9 | 0.652 | 0.051 | 0.004 | 0.553 | 0.751 |
| PDCD2 | 0.652 | 0.049 | 0.004 | 0.556 | 0.748 |
| NDUFA9 | 0.652 | 0.050 | 0.004 | 0.554 | 0.751 |
| ZNF304 | 0.652 | 0.049 | 0.004 | 0.555 | 0.749 |
| RPS26 | 0.652 | 0.049 | 0.004 | 0.556 | 0.749 |
| EPSTI1 | 0.652 | 0.050 | 0.004 | 0.554 | 0.750 |
| NIPA2 | 0.652 | 0.050 | 0.004 | 0.555 | 0.750 |
| CD80 | 0.652 | 0.051 | 0.004 | 0.552 | 0.751 |
| WNK1 | 0.652 | 0.051 | 0.004 | 0.552 | 0.752 |
| TUBA1B | 0.652 | 0.049 | 0.004 | 0.555 | 0.749 |
| BLM | 0.652 | 0.050 | 0.004 | 0.554 | 0.750 |
| MTMR4 | 0.652 | 0.050 | 0.004 | 0.554 | 0.749 |
| RELB | 0.651 | 0.051 | 0.004 | 0.551 | 0.751 |
| EDEM1 | 0.651 | 0.051 | 0.004 | 0.552 | 0.750 |
| MCRS1 | 0.651 | 0.051 | 0.004 | 0.550 | 0.751 |
| RAD51C | 0.650 | 0.051 | 0.005 | 0.550 | 0.751 |
| NME1 | 0.650 | 0.051 | 0.005 | 0.551 | 0.749 |
| DDX23 | 0.650 | 0.051 | 0.005 | 0.549 | 0.750 |
| RBM38 | 0.649 | 0.050 | 0.005 | 0.552 | 0.747 |
| ZNF525 | 0.649 | 0.051 | 0.005 | 0.550 | 0.749 |
| CHN1 | 0.649 | 0.049 | 0.005 | 0.553 | 0.746 |
| CST7 | 0.649 | 0.051 | 0.005 | 0.549 | 0.749 |
| ITGAE | 0.649 | 0.050 | 0.005 | 0.551 | 0.746 |
| CLEC7A | 0.649 | 0.050 | 0.005 | 0.551 | 0.747 |
| DNAH17 | 0.649 | 0.052 | 0.005 | 0.548 | 0.750 |
| NUP62 | 0.649 | 0.050 | 0.005 | 0.551 | 0.747 |
| ZNF180 | 0.649 | 0.050 | 0.005 | 0.551 | 0.746 |
| U2SURP | 0.648 | 0.050 | 0.005 | 0.551 | 0.746 |
| ANKRD33B | 0.648 | 0.050 | 0.005 | 0.550 | 0.747 |
| BTN2A2 | 0.648 | 0.050 | 0.005 | 0.550 | 0.746 |
| CCDC28B | 0.648 | 0.050 | 0.005 | 0.551 | 0.745 |
| GPR171 | 0.648 | 0.051 | 0.005 | 0.548 | 0.748 |
| CLPP | 0.648 | 0.050 | 0.005 | 0.550 | 0.746 |
| CHAF1A | 0.648 | 0.050 | 0.005 | 0.549 | 0.746 |
| URI1 | 0.647 | 0.052 | 0.005 | 0.545 | 0.749 |
| ST8SIA4 | 0.647 | 0.051 | 0.005 | 0.548 | 0.747 |
| ZNF672 | 0.647 | 0.051 | 0.005 | 0.548 | 0.746 |
| SLC5A6 | 0.647 | 0.050 | 0.005 | 0.549 | 0.745 |
| NUP50 | 0.647 | 0.051 | 0.005 | 0.548 | 0.746 |
| MYO7A | 0.647 | 0.050 | 0.006 | 0.548 | 0.746 |
| PTPN22 | 0.647 | 0.051 | 0.006 | 0.548 | 0.746 |
| RNU5F.1 | 0.647 | 0.050 | 0.006 | 0.550 | 0.744 |
| P2RY14 | 0.646 | 0.050 | 0.006 | 0.549 | 0.744 |
| RFX7 | 0.646 | 0.050 | 0.006 | 0.548 | 0.745 |
| MBNL1 | 0.646 | 0.051 | 0.006 | 0.546 | 0.747 |
| VASH2 | 0.646 | 0.051 | 0.006 | 0.545 | 0.747 |
| DESI1 | 0.646 | 0.050 | 0.006 | 0.547 | 0.745 |
| TPMT | 0.646 | 0.051 | 0.006 | 0.546 | 0.745 |
| FOXP3 | 0.646 | 0.050 | 0.006 | 0.547 | 0.745 |
| RAD54L | 0.645 | 0.050 | 0.006 | 0.547 | 0.744 |
| AKAP5 | 0.645 | 0.051 | 0.006 | 0.546 | 0.744 |
| PLEKHF1 | 0.645 | 0.050 | 0.006 | 0.546 | 0.744 |
| SAMD9L | 0.645 | 0.050 | 0.006 | 0.546 | 0.743 |
| IL12RB1 | 0.645 | 0.051 | 0.006 | 0.545 | 0.744 |
| DERA | 0.645 | 0.051 | 0.006 | 0.544 | 0.746 |
| ETV6 | 0.645 | 0.051 | 0.006 | 0.545 | 0.744 |
| GPR183 | 0.645 | 0.051 | 0.006 | 0.545 | 0.744 |
| ZUFSP | 0.645 | 0.050 | 0.006 | 0.547 | 0.742 |
| FAM32A | 0.645 | 0.050 | 0.006 | 0.546 | 0.743 |
| CHML | 0.645 | 0.052 | 0.006 | 0.543 | 0.746 |
| RPP30 | 0.644 | 0.049 | 0.006 | 0.548 | 0.741 |
| IFIT3 | 0.644 | 0.050 | 0.007 | 0.546 | 0.742 |
| POLA1 | 0.644 | 0.051 | 0.007 | 0.544 | 0.743 |
| LMNB2 | 0.643 | 0.050 | 0.007 | 0.546 | 0.741 |
| GLUD2 | 0.643 | 0.051 | 0.007 | 0.543 | 0.744 |
| CD38 | 0.643 | 0.050 | 0.007 | 0.545 | 0.742 |
| AP4B1.AS1 | 0.643 | 0.051 | 0.007 | 0.542 | 0.744 |
| RELT | 0.643 | 0.050 | 0.007 | 0.545 | 0.741 |
| PSMB1 | 0.643 | 0.050 | 0.007 | 0.545 | 0.741 |
| PIF1 | 0.643 | 0.050 | 0.007 | 0.544 | 0.742 |
| IL2RB | 0.643 | 0.051 | 0.007 | 0.543 | 0.742 |
| NCAPD2 | 0.643 | 0.050 | 0.007 | 0.544 | 0.741 |
| APOL3 | 0.643 | 0.050 | 0.007 | 0.544 | 0.741 |
| RNF121 | 0.643 | 0.051 | 0.007 | 0.543 | 0.742 |
| PRPF38A | 0.642 | 0.050 | 0.007 | 0.543 | 0.741 |
| ATP5E | 0.642 | 0.050 | 0.007 | 0.543 | 0.741 |
| CASP1 | 0.642 | 0.051 | 0.007 | 0.543 | 0.741 |
| RGS19 | 0.642 | 0.050 | 0.007 | 0.543 | 0.741 |
| E2F3 | 0.642 | 0.051 | 0.007 | 0.542 | 0.742 |
| IL18BP | 0.642 | 0.052 | 0.007 | 0.539 | 0.745 |
| ICAM2 | 0.642 | 0.050 | 0.008 | 0.543 | 0.740 |
| PDCD5 | 0.642 | 0.051 | 0.008 | 0.542 | 0.741 |
| PHF10 | 0.642 | 0.050 | 0.008 | 0.543 | 0.740 |
| MRPL12 | 0.642 | 0.052 | 0.008 | 0.539 | 0.744 |
| SPAG5 | 0.642 | 0.051 | 0.008 | 0.542 | 0.741 |
| MSL2 | 0.641 | 0.050 | 0.008 | 0.544 | 0.739 |
| CARS2 | 0.641 | 0.051 | 0.008 | 0.542 | 0.740 |
| DNAJC7 | 0.641 | 0.051 | 0.008 | 0.541 | 0.741 |
| NOP2 | 0.641 | 0.051 | 0.008 | 0.542 | 0.740 |
| KIF18A | 0.641 | 0.051 | 0.008 | 0.542 | 0.740 |
| STX11 | 0.641 | 0.052 | 0.008 | 0.538 | 0.744 |
| POLD3 | 0.641 | 0.051 | 0.008 | 0.541 | 0.741 |
| SLAMF1 | 0.641 | 0.052 | 0.008 | 0.539 | 0.742 |
| GTPBP1 | 0.641 | 0.051 | 0.008 | 0.541 | 0.741 |
| CTSW | 0.640 | 0.051 | 0.008 | 0.541 | 0.739 |
| PSMA7 | 0.640 | 0.050 | 0.008 | 0.543 | 0.738 |
| TCEANC | 0.640 | 0.051 | 0.008 | 0.541 | 0.740 |
| FNBP4 | 0.640 | 0.050 | 0.008 | 0.542 | 0.739 |
| POLD2 | 0.640 | 0.050 | 0.008 | 0.543 | 0.737 |
| ALYREF | 0.640 | 0.051 | 0.008 | 0.540 | 0.740 |
| CCR8 | 0.640 | 0.052 | 0.008 | 0.538 | 0.741 |
| CD3G | 0.640 | 0.051 | 0.008 | 0.540 | 0.739 |
| TGIF2 | 0.640 | 0.051 | 0.008 | 0.539 | 0.740 |
| LCP1 | 0.639 | 0.051 | 0.008 | 0.540 | 0.739 |
| MAGOH | 0.639 | 0.051 | 0.008 | 0.540 | 0.738 |
| P2RY10 | 0.639 | 0.050 | 0.008 | 0.540 | 0.738 |
| C1orf162 | 0.639 | 0.051 | 0.009 | 0.539 | 0.739 |
| C1QB | 0.639 | 0.052 | 0.009 | 0.537 | 0.741 |
| SCLT1 | 0.639 | 0.050 | 0.009 | 0.541 | 0.736 |
| ITGA4 | 0.639 | 0.051 | 0.009 | 0.539 | 0.738 |
| TLE4 | 0.639 | 0.051 | 0.009 | 0.538 | 0.739 |
| GTDC1 | 0.639 | 0.051 | 0.009 | 0.538 | 0.739 |
| SUV39H2 | 0.639 | 0.052 | 0.009 | 0.537 | 0.740 |
| KIF18B | 0.639 | 0.050 | 0.009 | 0.540 | 0.737 |
| S100A16 | 0.639 | 0.051 | 0.009 | 0.538 | 0.739 |
| KBTBD4 | 0.639 | 0.051 | 0.009 | 0.539 | 0.738 |
| DCP1B | 0.639 | 0.051 | 0.009 | 0.538 | 0.739 |
| MME | 0.639 | 0.051 | 0.009 | 0.538 | 0.739 |
| GABBR2 | 0.638 | 0.051 | 0.009 | 0.538 | 0.738 |
| BLNK | 0.638 | 0.051 | 0.009 | 0.538 | 0.739 |
| UBE2L6 | 0.638 | 0.051 | 0.009 | 0.539 | 0.738 |
| TRIM21 | 0.638 | 0.050 | 0.009 | 0.541 | 0.736 |
| TCP1 | 0.638 | 0.050 | 0.009 | 0.540 | 0.735 |
| SNRPD2 | 0.638 | 0.051 | 0.009 | 0.539 | 0.737 |
| LINC00996 | 0.637 | 0.050 | 0.009 | 0.539 | 0.736 |
| SCUBE3 | 0.637 | 0.051 | 0.009 | 0.538 | 0.737 |
| CDCA2 | 0.637 | 0.052 | 0.010 | 0.536 | 0.738 |
| APOL1 | 0.637 | 0.051 | 0.010 | 0.538 | 0.736 |
| ITK | 0.637 | 0.050 | 0.010 | 0.539 | 0.736 |
| POLR2D | 0.637 | 0.052 | 0.010 | 0.535 | 0.740 |
| FOXM1 | 0.637 | 0.050 | 0.010 | 0.539 | 0.735 |
| ILF3 | 0.637 | 0.050 | 0.010 | 0.538 | 0.736 |
| JAK2 | 0.637 | 0.051 | 0.010 | 0.537 | 0.736 |
| RUFY4 | 0.637 | 0.051 | 0.010 | 0.538 | 0.736 |
| GBP4 | 0.637 | 0.051 | 0.010 | 0.537 | 0.736 |
| ZFAT | 0.637 | 0.051 | 0.010 | 0.536 | 0.737 |
| DDIAS | 0.636 | 0.052 | 0.010 | 0.535 | 0.738 |
| LCP2 | 0.636 | 0.051 | 0.010 | 0.536 | 0.737 |
| GIMAP5 | 0.636 | 0.051 | 0.010 | 0.537 | 0.736 |
| PIK3AP1 | 0.636 | 0.051 | 0.010 | 0.536 | 0.737 |
| CYFIP2 | 0.636 | 0.051 | 0.010 | 0.536 | 0.737 |
| PFDN1 | 0.636 | 0.051 | 0.010 | 0.536 | 0.736 |
| CD27 | 0.636 | 0.050 | 0.010 | 0.537 | 0.735 |
| R3HCC1 | 0.636 | 0.052 | 0.010 | 0.534 | 0.738 |
| CDCA5 | 0.636 | 0.051 | 0.010 | 0.536 | 0.736 |
| SHMT2 | 0.636 | 0.050 | 0.010 | 0.538 | 0.734 |
| MIR645 | 0.636 | 0.050 | 0.010 | 0.537 | 0.735 |
| DDX47 | 0.636 | 0.051 | 0.010 | 0.535 | 0.736 |
| IKZF3 | 0.636 | 0.051 | 0.010 | 0.536 | 0.735 |
| BTAF1 | 0.635 | 0.050 | 0.010 | 0.537 | 0.734 |
| VPS18 | 0.635 | 0.050 | 0.010 | 0.537 | 0.734 |
| ZCCHC2 | 0.635 | 0.052 | 0.010 | 0.534 | 0.737 |
| DLGAP5 | 0.635 | 0.051 | 0.010 | 0.535 | 0.736 |
| KBTBD8 | 0.635 | 0.051 | 0.010 | 0.535 | 0.736 |
| ACP4 | 0.635 | 0.051 | 0.010 | 0.536 | 0.735 |
| HIGD1B | 0.635 | 0.050 | 0.010 | 0.537 | 0.734 |
| CWF19L1 | 0.635 | 0.051 | 0.011 | 0.535 | 0.736 |
| YARS2 | 0.635 | 0.051 | 0.011 | 0.535 | 0.735 |
| OXGR1 | 0.635 | 0.051 | 0.011 | 0.536 | 0.735 |
| BIRC5 | 0.635 | 0.051 | 0.011 | 0.535 | 0.735 |
| FAAP24 | 0.635 | 0.051 | 0.011 | 0.536 | 0.735 |
| MBNL1.AS1 | 0.635 | 0.051 | 0.011 | 0.535 | 0.736 |
| FJX1 | 0.635 | 0.051 | 0.011 | 0.535 | 0.735 |
| CD101 | 0.635 | 0.051 | 0.011 | 0.536 | 0.735 |
| ST6GALNAC4 | 0.635 | 0.050 | 0.011 | 0.538 | 0.733 |
| DDX11 | 0.635 | 0.051 | 0.011 | 0.535 | 0.735 |
| EIPR1 | 0.635 | 0.051 | 0.011 | 0.536 | 0.734 |
| ZNF256 | 0.635 | 0.050 | 0.011 | 0.536 | 0.734 |
| POLD1 | 0.635 | 0.051 | 0.011 | 0.535 | 0.734 |
| FRMD4A | 0.635 | 0.052 | 0.011 | 0.533 | 0.736 |
| SLF1 | 0.635 | 0.052 | 0.011 | 0.533 | 0.737 |
| CD86 | 0.635 | 0.052 | 0.011 | 0.533 | 0.736 |
| LCK | 0.635 | 0.050 | 0.011 | 0.536 | 0.733 |
| PREP | 0.634 | 0.051 | 0.011 | 0.534 | 0.735 |
| SLC25A19 | 0.634 | 0.051 | 0.011 | 0.534 | 0.735 |
| PMAIP1 | 0.634 | 0.052 | 0.011 | 0.533 | 0.736 |
| HAUS1 | 0.634 | 0.051 | 0.011 | 0.534 | 0.734 |
| CCNE1 | 0.634 | 0.051 | 0.011 | 0.533 | 0.735 |
| POLR1E | 0.634 | 0.051 | 0.011 | 0.533 | 0.735 |
| IL32 | 0.634 | 0.055 | 0.011 | 0.527 | 0.741 |
| SENP5 | 0.634 | 0.051 | 0.011 | 0.534 | 0.734 |
| NDUFAF8 | 0.634 | 0.052 | 0.011 | 0.532 | 0.736 |
| DGKZ | 0.634 | 0.051 | 0.011 | 0.534 | 0.733 |
| C1QC | 0.634 | 0.052 | 0.011 | 0.533 | 0.735 |
| PLEKHJ1 | 0.634 | 0.050 | 0.011 | 0.535 | 0.732 |
| DMWD | 0.634 | 0.052 | 0.012 | 0.532 | 0.735 |
| GPR174 | 0.634 | 0.051 | 0.012 | 0.534 | 0.733 |
| CHRNB2 | 0.634 | 0.050 | 0.012 | 0.536 | 0.732 |
| ZNF845 | 0.634 | 0.052 | 0.012 | 0.532 | 0.736 |
| ENTPD4 | 0.634 | 0.050 | 0.012 | 0.535 | 0.732 |
| NUP153 | 0.633 | 0.051 | 0.012 | 0.534 | 0.733 |
| ZBTB24 | 0.633 | 0.050 | 0.012 | 0.535 | 0.732 |
| SERPINB9 | 0.633 | 0.051 | 0.012 | 0.534 | 0.732 |
| SLC22A1 | 0.633 | 0.050 | 0.012 | 0.534 | 0.732 |
| APOL6 | 0.633 | 0.051 | 0.012 | 0.534 | 0.733 |
| LYZ | 0.633 | 0.051 | 0.012 | 0.533 | 0.733 |
| EZH2 | 0.633 | 0.050 | 0.012 | 0.535 | 0.731 |
| SAE1 | 0.633 | 0.052 | 0.012 | 0.532 | 0.734 |
| TOPBP1 | 0.633 | 0.051 | 0.012 | 0.534 | 0.732 |
| HSH2D | 0.633 | 0.050 | 0.012 | 0.535 | 0.731 |
| WNT5B | 0.633 | 0.054 | 0.012 | 0.528 | 0.739 |
| SELL | 0.633 | 0.050 | 0.012 | 0.534 | 0.732 |
| ERAP1 | 0.633 | 0.051 | 0.012 | 0.533 | 0.732 |
| ZNF695 | 0.633 | 0.052 | 0.012 | 0.531 | 0.734 |
| FCGR3A | 0.633 | 0.052 | 0.012 | 0.532 | 0.734 |
| DNM1L | 0.632 | 0.051 | 0.012 | 0.533 | 0.732 |
| RNF165 | 0.632 | 0.051 | 0.012 | 0.533 | 0.732 |
| RAF1 | 0.632 | 0.051 | 0.012 | 0.533 | 0.732 |
| TRIP13 | 0.632 | 0.050 | 0.012 | 0.534 | 0.731 |
| UNC119 | 0.632 | 0.051 | 0.012 | 0.533 | 0.732 |
| AC022075.1 | 0.632 | 0.051 | 0.012 | 0.533 | 0.731 |
| SAP30BP | 0.632 | 0.052 | 0.013 | 0.531 | 0.734 |
| ZNF93 | 0.632 | 0.051 | 0.013 | 0.532 | 0.732 |
| KPNA2 | 0.632 | 0.051 | 0.013 | 0.532 | 0.731 |
| HCK | 0.632 | 0.053 | 0.013 | 0.528 | 0.736 |
| PTPN7 | 0.632 | 0.051 | 0.013 | 0.532 | 0.732 |
| TRIM22 | 0.632 | 0.050 | 0.013 | 0.533 | 0.730 |
| RPS11 | 0.632 | 0.051 | 0.013 | 0.532 | 0.732 |
| M6PR | 0.632 | 0.051 | 0.013 | 0.531 | 0.732 |
| MCM3 | 0.631 | 0.051 | 0.013 | 0.531 | 0.732 |
| BEND5 | 0.631 | 0.051 | 0.013 | 0.531 | 0.732 |
| CLUHP3 | 0.631 | 0.051 | 0.013 | 0.531 | 0.731 |
| SIT1 | 0.631 | 0.051 | 0.013 | 0.532 | 0.731 |
| SIAH2 | 0.631 | 0.052 | 0.013 | 0.530 | 0.733 |
| GPRC5D | 0.631 | 0.052 | 0.013 | 0.530 | 0.732 |
| CCNT1 | 0.631 | 0.052 | 0.013 | 0.529 | 0.733 |
| SUPT4H1 | 0.631 | 0.051 | 0.013 | 0.532 | 0.730 |
| RNF41 | 0.631 | 0.051 | 0.013 | 0.531 | 0.731 |
| KDM1B | 0.631 | 0.052 | 0.013 | 0.530 | 0.732 |
| SAMD9 | 0.631 | 0.051 | 0.013 | 0.531 | 0.732 |
| CTSC | 0.631 | 0.051 | 0.013 | 0.530 | 0.732 |
| CDC6 | 0.631 | 0.051 | 0.013 | 0.530 | 0.732 |
| MFSD13A | 0.631 | 0.051 | 0.013 | 0.531 | 0.731 |
| FUT7 | 0.631 | 0.050 | 0.013 | 0.532 | 0.730 |
| KRT5 | 0.631 | 0.050 | 0.013 | 0.532 | 0.729 |
| NUP160 | 0.631 | 0.052 | 0.013 | 0.530 | 0.732 |
| TMEM131L | 0.631 | 0.052 | 0.014 | 0.528 | 0.733 |
| CD96 | 0.631 | 0.051 | 0.014 | 0.530 | 0.731 |
| PHB2 | 0.631 | 0.051 | 0.014 | 0.531 | 0.730 |
| CGAS | 0.631 | 0.052 | 0.014 | 0.528 | 0.733 |
| TUBGCP4 | 0.631 | 0.051 | 0.014 | 0.530 | 0.731 |
| AGMAT | 0.631 | 0.052 | 0.014 | 0.528 | 0.733 |
| API5 | 0.631 | 0.051 | 0.014 | 0.530 | 0.731 |
| S1PR3 | 0.631 | 0.051 | 0.014 | 0.531 | 0.731 |
| NLK | 0.630 | 0.051 | 0.014 | 0.530 | 0.731 |
| ARHGAP18 | 0.630 | 0.052 | 0.014 | 0.528 | 0.733 |
| IL2RG | 0.630 | 0.051 | 0.014 | 0.531 | 0.730 |
| DAPK2 | 0.630 | 0.052 | 0.014 | 0.528 | 0.733 |
| ADNP2 | 0.630 | 0.051 | 0.014 | 0.530 | 0.730 |
| GIMAP6 | 0.630 | 0.051 | 0.014 | 0.530 | 0.730 |
| PML | 0.630 | 0.050 | 0.014 | 0.531 | 0.729 |
| CARD9 | 0.630 | 0.050 | 0.014 | 0.531 | 0.728 |
| ADGRG5 | 0.630 | 0.051 | 0.014 | 0.531 | 0.729 |
| MED13L | 0.630 | 0.053 | 0.014 | 0.526 | 0.734 |
| FKBP5 | 0.630 | 0.052 | 0.014 | 0.529 | 0.731 |
| PARP14 | 0.630 | 0.051 | 0.014 | 0.530 | 0.729 |
| KLHDC7B | 0.630 | 0.051 | 0.014 | 0.529 | 0.730 |
| SMARCD2 | 0.630 | 0.051 | 0.014 | 0.530 | 0.729 |
| RUNX3 | 0.630 | 0.051 | 0.014 | 0.530 | 0.730 |
| CDKN2A | 0.629 | 0.051 | 0.014 | 0.530 | 0.729 |
| CDYL | 0.629 | 0.051 | 0.015 | 0.530 | 0.728 |
| PIK3C2A | 0.629 | 0.051 | 0.015 | 0.529 | 0.729 |
| RHNO1 | 0.629 | 0.051 | 0.015 | 0.530 | 0.728 |
| AQR | 0.629 | 0.051 | 0.015 | 0.530 | 0.729 |
| RAB8A | 0.629 | 0.050 | 0.015 | 0.530 | 0.728 |
| LAMP3 | 0.629 | 0.051 | 0.015 | 0.530 | 0.728 |
| UBA2 | 0.629 | 0.051 | 0.015 | 0.528 | 0.730 |
| CXorf38 | 0.629 | 0.051 | 0.015 | 0.530 | 0.728 |
| MX2 | 0.629 | 0.050 | 0.015 | 0.531 | 0.728 |
| LRP3 | 0.629 | 0.051 | 0.015 | 0.528 | 0.729 |
| CASC15 | 0.629 | 0.051 | 0.015 | 0.529 | 0.729 |
| SP140L | 0.629 | 0.051 | 0.015 | 0.530 | 0.728 |
| FGL2 | 0.629 | 0.051 | 0.015 | 0.528 | 0.730 |
| RFX5 | 0.629 | 0.051 | 0.015 | 0.529 | 0.729 |
| TMEM150B | 0.629 | 0.052 | 0.015 | 0.527 | 0.731 |
| MIR4482 | 0.629 | 0.051 | 0.015 | 0.529 | 0.728 |
| UBE2S | 0.629 | 0.050 | 0.015 | 0.530 | 0.727 |
| IL27RA | 0.629 | 0.053 | 0.015 | 0.526 | 0.732 |
| CD83 | 0.629 | 0.051 | 0.015 | 0.528 | 0.729 |
| NOLC1 | 0.629 | 0.051 | 0.015 | 0.528 | 0.730 |
| PARP11 | 0.629 | 0.051 | 0.015 | 0.529 | 0.728 |
| RGS1 | 0.629 | 0.052 | 0.015 | 0.527 | 0.730 |
| GPRC5D.AS1 | 0.628 | 0.051 | 0.015 | 0.528 | 0.729 |
| MIR3115 | 0.628 | 0.052 | 0.015 | 0.527 | 0.730 |
| BIRC3 | 0.628 | 0.052 | 0.015 | 0.527 | 0.730 |
| CEP55 | 0.628 | 0.050 | 0.015 | 0.530 | 0.727 |
| PSMD2 | 0.628 | 0.050 | 0.016 | 0.529 | 0.727 |
| ARPC2 | 0.628 | 0.051 | 0.016 | 0.528 | 0.728 |
| ITGAM | 0.628 | 0.052 | 0.016 | 0.526 | 0.731 |
| FOXI1 | 0.628 | 0.051 | 0.016 | 0.529 | 0.728 |
| SMIM12 | 0.628 | 0.051 | 0.016 | 0.528 | 0.729 |
| C1QA | 0.628 | 0.052 | 0.016 | 0.526 | 0.730 |
| SELPLG | 0.628 | 0.051 | 0.016 | 0.529 | 0.727 |
| SLA2 | 0.628 | 0.051 | 0.016 | 0.529 | 0.727 |
| PYROXD1 | 0.628 | 0.051 | 0.016 | 0.529 | 0.727 |
| TPI1 | 0.628 | 0.051 | 0.016 | 0.528 | 0.728 |
| MMS22L | 0.628 | 0.051 | 0.016 | 0.528 | 0.728 |
| MRPL51 | 0.628 | 0.051 | 0.016 | 0.528 | 0.728 |
| ORC1 | 0.628 | 0.051 | 0.016 | 0.529 | 0.727 |
| DEPDC1 | 0.628 | 0.051 | 0.016 | 0.528 | 0.727 |
| AC100861.1 | 0.628 | 0.050 | 0.016 | 0.529 | 0.727 |
| TRIM25 | 0.628 | 0.051 | 0.016 | 0.528 | 0.727 |
| STAP1 | 0.628 | 0.050 | 0.016 | 0.529 | 0.726 |
| TRIAP1 | 0.628 | 0.051 | 0.016 | 0.527 | 0.728 |
| MCRIP1 | 0.628 | 0.052 | 0.016 | 0.525 | 0.730 |
| JOSD1 | 0.628 | 0.051 | 0.016 | 0.527 | 0.728 |
| PFN1 | 0.627 | 0.051 | 0.016 | 0.528 | 0.727 |
| GIMAP4 | 0.627 | 0.051 | 0.016 | 0.528 | 0.727 |
| SLAMF8 | 0.627 | 0.053 | 0.016 | 0.523 | 0.731 |
| RCSD1 | 0.627 | 0.051 | 0.016 | 0.528 | 0.726 |
| RANBP1 | 0.627 | 0.051 | 0.016 | 0.526 | 0.728 |
| KLRB1 | 0.627 | 0.052 | 0.016 | 0.526 | 0.728 |
| CEP128 | 0.627 | 0.052 | 0.016 | 0.525 | 0.729 |
| MKI67 | 0.627 | 0.051 | 0.016 | 0.527 | 0.727 |
| PRDM1 | 0.627 | 0.052 | 0.017 | 0.526 | 0.728 |
| PHC1 | 0.627 | 0.051 | 0.017 | 0.527 | 0.727 |
| HACE1 | 0.626 | 0.051 | 0.017 | 0.527 | 0.726 |
| SLAMF6 | 0.626 | 0.051 | 0.017 | 0.526 | 0.727 |
| SLTM | 0.626 | 0.051 | 0.017 | 0.527 | 0.726 |
| GMEB1 | 0.626 | 0.051 | 0.017 | 0.527 | 0.726 |
| CARD16 | 0.626 | 0.052 | 0.017 | 0.525 | 0.728 |
| FIGN | 0.626 | 0.051 | 0.017 | 0.527 | 0.726 |
| GID8 | 0.626 | 0.051 | 0.017 | 0.527 | 0.726 |
| BATF | 0.626 | 0.051 | 0.017 | 0.527 | 0.726 |
| SPATS2 | 0.626 | 0.053 | 0.017 | 0.523 | 0.730 |
| MFHAS1 | 0.626 | 0.051 | 0.017 | 0.526 | 0.726 |
| TUBGCP3 | 0.626 | 0.051 | 0.017 | 0.526 | 0.726 |
| CTPS1 | 0.626 | 0.051 | 0.017 | 0.526 | 0.726 |
| TNFRSF1B | 0.626 | 0.051 | 0.017 | 0.525 | 0.726 |
| TMEM140 | 0.626 | 0.051 | 0.017 | 0.526 | 0.726 |
| UBE2O | 0.626 | 0.051 | 0.017 | 0.525 | 0.727 |
| CD2 | 0.626 | 0.051 | 0.017 | 0.525 | 0.727 |
| MTHFD1L | 0.626 | 0.052 | 0.018 | 0.524 | 0.727 |
| ZNF419 | 0.626 | 0.052 | 0.018 | 0.524 | 0.727 |
| MAK16 | 0.626 | 0.052 | 0.018 | 0.524 | 0.728 |
| ZNF45 | 0.626 | 0.052 | 0.018 | 0.524 | 0.727 |
| RAD51 | 0.626 | 0.052 | 0.018 | 0.523 | 0.728 |
| AC006504.5 | 0.625 | 0.051 | 0.018 | 0.526 | 0.725 |
| RNFT2 | 0.625 | 0.051 | 0.018 | 0.525 | 0.726 |
| XRN1 | 0.625 | 0.052 | 0.018 | 0.524 | 0.727 |
| DHX36 | 0.625 | 0.052 | 0.018 | 0.524 | 0.727 |
| COL4A4 | 0.625 | 0.050 | 0.018 | 0.527 | 0.724 |
| CCDC34 | 0.625 | 0.051 | 0.018 | 0.525 | 0.726 |
| RASSF2 | 0.625 | 0.052 | 0.018 | 0.524 | 0.727 |
| GBP2 | 0.625 | 0.052 | 0.018 | 0.522 | 0.728 |
| RPL23A | 0.625 | 0.052 | 0.018 | 0.523 | 0.727 |
| BICD1 | 0.625 | 0.051 | 0.018 | 0.524 | 0.726 |
| S1PR4 | 0.625 | 0.051 | 0.018 | 0.526 | 0.724 |
| AC015914.1 | 0.625 | 0.051 | 0.018 | 0.524 | 0.726 |
| ROBO1 | 0.625 | 0.051 | 0.018 | 0.525 | 0.725 |
| ANAPC11 | 0.625 | 0.052 | 0.018 | 0.523 | 0.727 |
| CELF2.AS1 | 0.625 | 0.051 | 0.018 | 0.524 | 0.726 |
| SENP1 | 0.625 | 0.051 | 0.018 | 0.525 | 0.725 |
| TATDN2 | 0.625 | 0.051 | 0.018 | 0.524 | 0.726 |
| ZBP1 | 0.625 | 0.051 | 0.018 | 0.526 | 0.724 |
| AC007622.2 | 0.625 | 0.052 | 0.018 | 0.523 | 0.726 |
| CNOT7 | 0.625 | 0.051 | 0.018 | 0.525 | 0.725 |
| KIF4A | 0.625 | 0.051 | 0.018 | 0.525 | 0.725 |
| NUSAP1 | 0.625 | 0.051 | 0.018 | 0.525 | 0.724 |
| MCOLN2 | 0.625 | 0.051 | 0.018 | 0.526 | 0.724 |
| ESPL1 | 0.625 | 0.051 | 0.018 | 0.525 | 0.724 |
| ALKBH3 | 0.625 | 0.050 | 0.019 | 0.526 | 0.723 |
| FASLG | 0.625 | 0.051 | 0.019 | 0.524 | 0.725 |
| CD4 | 0.625 | 0.052 | 0.019 | 0.522 | 0.727 |
| FYB1 | 0.625 | 0.052 | 0.019 | 0.523 | 0.726 |
| CELF2 | 0.625 | 0.052 | 0.019 | 0.522 | 0.727 |
| MCM10 | 0.624 | 0.052 | 0.019 | 0.523 | 0.725 |
| DOK2 | 0.624 | 0.052 | 0.019 | 0.523 | 0.726 |
| ADPGK.AS1 | 0.624 | 0.052 | 0.019 | 0.523 | 0.726 |
| PIMREG | 0.624 | 0.051 | 0.019 | 0.524 | 0.725 |
| NME2 | 0.624 | 0.052 | 0.019 | 0.523 | 0.725 |
| MCM5 | 0.624 | 0.052 | 0.019 | 0.523 | 0.725 |
| QSER1 | 0.624 | 0.052 | 0.019 | 0.523 | 0.725 |
| SLFN12L | 0.624 | 0.051 | 0.019 | 0.524 | 0.724 |
| RABGGTB | 0.624 | 0.051 | 0.019 | 0.523 | 0.725 |
| TOMM40 | 0.624 | 0.051 | 0.019 | 0.524 | 0.724 |
| HEPH | 0.624 | 0.050 | 0.019 | 0.525 | 0.723 |
| CASP8AP2 | 0.624 | 0.051 | 0.019 | 0.523 | 0.724 |
| ZNF567 | 0.624 | 0.051 | 0.019 | 0.524 | 0.724 |
| SAMSN1 | 0.624 | 0.052 | 0.019 | 0.522 | 0.725 |
| RPTOR | 0.624 | 0.053 | 0.019 | 0.521 | 0.727 |
| C2CD5 | 0.624 | 0.051 | 0.019 | 0.524 | 0.724 |
| NOSIP | 0.624 | 0.052 | 0.019 | 0.521 | 0.727 |
| ZBTB39 | 0.624 | 0.051 | 0.019 | 0.525 | 0.723 |
| IPO5 | 0.624 | 0.050 | 0.019 | 0.525 | 0.723 |
| STAG1 | 0.623 | 0.051 | 0.020 | 0.523 | 0.724 |
| SF3A3 | 0.623 | 0.051 | 0.020 | 0.523 | 0.724 |
| AADAT | 0.623 | 0.051 | 0.020 | 0.523 | 0.724 |
| CXCR4 | 0.623 | 0.051 | 0.020 | 0.523 | 0.724 |
| TMEM97 | 0.623 | 0.052 | 0.020 | 0.521 | 0.726 |
| HS3ST3A1 | 0.623 | 0.051 | 0.020 | 0.524 | 0.723 |
| KANSL2 | 0.623 | 0.050 | 0.020 | 0.525 | 0.722 |
| CD226 | 0.623 | 0.051 | 0.020 | 0.523 | 0.724 |
| ARHGAP30 | 0.623 | 0.051 | 0.020 | 0.523 | 0.724 |
| MEX3C | 0.623 | 0.051 | 0.020 | 0.524 | 0.723 |
| MIR646 | 0.623 | 0.051 | 0.020 | 0.524 | 0.723 |
| ATP11B | 0.623 | 0.052 | 0.020 | 0.521 | 0.725 |
| LAP3 | 0.623 | 0.051 | 0.020 | 0.523 | 0.723 |
| LMNB1 | 0.623 | 0.051 | 0.020 | 0.524 | 0.722 |
| EIF2S3 | 0.623 | 0.051 | 0.020 | 0.523 | 0.723 |
| LMO3 | 0.623 | 0.051 | 0.020 | 0.523 | 0.724 |
| PPP6R1 | 0.623 | 0.052 | 0.020 | 0.522 | 0.725 |
| ADSL | 0.623 | 0.052 | 0.020 | 0.522 | 0.725 |
| CCDC43 | 0.623 | 0.051 | 0.020 | 0.522 | 0.723 |
| CXCL10 | 0.623 | 0.052 | 0.020 | 0.520 | 0.726 |
| OAS3 | 0.623 | 0.051 | 0.020 | 0.524 | 0.722 |
| KIF11 | 0.623 | 0.051 | 0.020 | 0.524 | 0.722 |
| FOXP4.AS1 | 0.623 | 0.053 | 0.020 | 0.520 | 0.726 |
| PIK3R3 | 0.623 | 0.051 | 0.020 | 0.523 | 0.723 |
| OPTN | 0.623 | 0.053 | 0.021 | 0.518 | 0.727 |
| COX6A1 | 0.623 | 0.051 | 0.021 | 0.522 | 0.723 |
| PGAM1 | 0.623 | 0.051 | 0.021 | 0.522 | 0.723 |
| MIR505 | 0.623 | 0.052 | 0.021 | 0.521 | 0.724 |
| LRCH3 | 0.623 | 0.053 | 0.021 | 0.519 | 0.726 |
| RNF169 | 0.623 | 0.051 | 0.021 | 0.522 | 0.723 |
| NCKAP1L | 0.623 | 0.052 | 0.021 | 0.521 | 0.724 |
| PCNX1 | 0.623 | 0.052 | 0.021 | 0.521 | 0.725 |
| CYTH4 | 0.623 | 0.052 | 0.021 | 0.521 | 0.724 |
| CSRNP1 | 0.623 | 0.051 | 0.021 | 0.522 | 0.723 |
| ZNF551 | 0.623 | 0.051 | 0.021 | 0.523 | 0.722 |
| MRPL37 | 0.622 | 0.051 | 0.021 | 0.523 | 0.721 |
| MRPL11 | 0.622 | 0.053 | 0.021 | 0.519 | 0.726 |
| ACOT7 | 0.622 | 0.051 | 0.021 | 0.522 | 0.723 |
| PSMA1 | 0.622 | 0.051 | 0.021 | 0.522 | 0.723 |
| SMCHD1 | 0.622 | 0.051 | 0.021 | 0.522 | 0.723 |
| LRR1 | 0.622 | 0.053 | 0.021 | 0.518 | 0.727 |
| ISG15 | 0.622 | 0.051 | 0.021 | 0.523 | 0.722 |
| BCL2A1 | 0.622 | 0.052 | 0.021 | 0.520 | 0.724 |
| NOCT | 0.622 | 0.052 | 0.021 | 0.520 | 0.724 |
| CLDN20 | 0.622 | 0.051 | 0.021 | 0.523 | 0.722 |
| TAGAP | 0.622 | 0.051 | 0.021 | 0.522 | 0.722 |
| TIPIN | 0.622 | 0.052 | 0.021 | 0.520 | 0.724 |
| RECQL | 0.622 | 0.052 | 0.021 | 0.520 | 0.724 |
| ANKRD27 | 0.622 | 0.052 | 0.021 | 0.519 | 0.724 |
| CLSPN | 0.622 | 0.051 | 0.021 | 0.521 | 0.722 |
| MTA2 | 0.622 | 0.051 | 0.021 | 0.522 | 0.722 |
| BID | 0.622 | 0.051 | 0.022 | 0.521 | 0.722 |
| PIM2 | 0.622 | 0.051 | 0.022 | 0.521 | 0.722 |
| TFDP1 | 0.622 | 0.051 | 0.022 | 0.522 | 0.721 |
| TUBB1 | 0.622 | 0.051 | 0.022 | 0.521 | 0.722 |
| STK24 | 0.622 | 0.051 | 0.022 | 0.521 | 0.722 |
| IDO1 | 0.622 | 0.052 | 0.022 | 0.520 | 0.723 |
| OASL | 0.622 | 0.051 | 0.022 | 0.522 | 0.721 |
| GFI1 | 0.622 | 0.051 | 0.022 | 0.521 | 0.722 |
| MIR6807 | 0.621 | 0.051 | 0.022 | 0.522 | 0.721 |
| ZC3H12D | 0.621 | 0.051 | 0.022 | 0.521 | 0.722 |
| WARS | 0.621 | 0.052 | 0.022 | 0.520 | 0.723 |
| PLEK | 0.621 | 0.052 | 0.022 | 0.519 | 0.723 |
| MIR4439 | 0.621 | 0.051 | 0.022 | 0.521 | 0.722 |
| LINC02362 | 0.621 | 0.052 | 0.022 | 0.520 | 0.722 |
| HDAC2 | 0.621 | 0.052 | 0.022 | 0.520 | 0.722 |
| SLCO5A1 | 0.621 | 0.052 | 0.022 | 0.519 | 0.723 |
| LINC00426 | 0.621 | 0.051 | 0.022 | 0.521 | 0.721 |
| SVIP | 0.621 | 0.051 | 0.022 | 0.520 | 0.722 |
| LINC01364 | 0.621 | 0.054 | 0.022 | 0.516 | 0.726 |
| LIPG | 0.621 | 0.052 | 0.023 | 0.518 | 0.723 |
| YTHDC2 | 0.621 | 0.052 | 0.023 | 0.518 | 0.723 |
| SYTL3 | 0.621 | 0.050 | 0.023 | 0.522 | 0.719 |
| SLC8A1 | 0.621 | 0.051 | 0.023 | 0.520 | 0.722 |
| CAMK4 | 0.621 | 0.051 | 0.023 | 0.520 | 0.721 |
| ZBTB40 | 0.621 | 0.053 | 0.023 | 0.517 | 0.724 |
| TPD52L1 | 0.621 | 0.050 | 0.023 | 0.522 | 0.719 |
| ZNF101 | 0.621 | 0.051 | 0.023 | 0.520 | 0.721 |
| EYA3 | 0.620 | 0.052 | 0.023 | 0.519 | 0.722 |
| SH2D1A | 0.620 | 0.052 | 0.023 | 0.519 | 0.721 |
| HAUS2 | 0.620 | 0.052 | 0.023 | 0.519 | 0.722 |
| NFKB2 | 0.620 | 0.051 | 0.023 | 0.520 | 0.721 |
| TUSC3 | 0.620 | 0.051 | 0.023 | 0.521 | 0.720 |
| APIP | 0.620 | 0.050 | 0.023 | 0.522 | 0.719 |
| UBASH3A | 0.620 | 0.051 | 0.023 | 0.521 | 0.720 |
| TNFAIP3 | 0.620 | 0.054 | 0.023 | 0.515 | 0.726 |
| GTF2E2 | 0.620 | 0.052 | 0.023 | 0.518 | 0.722 |
| CHMP7 | 0.620 | 0.051 | 0.023 | 0.521 | 0.719 |
| ADGRE5 | 0.620 | 0.051 | 0.023 | 0.520 | 0.721 |
| PCNX2 | 0.620 | 0.052 | 0.023 | 0.518 | 0.722 |
| RRAS2 | 0.620 | 0.051 | 0.023 | 0.520 | 0.720 |
| TCERG1 | 0.620 | 0.052 | 0.024 | 0.519 | 0.721 |
| JMJD6 | 0.620 | 0.052 | 0.024 | 0.518 | 0.722 |
| ZNF544 | 0.620 | 0.051 | 0.024 | 0.520 | 0.720 |
| CD69 | 0.620 | 0.052 | 0.024 | 0.517 | 0.723 |
| CDC42SE2 | 0.620 | 0.052 | 0.024 | 0.519 | 0.721 |
| WNT10A | 0.620 | 0.052 | 0.024 | 0.518 | 0.722 |
| TFCP2 | 0.620 | 0.051 | 0.024 | 0.520 | 0.720 |
| NCK2 | 0.620 | 0.051 | 0.024 | 0.519 | 0.721 |
| NUS1 | 0.620 | 0.051 | 0.024 | 0.519 | 0.720 |
| NCF4 | 0.620 | 0.052 | 0.024 | 0.518 | 0.721 |
| COL4A2.AS1 | 0.620 | 0.050 | 0.024 | 0.521 | 0.719 |
| MCL1 | 0.620 | 0.052 | 0.024 | 0.518 | 0.722 |
| PDK1 | 0.620 | 0.052 | 0.024 | 0.519 | 0.721 |
| LINC00852 | 0.620 | 0.051 | 0.024 | 0.519 | 0.721 |
| TUBA1C | 0.620 | 0.051 | 0.024 | 0.520 | 0.719 |
| LSM7 | 0.620 | 0.051 | 0.024 | 0.520 | 0.720 |
| FERMT3 | 0.619 | 0.052 | 0.024 | 0.518 | 0.721 |
| NCAPH2 | 0.619 | 0.051 | 0.024 | 0.518 | 0.720 |
| HAUS6 | 0.619 | 0.053 | 0.024 | 0.516 | 0.722 |
| RARRES3 | 0.619 | 0.052 | 0.024 | 0.518 | 0.720 |
| KIF20A | 0.619 | 0.051 | 0.024 | 0.518 | 0.720 |
| EPHA4 | 0.619 | 0.052 | 0.024 | 0.517 | 0.721 |
| HSPBP1 | 0.619 | 0.052 | 0.024 | 0.518 | 0.720 |
| FGD2 | 0.619 | 0.051 | 0.024 | 0.519 | 0.719 |
| IL15RA | 0.619 | 0.051 | 0.024 | 0.519 | 0.719 |
| TRAT1 | 0.619 | 0.052 | 0.024 | 0.518 | 0.720 |
| CD74 | 0.619 | 0.051 | 0.025 | 0.519 | 0.719 |
| FDCSP | 0.619 | 0.051 | 0.025 | 0.519 | 0.719 |
| BTN3A3 | 0.619 | 0.051 | 0.025 | 0.518 | 0.719 |
| GNLY | 0.619 | 0.051 | 0.025 | 0.519 | 0.718 |
| CENPU | 0.619 | 0.051 | 0.025 | 0.519 | 0.719 |
| LRRC61 | 0.619 | 0.051 | 0.025 | 0.520 | 0.718 |
| SEMA4D | 0.619 | 0.051 | 0.025 | 0.518 | 0.719 |
| IL10RA | 0.618 | 0.052 | 0.025 | 0.516 | 0.720 |
| VAMP1 | 0.618 | 0.051 | 0.025 | 0.518 | 0.719 |
| MIR205HG | 0.618 | 0.052 | 0.025 | 0.517 | 0.720 |
| S1PR2 | 0.618 | 0.051 | 0.025 | 0.518 | 0.719 |
| TRPV2 | 0.618 | 0.052 | 0.026 | 0.517 | 0.719 |
| TESPA1 | 0.618 | 0.051 | 0.026 | 0.518 | 0.718 |
| DOCK8 | 0.618 | 0.052 | 0.026 | 0.517 | 0.719 |
| PDE4B | 0.618 | 0.052 | 0.026 | 0.516 | 0.720 |
| EXO1 | 0.618 | 0.052 | 0.026 | 0.516 | 0.720 |
| ZDHHC13 | 0.618 | 0.051 | 0.026 | 0.517 | 0.719 |
| CDV3 | 0.618 | 0.051 | 0.026 | 0.517 | 0.718 |
| SUMO2 | 0.618 | 0.052 | 0.026 | 0.517 | 0.719 |
| USP13 | 0.618 | 0.052 | 0.026 | 0.515 | 0.720 |
| SNX20 | 0.618 | 0.052 | 0.026 | 0.517 | 0.719 |
| TRIM28 | 0.618 | 0.051 | 0.026 | 0.518 | 0.718 |
| TRA2B | 0.618 | 0.051 | 0.026 | 0.517 | 0.718 |
| CYTH1 | 0.618 | 0.051 | 0.026 | 0.517 | 0.718 |
| PRR16 | 0.618 | 0.051 | 0.026 | 0.518 | 0.717 |
| SAAL1 | 0.618 | 0.052 | 0.026 | 0.516 | 0.720 |
| MRPL47 | 0.618 | 0.052 | 0.026 | 0.517 | 0.719 |
| CEP135 | 0.617 | 0.051 | 0.027 | 0.517 | 0.718 |
| SMC4 | 0.617 | 0.051 | 0.027 | 0.517 | 0.717 |
| MMP12 | 0.617 | 0.053 | 0.027 | 0.514 | 0.721 |
| ARHGEF7 | 0.617 | 0.051 | 0.027 | 0.517 | 0.718 |
| PIAS2 | 0.617 | 0.051 | 0.027 | 0.518 | 0.717 |
| SP140 | 0.617 | 0.051 | 0.027 | 0.518 | 0.717 |
| SASH3 | 0.617 | 0.051 | 0.027 | 0.517 | 0.717 |
| FIGNL1 | 0.617 | 0.051 | 0.027 | 0.517 | 0.718 |
| MAP6D1 | 0.617 | 0.052 | 0.027 | 0.516 | 0.719 |
| CEP89 | 0.617 | 0.052 | 0.027 | 0.515 | 0.719 |
| MIR6774 | 0.617 | 0.050 | 0.027 | 0.518 | 0.716 |
| EBI3 | 0.617 | 0.052 | 0.027 | 0.516 | 0.718 |
| SPIB | 0.617 | 0.051 | 0.027 | 0.517 | 0.717 |
| NCBP2 | 0.617 | 0.051 | 0.027 | 0.517 | 0.718 |
| UNC13D | 0.617 | 0.051 | 0.027 | 0.518 | 0.716 |
| MIR181B1 | 0.617 | 0.052 | 0.027 | 0.515 | 0.719 |
| IRF4 | 0.617 | 0.051 | 0.027 | 0.516 | 0.718 |
| KIF23 | 0.617 | 0.052 | 0.027 | 0.516 | 0.718 |
| FAM24B | 0.617 | 0.052 | 0.027 | 0.516 | 0.718 |
| E2F2 | 0.617 | 0.052 | 0.027 | 0.516 | 0.718 |
| TUBE1 | 0.617 | 0.051 | 0.027 | 0.518 | 0.716 |
| TBX21 | 0.617 | 0.051 | 0.028 | 0.516 | 0.717 |
| MARS | 0.617 | 0.051 | 0.028 | 0.516 | 0.718 |
| TBCD | 0.617 | 0.051 | 0.028 | 0.516 | 0.718 |
| RCL1 | 0.617 | 0.052 | 0.028 | 0.516 | 0.718 |
| ZNF776 | 0.617 | 0.051 | 0.028 | 0.517 | 0.716 |
| MAP3K14 | 0.617 | 0.051 | 0.028 | 0.516 | 0.717 |
| RRM1 | 0.617 | 0.052 | 0.028 | 0.514 | 0.719 |
| CCR4 | 0.617 | 0.051 | 0.028 | 0.516 | 0.717 |
| TIMM50 | 0.617 | 0.052 | 0.028 | 0.515 | 0.718 |
| CD53 | 0.616 | 0.052 | 0.028 | 0.514 | 0.719 |
| KIF2C | 0.616 | 0.051 | 0.028 | 0.516 | 0.717 |
| SFT2D1 | 0.616 | 0.050 | 0.028 | 0.518 | 0.715 |
| DCBLD1 | 0.616 | 0.051 | 0.028 | 0.516 | 0.716 |
| HCST | 0.616 | 0.052 | 0.028 | 0.514 | 0.718 |
| WTAP | 0.616 | 0.052 | 0.028 | 0.515 | 0.718 |
| ZNF71 | 0.616 | 0.051 | 0.028 | 0.517 | 0.716 |
| ZNF888 | 0.616 | 0.052 | 0.028 | 0.515 | 0.718 |
| P3H2 | 0.616 | 0.051 | 0.028 | 0.517 | 0.716 |
| OTULIN | 0.616 | 0.052 | 0.028 | 0.515 | 0.718 |
| HLA.DRB5 | 0.616 | 0.052 | 0.028 | 0.515 | 0.717 |
| EXO5 | 0.616 | 0.051 | 0.028 | 0.515 | 0.717 |
| STAT4 | 0.616 | 0.052 | 0.028 | 0.514 | 0.718 |
| HMGB2 | 0.616 | 0.052 | 0.028 | 0.515 | 0.718 |
| GTF3C6 | 0.616 | 0.051 | 0.028 | 0.517 | 0.715 |
| PSIP1 | 0.616 | 0.052 | 0.028 | 0.514 | 0.718 |
| ADAM8 | 0.616 | 0.051 | 0.028 | 0.515 | 0.717 |
| PCGF5 | 0.616 | 0.051 | 0.028 | 0.515 | 0.717 |
| CAPN6 | 0.616 | 0.052 | 0.028 | 0.515 | 0.718 |
| ECT2 | 0.616 | 0.052 | 0.028 | 0.514 | 0.718 |
| RNF138 | 0.616 | 0.051 | 0.029 | 0.515 | 0.717 |
| ADIPOR2 | 0.616 | 0.052 | 0.029 | 0.514 | 0.718 |
| RRM2 | 0.616 | 0.053 | 0.029 | 0.513 | 0.719 |
| CYTIP | 0.616 | 0.051 | 0.029 | 0.516 | 0.716 |
| ZNF106 | 0.616 | 0.051 | 0.029 | 0.516 | 0.716 |
| ZNF496 | 0.616 | 0.052 | 0.029 | 0.513 | 0.718 |
| IKBKE | 0.616 | 0.051 | 0.029 | 0.515 | 0.716 |
| C21orf91.OT1 | 0.616 | 0.052 | 0.029 | 0.513 | 0.718 |
| AUNIP | 0.616 | 0.051 | 0.029 | 0.516 | 0.716 |
| PHF5A | 0.616 | 0.052 | 0.029 | 0.514 | 0.717 |
| PRKD2 | 0.616 | 0.051 | 0.029 | 0.515 | 0.716 |
| ACSL5 | 0.616 | 0.052 | 0.029 | 0.514 | 0.717 |
| KLHL42 | 0.616 | 0.051 | 0.029 | 0.515 | 0.716 |
| COIL | 0.616 | 0.051 | 0.029 | 0.516 | 0.715 |
| FRAS1 | 0.616 | 0.051 | 0.029 | 0.515 | 0.716 |
| ABI3 | 0.615 | 0.051 | 0.029 | 0.516 | 0.715 |
| IRF2BP1 | 0.615 | 0.052 | 0.029 | 0.513 | 0.718 |
| SLC2A3 | 0.615 | 0.053 | 0.029 | 0.512 | 0.718 |
| LRMP | 0.615 | 0.052 | 0.029 | 0.514 | 0.716 |
| ANKRD10 | 0.615 | 0.051 | 0.029 | 0.516 | 0.714 |
| ATP11C | 0.615 | 0.052 | 0.029 | 0.514 | 0.716 |
| MED14 | 0.615 | 0.052 | 0.029 | 0.514 | 0.717 |
| ODC1 | 0.615 | 0.052 | 0.029 | 0.514 | 0.717 |
| HELZ | 0.615 | 0.052 | 0.029 | 0.513 | 0.718 |
| USP39 | 0.615 | 0.051 | 0.029 | 0.515 | 0.716 |
| DYM | 0.615 | 0.051 | 0.029 | 0.516 | 0.715 |
| RAD54L2 | 0.615 | 0.052 | 0.029 | 0.513 | 0.718 |
| ZNF384 | 0.615 | 0.052 | 0.029 | 0.514 | 0.717 |
| OIP5 | 0.615 | 0.052 | 0.029 | 0.513 | 0.717 |
| FBXO6 | 0.615 | 0.051 | 0.029 | 0.516 | 0.715 |
| LATS1 | 0.615 | 0.052 | 0.029 | 0.514 | 0.717 |
| AC010809.2 | 0.615 | 0.053 | 0.030 | 0.511 | 0.719 |
| STK17A | 0.615 | 0.052 | 0.030 | 0.514 | 0.716 |
| FAM208B | 0.615 | 0.051 | 0.030 | 0.514 | 0.716 |
| KDM5A | 0.615 | 0.052 | 0.030 | 0.513 | 0.717 |
| AKNA | 0.615 | 0.051 | 0.030 | 0.515 | 0.715 |
| STAC | 0.615 | 0.051 | 0.030 | 0.516 | 0.714 |
| PSMC3IP | 0.615 | 0.051 | 0.030 | 0.515 | 0.715 |
| SNORD83A | 0.615 | 0.052 | 0.030 | 0.513 | 0.717 |
| UBA7 | 0.615 | 0.051 | 0.030 | 0.516 | 0.714 |
| MIR4420 | 0.615 | 0.052 | 0.030 | 0.512 | 0.718 |
| FAU | 0.615 | 0.051 | 0.030 | 0.514 | 0.715 |
| P2RY13 | 0.615 | 0.053 | 0.030 | 0.512 | 0.718 |
| MEX3A | 0.615 | 0.052 | 0.030 | 0.513 | 0.716 |
| ZNF831 | 0.615 | 0.051 | 0.030 | 0.514 | 0.716 |
| OPA1 | 0.615 | 0.052 | 0.030 | 0.513 | 0.716 |
| ETV7 | 0.615 | 0.053 | 0.030 | 0.512 | 0.718 |
| TIMELESS | 0.615 | 0.051 | 0.030 | 0.515 | 0.715 |
| CBX2 | 0.614 | 0.052 | 0.031 | 0.513 | 0.716 |
| ARPP19 | 0.614 | 0.051 | 0.031 | 0.514 | 0.715 |
| SLC35B1 | 0.614 | 0.051 | 0.031 | 0.514 | 0.715 |
| MED26 | 0.614 | 0.051 | 0.031 | 0.514 | 0.715 |
| RTKN2 | 0.614 | 0.053 | 0.031 | 0.510 | 0.718 |
| NFKBIE | 0.614 | 0.054 | 0.031 | 0.509 | 0.720 |
| SRP14 | 0.614 | 0.052 | 0.031 | 0.512 | 0.717 |
| FAM78A | 0.614 | 0.052 | 0.031 | 0.513 | 0.715 |
| BORA | 0.614 | 0.051 | 0.031 | 0.514 | 0.715 |
| ZDHHC6 | 0.614 | 0.052 | 0.031 | 0.512 | 0.716 |
| BUB1 | 0.614 | 0.051 | 0.031 | 0.513 | 0.715 |
| PI4K2A | 0.614 | 0.053 | 0.031 | 0.511 | 0.718 |
| QKI | 0.614 | 0.052 | 0.031 | 0.512 | 0.716 |
| NCAPH | 0.614 | 0.052 | 0.031 | 0.513 | 0.716 |
| NLRC5 | 0.614 | 0.052 | 0.031 | 0.513 | 0.715 |
| HIST1H1B | 0.614 | 0.051 | 0.031 | 0.514 | 0.714 |
| ENC1 | 0.614 | 0.052 | 0.031 | 0.512 | 0.716 |
| OAS2 | 0.614 | 0.052 | 0.031 | 0.513 | 0.715 |
| RSAD2 | 0.614 | 0.052 | 0.031 | 0.513 | 0.715 |
| UBASH3B | 0.614 | 0.052 | 0.031 | 0.512 | 0.716 |
| CD247 | 0.614 | 0.051 | 0.031 | 0.514 | 0.714 |
| CXCL11 | 0.614 | 0.052 | 0.031 | 0.513 | 0.715 |
| MDN1 | 0.614 | 0.051 | 0.031 | 0.514 | 0.713 |
| SLA | 0.614 | 0.052 | 0.031 | 0.511 | 0.717 |
| DYRK4 | 0.614 | 0.052 | 0.032 | 0.512 | 0.715 |
| KATNBL1 | 0.614 | 0.052 | 0.032 | 0.513 | 0.715 |
| SRSF9 | 0.614 | 0.052 | 0.032 | 0.512 | 0.715 |
| TNFSF8 | 0.614 | 0.052 | 0.032 | 0.511 | 0.716 |
| CARD8.AS1 | 0.614 | 0.051 | 0.032 | 0.513 | 0.714 |
| GZMA | 0.614 | 0.052 | 0.032 | 0.512 | 0.716 |
| AKR1B1 | 0.614 | 0.052 | 0.032 | 0.511 | 0.716 |
| FUT4 | 0.614 | 0.052 | 0.032 | 0.511 | 0.716 |
| IGLL5 | 0.614 | 0.052 | 0.032 | 0.512 | 0.715 |
| IL7R | 0.614 | 0.051 | 0.032 | 0.513 | 0.714 |
| BATF2 | 0.614 | 0.052 | 0.032 | 0.511 | 0.716 |
| ELP4 | 0.613 | 0.052 | 0.032 | 0.512 | 0.715 |
| MIR155HG | 0.613 | 0.053 | 0.032 | 0.509 | 0.717 |
| TAPBPL | 0.613 | 0.051 | 0.032 | 0.514 | 0.713 |
| RIMS3 | 0.613 | 0.052 | 0.032 | 0.512 | 0.714 |
| OXTR | 0.613 | 0.052 | 0.032 | 0.512 | 0.715 |
| SNHG12 | 0.613 | 0.052 | 0.032 | 0.512 | 0.715 |
| CD40 | 0.613 | 0.052 | 0.032 | 0.512 | 0.714 |
| INPP5B | 0.613 | 0.051 | 0.032 | 0.514 | 0.713 |
| RPL17 | 0.613 | 0.051 | 0.032 | 0.513 | 0.714 |
| SEL1L3 | 0.613 | 0.051 | 0.032 | 0.513 | 0.714 |
| GATAD2A | 0.613 | 0.052 | 0.033 | 0.512 | 0.714 |
| SIGLEC10 | 0.613 | 0.053 | 0.033 | 0.510 | 0.716 |
| MCM8 | 0.613 | 0.052 | 0.033 | 0.512 | 0.714 |
| USF1 | 0.613 | 0.051 | 0.033 | 0.514 | 0.713 |
| NUP98 | 0.613 | 0.053 | 0.033 | 0.510 | 0.716 |
| CD3E | 0.613 | 0.051 | 0.033 | 0.514 | 0.713 |
| IFNG.AS1 | 0.613 | 0.052 | 0.033 | 0.512 | 0.714 |
| CCR5 | 0.613 | 0.052 | 0.033 | 0.512 | 0.714 |
| CD48 | 0.613 | 0.051 | 0.033 | 0.512 | 0.714 |
| AKAP7 | 0.613 | 0.052 | 0.033 | 0.511 | 0.715 |
| TGFBRAP1 | 0.613 | 0.051 | 0.033 | 0.513 | 0.712 |
| UBA52 | 0.613 | 0.051 | 0.033 | 0.512 | 0.713 |
| CHD4 | 0.613 | 0.052 | 0.034 | 0.510 | 0.715 |
| GPS1 | 0.613 | 0.053 | 0.034 | 0.509 | 0.716 |
| MGAT3 | 0.613 | 0.052 | 0.034 | 0.510 | 0.715 |
| XXYLT1.AS2 | 0.613 | 0.052 | 0.034 | 0.511 | 0.714 |
| CDC23 | 0.613 | 0.052 | 0.034 | 0.510 | 0.715 |
| PPIF | 0.613 | 0.052 | 0.034 | 0.511 | 0.714 |
| LDLRAD3 | 0.613 | 0.052 | 0.034 | 0.512 | 0.713 |
| FLNA | 0.612 | 0.051 | 0.034 | 0.512 | 0.712 |
| ATF5 | 0.612 | 0.051 | 0.034 | 0.512 | 0.712 |
| EMC6 | 0.612 | 0.052 | 0.034 | 0.510 | 0.714 |
| ABCD2 | 0.612 | 0.052 | 0.034 | 0.511 | 0.714 |
| IRF8 | 0.612 | 0.052 | 0.034 | 0.510 | 0.714 |
| KCNMB1 | 0.612 | 0.051 | 0.034 | 0.512 | 0.713 |
| NDUFS5 | 0.612 | 0.051 | 0.034 | 0.512 | 0.712 |
| DOCK10 | 0.612 | 0.051 | 0.034 | 0.511 | 0.713 |
| USP18 | 0.612 | 0.052 | 0.034 | 0.511 | 0.713 |
| SLC25A51 | 0.612 | 0.052 | 0.034 | 0.511 | 0.713 |
| ZNF331 | 0.612 | 0.051 | 0.034 | 0.512 | 0.712 |
| BTLA | 0.612 | 0.051 | 0.034 | 0.512 | 0.712 |
| COA1 | 0.612 | 0.051 | 0.034 | 0.512 | 0.712 |
| ZNF550 | 0.612 | 0.051 | 0.034 | 0.513 | 0.711 |
| CDC42EP4 | 0.612 | 0.051 | 0.034 | 0.511 | 0.713 |
| CSTF3 | 0.612 | 0.052 | 0.034 | 0.510 | 0.714 |
| C15orf41 | 0.612 | 0.052 | 0.035 | 0.510 | 0.713 |
| PEF1 | 0.612 | 0.051 | 0.035 | 0.512 | 0.712 |
| ARHGAP9 | 0.612 | 0.052 | 0.035 | 0.510 | 0.713 |
| CD72 | 0.612 | 0.051 | 0.035 | 0.512 | 0.712 |
| HIST1H2BO | 0.612 | 0.051 | 0.035 | 0.511 | 0.713 |
| TLR1 | 0.612 | 0.051 | 0.035 | 0.511 | 0.712 |
| MIR4659A | 0.612 | 0.051 | 0.035 | 0.512 | 0.711 |
| KLK7 | 0.612 | 0.053 | 0.035 | 0.507 | 0.716 |
| CEBPG | 0.611 | 0.053 | 0.035 | 0.508 | 0.714 |
| TAB2 | 0.611 | 0.052 | 0.035 | 0.510 | 0.713 |
| RNF19B | 0.611 | 0.051 | 0.035 | 0.511 | 0.712 |
| ORMDL1 | 0.611 | 0.051 | 0.035 | 0.511 | 0.711 |
| ANKRD44.IT1 | 0.611 | 0.051 | 0.035 | 0.511 | 0.712 |
| ZNF549 | 0.611 | 0.051 | 0.035 | 0.511 | 0.711 |
| PARVG | 0.611 | 0.052 | 0.035 | 0.510 | 0.713 |
| IFI30 | 0.611 | 0.053 | 0.036 | 0.508 | 0.714 |
| CENPM | 0.611 | 0.052 | 0.036 | 0.509 | 0.714 |
| AC006252.1 | 0.611 | 0.051 | 0.036 | 0.512 | 0.710 |
| MX1 | 0.611 | 0.052 | 0.036 | 0.509 | 0.713 |
| PSMB10 | 0.611 | 0.051 | 0.036 | 0.512 | 0.711 |
| BTK | 0.611 | 0.052 | 0.036 | 0.509 | 0.713 |
| SRSF7 | 0.611 | 0.052 | 0.036 | 0.510 | 0.713 |
| COL4A2 | 0.611 | 0.052 | 0.036 | 0.509 | 0.712 |
| ALMS1 | 0.611 | 0.052 | 0.036 | 0.510 | 0.712 |
| FIP1L1 | 0.611 | 0.053 | 0.036 | 0.506 | 0.716 |
| C6orf141 | 0.611 | 0.052 | 0.036 | 0.508 | 0.714 |
| ABCE1 | 0.611 | 0.052 | 0.036 | 0.510 | 0.712 |
| BTN3A2 | 0.611 | 0.052 | 0.036 | 0.510 | 0.712 |
| FGR | 0.611 | 0.053 | 0.036 | 0.507 | 0.715 |
| GZMH | 0.611 | 0.052 | 0.036 | 0.509 | 0.712 |
| ATAD5 | 0.611 | 0.052 | 0.036 | 0.509 | 0.713 |
| POU2AF1 | 0.611 | 0.051 | 0.036 | 0.510 | 0.711 |
| KPNB1 | 0.611 | 0.052 | 0.037 | 0.509 | 0.713 |
| BAK1 | 0.611 | 0.051 | 0.037 | 0.510 | 0.711 |
| SNRPF | 0.611 | 0.051 | 0.037 | 0.510 | 0.711 |
| ISY1 | 0.611 | 0.052 | 0.037 | 0.509 | 0.713 |
| FAM111B | 0.611 | 0.051 | 0.037 | 0.510 | 0.711 |
| FGFR1OP | 0.611 | 0.051 | 0.037 | 0.511 | 0.710 |
| CA5BP1 | 0.611 | 0.051 | 0.037 | 0.510 | 0.711 |
| EGR2 | 0.611 | 0.052 | 0.037 | 0.509 | 0.712 |
| CLEC12A | 0.611 | 0.052 | 0.037 | 0.509 | 0.712 |
| ARHGEF6 | 0.611 | 0.052 | 0.037 | 0.509 | 0.712 |
| MCUB | 0.610 | 0.051 | 0.037 | 0.510 | 0.711 |
| MLF2 | 0.610 | 0.051 | 0.037 | 0.509 | 0.711 |
| OAS1 | 0.610 | 0.051 | 0.037 | 0.510 | 0.711 |
| LAPTM5 | 0.610 | 0.052 | 0.037 | 0.507 | 0.713 |
| RAVER2 | 0.610 | 0.051 | 0.037 | 0.510 | 0.711 |
| CERKL | 0.610 | 0.052 | 0.037 | 0.508 | 0.712 |
| NEMP1 | 0.610 | 0.052 | 0.038 | 0.507 | 0.713 |
| GPR19 | 0.610 | 0.052 | 0.038 | 0.508 | 0.712 |
| STARD7 | 0.610 | 0.051 | 0.038 | 0.510 | 0.710 |
| TOMM22 | 0.610 | 0.054 | 0.038 | 0.504 | 0.716 |
| EIF2AK2 | 0.610 | 0.052 | 0.038 | 0.508 | 0.713 |
| FKRP | 0.610 | 0.053 | 0.038 | 0.507 | 0.713 |
| FGD6 | 0.610 | 0.053 | 0.038 | 0.506 | 0.715 |
| SRSF2 | 0.610 | 0.051 | 0.038 | 0.509 | 0.711 |
| MAX | 0.610 | 0.051 | 0.038 | 0.509 | 0.711 |
| IL15 | 0.610 | 0.051 | 0.038 | 0.509 | 0.710 |
| RPL13AP5 | 0.610 | 0.052 | 0.038 | 0.508 | 0.711 |
| RPS19 | 0.610 | 0.052 | 0.038 | 0.507 | 0.712 |
| CCL2 | 0.610 | 0.052 | 0.038 | 0.508 | 0.712 |
| ZNF124 | 0.610 | 0.052 | 0.038 | 0.507 | 0.712 |
| SLFN11 | 0.610 | 0.054 | 0.038 | 0.504 | 0.716 |
| HAVCR2 | 0.610 | 0.052 | 0.039 | 0.508 | 0.711 |
| TYROBP | 0.610 | 0.052 | 0.039 | 0.508 | 0.711 |
| PIKFYVE | 0.610 | 0.052 | 0.039 | 0.508 | 0.711 |
| PIK3CG | 0.610 | 0.051 | 0.039 | 0.509 | 0.710 |
| OCIAD2 | 0.610 | 0.051 | 0.039 | 0.509 | 0.710 |
| CYBB | 0.610 | 0.052 | 0.039 | 0.508 | 0.711 |
| TRIM35 | 0.610 | 0.051 | 0.039 | 0.510 | 0.709 |
| KCNAB2 | 0.610 | 0.052 | 0.039 | 0.507 | 0.712 |
| PPP1R16B | 0.610 | 0.052 | 0.039 | 0.508 | 0.711 |
| CNOT9 | 0.610 | 0.052 | 0.039 | 0.507 | 0.712 |
| PCP4L1 | 0.610 | 0.051 | 0.039 | 0.510 | 0.709 |
| FBXO25 | 0.610 | 0.052 | 0.039 | 0.508 | 0.711 |
| CDCA8 | 0.609 | 0.051 | 0.039 | 0.509 | 0.709 |
| U2AF2 | 0.609 | 0.052 | 0.039 | 0.507 | 0.712 |
| EIF3M | 0.609 | 0.052 | 0.039 | 0.507 | 0.712 |
| C5orf56 | 0.609 | 0.051 | 0.039 | 0.508 | 0.710 |
| TEX2 | 0.609 | 0.051 | 0.039 | 0.509 | 0.710 |
| TMEM158 | 0.609 | 0.052 | 0.039 | 0.508 | 0.711 |
| IKZF1 | 0.609 | 0.052 | 0.040 | 0.507 | 0.711 |
| HK3 | 0.609 | 0.053 | 0.040 | 0.505 | 0.713 |
| FN3KRP | 0.609 | 0.052 | 0.040 | 0.508 | 0.710 |
| ATP6V1B2 | 0.609 | 0.053 | 0.040 | 0.506 | 0.712 |
| VCAM1 | 0.609 | 0.052 | 0.040 | 0.507 | 0.711 |
| GYG1 | 0.609 | 0.052 | 0.040 | 0.508 | 0.710 |
| TNFRSF9 | 0.609 | 0.052 | 0.040 | 0.507 | 0.711 |
| ZAP70 | 0.609 | 0.051 | 0.040 | 0.508 | 0.709 |
| ZFP92 | 0.609 | 0.052 | 0.040 | 0.507 | 0.711 |
| ZNF714 | 0.609 | 0.051 | 0.040 | 0.509 | 0.709 |
| MED21 | 0.609 | 0.051 | 0.040 | 0.509 | 0.709 |
| ZNF724 | 0.609 | 0.051 | 0.040 | 0.508 | 0.709 |
| CIITA | 0.609 | 0.052 | 0.040 | 0.506 | 0.711 |
| MS4A6A | 0.609 | 0.052 | 0.040 | 0.507 | 0.711 |
| CMAS | 0.609 | 0.052 | 0.040 | 0.507 | 0.710 |
| GK | 0.609 | 0.053 | 0.040 | 0.504 | 0.713 |
| HDX | 0.609 | 0.052 | 0.040 | 0.507 | 0.711 |
| RBBP7 | 0.609 | 0.052 | 0.040 | 0.506 | 0.712 |
| CUEDC2 | 0.609 | 0.053 | 0.040 | 0.504 | 0.713 |
| STIL | 0.609 | 0.051 | 0.040 | 0.508 | 0.709 |
| AP2M1 | 0.609 | 0.051 | 0.040 | 0.509 | 0.709 |
| JAK3 | 0.609 | 0.051 | 0.040 | 0.509 | 0.708 |
| CXorf21 | 0.608 | 0.052 | 0.041 | 0.506 | 0.711 |
| L3MBTL4 | 0.608 | 0.053 | 0.041 | 0.505 | 0.712 |
| UST | 0.608 | 0.052 | 0.041 | 0.507 | 0.710 |
| DDX49 | 0.608 | 0.051 | 0.041 | 0.508 | 0.709 |
| CEP78 | 0.608 | 0.052 | 0.041 | 0.506 | 0.711 |
| ITGB7 | 0.608 | 0.051 | 0.041 | 0.509 | 0.708 |
| ZWILCH | 0.608 | 0.052 | 0.041 | 0.507 | 0.710 |
| PABPC4L | 0.608 | 0.052 | 0.041 | 0.506 | 0.711 |
| CKAP5 | 0.608 | 0.052 | 0.041 | 0.506 | 0.710 |
| PSMA2 | 0.608 | 0.051 | 0.041 | 0.508 | 0.708 |
| SH2D2A | 0.608 | 0.052 | 0.041 | 0.507 | 0.710 |
| EFNB2 | 0.608 | 0.052 | 0.041 | 0.506 | 0.710 |
| LSM3 | 0.608 | 0.052 | 0.041 | 0.507 | 0.709 |
| TSPOAP1.AS1 | 0.608 | 0.052 | 0.042 | 0.507 | 0.709 |
| MAD2L1 | 0.608 | 0.051 | 0.042 | 0.507 | 0.708 |
| SCIMP | 0.608 | 0.051 | 0.042 | 0.507 | 0.709 |
| LAT2 | 0.608 | 0.051 | 0.042 | 0.508 | 0.708 |
| PDCD7 | 0.608 | 0.052 | 0.042 | 0.506 | 0.709 |
| EIF4G2 | 0.608 | 0.052 | 0.042 | 0.507 | 0.709 |
| CD79A | 0.608 | 0.051 | 0.042 | 0.508 | 0.708 |
| CNIH2 | 0.608 | 0.052 | 0.042 | 0.505 | 0.710 |
| SPC24 | 0.608 | 0.052 | 0.042 | 0.507 | 0.709 |
| CHST11 | 0.608 | 0.052 | 0.042 | 0.505 | 0.710 |
| NKG7 | 0.608 | 0.052 | 0.042 | 0.506 | 0.709 |
| UGT8 | 0.608 | 0.052 | 0.042 | 0.506 | 0.709 |
| STARD4 | 0.608 | 0.052 | 0.042 | 0.505 | 0.710 |
| FCER1G | 0.608 | 0.052 | 0.042 | 0.505 | 0.710 |
| MIR200C | 0.608 | 0.053 | 0.042 | 0.504 | 0.711 |
| ZC4H2 | 0.608 | 0.053 | 0.042 | 0.504 | 0.712 |
| DDHD1 | 0.608 | 0.052 | 0.042 | 0.505 | 0.710 |
| ACTRT3 | 0.608 | 0.052 | 0.042 | 0.506 | 0.709 |
| ZNF777 | 0.608 | 0.051 | 0.042 | 0.508 | 0.707 |
| JPT1 | 0.607 | 0.052 | 0.043 | 0.506 | 0.709 |
| NASP | 0.607 | 0.052 | 0.043 | 0.506 | 0.708 |
| ZNF98 | 0.607 | 0.052 | 0.043 | 0.505 | 0.710 |
| H2AFY2 | 0.607 | 0.053 | 0.043 | 0.503 | 0.711 |
| PTPRO | 0.607 | 0.052 | 0.043 | 0.505 | 0.710 |
| TTLL4 | 0.607 | 0.052 | 0.043 | 0.506 | 0.709 |
| PCID2 | 0.607 | 0.052 | 0.043 | 0.506 | 0.709 |
| ZNF468 | 0.607 | 0.053 | 0.043 | 0.504 | 0.711 |
| HVCN1 | 0.607 | 0.052 | 0.043 | 0.505 | 0.709 |
| TMSB10 | 0.607 | 0.051 | 0.043 | 0.506 | 0.708 |
| CD3EAP | 0.607 | 0.052 | 0.043 | 0.504 | 0.710 |
| PABPC4 | 0.607 | 0.051 | 0.043 | 0.507 | 0.707 |
| EPS8 | 0.607 | 0.051 | 0.043 | 0.507 | 0.707 |
| CCR1 | 0.607 | 0.052 | 0.043 | 0.504 | 0.710 |
| SUGP2 | 0.607 | 0.052 | 0.044 | 0.505 | 0.708 |
| RPL22L1 | 0.607 | 0.054 | 0.044 | 0.501 | 0.712 |
| CASP14 | 0.607 | 0.052 | 0.044 | 0.504 | 0.709 |
| CD180 | 0.607 | 0.052 | 0.044 | 0.505 | 0.708 |
| AL121944.1 | 0.607 | 0.051 | 0.044 | 0.506 | 0.707 |
| ZNF529.AS1 | 0.607 | 0.052 | 0.044 | 0.505 | 0.708 |
| ADAMDEC1 | 0.607 | 0.053 | 0.044 | 0.503 | 0.710 |
| PRKCQ | 0.607 | 0.052 | 0.044 | 0.505 | 0.708 |
| SCML4 | 0.607 | 0.051 | 0.044 | 0.506 | 0.707 |
| ZNF473 | 0.607 | 0.052 | 0.044 | 0.505 | 0.708 |
| NCAPG2 | 0.607 | 0.052 | 0.044 | 0.504 | 0.709 |
| RPL13A | 0.606 | 0.051 | 0.045 | 0.506 | 0.707 |
| ASF1B | 0.606 | 0.051 | 0.045 | 0.505 | 0.707 |
| GTF2A2 | 0.606 | 0.052 | 0.045 | 0.505 | 0.708 |
| LSM6 | 0.606 | 0.053 | 0.045 | 0.503 | 0.710 |
| MARK1 | 0.606 | 0.052 | 0.045 | 0.505 | 0.707 |
| MIR6821 | 0.606 | 0.053 | 0.045 | 0.503 | 0.709 |
| HCLS1 | 0.606 | 0.051 | 0.045 | 0.505 | 0.707 |
| ERI1 | 0.606 | 0.054 | 0.045 | 0.501 | 0.711 |
| ZNF530 | 0.606 | 0.051 | 0.045 | 0.506 | 0.706 |
| AC124319.2 | 0.606 | 0.051 | 0.045 | 0.507 | 0.706 |
| ZNF689 | 0.606 | 0.052 | 0.045 | 0.505 | 0.707 |
| FARSA.AS1 | 0.606 | 0.051 | 0.045 | 0.505 | 0.707 |
| RFTN1 | 0.606 | 0.052 | 0.045 | 0.505 | 0.707 |
| IFI44L | 0.606 | 0.051 | 0.045 | 0.505 | 0.707 |
| AURKA | 0.606 | 0.052 | 0.045 | 0.505 | 0.707 |
| MBD1 | 0.606 | 0.052 | 0.045 | 0.504 | 0.708 |
| TROAP | 0.606 | 0.051 | 0.045 | 0.505 | 0.707 |
| SKA2 | 0.606 | 0.051 | 0.046 | 0.506 | 0.706 |
| EVI2B | 0.606 | 0.052 | 0.046 | 0.503 | 0.708 |
| TMEM14EP | 0.606 | 0.052 | 0.046 | 0.504 | 0.707 |
| CCND2 | 0.606 | 0.052 | 0.046 | 0.503 | 0.708 |
| FCRL5 | 0.606 | 0.052 | 0.046 | 0.504 | 0.707 |
| PTPRC | 0.606 | 0.052 | 0.046 | 0.504 | 0.707 |
| CCDC77 | 0.606 | 0.052 | 0.046 | 0.504 | 0.707 |
| S100A14 | 0.606 | 0.052 | 0.046 | 0.503 | 0.709 |
| CDC20 | 0.606 | 0.052 | 0.046 | 0.504 | 0.708 |
| GAPDH | 0.606 | 0.051 | 0.046 | 0.505 | 0.706 |
| LEPROTL1 | 0.606 | 0.051 | 0.046 | 0.506 | 0.705 |
| POLR1A | 0.605 | 0.051 | 0.046 | 0.505 | 0.706 |
| EXOSC5 | 0.605 | 0.053 | 0.046 | 0.501 | 0.710 |
| ZNF43 | 0.605 | 0.051 | 0.046 | 0.505 | 0.706 |
| RALY | 0.605 | 0.052 | 0.046 | 0.504 | 0.707 |
| SMARCD1 | 0.605 | 0.052 | 0.046 | 0.504 | 0.707 |
| UBE2V1 | 0.605 | 0.052 | 0.046 | 0.504 | 0.707 |
| SLC20A1 | 0.605 | 0.051 | 0.046 | 0.505 | 0.706 |
| RUVBL2 | 0.605 | 0.053 | 0.046 | 0.502 | 0.709 |
| DDX60 | 0.605 | 0.052 | 0.046 | 0.504 | 0.707 |
| DTX3L | 0.605 | 0.052 | 0.047 | 0.503 | 0.708 |
| SKP2 | 0.605 | 0.053 | 0.047 | 0.501 | 0.709 |
| FPR1 | 0.605 | 0.053 | 0.047 | 0.502 | 0.708 |
| NLE1 | 0.605 | 0.053 | 0.047 | 0.501 | 0.709 |
| CD300A | 0.605 | 0.052 | 0.047 | 0.503 | 0.707 |
| OSM | 0.605 | 0.053 | 0.047 | 0.501 | 0.709 |
| TOMM5 | 0.605 | 0.051 | 0.047 | 0.505 | 0.705 |
| ZNF639 | 0.605 | 0.052 | 0.047 | 0.504 | 0.707 |
| SLAMF7 | 0.605 | 0.051 | 0.047 | 0.504 | 0.706 |
| NDUFS6 | 0.605 | 0.051 | 0.047 | 0.505 | 0.706 |
| C3orf58 | 0.605 | 0.052 | 0.048 | 0.502 | 0.708 |
| PIWIL4 | 0.605 | 0.052 | 0.048 | 0.503 | 0.706 |
| IFI16 | 0.605 | 0.051 | 0.048 | 0.505 | 0.705 |
| ESCO2 | 0.605 | 0.053 | 0.048 | 0.501 | 0.708 |
| E2F1 | 0.605 | 0.052 | 0.048 | 0.502 | 0.707 |
| C12orf43 | 0.605 | 0.053 | 0.048 | 0.500 | 0.709 |
| ENAH | 0.605 | 0.051 | 0.048 | 0.504 | 0.705 |
| KPNA4 | 0.605 | 0.052 | 0.048 | 0.503 | 0.706 |
| HIVEP1 | 0.605 | 0.052 | 0.048 | 0.503 | 0.706 |
| ANKLE2 | 0.605 | 0.052 | 0.048 | 0.502 | 0.707 |
| CMPK2 | 0.605 | 0.052 | 0.048 | 0.503 | 0.706 |
| CLEC2B | 0.605 | 0.052 | 0.048 | 0.503 | 0.707 |
| ASCC3 | 0.605 | 0.051 | 0.048 | 0.504 | 0.705 |
| CCR2 | 0.605 | 0.052 | 0.048 | 0.503 | 0.706 |
| SNHG22 | 0.604 | 0.053 | 0.049 | 0.501 | 0.707 |
| LINC00839 | 0.604 | 0.052 | 0.049 | 0.503 | 0.706 |
| ZNF32.AS2 | 0.604 | 0.052 | 0.049 | 0.502 | 0.706 |
| UBE2J1 | 0.604 | 0.052 | 0.049 | 0.503 | 0.706 |
| XXYLT1 | 0.604 | 0.053 | 0.049 | 0.501 | 0.708 |
| TPM3 | 0.604 | 0.052 | 0.049 | 0.503 | 0.705 |
| RPL6 | 0.604 | 0.052 | 0.049 | 0.502 | 0.706 |
| TEPSIN | 0.604 | 0.053 | 0.049 | 0.500 | 0.708 |
| BRMS1 | 0.604 | 0.052 | 0.049 | 0.502 | 0.706 |
| TCN2 | 0.604 | 0.052 | 0.049 | 0.502 | 0.706 |
| MCC | 0.604 | 0.052 | 0.049 | 0.502 | 0.706 |
| TBL1X | 0.604 | 0.052 | 0.049 | 0.503 | 0.705 |
| ZNF579 | 0.604 | 0.053 | 0.049 | 0.500 | 0.708 |
| EPB41 | 0.604 | 0.051 | 0.049 | 0.505 | 0.703 |
| RNF111 | 0.604 | 0.052 | 0.049 | 0.503 | 0.705 |
| UBE3A | 0.604 | 0.052 | 0.049 | 0.503 | 0.705 |
| FIRRE | 0.604 | 0.052 | 0.049 | 0.502 | 0.707 |
| ZNF576 | 0.604 | 0.052 | 0.049 | 0.502 | 0.706 |
| CLCN4 | 0.604 | 0.052 | 0.049 | 0.502 | 0.706 |
| LIMS1.AS1 | 0.604 | 0.052 | 0.049 | 0.503 | 0.705 |

TABLE 4
Genes predictive of RD from the GSE164458_B cohort.
Area under the curve

| | | | | Asymptotic 95% | |
|---|---|---|---|---|---|
| | | | | Confidence | |
| Test | | | | Interval | |
| Result | | Std. | Asymptotic | Lower | Upper |
| Variable(s) | Area | Error^{a} | Sig.^{b} | Bound | Bound |
| ADCY9 | 0.717 | 0.048 | 0.000 | 0.623 | 0.811 |
| SUMF2 | 0.713 | 0.050 | 0.000 | 0.615 | 0.811 |
| CTSF | 0.709 | 0.047 | 0.000 | 0.616 | 0.802 |
| ANG | 0.702 | 0.048 | 0.000 | 0.608 | 0.795 |
| DHRS1 | 0.697 | 0.048 | 0.000 | 0.602 | 0.792 |
| JHDM1D.AS1 | 0.692 | 0.048 | 0.000 | 0.598 | 0.786 |
| SIAE | 0.691 | 0.048 | 0.000 | 0.598 | 0.784 |
| SERINC3 | 0.691 | 0.048 | 0.000 | 0.597 | 0.784 |
| STXBP1 | 0.688 | 0.049 | 0.000 | 0.592 | 0.784 |
| ZKSCAN1 | 0.687 | 0.048 | 0.000 | 0.594 | 0.781 |
| EXD3 | 0.686 | 0.049 | 0.000 | 0.590 | 0.782 |
| EPN2 | 0.684 | 0.049 | 0.001 | 0.589 | 0.779 |
| NEK11 | 0.682 | 0.048 | 0.001 | 0.587 | 0.777 |
| CPEB2 | 0.681 | 0.050 | 0.001 | 0.584 | 0.779 |
| TRIM4 | 0.680 | 0.049 | 0.001 | 0.583 | 0.777 |
| ZBTB42 | 0.677 | 0.048 | 0.001 | 0.583 | 0.772 |
| CRAT | 0.677 | 0.049 | 0.001 | 0.581 | 0.773 |
| PAFAH2 | 0.675 | 0.050 | 0.001 | 0.577 | 0.773 |
| DBNDD2 | 0.675 | 0.049 | 0.001 | 0.580 | 0.771 |
| F2RL1 | 0.674 | 0.049 | 0.001 | 0.578 | 0.770 |
| CRYL1 | 0.672 | 0.049 | 0.001 | 0.576 | 0.769 |
| HSF4 | 0.670 | 0.049 | 0.001 | 0.574 | 0.767 |
| SPATA25 | 0.670 | 0.050 | 0.001 | 0.573 | 0.768 |
| CFAP70 | 0.670 | 0.049 | 0.001 | 0.574 | 0.765 |
| GAS8 | 0.667 | 0.051 | 0.002 | 0.568 | 0.767 |
| EPN2.AS1 | 0.667 | 0.049 | 0.002 | 0.570 | 0.764 |
| ATP2C2.AS1 | 0.665 | 0.050 | 0.002 | 0.567 | 0.764 |
| PCP2 | 0.665 | 0.050 | 0.002 | 0.568 | 0.762 |
| RMDN1 | 0.662 | 0.050 | 0.002 | 0.564 | 0.760 |
| FGFRL1 | 0.662 | 0.051 | 0.002 | 0.563 | 0.762 |
| CCDC149 | 0.662 | 0.049 | 0.002 | 0.566 | 0.757 |
| DIP2A.IT1 | 0.661 | 0.050 | 0.002 | 0.564 | 0.759 |
| LMF1 | 0.661 | 0.049 | 0.002 | 0.566 | 0.757 |
| NQO1 | 0.661 | 0.050 | 0.002 | 0.563 | 0.759 |
| SMIM14 | 0.660 | 0.049 | 0.002 | 0.565 | 0.756 |
| ATP6AP1 | 0.660 | 0.050 | 0.002 | 0.563 | 0.758 |
| PCDHGB5 | 0.660 | 0.051 | 0.003 | 0.561 | 0.759 |
| IFT88 | 0.659 | 0.050 | 0.003 | 0.561 | 0.757 |
| ANXA4 | 0.658 | 0.051 | 0.003 | 0.559 | 0.758 |
| CCDC191 | 0.658 | 0.050 | 0.003 | 0.560 | 0.755 |
| PTK6 | 0.657 | 0.050 | 0.003 | 0.559 | 0.756 |
| AKR1C2 | 0.657 | 0.050 | 0.003 | 0.558 | 0.756 |
| GLB1L | 0.657 | 0.052 | 0.003 | 0.555 | 0.758 |
| OARD1 | 0.657 | 0.051 | 0.003 | 0.556 | 0.757 |
| CEBPD | 0.657 | 0.050 | 0.003 | 0.559 | 0.754 |
| GRID2IP | 0.656 | 0.051 | 0.003 | 0.556 | 0.756 |
| MTHFSD | 0.656 | 0.051 | 0.003 | 0.556 | 0.756 |
| SHC2 | 0.656 | 0.050 | 0.003 | 0.557 | 0.755 |
| RIN2 | 0.655 | 0.051 | 0.003 | 0.556 | 0.755 |
| FAM177A1 | 0.655 | 0.050 | 0.003 | 0.558 | 0.753 |
| HYAL1 | 0.655 | 0.049 | 0.003 | 0.559 | 0.751 |
| SDC4 | 0.655 | 0.049 | 0.003 | 0.558 | 0.751 |
| YBX2 | 0.654 | 0.050 | 0.004 | 0.557 | 0.752 |
| NOXA1 | 0.654 | 0.050 | 0.004 | 0.556 | 0.752 |
| TBC1D8B | 0.654 | 0.051 | 0.004 | 0.554 | 0.754 |
| KYAT1 | 0.654 | 0.050 | 0.004 | 0.555 | 0.753 |
| TXNRD2 | 0.654 | 0.050 | 0.004 | 0.556 | 0.751 |
| ZMAT1 | 0.653 | 0.050 | 0.004 | 0.554 | 0.752 |
| ISPD | 0.652 | 0.051 | 0.004 | 0.551 | 0.753 |
| SLC35F5 | 0.652 | 0.051 | 0.004 | 0.553 | 0.751 |
| ZSCAN21 | 0.651 | 0.050 | 0.004 | 0.554 | 0.749 |
| FITM2 | 0.651 | 0.050 | 0.004 | 0.552 | 0.749 |
| AC025171.1 | 0.651 | 0.051 | 0.004 | 0.551 | 0.751 |
| AZGP1 | 0.651 | 0.050 | 0.004 | 0.552 | 0.749 |
| MON1B | 0.650 | 0.051 | 0.005 | 0.550 | 0.751 |
| GPT | 0.650 | 0.050 | 0.005 | 0.551 | 0.749 |
| ENDOG | 0.649 | 0.050 | 0.005 | 0.551 | 0.747 |
| MIR33B | 0.649 | 0.050 | 0.005 | 0.551 | 0.747 |
| NRBP2 | 0.648 | 0.050 | 0.005 | 0.550 | 0.746 |
| ILDR1 | 0.648 | 0.051 | 0.005 | 0.547 | 0.749 |
| APOBEC2 | 0.648 | 0.052 | 0.005 | 0.547 | 0.749 |
| ST3GAL6 | 0.648 | 0.051 | 0.005 | 0.548 | 0.747 |
| NR2F2 | 0.648 | 0.050 | 0.005 | 0.549 | 0.746 |
| MAGED2 | 0.648 | 0.050 | 0.005 | 0.549 | 0.746 |
| TM9SF1 | 0.647 | 0.050 | 0.005 | 0.549 | 0.745 |
| TLDC1 | 0.647 | 0.053 | 0.006 | 0.543 | 0.750 |
| SOX12 | 0.646 | 0.051 | 0.006 | 0.547 | 0.746 |
| HBP1 | 0.646 | 0.050 | 0.006 | 0.549 | 0.743 |
| SUN1 | 0.646 | 0.050 | 0.006 | 0.547 | 0.745 |
| WWP1 | 0.645 | 0.051 | 0.006 | 0.546 | 0.744 |
| HORMAD1 | 0.645 | 0.053 | 0.006 | 0.541 | 0.748 |
| ABTB1 | 0.645 | 0.050 | 0.006 | 0.546 | 0.743 |
| DCST2 | 0.644 | 0.051 | 0.006 | 0.543 | 0.745 |
| PDXDC2P.NPIPB14P | 0.643 | 0.050 | 0.007 | 0.545 | 0.741 |
| PPP2R5A | 0.643 | 0.050 | 0.007 | 0.544 | 0.741 |
| GPR107 | 0.643 | 0.051 | 0.007 | 0.543 | 0.742 |
| HHAT | 0.642 | 0.051 | 0.007 | 0.543 | 0.742 |
| WDR5B | 0.642 | 0.051 | 0.007 | 0.542 | 0.742 |
| GATAD1 | 0.642 | 0.050 | 0.007 | 0.543 | 0.741 |
| NIPAL3 | 0.642 | 0.052 | 0.007 | 0.540 | 0.744 |
| PHF7 | 0.642 | 0.051 | 0.007 | 0.543 | 0.741 |
| COQ4 | 0.641 | 0.050 | 0.008 | 0.542 | 0.740 |
| D2HGDH | 0.641 | 0.051 | 0.008 | 0.542 | 0.741 |
| KLC4 | 0.641 | 0.051 | 0.008 | 0.541 | 0.741 |
| CENPBD1 | 0.641 | 0.051 | 0.008 | 0.540 | 0.741 |
| B4GAT1 | 0.641 | 0.053 | 0.008 | 0.538 | 0.744 |
| ALG1 | 0.641 | 0.051 | 0.008 | 0.541 | 0.741 |
| GRAMD1C | 0.641 | 0.051 | 0.008 | 0.542 | 0.740 |
| MIR3162 | 0.640 | 0.051 | 0.008 | 0.541 | 0.740 |
| PTGR2 | 0.640 | 0.052 | 0.008 | 0.538 | 0.742 |
| ZFYVE21 | 0.640 | 0.051 | 0.008 | 0.539 | 0.740 |
| ZDHHC7 | 0.640 | 0.050 | 0.008 | 0.541 | 0.739 |
| CLDN7 | 0.640 | 0.052 | 0.008 | 0.537 | 0.743 |
| TRIQK | 0.640 | 0.051 | 0.008 | 0.540 | 0.739 |
| GOLGA2 | 0.639 | 0.050 | 0.008 | 0.541 | 0.737 |
| PIEZO2 | 0.639 | 0.052 | 0.009 | 0.538 | 0.740 |
| TECPR2 | 0.639 | 0.051 | 0.009 | 0.539 | 0.739 |
| SPACA9 | 0.639 | 0.050 | 0.009 | 0.540 | 0.738 |
| PCDH1 | 0.639 | 0.052 | 0.009 | 0.537 | 0.742 |
| TCEAL4 | 0.639 | 0.051 | 0.009 | 0.538 | 0.739 |
| CUEDC1 | 0.638 | 0.051 | 0.009 | 0.538 | 0.738 |
| MPPE1 | 0.638 | 0.050 | 0.009 | 0.540 | 0.736 |
| PPP3CA | 0.638 | 0.051 | 0.009 | 0.538 | 0.737 |
| MAP1LC3B | 0.637 | 0.051 | 0.010 | 0.536 | 0.738 |
| BAIAP2L1 | 0.637 | 0.051 | 0.010 | 0.538 | 0.737 |
| AL139353.2 | 0.637 | 0.050 | 0.010 | 0.539 | 0.735 |
| IFT140 | 0.637 | 0.051 | 0.010 | 0.538 | 0.736 |
| AC016876.1 | 0.637 | 0.051 | 0.010 | 0.537 | 0.736 |
| FSTL3 | 0.637 | 0.051 | 0.010 | 0.536 | 0.737 |
| RTN2 | 0.637 | 0.052 | 0.010 | 0.535 | 0.738 |
| ADAMTS13 | 0.636 | 0.052 | 0.010 | 0.535 | 0.738 |
| TCEAL1 | 0.636 | 0.052 | 0.010 | 0.535 | 0.738 |
| CYP51A1 | 0.636 | 0.050 | 0.010 | 0.538 | 0.734 |
| ARHGEF5 | 0.636 | 0.051 | 0.010 | 0.537 | 0.736 |
| AHNAK2 | 0.636 | 0.051 | 0.010 | 0.537 | 0.735 |
| RCOR3 | 0.636 | 0.051 | 0.010 | 0.537 | 0.735 |
| DICER1.AS1 | 0.636 | 0.052 | 0.010 | 0.534 | 0.738 |
| ATP6V1H | 0.636 | 0.052 | 0.010 | 0.533 | 0.738 |
| FBXL8 | 0.635 | 0.051 | 0.010 | 0.536 | 0.735 |
| TM4SF1.AS1 | 0.635 | 0.051 | 0.010 | 0.535 | 0.736 |
| DHRS7 | 0.635 | 0.052 | 0.011 | 0.534 | 0.737 |
| NIPAL2 | 0.635 | 0.052 | 0.011 | 0.533 | 0.737 |
| LRSAM1 | 0.635 | 0.052 | 0.011 | 0.533 | 0.736 |
| TCAM1P | 0.635 | 0.050 | 0.011 | 0.536 | 0.733 |
| FAM200A | 0.634 | 0.051 | 0.011 | 0.535 | 0.734 |
| SOX8 | 0.634 | 0.050 | 0.011 | 0.537 | 0.732 |
| AC105285.1 | 0.634 | 0.051 | 0.011 | 0.535 | 0.733 |
| INHBB | 0.634 | 0.051 | 0.011 | 0.535 | 0.733 |
| METAP1D | 0.634 | 0.051 | 0.012 | 0.533 | 0.734 |
| SREBF1 | 0.633 | 0.051 | 0.012 | 0.533 | 0.733 |
| HSD11B1L | 0.633 | 0.051 | 0.012 | 0.533 | 0.733 |
| DNASE1L2 | 0.633 | 0.052 | 0.012 | 0.532 | 0.735 |
| GMPR2 | 0.633 | 0.052 | 0.012 | 0.531 | 0.735 |
| ALS2CL | 0.633 | 0.050 | 0.012 | 0.534 | 0.732 |
| MC1R | 0.633 | 0.051 | 0.012 | 0.532 | 0.734 |
| B4GALT4 | 0.633 | 0.051 | 0.012 | 0.532 | 0.734 |
| ARFIP1 | 0.633 | 0.051 | 0.012 | 0.533 | 0.733 |
| ZNHIT1 | 0.632 | 0.050 | 0.012 | 0.534 | 0.731 |
| CBR3.AS1 | 0.632 | 0.052 | 0.012 | 0.531 | 0.734 |
| ATRN | 0.632 | 0.052 | 0.013 | 0.531 | 0.734 |
| MGLL | 0.632 | 0.051 | 0.013 | 0.533 | 0.731 |
| ALDH3A2 | 0.632 | 0.052 | 0.013 | 0.531 | 0.733 |
| SDHAF4 | 0.632 | 0.050 | 0.013 | 0.533 | 0.730 |
| NR2F2.AS1 | 0.632 | 0.050 | 0.013 | 0.533 | 0.731 |
| CH17.340M24.3 | 0.631 | 0.051 | 0.013 | 0.532 | 0.731 |
| EIF4G3 | 0.631 | 0.052 | 0.013 | 0.529 | 0.733 |
| SLC52A1 | 0.631 | 0.051 | 0.013 | 0.531 | 0.731 |
| PDP1 | 0.631 | 0.052 | 0.013 | 0.529 | 0.734 |
| AOX1 | 0.631 | 0.052 | 0.013 | 0.530 | 0.732 |
| SYPL1 | 0.631 | 0.051 | 0.013 | 0.531 | 0.731 |
| MIR6777 | 0.631 | 0.051 | 0.013 | 0.531 | 0.731 |
| NIPSNAP2 | 0.631 | 0.052 | 0.014 | 0.529 | 0.732 |
| LMF1.AS1 | 0.631 | 0.051 | 0.014 | 0.531 | 0.730 |
| ABCA3 | 0.630 | 0.050 | 0.014 | 0.532 | 0.728 |
| CKB | 0.630 | 0.051 | 0.014 | 0.530 | 0.730 |
| HPS4 | 0.630 | 0.050 | 0.014 | 0.532 | 0.729 |
| AL133297.2 | 0.630 | 0.052 | 0.014 | 0.529 | 0.731 |
| HHLA3 | 0.630 | 0.052 | 0.014 | 0.528 | 0.732 |
| CBX6 | 0.630 | 0.051 | 0.014 | 0.530 | 0.730 |
| C14orf93 | 0.630 | 0.051 | 0.014 | 0.529 | 0.731 |
| QSOX1 | 0.630 | 0.053 | 0.014 | 0.527 | 0.733 |
| MESP2 | 0.630 | 0.051 | 0.014 | 0.529 | 0.730 |
| PIGV | 0.630 | 0.052 | 0.014 | 0.528 | 0.731 |
| AMT | 0.629 | 0.050 | 0.014 | 0.531 | 0.728 |
| CLMN | 0.629 | 0.051 | 0.014 | 0.529 | 0.730 |
| TCTA | 0.629 | 0.050 | 0.014 | 0.531 | 0.728 |
| CHST1 | 0.629 | 0.052 | 0.015 | 0.527 | 0.731 |
| TCEA3 | 0.629 | 0.052 | 0.015 | 0.528 | 0.731 |
| DBNDD1 | 0.629 | 0.051 | 0.015 | 0.528 | 0.730 |
| POFUT2 | 0.629 | 0.051 | 0.015 | 0.530 | 0.728 |
| GJC3 | 0.629 | 0.052 | 0.015 | 0.527 | 0.731 |
| BCL2L2 | 0.629 | 0.051 | 0.015 | 0.529 | 0.729 |
| CCS | 0.628 | 0.051 | 0.015 | 0.529 | 0.728 |
| EPOR | 0.628 | 0.051 | 0.015 | 0.529 | 0.728 |
| CDNF | 0.628 | 0.051 | 0.016 | 0.528 | 0.728 |
| MIR2355 | 0.628 | 0.051 | 0.016 | 0.528 | 0.728 |
| STON1 | 0.628 | 0.051 | 0.016 | 0.529 | 0.727 |
| PNPLA7 | 0.628 | 0.051 | 0.016 | 0.527 | 0.728 |
| HMGCL | 0.628 | 0.052 | 0.016 | 0.526 | 0.729 |
| SLC2A10 | 0.628 | 0.051 | 0.016 | 0.527 | 0.728 |
| FAM109B | 0.627 | 0.052 | 0.016 | 0.526 | 0.729 |
| RGL3 | 0.627 | 0.050 | 0.016 | 0.528 | 0.726 |
| MIR499A | 0.627 | 0.051 | 0.016 | 0.527 | 0.727 |
| SNX21 | 0.627 | 0.052 | 0.016 | 0.526 | 0.729 |
| SWI5 | 0.627 | 0.051 | 0.016 | 0.528 | 0.726 |
| SMTN | 0.627 | 0.051 | 0.016 | 0.526 | 0.728 |
| GNPTG | 0.627 | 0.051 | 0.016 | 0.527 | 0.727 |
| SPIRE2 | 0.627 | 0.051 | 0.016 | 0.527 | 0.727 |
| TRPM4 | 0.627 | 0.052 | 0.017 | 0.525 | 0.728 |
| AC009961.1 | 0.627 | 0.053 | 0.017 | 0.523 | 0.730 |
| EEF1AKMT1 | 0.627 | 0.052 | 0.017 | 0.525 | 0.728 |
| NSUN7 | 0.627 | 0.051 | 0.017 | 0.527 | 0.727 |
| EPHX1 | 0.627 | 0.052 | 0.017 | 0.525 | 0.729 |
| SPG7 | 0.626 | 0.051 | 0.017 | 0.527 | 0.726 |
| TOM1L2 | 0.626 | 0.051 | 0.017 | 0.526 | 0.726 |
| VPS9D1 | 0.626 | 0.051 | 0.017 | 0.527 | 0.725 |
| ARPIN | 0.626 | 0.051 | 0.017 | 0.527 | 0.725 |
| CGREF1 | 0.626 | 0.051 | 0.017 | 0.526 | 0.726 |
| NUDT9 | 0.626 | 0.051 | 0.017 | 0.526 | 0.726 |
| PHKG1 | 0.626 | 0.052 | 0.017 | 0.524 | 0.727 |
| ACAD10 | 0.626 | 0.051 | 0.017 | 0.526 | 0.725 |
| MOSPD3 | 0.626 | 0.051 | 0.018 | 0.525 | 0.726 |
| ANO10 | 0.626 | 0.052 | 0.018 | 0.523 | 0.728 |
| TCF25 | 0.626 | 0.050 | 0.018 | 0.527 | 0.725 |
| CDHR3 | 0.625 | 0.051 | 0.018 | 0.526 | 0.725 |
| ACTR10 | 0.625 | 0.052 | 0.018 | 0.522 | 0.728 |
| LTB4R | 0.625 | 0.052 | 0.018 | 0.523 | 0.727 |
| ZCCHC14 | 0.625 | 0.051 | 0.019 | 0.525 | 0.724 |
| GPRC5B | 0.625 | 0.051 | 0.019 | 0.524 | 0.725 |
| SHROOM1 | 0.625 | 0.052 | 0.019 | 0.523 | 0.726 |
| NEIL1 | 0.624 | 0.051 | 0.019 | 0.524 | 0.724 |
| ZNF655 | 0.624 | 0.052 | 0.019 | 0.523 | 0.725 |
| PPP1R35 | 0.624 | 0.051 | 0.019 | 0.524 | 0.724 |
| GABARAPL2 | 0.624 | 0.051 | 0.019 | 0.523 | 0.725 |
| DIXDC1 | 0.624 | 0.050 | 0.019 | 0.525 | 0.723 |
| KDM7A | 0.624 | 0.053 | 0.019 | 0.520 | 0.728 |
| RRNAD1 | 0.624 | 0.052 | 0.019 | 0.522 | 0.726 |
| TPRG1L | 0.624 | 0.051 | 0.019 | 0.524 | 0.723 |
| KIF16B | 0.624 | 0.051 | 0.019 | 0.523 | 0.724 |
| SPRED2 | 0.624 | 0.052 | 0.019 | 0.523 | 0.725 |
| ATP6V1C1 | 0.624 | 0.051 | 0.019 | 0.524 | 0.723 |
| AFG3L1P | 0.624 | 0.052 | 0.019 | 0.523 | 0.725 |
| CEP164 | 0.624 | 0.051 | 0.019 | 0.524 | 0.723 |
| GSK3B | 0.623 | 0.051 | 0.020 | 0.523 | 0.724 |
| ATP9A | 0.623 | 0.052 | 0.020 | 0.522 | 0.725 |
| GRK4 | 0.623 | 0.050 | 0.020 | 0.524 | 0.722 |
| IGIP | 0.623 | 0.051 | 0.020 | 0.522 | 0.723 |
| RCN3 | 0.623 | 0.051 | 0.020 | 0.523 | 0.722 |
| FBXL16 | 0.623 | 0.051 | 0.020 | 0.522 | 0.724 |
| PPP1R13B | 0.623 | 0.052 | 0.021 | 0.521 | 0.724 |
| AP1M2 | 0.623 | 0.053 | 0.021 | 0.520 | 0.726 |
| PTCH2 | 0.623 | 0.051 | 0.021 | 0.522 | 0.724 |
| WASL | 0.622 | 0.051 | 0.021 | 0.523 | 0.722 |
| ASPH | 0.622 | 0.052 | 0.021 | 0.519 | 0.725 |
| SLC12A9.AS1 | 0.622 | 0.052 | 0.021 | 0.519 | 0.725 |
| DHX29 | 0.622 | 0.052 | 0.021 | 0.520 | 0.724 |
| CNPY4 | 0.622 | 0.051 | 0.021 | 0.521 | 0.723 |
| CPNE7 | 0.622 | 0.051 | 0.021 | 0.521 | 0.723 |
| AC108062.1 | 0.622 | 0.051 | 0.021 | 0.522 | 0.722 |
| P4HA3 | 0.622 | 0.051 | 0.021 | 0.521 | 0.722 |
| STXBP2 | 0.622 | 0.051 | 0.021 | 0.522 | 0.722 |
| USP40 | 0.622 | 0.051 | 0.022 | 0.522 | 0.721 |
| PYGL | 0.621 | 0.051 | 0.022 | 0.521 | 0.722 |
| SPIDR | 0.621 | 0.052 | 0.022 | 0.519 | 0.723 |
| SCARNA6 | 0.621 | 0.051 | 0.022 | 0.521 | 0.721 |
| BAMBI | 0.621 | 0.052 | 0.022 | 0.519 | 0.723 |
| NOL3 | 0.621 | 0.051 | 0.022 | 0.520 | 0.722 |
| UNC5CL | 0.621 | 0.051 | 0.022 | 0.521 | 0.721 |
| CNTD2 | 0.621 | 0.051 | 0.022 | 0.520 | 0.721 |
| PLLP | 0.621 | 0.051 | 0.023 | 0.520 | 0.721 |
| ALKBH4 | 0.621 | 0.051 | 0.023 | 0.521 | 0.720 |
| TCEAL3 | 0.620 | 0.051 | 0.023 | 0.519 | 0.721 |
| OCEL1 | 0.620 | 0.052 | 0.023 | 0.518 | 0.722 |
| BCORL1 | 0.620 | 0.052 | 0.023 | 0.518 | 0.723 |
| PNMA3 | 0.620 | 0.052 | 0.023 | 0.519 | 0.722 |
| SLC25A16 | 0.620 | 0.052 | 0.023 | 0.519 | 0.722 |
| MPZL3 | 0.620 | 0.052 | 0.023 | 0.518 | 0.723 |
| CCDC15 | 0.620 | 0.052 | 0.023 | 0.518 | 0.723 |
| DIRC2 | 0.620 | 0.052 | 0.024 | 0.519 | 0.721 |
| C11orf54 | 0.620 | 0.051 | 0.024 | 0.520 | 0.720 |
| DOCK6 | 0.620 | 0.051 | 0.024 | 0.520 | 0.720 |
| MOCS1 | 0.620 | 0.052 | 0.024 | 0.518 | 0.722 |
| EEF1A2 | 0.620 | 0.051 | 0.024 | 0.520 | 0.719 |
| PDGFRL | 0.620 | 0.052 | 0.024 | 0.518 | 0.721 |
| FAM129B | 0.619 | 0.051 | 0.024 | 0.519 | 0.719 |
| SRSF6 | 0.619 | 0.052 | 0.024 | 0.518 | 0.721 |
| CPNE3 | 0.619 | 0.053 | 0.024 | 0.516 | 0.722 |
| RHBDF1 | 0.619 | 0.051 | 0.025 | 0.519 | 0.719 |
| STPG1 | 0.619 | 0.052 | 0.025 | 0.517 | 0.721 |
| ACY1 | 0.619 | 0.052 | 0.025 | 0.517 | 0.720 |
| NME6 | 0.619 | 0.051 | 0.025 | 0.519 | 0.719 |
| MAFK | 0.619 | 0.052 | 0.025 | 0.517 | 0.721 |
| ZDHHC1 | 0.619 | 0.051 | 0.025 | 0.518 | 0.719 |
| DDX19B | 0.619 | 0.052 | 0.025 | 0.516 | 0.721 |
| NOTCH1 | 0.619 | 0.051 | 0.025 | 0.518 | 0.719 |
| SRRM2 | 0.619 | 0.051 | 0.025 | 0.518 | 0.719 |
| TMEM198B | 0.619 | 0.052 | 0.025 | 0.516 | 0.721 |
| OSER1.AS1 | 0.619 | 0.052 | 0.025 | 0.517 | 0.720 |
| MBTPS1 | 0.618 | 0.052 | 0.025 | 0.516 | 0.720 |
| LONP2 | 0.618 | 0.052 | 0.025 | 0.517 | 0.719 |
| FGGY | 0.618 | 0.051 | 0.025 | 0.519 | 0.718 |
| ATXN7L1 | 0.618 | 0.051 | 0.026 | 0.517 | 0.719 |
| PRKAA1 | 0.618 | 0.052 | 0.026 | 0.515 | 0.721 |
| CPQ | 0.618 | 0.052 | 0.026 | 0.516 | 0.720 |
| GPC1 | 0.618 | 0.053 | 0.026 | 0.515 | 0.721 |
| SLC25A35 | 0.618 | 0.051 | 0.026 | 0.518 | 0.718 |
| SLC27A1 | 0.618 | 0.051 | 0.026 | 0.518 | 0.718 |
| MIR4698 | 0.618 | 0.051 | 0.026 | 0.517 | 0.719 |
| DMKN | 0.618 | 0.051 | 0.026 | 0.517 | 0.718 |
| ATP2C2 | 0.618 | 0.052 | 0.026 | 0.516 | 0.720 |
| ATG10 | 0.618 | 0.052 | 0.026 | 0.516 | 0.719 |
| LPIN3 | 0.618 | 0.051 | 0.026 | 0.518 | 0.718 |
| ATP1B1 | 0.617 | 0.053 | 0.027 | 0.514 | 0.721 |
| CMTM4 | 0.617 | 0.053 | 0.027 | 0.515 | 0.720 |
| FSIP2 | 0.617 | 0.052 | 0.027 | 0.516 | 0.719 |
| ASL | 0.617 | 0.052 | 0.027 | 0.516 | 0.719 |
| ZMAT5 | 0.617 | 0.052 | 0.027 | 0.515 | 0.719 |
| MARVELD1 | 0.617 | 0.052 | 0.027 | 0.514 | 0.720 |
| ABHD14B | 0.617 | 0.052 | 0.027 | 0.515 | 0.719 |
| WDR24 | 0.617 | 0.052 | 0.027 | 0.515 | 0.720 |
| DNASE1 | 0.617 | 0.052 | 0.027 | 0.515 | 0.719 |
| PON2 | 0.617 | 0.051 | 0.027 | 0.517 | 0.717 |
| SCYL3 | 0.617 | 0.051 | 0.027 | 0.516 | 0.718 |
| FAHD2B | 0.617 | 0.052 | 0.027 | 0.516 | 0.718 |
| CD46 | 0.617 | 0.051 | 0.028 | 0.517 | 0.717 |
| LACTB2 | 0.617 | 0.052 | 0.028 | 0.514 | 0.719 |
| RAB2A | 0.617 | 0.052 | 0.028 | 0.514 | 0.719 |
| MOK | 0.617 | 0.052 | 0.028 | 0.515 | 0.719 |
| STKLD1 | 0.616 | 0.051 | 0.028 | 0.516 | 0.717 |
| AC092902.2 | 0.616 | 0.051 | 0.028 | 0.516 | 0.716 |
| TPPP3 | 0.616 | 0.053 | 0.028 | 0.513 | 0.719 |
| TAPT1 | 0.616 | 0.052 | 0.029 | 0.515 | 0.717 |
| DEPTOR | 0.616 | 0.052 | 0.029 | 0.513 | 0.718 |
| CCDC154 | 0.616 | 0.051 | 0.029 | 0.516 | 0.716 |
| SNORD8 | 0.616 | 0.052 | 0.029 | 0.514 | 0.718 |
| BACE1 | 0.616 | 0.051 | 0.029 | 0.516 | 0.716 |
| LDHD | 0.616 | 0.051 | 0.029 | 0.515 | 0.716 |
| AC011472.1 | 0.616 | 0.051 | 0.029 | 0.516 | 0.715 |
| ANKMY2 | 0.616 | 0.052 | 0.029 | 0.513 | 0.718 |
| MVD | 0.615 | 0.051 | 0.029 | 0.516 | 0.715 |
| TAZ | 0.615 | 0.052 | 0.029 | 0.514 | 0.716 |
| HSDL1 | 0.615 | 0.052 | 0.030 | 0.513 | 0.717 |
| EIF2AK1 | 0.615 | 0.053 | 0.030 | 0.512 | 0.718 |
| FAM174B | 0.615 | 0.051 | 0.030 | 0.515 | 0.715 |
| ARHGAP32 | 0.615 | 0.052 | 0.030 | 0.513 | 0.717 |
| ADCK1 | 0.615 | 0.052 | 0.030 | 0.514 | 0.716 |
| DCST1 | 0.615 | 0.053 | 0.030 | 0.511 | 0.719 |
| BICRAL | 0.615 | 0.052 | 0.030 | 0.513 | 0.717 |
| NUMB | 0.615 | 0.052 | 0.030 | 0.513 | 0.717 |
| SPATA12 | 0.614 | 0.051 | 0.031 | 0.514 | 0.714 |
| ATXN3 | 0.614 | 0.052 | 0.031 | 0.513 | 0.715 |
| SOD3 | 0.614 | 0.051 | 0.031 | 0.513 | 0.715 |
| SH3D19 | 0.614 | 0.051 | 0.031 | 0.515 | 0.714 |
| SLC9A5 | 0.614 | 0.051 | 0.031 | 0.514 | 0.714 |
| IQCK | 0.614 | 0.052 | 0.031 | 0.511 | 0.717 |
| PLPPR2 | 0.614 | 0.052 | 0.031 | 0.512 | 0.716 |
| PIGT | 0.614 | 0.051 | 0.032 | 0.514 | 0.713 |
| BOK | 0.614 | 0.052 | 0.032 | 0.511 | 0.716 |
| EPB41L5 | 0.614 | 0.051 | 0.032 | 0.513 | 0.714 |
| BAIAP3 | 0.613 | 0.053 | 0.032 | 0.510 | 0.716 |
| FOXL1 | 0.613 | 0.052 | 0.032 | 0.511 | 0.716 |
| C16orf70 | 0.613 | 0.053 | 0.032 | 0.509 | 0.718 |
| MIR644A | 0.613 | 0.053 | 0.032 | 0.510 | 0.717 |
| CASD1 | 0.613 | 0.051 | 0.033 | 0.513 | 0.713 |
| AC025171.2 | 0.613 | 0.051 | 0.033 | 0.512 | 0.714 |
| PCIF1 | 0.613 | 0.051 | 0.033 | 0.514 | 0.713 |
| CROT | 0.613 | 0.053 | 0.033 | 0.510 | 0.716 |
| PTPA | 0.613 | 0.051 | 0.033 | 0.513 | 0.712 |
| GSTM3 | 0.613 | 0.053 | 0.034 | 0.509 | 0.716 |
| ANKS3 | 0.613 | 0.052 | 0.034 | 0.511 | 0.714 |
| ANKRD9 | 0.613 | 0.052 | 0.034 | 0.511 | 0.714 |
| THBS4 | 0.613 | 0.053 | 0.034 | 0.508 | 0.717 |
| CTDSP1 | 0.613 | 0.052 | 0.034 | 0.511 | 0.714 |
| CUL7 | 0.612 | 0.052 | 0.034 | 0.510 | 0.714 |
| LGALS3 | 0.612 | 0.053 | 0.034 | 0.509 | 0.716 |
| YAP1 | 0.612 | 0.052 | 0.034 | 0.511 | 0.714 |
| EEF2K | 0.612 | 0.052 | 0.034 | 0.511 | 0.713 |
| TMBIM4 | 0.612 | 0.052 | 0.034 | 0.510 | 0.714 |
| FRAT1 | 0.612 | 0.052 | 0.034 | 0.511 | 0.713 |
| PLAT | 0.612 | 0.051 | 0.034 | 0.512 | 0.712 |
| SCRG1 | 0.612 | 0.051 | 0.034 | 0.512 | 0.712 |
| ZNF337.AS1 | 0.612 | 0.051 | 0.034 | 0.511 | 0.713 |
| SCARNA5 | 0.612 | 0.052 | 0.035 | 0.510 | 0.713 |
| APOD | 0.612 | 0.052 | 0.035 | 0.510 | 0.714 |
| NQO2 | 0.611 | 0.051 | 0.035 | 0.512 | 0.711 |
| FAM53A | 0.611 | 0.051 | 0.035 | 0.511 | 0.712 |
| AMIGO1 | 0.611 | 0.051 | 0.036 | 0.511 | 0.712 |
| MATN3 | 0.611 | 0.052 | 0.036 | 0.510 | 0.712 |
| PURA | 0.611 | 0.052 | 0.036 | 0.510 | 0.712 |
| BCAR3 | 0.611 | 0.052 | 0.036 | 0.509 | 0.713 |
| C6orf89 | 0.611 | 0.052 | 0.036 | 0.510 | 0.712 |
| RORC | 0.611 | 0.051 | 0.037 | 0.510 | 0.711 |
| LAMB2 | 0.611 | 0.051 | 0.037 | 0.511 | 0.710 |
| PTPN12 | 0.611 | 0.052 | 0.037 | 0.509 | 0.712 |
| HEATR5A | 0.610 | 0.053 | 0.037 | 0.507 | 0.714 |
| TTC3 | 0.610 | 0.052 | 0.037 | 0.509 | 0.712 |
| BCL6 | 0.610 | 0.052 | 0.037 | 0.509 | 0.712 |
| MIR126 | 0.610 | 0.052 | 0.037 | 0.508 | 0.712 |
| RAB7A | 0.610 | 0.052 | 0.037 | 0.508 | 0.712 |
| MPV17 | 0.610 | 0.051 | 0.037 | 0.510 | 0.711 |
| SRPK2 | 0.610 | 0.052 | 0.037 | 0.509 | 0.712 |
| MIR4786 | 0.610 | 0.053 | 0.038 | 0.507 | 0.713 |
| PPL | 0.610 | 0.052 | 0.038 | 0.509 | 0.711 |
| WDR78 | 0.610 | 0.051 | 0.038 | 0.509 | 0.711 |
| ZNF219 | 0.610 | 0.052 | 0.038 | 0.508 | 0.712 |
| KCTD1 | 0.610 | 0.051 | 0.038 | 0.510 | 0.710 |
| ARHGEF12 | 0.610 | 0.051 | 0.038 | 0.510 | 0.710 |
| PCLO | 0.610 | 0.052 | 0.039 | 0.507 | 0.712 |
| LINC00174 | 0.610 | 0.051 | 0.039 | 0.509 | 0.710 |
| AKR1C1 | 0.610 | 0.051 | 0.039 | 0.509 | 0.710 |
| SRMS | 0.610 | 0.052 | 0.039 | 0.507 | 0.712 |
| TMCO4 | 0.610 | 0.052 | 0.039 | 0.508 | 0.711 |
| MCEE | 0.610 | 0.053 | 0.039 | 0.506 | 0.713 |
| C8orf37 | 0.610 | 0.051 | 0.039 | 0.509 | 0.710 |
| TBC1D24 | 0.610 | 0.051 | 0.039 | 0.510 | 0.709 |
| CLUAP1 | 0.610 | 0.053 | 0.039 | 0.506 | 0.713 |
| ENTPD3 | 0.609 | 0.052 | 0.039 | 0.508 | 0.710 |
| CCDC113 | 0.609 | 0.052 | 0.039 | 0.508 | 0.711 |
| BAZ2B | 0.609 | 0.052 | 0.039 | 0.507 | 0.712 |
| SGK3 | 0.609 | 0.053 | 0.039 | 0.505 | 0.714 |
| TTN.AS1 | 0.609 | 0.052 | 0.039 | 0.507 | 0.711 |
| LINC01909 | 0.609 | 0.053 | 0.040 | 0.506 | 0.712 |
| AL451165.2 | 0.609 | 0.051 | 0.040 | 0.508 | 0.710 |
| PHYH | 0.609 | 0.051 | 0.040 | 0.509 | 0.709 |
| BCAR1 | 0.609 | 0.051 | 0.040 | 0.509 | 0.709 |
| TTC3.AS1 | 0.609 | 0.053 | 0.040 | 0.506 | 0.712 |
| CD164L2 | 0.609 | 0.052 | 0.040 | 0.507 | 0.711 |
| PRKAR2A | 0.609 | 0.052 | 0.040 | 0.507 | 0.710 |
| MUC1 | 0.609 | 0.053 | 0.040 | 0.504 | 0.714 |
| C16orf71 | 0.609 | 0.052 | 0.040 | 0.507 | 0.710 |
| TMEM80 | 0.609 | 0.052 | 0.040 | 0.507 | 0.711 |
| TOR1AIP1 | 0.609 | 0.052 | 0.040 | 0.507 | 0.711 |
| INPPL1 | 0.609 | 0.052 | 0.040 | 0.506 | 0.711 |
| EPB41L1 | 0.609 | 0.052 | 0.040 | 0.507 | 0.710 |
| MIR3155A | 0.608 | 0.051 | 0.041 | 0.508 | 0.708 |
| C1GALT1C1 | 0.608 | 0.052 | 0.041 | 0.506 | 0.711 |
| ZBTB10 | 0.608 | 0.052 | 0.041 | 0.507 | 0.709 |
| SLC2A1.AS1 | 0.608 | 0.052 | 0.042 | 0.506 | 0.710 |
| SCD | 0.608 | 0.051 | 0.042 | 0.508 | 0.708 |
| PSCA | 0.608 | 0.052 | 0.042 | 0.505 | 0.710 |
| NORAD | 0.608 | 0.052 | 0.042 | 0.507 | 0.709 |
| UGDH | 0.608 | 0.052 | 0.042 | 0.507 | 0.709 |
| CYB5D2 | 0.608 | 0.052 | 0.042 | 0.506 | 0.710 |
| NAGPA | 0.608 | 0.051 | 0.042 | 0.507 | 0.708 |
| ATL3 | 0.608 | 0.052 | 0.042 | 0.506 | 0.709 |
| HIBADH | 0.608 | 0.051 | 0.042 | 0.507 | 0.708 |
| H19 | 0.608 | 0.051 | 0.042 | 0.507 | 0.708 |
| LINC00638 | 0.608 | 0.052 | 0.042 | 0.505 | 0.710 |
| C2CD2L | 0.608 | 0.051 | 0.042 | 0.507 | 0.708 |
| PKN3 | 0.607 | 0.052 | 0.043 | 0.506 | 0.709 |
| GSTZ1 | 0.607 | 0.052 | 0.043 | 0.505 | 0.709 |
| TP53INP2 | 0.607 | 0.052 | 0.043 | 0.505 | 0.710 |
| CAMKK1 | 0.607 | 0.052 | 0.043 | 0.506 | 0.709 |
| PPIL2 | 0.607 | 0.051 | 0.043 | 0.507 | 0.707 |
| CRIP2 | 0.607 | 0.051 | 0.043 | 0.506 | 0.708 |
| YIPF3 | 0.607 | 0.052 | 0.043 | 0.505 | 0.709 |
| RAD51B | 0.607 | 0.052 | 0.043 | 0.505 | 0.709 |
| SIGLEC15 | 0.607 | 0.052 | 0.043 | 0.505 | 0.709 |
| TMEM68 | 0.607 | 0.053 | 0.043 | 0.504 | 0.711 |
| ACVR1 | 0.607 | 0.052 | 0.044 | 0.505 | 0.709 |
| WWP2 | 0.607 | 0.052 | 0.044 | 0.505 | 0.708 |
| SBSPON | 0.607 | 0.052 | 0.044 | 0.505 | 0.708 |
| LMAN2 | 0.607 | 0.052 | 0.044 | 0.504 | 0.709 |
| ATG4B | 0.607 | 0.052 | 0.044 | 0.506 | 0.707 |
| ASIC3 | 0.607 | 0.051 | 0.044 | 0.507 | 0.706 |
| SIRPA | 0.607 | 0.052 | 0.044 | 0.504 | 0.709 |
| SYNPO | 0.607 | 0.053 | 0.044 | 0.503 | 0.710 |
| PCED1A | 0.607 | 0.051 | 0.044 | 0.506 | 0.707 |
| KLHL35 | 0.607 | 0.052 | 0.044 | 0.505 | 0.708 |
| DHODH | 0.606 | 0.052 | 0.045 | 0.503 | 0.709 |
| MIR3936HG | 0.606 | 0.053 | 0.045 | 0.503 | 0.709 |
| ZKSCAN5 | 0.606 | 0.052 | 0.045 | 0.504 | 0.708 |
| CC2D2A | 0.606 | 0.051 | 0.045 | 0.506 | 0.706 |
| NMNAT1 | 0.606 | 0.052 | 0.045 | 0.504 | 0.708 |
| GSTM4 | 0.606 | 0.052 | 0.045 | 0.504 | 0.708 |
| MIR5092 | 0.606 | 0.051 | 0.046 | 0.505 | 0.706 |
| DPY19L4 | 0.605 | 0.053 | 0.046 | 0.502 | 0.709 |
| HCFC1R1 | 0.605 | 0.051 | 0.048 | 0.505 | 0.705 |
| RASL11B | 0.605 | 0.052 | 0.048 | 0.504 | 0.706 |
| TMEM267 | 0.605 | 0.053 | 0.048 | 0.502 | 0.708 |
| TSPO2 | 0.605 | 0.052 | 0.048 | 0.503 | 0.707 |
| SIRT3 | 0.605 | 0.051 | 0.048 | 0.504 | 0.705 |
| ZNF185 | 0.605 | 0.051 | 0.048 | 0.504 | 0.706 |
| SHC1 | 0.605 | 0.052 | 0.048 | 0.503 | 0.706 |
| TOX4 | 0.605 | 0.052 | 0.048 | 0.503 | 0.707 |
| TMEM187 | 0.605 | 0.052 | 0.048 | 0.502 | 0.707 |
| FMO5 | 0.605 | 0.052 | 0.048 | 0.503 | 0.707 |
| MIR4642 | 0.605 | 0.052 | 0.048 | 0.502 | 0.707 |
| PLD2 | 0.604 | 0.053 | 0.049 | 0.501 | 0.708 |
| KLF3.AS1 | 0.604 | 0.054 | 0.049 | 0.499 | 0.710 |
| KIFC2 | 0.604 | 0.052 | 0.049 | 0.502 | 0.707 |
| MTURN | 0.604 | 0.052 | 0.049 | 0.503 | 0.705 |
| METTL18 | 0.604 | 0.053 | 0.049 | 0.500 | 0.708 |
| MRPL41 | 0.604 | 0.052 | 0.049 | 0.502 | 0.706 |
| CLDN12 | 0.604 | 0.051 | 0.049 | 0.504 | 0.704 |
| PLXNA3 | 0.604 | 0.051 | 0.049 | 0.503 | 0.705 |
| TK2 | 0.604 | 0.052 | 0.049 | 0.503 | 0.705 |

TABLE 5
Genes predictive of pCR from the GSE164458_C cohort.
Area under the curve

| | | | | Asymptotic 95% | |
|---|---|---|---|---|---|
| | | | | Confidence | |
| Test | | | | Interval | |
| Result | | Std. | Asymptotic | Lower | Upper |
| Variable(s) | Area | Error^{a} | Sig.^{b} | Bound | Bound |
| TUBGCP3 | 0.731 | 0.050 | 0.000 | 0.634 | 0.829 |
| GGA3 | 0.700 | 0.051 | 0.000 | 0.599 | 0.801 |
| DCLRE1B | 0.700 | 0.052 | 0.000 | 0.599 | 0.802 |
| TCN2 | 0.699 | 0.052 | 0.000 | 0.598 | 0.800 |
| HIST1H2AL | 0.695 | 0.052 | 0.000 | 0.593 | 0.798 |
| RHOG | 0.694 | 0.054 | 0.001 | 0.588 | 0.799 |
| GRB2 | 0.693 | 0.050 | 0.001 | 0.595 | 0.791 |
| UBALD2 | 0.692 | 0.049 | 0.001 | 0.596 | 0.788 |
| PPM1D | 0.691 | 0.048 | 0.001 | 0.596 | 0.786 |
| APOC1 | 0.690 | 0.051 | 0.001 | 0.590 | 0.790 |
| ACE | 0.689 | 0.050 | 0.001 | 0.591 | 0.788 |
| C17orf62 | 0.689 | 0.050 | 0.001 | 0.590 | 0.788 |
| DNAJC7 | 0.689 | 0.052 | 0.001 | 0.586 | 0.791 |
| RBM15 | 0.689 | 0.052 | 0.001 | 0.587 | 0.790 |
| RANBP3 | 0.687 | 0.053 | 0.001 | 0.584 | 0.790 |
| DCAF7 | 0.686 | 0.050 | 0.001 | 0.589 | 0.783 |
| HIST1H2BJ | 0.686 | 0.052 | 0.001 | 0.583 | 0.788 |
| LRRC45 | 0.686 | 0.053 | 0.001 | 0.582 | 0.789 |
| DTNBP1 | 0.686 | 0.048 | 0.001 | 0.591 | 0.780 |
| TEPSIN | 0.686 | 0.052 | 0.001 | 0.583 | 0.788 |
| PFDN1 | 0.683 | 0.050 | 0.001 | 0.584 | 0.782 |
| LAG3 | 0.680 | 0.050 | 0.001 | 0.582 | 0.779 |
| SUMO2 | 0.680 | 0.052 | 0.001 | 0.578 | 0.783 |
| TTI2 | 0.680 | 0.051 | 0.001 | 0.581 | 0.780 |
| CCNE2 | 0.680 | 0.053 | 0.001 | 0.575 | 0.784 |
| SLC25A28 | 0.680 | 0.050 | 0.001 | 0.583 | 0.777 |
| CHAMP1 | 0.679 | 0.053 | 0.001 | 0.575 | 0.783 |
| LIPA | 0.679 | 0.052 | 0.001 | 0.577 | 0.782 |
| DNMT1 | 0.679 | 0.052 | 0.001 | 0.578 | 0.780 |
| MTMR1 | 0.678 | 0.050 | 0.001 | 0.579 | 0.777 |
| TMEM106C | 0.676 | 0.053 | 0.002 | 0.571 | 0.781 |
| MAP3K3 | 0.675 | 0.050 | 0.002 | 0.577 | 0.774 |
| C11orf45 | 0.675 | 0.050 | 0.002 | 0.578 | 0.773 |
| TOP3A | 0.675 | 0.051 | 0.002 | 0.574 | 0.775 |
| PLD3 | 0.674 | 0.051 | 0.002 | 0.574 | 0.774 |
| CYTH1 | 0.674 | 0.051 | 0.002 | 0.575 | 0.773 |
| LIMD2 | 0.674 | 0.050 | 0.002 | 0.576 | 0.771 |
| SMIM5 | 0.674 | 0.054 | 0.002 | 0.569 | 0.779 |
| UPF3A | 0.673 | 0.051 | 0.002 | 0.572 | 0.774 |
| MIF4GD | 0.672 | 0.052 | 0.002 | 0.570 | 0.775 |
| SLC35B1 | 0.672 | 0.052 | 0.002 | 0.571 | 0.773 |
| SIGLEC1 | 0.672 | 0.054 | 0.002 | 0.566 | 0.777 |
| CENPK | 0.672 | 0.054 | 0.002 | 0.567 | 0.777 |
| ARMC7 | 0.671 | 0.054 | 0.002 | 0.566 | 0.777 |
| SLC25A14 | 0.671 | 0.049 | 0.002 | 0.575 | 0.768 |
| HIGD1B | 0.671 | 0.052 | 0.002 | 0.569 | 0.774 |
| MIR3973 | 0.671 | 0.052 | 0.002 | 0.570 | 0.772 |
| COX14 | 0.670 | 0.052 | 0.002 | 0.569 | 0.771 |
| ADPGK.AS1 | 0.669 | 0.054 | 0.002 | 0.564 | 0.775 |
| LNP1 | 0.669 | 0.049 | 0.002 | 0.573 | 0.766 |
| SLC15A3 | 0.669 | 0.055 | 0.002 | 0.561 | 0.777 |
| CEP97 | 0.669 | 0.053 | 0.002 | 0.566 | 0.772 |
| NARF | 0.668 | 0.054 | 0.003 | 0.563 | 0.773 |
| CDKN2C | 0.668 | 0.052 | 0.003 | 0.567 | 0.770 |
| RAD51D | 0.668 | 0.052 | 0.003 | 0.566 | 0.770 |
| SLC25A19 | 0.668 | 0.053 | 0.003 | 0.565 | 0.771 |
| NMT1 | 0.668 | 0.051 | 0.003 | 0.568 | 0.768 |
| ING1 | 0.667 | 0.052 | 0.003 | 0.564 | 0.770 |
| RPTOR | 0.667 | 0.052 | 0.003 | 0.565 | 0.768 |
| RRM1 | 0.667 | 0.055 | 0.003 | 0.559 | 0.774 |
| CNTRL | 0.667 | 0.051 | 0.003 | 0.567 | 0.766 |
| HELZ | 0.666 | 0.050 | 0.003 | 0.568 | 0.765 |
| RECQL5 | 0.666 | 0.054 | 0.003 | 0.560 | 0.772 |
| CHAF1A | 0.666 | 0.056 | 0.003 | 0.556 | 0.775 |
| TMEM104 | 0.666 | 0.053 | 0.003 | 0.563 | 0.769 |
| E2F8 | 0.666 | 0.054 | 0.003 | 0.559 | 0.772 |
| MFSD11 | 0.665 | 0.051 | 0.003 | 0.565 | 0.766 |
| APOE | 0.665 | 0.054 | 0.003 | 0.560 | 0.771 |
| SP2 | 0.665 | 0.051 | 0.003 | 0.565 | 0.765 |
| SUPT4H1 | 0.665 | 0.052 | 0.003 | 0.564 | 0.766 |
| OGFOD3 | 0.664 | 0.055 | 0.003 | 0.557 | 0.772 |
| HIST1H2BO | 0.664 | 0.054 | 0.003 | 0.558 | 0.770 |
| CTSD | 0.664 | 0.053 | 0.003 | 0.559 | 0.768 |
| SSH2 | 0.664 | 0.050 | 0.003 | 0.565 | 0.763 |
| TAF5 | 0.664 | 0.050 | 0.003 | 0.565 | 0.762 |
| MPP1 | 0.664 | 0.054 | 0.003 | 0.557 | 0.770 |
| SNORA1B | 0.664 | 0.048 | 0.003 | 0.569 | 0.758 |
| RHNO1 | 0.664 | 0.051 | 0.003 | 0.564 | 0.763 |
| PIGS | 0.664 | 0.052 | 0.003 | 0.563 | 0.765 |
| SRSF2 | 0.663 | 0.055 | 0.003 | 0.556 | 0.770 |
| C1QC | 0.663 | 0.056 | 0.003 | 0.554 | 0.772 |
| NUP85 | 0.663 | 0.053 | 0.003 | 0.559 | 0.767 |
| ASB16.AS1 | 0.663 | 0.053 | 0.004 | 0.559 | 0.766 |
| GGA2 | 0.663 | 0.051 | 0.004 | 0.562 | 0.763 |
| KPNB1 | 0.662 | 0.052 | 0.004 | 0.561 | 0.763 |
| HIST1H2AD | 0.662 | 0.053 | 0.004 | 0.559 | 0.766 |
| ATP6V0A1 | 0.662 | 0.052 | 0.004 | 0.559 | 0.765 |
| TFDP1 | 0.661 | 0.056 | 0.004 | 0.553 | 0.770 |
| HIST1H2AG | 0.661 | 0.052 | 0.004 | 0.560 | 0.763 |
| CHAF1B | 0.661 | 0.057 | 0.004 | 0.550 | 0.773 |
| AC022211.2 | 0.661 | 0.053 | 0.004 | 0.557 | 0.766 |
| PTPN6 | 0.661 | 0.051 | 0.004 | 0.562 | 0.761 |
| OLR1 | 0.661 | 0.053 | 0.004 | 0.557 | 0.765 |
| C1QB | 0.661 | 0.055 | 0.004 | 0.553 | 0.770 |
| FUCA1 | 0.661 | 0.056 | 0.004 | 0.552 | 0.770 |
| BARD1 | 0.661 | 0.053 | 0.004 | 0.557 | 0.764 |
| ZMYND15 | 0.660 | 0.053 | 0.004 | 0.556 | 0.764 |
| KIF18B | 0.660 | 0.055 | 0.004 | 0.553 | 0.767 |
| SNORA65 | 0.660 | 0.052 | 0.004 | 0.559 | 0.761 |
| SMARCA2 | 0.660 | 0.051 | 0.004 | 0.560 | 0.760 |
| ACRBP | 0.660 | 0.050 | 0.004 | 0.561 | 0.759 |
| TMEM150B | 0.660 | 0.054 | 0.004 | 0.554 | 0.766 |
| IL23A | 0.660 | 0.054 | 0.004 | 0.554 | 0.765 |
| GRN | 0.659 | 0.054 | 0.004 | 0.554 | 0.764 |
| TLR6 | 0.659 | 0.053 | 0.004 | 0.556 | 0.763 |
| FOXK2 | 0.659 | 0.053 | 0.004 | 0.555 | 0.762 |
| CYP27A1 | 0.659 | 0.052 | 0.004 | 0.557 | 0.760 |
| TRIM25 | 0.658 | 0.052 | 0.005 | 0.557 | 0.759 |
| TTF2 | 0.658 | 0.054 | 0.005 | 0.553 | 0.763 |
| OXLD1 | 0.658 | 0.053 | 0.005 | 0.553 | 0.763 |
| HCST | 0.658 | 0.054 | 0.005 | 0.553 | 0.763 |
| ELMO1.AS1 | 0.658 | 0.052 | 0.005 | 0.556 | 0.760 |
| SAP30BP | 0.658 | 0.056 | 0.005 | 0.548 | 0.768 |
| UHRF1 | 0.658 | 0.057 | 0.005 | 0.546 | 0.769 |
| HPS1 | 0.657 | 0.051 | 0.005 | 0.557 | 0.757 |
| HIST1H4I | 0.657 | 0.054 | 0.005 | 0.550 | 0.764 |
| TMPO.AS1 | 0.657 | 0.051 | 0.005 | 0.557 | 0.756 |
| TIAF1 | 0.657 | 0.055 | 0.005 | 0.548 | 0.765 |
| IL2RA | 0.656 | 0.052 | 0.005 | 0.554 | 0.758 |
| IL4I1 | 0.656 | 0.053 | 0.005 | 0.553 | 0.759 |
| SOWAHD | 0.656 | 0.053 | 0.005 | 0.552 | 0.760 |
| EIF4EBP3 | 0.656 | 0.053 | 0.005 | 0.552 | 0.760 |
| FRMD4A | 0.656 | 0.052 | 0.005 | 0.554 | 0.758 |
| ELAVL1 | 0.655 | 0.054 | 0.005 | 0.550 | 0.761 |
| PRMT6 | 0.655 | 0.051 | 0.005 | 0.556 | 0.754 |
| HPSE | 0.655 | 0.057 | 0.005 | 0.544 | 0.766 |
| COG1 | 0.655 | 0.052 | 0.005 | 0.552 | 0.758 |
| NAGK | 0.655 | 0.051 | 0.005 | 0.556 | 0.754 |
| DOK3 | 0.655 | 0.053 | 0.006 | 0.551 | 0.759 |
| CENPX | 0.655 | 0.053 | 0.006 | 0.551 | 0.758 |
| ICAM2 | 0.655 | 0.053 | 0.006 | 0.550 | 0.759 |
| CDC16 | 0.654 | 0.055 | 0.006 | 0.547 | 0.761 |
| AP4B1 | 0.653 | 0.056 | 0.006 | 0.543 | 0.763 |
| SNRPA | 0.653 | 0.055 | 0.006 | 0.546 | 0.761 |
| DEF6 | 0.653 | 0.050 | 0.006 | 0.556 | 0.751 |
| PCTP | 0.653 | 0.053 | 0.006 | 0.550 | 0.756 |
| BRIP1 | 0.653 | 0.055 | 0.006 | 0.545 | 0.760 |
| SRP68 | 0.653 | 0.051 | 0.006 | 0.554 | 0.752 |
| UHRF1BP1 | 0.653 | 0.051 | 0.006 | 0.554 | 0.752 |
| MIS18BP1 | 0.652 | 0.055 | 0.006 | 0.545 | 0.759 |
| IRF5 | 0.652 | 0.053 | 0.006 | 0.549 | 0.756 |
| SPC24 | 0.652 | 0.054 | 0.006 | 0.547 | 0.758 |
| MAFG | 0.652 | 0.052 | 0.006 | 0.551 | 0.753 |
| FEN1 | 0.652 | 0.054 | 0.006 | 0.547 | 0.757 |
| ICE2 | 0.652 | 0.050 | 0.007 | 0.553 | 0.750 |
| MSL2 | 0.652 | 0.050 | 0.007 | 0.554 | 0.750 |
| DCUN1D2 | 0.652 | 0.053 | 0.007 | 0.548 | 0.755 |
| GPR18 | 0.652 | 0.054 | 0.007 | 0.546 | 0.757 |
| TLR1 | 0.652 | 0.053 | 0.007 | 0.548 | 0.755 |
| SNORD9 | 0.651 | 0.053 | 0.007 | 0.548 | 0.755 |
| CD180 | 0.651 | 0.054 | 0.007 | 0.545 | 0.756 |
| FN3KRP | 0.651 | 0.052 | 0.007 | 0.549 | 0.753 |
| AC022098.1 | 0.651 | 0.052 | 0.007 | 0.550 | 0.752 |
| IKZF4 | 0.651 | 0.051 | 0.007 | 0.551 | 0.751 |
| MXD3 | 0.651 | 0.053 | 0.007 | 0.547 | 0.755 |
| SNX11 | 0.651 | 0.052 | 0.007 | 0.549 | 0.752 |
| TRIM14 | 0.651 | 0.054 | 0.007 | 0.546 | 0.756 |
| SENP1 | 0.650 | 0.050 | 0.007 | 0.552 | 0.749 |
| STRADA | 0.650 | 0.053 | 0.007 | 0.547 | 0.754 |
| TP73 | 0.650 | 0.054 | 0.007 | 0.545 | 0.755 |
| EPB41L3 | 0.650 | 0.053 | 0.007 | 0.547 | 0.753 |
| GNA13 | 0.650 | 0.052 | 0.007 | 0.548 | 0.752 |
| RFC1 | 0.650 | 0.054 | 0.007 | 0.544 | 0.755 |
| RAB27A | 0.650 | 0.054 | 0.007 | 0.544 | 0.756 |
| HIST1H2AH | 0.650 | 0.052 | 0.007 | 0.547 | 0.753 |
| CEP131 | 0.650 | 0.054 | 0.007 | 0.543 | 0.756 |
| USP36 | 0.649 | 0.052 | 0.007 | 0.547 | 0.751 |
| CUL4A | 0.649 | 0.055 | 0.007 | 0.542 | 0.757 |
| NR1H3 | 0.649 | 0.053 | 0.007 | 0.545 | 0.754 |
| GPR19 | 0.649 | 0.052 | 0.008 | 0.547 | 0.751 |
| ADGRL1 | 0.649 | 0.054 | 0.008 | 0.544 | 0.754 |
| KLRB1 | 0.649 | 0.051 | 0.008 | 0.548 | 0.750 |
| LMF2 | 0.649 | 0.054 | 0.008 | 0.544 | 0.754 |
| CACTIN | 0.649 | 0.054 | 0.008 | 0.544 | 0.754 |
| HIST1H4B | 0.649 | 0.053 | 0.008 | 0.544 | 0.754 |
| HMGB2 | 0.648 | 0.056 | 0.008 | 0.539 | 0.758 |
| RIBC2 | 0.648 | 0.056 | 0.008 | 0.538 | 0.759 |
| STK24 | 0.648 | 0.051 | 0.008 | 0.549 | 0.748 |
| HAVCR2 | 0.648 | 0.056 | 0.008 | 0.538 | 0.759 |
| RAPGEF1 | 0.648 | 0.051 | 0.008 | 0.548 | 0.749 |
| MYO18A | 0.648 | 0.055 | 0.008 | 0.541 | 0.755 |
| ANKRD33B | 0.648 | 0.052 | 0.008 | 0.547 | 0.749 |
| ZNF619 | 0.648 | 0.052 | 0.008 | 0.545 | 0.750 |
| HIST1H2BK | 0.648 | 0.054 | 0.008 | 0.541 | 0.754 |
| XAB2 | 0.648 | 0.053 | 0.008 | 0.543 | 0.752 |
| HIST1H1C | 0.647 | 0.054 | 0.008 | 0.541 | 0.753 |
| DHX58 | 0.647 | 0.054 | 0.008 | 0.541 | 0.753 |
| PI4K2A | 0.647 | 0.055 | 0.008 | 0.539 | 0.755 |
| TUBA1B | 0.647 | 0.053 | 0.008 | 0.543 | 0.751 |
| CD83 | 0.647 | 0.051 | 0.009 | 0.548 | 0.746 |
| CDK5RAP3 | 0.647 | 0.054 | 0.009 | 0.542 | 0.752 |
| R3HCC1L | 0.647 | 0.054 | 0.009 | 0.541 | 0.753 |
| RAD51AP1 | 0.647 | 0.053 | 0.009 | 0.542 | 0.751 |
| ZNF576 | 0.646 | 0.049 | 0.009 | 0.550 | 0.743 |
| CD7 | 0.646 | 0.052 | 0.009 | 0.544 | 0.748 |
| ATP11A | 0.646 | 0.053 | 0.009 | 0.542 | 0.751 |
| POLE | 0.646 | 0.057 | 0.009 | 0.535 | 0.758 |
| SPATS2 | 0.646 | 0.052 | 0.009 | 0.545 | 0.747 |
| PSMD3 | 0.646 | 0.053 | 0.009 | 0.542 | 0.751 |
| SLC35A4 | 0.646 | 0.054 | 0.009 | 0.539 | 0.753 |
| HIST1H2AB | 0.646 | 0.054 | 0.009 | 0.539 | 0.752 |
| IRF8 | 0.646 | 0.054 | 0.009 | 0.539 | 0.752 |
| TNFAIP8L2 | 0.645 | 0.054 | 0.009 | 0.540 | 0.751 |
| DNAJC4 | 0.645 | 0.052 | 0.009 | 0.544 | 0.747 |
| ERCC5 | 0.645 | 0.054 | 0.009 | 0.540 | 0.751 |
| PGS1 | 0.645 | 0.054 | 0.009 | 0.540 | 0.751 |
| FAM96A | 0.645 | 0.051 | 0.009 | 0.546 | 0.745 |
| DAPK2 | 0.645 | 0.053 | 0.009 | 0.541 | 0.750 |
| KHSRP | 0.645 | 0.056 | 0.009 | 0.535 | 0.755 |
| ZNF618 | 0.645 | 0.054 | 0.009 | 0.539 | 0.751 |
| CD27 | 0.645 | 0.052 | 0.009 | 0.544 | 0.747 |
| ZNF385A | 0.645 | 0.054 | 0.009 | 0.539 | 0.750 |
| AC016245.1 | 0.645 | 0.054 | 0.009 | 0.538 | 0.751 |
| FANCA | 0.645 | 0.055 | 0.009 | 0.537 | 0.753 |
| FCER1G | 0.645 | 0.057 | 0.010 | 0.533 | 0.756 |
| FOXM1 | 0.645 | 0.053 | 0.010 | 0.540 | 0.749 |
| CTSS | 0.644 | 0.054 | 0.010 | 0.538 | 0.750 |
| MED24 | 0.644 | 0.054 | 0.010 | 0.539 | 0.749 |
| ARHGAP33 | 0.644 | 0.056 | 0.010 | 0.535 | 0.753 |
| LRRC8C | 0.644 | 0.054 | 0.010 | 0.538 | 0.749 |
| REXO1 | 0.643 | 0.051 | 0.010 | 0.543 | 0.744 |
| ZFYVE26 | 0.643 | 0.054 | 0.010 | 0.538 | 0.749 |
| UNK | 0.643 | 0.056 | 0.010 | 0.534 | 0.753 |
| CD300LF | 0.643 | 0.054 | 0.010 | 0.536 | 0.750 |
| GK | 0.643 | 0.052 | 0.010 | 0.542 | 0.744 |
| ZBTB11.AS1 | 0.643 | 0.053 | 0.010 | 0.538 | 0.748 |
| SLC16A11 | 0.643 | 0.053 | 0.010 | 0.539 | 0.747 |
| SLC24A1 | 0.642 | 0.054 | 0.011 | 0.537 | 0.748 |
| ARHGAP27P1.BPTFP1.KP | 0.642 | 0.050 | 0.011 | 0.544 | 0.741 |
| FICD | 0.642 | 0.051 | 0.011 | 0.543 | 0.742 |
| SKA2 | 0.642 | 0.053 | 0.011 | 0.539 | 0.746 |
| SP1 | 0.642 | 0.053 | 0.011 | 0.539 | 0.746 |
| NUPR1 | 0.642 | 0.053 | 0.011 | 0.538 | 0.747 |
| SIRT7 | 0.642 | 0.052 | 0.011 | 0.540 | 0.745 |
| IGSF6 | 0.642 | 0.052 | 0.011 | 0.541 | 0.743 |
| SMURF2 | 0.642 | 0.052 | 0.011 | 0.540 | 0.744 |
| SS18 | 0.642 | 0.051 | 0.011 | 0.542 | 0.742 |
| CABLES2 | 0.642 | 0.055 | 0.011 | 0.533 | 0.750 |
| ADAMDEC1 | 0.642 | 0.053 | 0.011 | 0.538 | 0.745 |
| PRSS36 | 0.642 | 0.054 | 0.011 | 0.535 | 0.748 |
| SEC14L1 | 0.641 | 0.052 | 0.011 | 0.539 | 0.744 |
| MARCO | 0.641 | 0.054 | 0.011 | 0.536 | 0.746 |
| GDAP2 | 0.641 | 0.051 | 0.011 | 0.541 | 0.741 |
| PATL1 | 0.641 | 0.053 | 0.011 | 0.538 | 0.745 |
| MIR3162 | 0.641 | 0.054 | 0.011 | 0.536 | 0.746 |
| CLN6 | 0.641 | 0.050 | 0.012 | 0.543 | 0.739 |
| NAGLU | 0.641 | 0.054 | 0.012 | 0.536 | 0.746 |
| LINC00847 | 0.641 | 0.053 | 0.012 | 0.537 | 0.744 |
| LY86 | 0.641 | 0.055 | 0.012 | 0.533 | 0.749 |
| CDT1 | 0.641 | 0.055 | 0.012 | 0.533 | 0.749 |
| MAP2K7 | 0.641 | 0.054 | 0.012 | 0.534 | 0.747 |
| DUS1L | 0.640 | 0.055 | 0.012 | 0.533 | 0.747 |
| NPLOC4 | 0.640 | 0.054 | 0.012 | 0.535 | 0.746 |
| TRIB1 | 0.640 | 0.053 | 0.012 | 0.537 | 0.743 |
| FAM111B | 0.640 | 0.057 | 0.012 | 0.529 | 0.752 |
| MIR3691 | 0.640 | 0.054 | 0.012 | 0.534 | 0.746 |
| E2F7 | 0.640 | 0.054 | 0.012 | 0.535 | 0.746 |
| RBM14 | 0.640 | 0.050 | 0.012 | 0.541 | 0.739 |
| MS4A14 | 0.640 | 0.055 | 0.012 | 0.532 | 0.748 |
| MED26 | 0.640 | 0.051 | 0.012 | 0.540 | 0.740 |
| RNF213 | 0.640 | 0.053 | 0.012 | 0.536 | 0.744 |
| HIST1H3D | 0.640 | 0.052 | 0.012 | 0.538 | 0.741 |
| HIST1H2BG | 0.639 | 0.055 | 0.012 | 0.531 | 0.748 |
| PDCD1LG2 | 0.639 | 0.053 | 0.012 | 0.535 | 0.744 |
| H2AFY | 0.639 | 0.053 | 0.013 | 0.535 | 0.744 |
| LEPROTL1 | 0.639 | 0.053 | 0.013 | 0.535 | 0.743 |
| KEAP1 | 0.639 | 0.052 | 0.013 | 0.536 | 0.741 |
| VPS29 | 0.639 | 0.055 | 0.013 | 0.530 | 0.747 |
| RASGEF1B | 0.639 | 0.050 | 0.013 | 0.541 | 0.736 |
| TYROBP | 0.639 | 0.056 | 0.013 | 0.528 | 0.749 |
| SLC7A7 | 0.639 | 0.056 | 0.013 | 0.530 | 0.747 |
| CCL8 | 0.639 | 0.054 | 0.013 | 0.534 | 0.744 |
| RAD54L | 0.639 | 0.058 | 0.013 | 0.526 | 0.751 |
| ARHGEF7 | 0.639 | 0.053 | 0.013 | 0.535 | 0.742 |
| ADGRF3 | 0.639 | 0.054 | 0.013 | 0.533 | 0.744 |
| FAM122B | 0.638 | 0.051 | 0.013 | 0.538 | 0.738 |
| HIST1H2BF | 0.638 | 0.055 | 0.013 | 0.531 | 0.745 |
| CLCC1 | 0.638 | 0.054 | 0.013 | 0.532 | 0.744 |
| RAVER1 | 0.638 | 0.051 | 0.013 | 0.537 | 0.739 |
| NEMP1 | 0.638 | 0.054 | 0.013 | 0.532 | 0.744 |
| C1QA | 0.638 | 0.055 | 0.013 | 0.529 | 0.747 |
| MDM2 | 0.638 | 0.054 | 0.013 | 0.532 | 0.744 |
| C17orf53 | 0.638 | 0.057 | 0.014 | 0.527 | 0.749 |
| CARS2 | 0.638 | 0.053 | 0.014 | 0.534 | 0.742 |
| DAPK1.IT1 | 0.638 | 0.054 | 0.014 | 0.532 | 0.743 |
| ZC3H12C | 0.638 | 0.053 | 0.014 | 0.535 | 0.741 |
| PILRA | 0.637 | 0.053 | 0.014 | 0.534 | 0.741 |
| TAPBPL | 0.637 | 0.054 | 0.014 | 0.532 | 0.742 |
| ZNF384 | 0.637 | 0.051 | 0.014 | 0.536 | 0.738 |
| ELF4 | 0.637 | 0.054 | 0.014 | 0.531 | 0.744 |
| CIP2A | 0.637 | 0.053 | 0.014 | 0.533 | 0.742 |
| PCK2 | 0.637 | 0.055 | 0.014 | 0.530 | 0.744 |
| ZNF706 | 0.637 | 0.052 | 0.014 | 0.534 | 0.740 |
| LAMP1 | 0.637 | 0.050 | 0.014 | 0.538 | 0.736 |
| TBC1D1 | 0.637 | 0.051 | 0.014 | 0.537 | 0.737 |
| SLBP | 0.637 | 0.053 | 0.014 | 0.532 | 0.742 |
| MLLT1 | 0.637 | 0.056 | 0.014 | 0.528 | 0.746 |
| FBF1 | 0.637 | 0.055 | 0.014 | 0.529 | 0.745 |
| SLC2A9 | 0.637 | 0.051 | 0.014 | 0.538 | 0.736 |
| CENPM | 0.637 | 0.054 | 0.014 | 0.531 | 0.742 |
| AC127496.1 | 0.637 | 0.055 | 0.014 | 0.529 | 0.745 |
| MRPL58 | 0.636 | 0.054 | 0.014 | 0.531 | 0.742 |
| TEX30 | 0.636 | 0.055 | 0.014 | 0.529 | 0.744 |
| BORA | 0.636 | 0.053 | 0.014 | 0.532 | 0.741 |
| TREM2 | 0.636 | 0.057 | 0.015 | 0.525 | 0.747 |
| GPR171 | 0.636 | 0.052 | 0.015 | 0.533 | 0.739 |
| HEXDC | 0.636 | 0.053 | 0.015 | 0.532 | 0.740 |
| RAB8A | 0.636 | 0.053 | 0.015 | 0.533 | 0.739 |
| CALML4 | 0.636 | 0.053 | 0.015 | 0.532 | 0.741 |
| FAR1 | 0.636 | 0.052 | 0.015 | 0.533 | 0.739 |
| TMEM140 | 0.636 | 0.052 | 0.015 | 0.533 | 0.739 |
| FAM78A | 0.636 | 0.055 | 0.015 | 0.528 | 0.743 |
| PIK3AP1 | 0.636 | 0.055 | 0.015 | 0.528 | 0.744 |
| FERMT3 | 0.636 | 0.056 | 0.015 | 0.527 | 0.745 |
| HIST1H4E | 0.636 | 0.053 | 0.015 | 0.531 | 0.740 |
| ZNF740 | 0.635 | 0.051 | 0.015 | 0.535 | 0.736 |
| PSPC1 | 0.635 | 0.054 | 0.015 | 0.530 | 0.740 |
| TNFAIP2 | 0.635 | 0.050 | 0.015 | 0.537 | 0.734 |
| C12orf4 | 0.635 | 0.053 | 0.016 | 0.532 | 0.738 |
| C6orf106 | 0.635 | 0.051 | 0.016 | 0.535 | 0.735 |
| ZNF324B | 0.635 | 0.055 | 0.016 | 0.526 | 0.743 |
| PDK2 | 0.634 | 0.054 | 0.016 | 0.528 | 0.741 |
| VEGFB | 0.634 | 0.052 | 0.016 | 0.532 | 0.736 |
| NFKBIE | 0.634 | 0.051 | 0.016 | 0.534 | 0.735 |
| ASB16 | 0.634 | 0.056 | 0.016 | 0.524 | 0.744 |
| TNFSF15 | 0.634 | 0.055 | 0.016 | 0.526 | 0.743 |
| ALG6 | 0.634 | 0.051 | 0.016 | 0.535 | 0.734 |
| MCOLN1 | 0.634 | 0.053 | 0.016 | 0.530 | 0.738 |
| HIST2H2BE | 0.634 | 0.055 | 0.016 | 0.525 | 0.743 |
| HIST1H2BL | 0.634 | 0.055 | 0.016 | 0.526 | 0.743 |
| TNFSF13B | 0.634 | 0.055 | 0.016 | 0.527 | 0.741 |
| GMIP | 0.634 | 0.052 | 0.017 | 0.532 | 0.736 |
| LAPTM5 | 0.634 | 0.056 | 0.017 | 0.523 | 0.744 |
| MTRR | 0.634 | 0.054 | 0.017 | 0.528 | 0.739 |
| C17orf113 | 0.634 | 0.055 | 0.017 | 0.527 | 0.741 |
| ZNF438 | 0.634 | 0.057 | 0.017 | 0.522 | 0.745 |
| SCIMP | 0.634 | 0.055 | 0.017 | 0.527 | 0.741 |
| HNRNPUL1 | 0.633 | 0.054 | 0.017 | 0.527 | 0.740 |
| LGMN | 0.633 | 0.053 | 0.017 | 0.529 | 0.737 |
| JMJD6 | 0.633 | 0.053 | 0.017 | 0.529 | 0.738 |
| INTS9 | 0.633 | 0.053 | 0.017 | 0.529 | 0.738 |
| FDXR | 0.633 | 0.055 | 0.017 | 0.526 | 0.741 |
| RAB35 | 0.633 | 0.052 | 0.017 | 0.532 | 0.735 |
| CDC42EP4 | 0.633 | 0.056 | 0.017 | 0.523 | 0.743 |
| ANAPC11 | 0.633 | 0.052 | 0.017 | 0.532 | 0.734 |
| KIF18A | 0.633 | 0.055 | 0.017 | 0.525 | 0.741 |
| PSMC5 | 0.633 | 0.056 | 0.017 | 0.524 | 0.742 |
| FMNL1 | 0.633 | 0.054 | 0.017 | 0.527 | 0.739 |
| LINC00996 | 0.633 | 0.051 | 0.017 | 0.532 | 0.733 |
| HAUS4 | 0.633 | 0.054 | 0.017 | 0.527 | 0.739 |
| CDC25C | 0.633 | 0.056 | 0.017 | 0.524 | 0.742 |
| ACP2 | 0.633 | 0.056 | 0.017 | 0.523 | 0.742 |
| KCTD2 | 0.633 | 0.054 | 0.017 | 0.527 | 0.739 |
| HEXA | 0.633 | 0.053 | 0.017 | 0.528 | 0.737 |
| SFMBT2 | 0.633 | 0.054 | 0.018 | 0.528 | 0.737 |
| MS4A4A | 0.633 | 0.056 | 0.018 | 0.523 | 0.742 |
| FAM19A3 | 0.633 | 0.055 | 0.018 | 0.524 | 0.741 |
| TIGD6 | 0.633 | 0.055 | 0.018 | 0.525 | 0.740 |
| IFI35 | 0.632 | 0.054 | 0.018 | 0.526 | 0.739 |
| ABCG1 | 0.632 | 0.054 | 0.018 | 0.526 | 0.738 |
| PSAP | 0.632 | 0.055 | 0.018 | 0.525 | 0.740 |
| MTMR4 | 0.632 | 0.052 | 0.018 | 0.531 | 0.734 |
| ASF1B | 0.632 | 0.055 | 0.018 | 0.525 | 0.739 |
| NUFIP2 | 0.632 | 0.057 | 0.018 | 0.521 | 0.743 |
| NRROS | 0.632 | 0.055 | 0.018 | 0.523 | 0.741 |
| ZNF395 | 0.632 | 0.054 | 0.018 | 0.526 | 0.737 |
| NT5C | 0.632 | 0.056 | 0.018 | 0.522 | 0.741 |
| TMEM94 | 0.632 | 0.056 | 0.018 | 0.521 | 0.742 |
| AP2A2 | 0.632 | 0.052 | 0.018 | 0.529 | 0.734 |
| WBP2 | 0.632 | 0.053 | 0.018 | 0.528 | 0.735 |
| SGSH | 0.631 | 0.053 | 0.019 | 0.528 | 0.735 |
| UBE2Z | 0.631 | 0.054 | 0.019 | 0.526 | 0.737 |
| C17orf100 | 0.631 | 0.057 | 0.019 | 0.520 | 0.742 |
| NME1 | 0.631 | 0.055 | 0.019 | 0.523 | 0.740 |
| FAM104A | 0.631 | 0.055 | 0.019 | 0.524 | 0.739 |
| IL10 | 0.631 | 0.054 | 0.019 | 0.525 | 0.738 |
| SKAP1 | 0.631 | 0.053 | 0.019 | 0.527 | 0.736 |
| PNPLA6 | 0.631 | 0.051 | 0.019 | 0.532 | 0.730 |
| ATG3 | 0.631 | 0.053 | 0.019 | 0.528 | 0.734 |
| HAUS5 | 0.631 | 0.056 | 0.019 | 0.522 | 0.740 |
| SEC22A | 0.631 | 0.053 | 0.019 | 0.527 | 0.735 |
| HELLS | 0.631 | 0.055 | 0.019 | 0.524 | 0.738 |
| IL10RA | 0.631 | 0.054 | 0.019 | 0.524 | 0.738 |
| SOAT1 | 0.631 | 0.054 | 0.019 | 0.525 | 0.737 |
| FCGR3A | 0.631 | 0.055 | 0.019 | 0.524 | 0.738 |
| MSL1 | 0.631 | 0.054 | 0.019 | 0.526 | 0.736 |
| ANKRD22 | 0.631 | 0.053 | 0.019 | 0.526 | 0.735 |
| TSPAN4 | 0.631 | 0.055 | 0.019 | 0.523 | 0.739 |
| KIF2A | 0.631 | 0.056 | 0.019 | 0.520 | 0.741 |
| FCGR1A | 0.630 | 0.055 | 0.019 | 0.523 | 0.738 |
| SLAMF8 | 0.630 | 0.056 | 0.019 | 0.521 | 0.740 |
| CSF3R | 0.630 | 0.053 | 0.019 | 0.527 | 0.734 |
| FUT10 | 0.630 | 0.055 | 0.019 | 0.523 | 0.738 |
| ANO9 | 0.630 | 0.053 | 0.020 | 0.526 | 0.734 |
| EXOC7 | 0.630 | 0.057 | 0.020 | 0.519 | 0.741 |
| DENND1A | 0.630 | 0.055 | 0.020 | 0.522 | 0.737 |
| DCK | 0.630 | 0.053 | 0.020 | 0.527 | 0.733 |
| C3 | 0.630 | 0.053 | 0.020 | 0.526 | 0.734 |
| TCAM1P | 0.630 | 0.055 | 0.020 | 0.521 | 0.738 |
| KIAA1551 | 0.630 | 0.051 | 0.020 | 0.529 | 0.730 |
| NCAPH2 | 0.630 | 0.052 | 0.020 | 0.527 | 0.732 |
| ALKBH6 | 0.629 | 0.054 | 0.021 | 0.523 | 0.736 |
| TMEM97 | 0.629 | 0.056 | 0.021 | 0.520 | 0.738 |
| MRPL27 | 0.629 | 0.056 | 0.021 | 0.520 | 0.738 |
| PCID2 | 0.629 | 0.056 | 0.021 | 0.520 | 0.739 |
| GZMB | 0.629 | 0.053 | 0.021 | 0.526 | 0.732 |
| CD81 | 0.629 | 0.052 | 0.021 | 0.527 | 0.731 |
| LAMP3 | 0.629 | 0.051 | 0.021 | 0.528 | 0.729 |
| ATP11A.AS1 | 0.629 | 0.054 | 0.021 | 0.523 | 0.734 |
| SGF29 | 0.628 | 0.055 | 0.021 | 0.521 | 0.735 |
| GTF2A2 | 0.628 | 0.052 | 0.021 | 0.526 | 0.730 |
| DRAM2 | 0.628 | 0.054 | 0.021 | 0.522 | 0.734 |
| CARMIL2 | 0.628 | 0.053 | 0.022 | 0.524 | 0.732 |
| PPP1R16B | 0.628 | 0.056 | 0.022 | 0.519 | 0.737 |
| ASPSCR1 | 0.628 | 0.054 | 0.022 | 0.521 | 0.735 |
| ARHGAP31.AS1 | 0.628 | 0.052 | 0.022 | 0.525 | 0.731 |
| AC124319.2 | 0.628 | 0.053 | 0.022 | 0.524 | 0.732 |
| PYY2 | 0.628 | 0.051 | 0.022 | 0.527 | 0.729 |
| TPP2 | 0.628 | 0.056 | 0.022 | 0.518 | 0.738 |
| CPM | 0.628 | 0.056 | 0.022 | 0.519 | 0.737 |
| RPS27L | 0.628 | 0.050 | 0.022 | 0.530 | 0.726 |
| SLC25A22 | 0.627 | 0.057 | 0.022 | 0.516 | 0.739 |
| SEMA4D | 0.627 | 0.051 | 0.022 | 0.527 | 0.728 |
| MIR199A1 | 0.627 | 0.055 | 0.022 | 0.519 | 0.736 |
| TBC1D22A | 0.627 | 0.054 | 0.022 | 0.522 | 0.732 |
| AHCYL1 | 0.627 | 0.055 | 0.022 | 0.520 | 0.735 |
| ARHGAP17 | 0.627 | 0.053 | 0.022 | 0.524 | 0.731 |
| C3AR1 | 0.627 | 0.057 | 0.022 | 0.516 | 0.739 |
| ESCO2 | 0.627 | 0.056 | 0.023 | 0.518 | 0.736 |
| IL12RB1 | 0.627 | 0.054 | 0.023 | 0.522 | 0.732 |
| AC015914.1 | 0.627 | 0.055 | 0.023 | 0.519 | 0.735 |
| PLA2G2D | 0.627 | 0.051 | 0.023 | 0.526 | 0.728 |
| CD72 | 0.627 | 0.054 | 0.023 | 0.521 | 0.733 |
| PSMD12 | 0.627 | 0.055 | 0.023 | 0.519 | 0.735 |
| HIST2H2AC | 0.627 | 0.053 | 0.023 | 0.522 | 0.731 |
| MBNL3 | 0.627 | 0.054 | 0.023 | 0.522 | 0.732 |
| LAP3 | 0.627 | 0.054 | 0.023 | 0.520 | 0.733 |
| DENND2D | 0.627 | 0.053 | 0.023 | 0.523 | 0.730 |
| POLA2 | 0.627 | 0.054 | 0.023 | 0.521 | 0.732 |
| FOXP3 | 0.627 | 0.052 | 0.023 | 0.524 | 0.729 |
| SEMA4A | 0.627 | 0.053 | 0.023 | 0.524 | 0.730 |
| CCL22 | 0.627 | 0.053 | 0.023 | 0.523 | 0.730 |
| CYB5R4 | 0.627 | 0.054 | 0.023 | 0.521 | 0.732 |
| SNX20 | 0.627 | 0.054 | 0.023 | 0.521 | 0.732 |
| RTP4 | 0.627 | 0.056 | 0.023 | 0.517 | 0.736 |
| ANKRD10 | 0.626 | 0.055 | 0.024 | 0.519 | 0.733 |
| MOB2 | 0.626 | 0.053 | 0.024 | 0.523 | 0.730 |
| PLGRKT | 0.626 | 0.052 | 0.024 | 0.523 | 0.729 |
| EFL1 | 0.626 | 0.053 | 0.024 | 0.523 | 0.729 |
| CNTLN | 0.626 | 0.052 | 0.024 | 0.524 | 0.729 |
| CMKLR1 | 0.626 | 0.055 | 0.024 | 0.518 | 0.734 |
| DZIP3 | 0.626 | 0.052 | 0.024 | 0.525 | 0.728 |
| OR2B2 | 0.626 | 0.054 | 0.024 | 0.521 | 0.731 |
| EVI5L | 0.626 | 0.055 | 0.024 | 0.519 | 0.733 |
| SH2B3 | 0.626 | 0.054 | 0.024 | 0.520 | 0.731 |
| TACC3 | 0.626 | 0.052 | 0.024 | 0.523 | 0.728 |
| RRP8 | 0.626 | 0.055 | 0.024 | 0.517 | 0.734 |
| SMC4 | 0.626 | 0.055 | 0.024 | 0.519 | 0.733 |
| TMEM161B | 0.626 | 0.055 | 0.024 | 0.518 | 0.733 |
| OSBPL7 | 0.626 | 0.058 | 0.024 | 0.513 | 0.738 |
| SRA1 | 0.626 | 0.056 | 0.024 | 0.516 | 0.736 |
| CCDC174 | 0.626 | 0.055 | 0.024 | 0.518 | 0.733 |
| ITGB3BP | 0.626 | 0.053 | 0.024 | 0.522 | 0.729 |
| APIP | 0.626 | 0.054 | 0.024 | 0.520 | 0.731 |
| SLC29A3 | 0.625 | 0.057 | 0.025 | 0.513 | 0.737 |
| STAT5A | 0.625 | 0.054 | 0.025 | 0.520 | 0.731 |
| GHDC | 0.625 | 0.054 | 0.025 | 0.519 | 0.732 |
| TMCO6 | 0.625 | 0.053 | 0.025 | 0.521 | 0.730 |
| CALM3 | 0.625 | 0.050 | 0.025 | 0.526 | 0.724 |
| SFXN2 | 0.625 | 0.052 | 0.025 | 0.523 | 0.727 |
| HIST1H1E | 0.625 | 0.053 | 0.025 | 0.522 | 0.728 |
| RENBP | 0.625 | 0.057 | 0.025 | 0.513 | 0.737 |
| RUBCN | 0.625 | 0.052 | 0.025 | 0.523 | 0.727 |
| RMI1 | 0.625 | 0.054 | 0.025 | 0.519 | 0.731 |
| FAM196B | 0.625 | 0.053 | 0.025 | 0.520 | 0.730 |
| C19orf38 | 0.625 | 0.058 | 0.025 | 0.512 | 0.738 |
| ARRB2 | 0.625 | 0.056 | 0.025 | 0.516 | 0.733 |
| CBY3 | 0.625 | 0.056 | 0.025 | 0.515 | 0.735 |
| SCAMP5 | 0.625 | 0.056 | 0.025 | 0.516 | 0.734 |
| TRAPPC2B | 0.625 | 0.051 | 0.025 | 0.524 | 0.725 |
| ATP6V1B2 | 0.625 | 0.053 | 0.025 | 0.522 | 0.728 |
| NAPSB | 0.624 | 0.052 | 0.026 | 0.522 | 0.726 |
| E2F1 | 0.624 | 0.055 | 0.026 | 0.516 | 0.732 |
| MCM2 | 0.624 | 0.054 | 0.026 | 0.518 | 0.731 |
| KNTC1 | 0.624 | 0.053 | 0.026 | 0.520 | 0.729 |
| MAP3K14 | 0.624 | 0.052 | 0.026 | 0.523 | 0.726 |
| ZNF672 | 0.624 | 0.051 | 0.026 | 0.525 | 0.724 |
| C15orf48 | 0.624 | 0.052 | 0.026 | 0.522 | 0.727 |
| DGKE | 0.624 | 0.057 | 0.026 | 0.513 | 0.736 |
| CD74 | 0.624 | 0.056 | 0.026 | 0.515 | 0.734 |
| GPS1 | 0.624 | 0.054 | 0.026 | 0.519 | 0.730 |
| SH2D2A | 0.624 | 0.054 | 0.026 | 0.518 | 0.730 |
| IQCB1 | 0.624 | 0.054 | 0.026 | 0.518 | 0.730 |
| CD47 | 0.624 | 0.052 | 0.026 | 0.523 | 0.725 |
| FKBP15 | 0.624 | 0.052 | 0.026 | 0.523 | 0.725 |
| ADAR | 0.624 | 0.053 | 0.026 | 0.521 | 0.728 |
| FUT7 | 0.624 | 0.053 | 0.026 | 0.521 | 0.728 |
| CDKN1B | 0.624 | 0.055 | 0.026 | 0.517 | 0.731 |
| EPSTI1 | 0.624 | 0.055 | 0.026 | 0.517 | 0.731 |
| DENND1C | 0.624 | 0.052 | 0.026 | 0.523 | 0.725 |
| CDK5RAP2 | 0.624 | 0.054 | 0.026 | 0.518 | 0.730 |
| MED9 | 0.624 | 0.054 | 0.026 | 0.518 | 0.730 |
| BCKDHA | 0.624 | 0.053 | 0.026 | 0.519 | 0.728 |
| FGD2 | 0.624 | 0.055 | 0.026 | 0.516 | 0.732 |
| CDC6 | 0.624 | 0.054 | 0.026 | 0.518 | 0.730 |
| NUP210 | 0.623 | 0.051 | 0.027 | 0.523 | 0.724 |
| CD52 | 0.623 | 0.054 | 0.027 | 0.518 | 0.729 |
| GABRP | 0.623 | 0.053 | 0.027 | 0.521 | 0.726 |
| MAGOH | 0.623 | 0.052 | 0.027 | 0.521 | 0.726 |
| DEK | 0.623 | 0.053 | 0.027 | 0.519 | 0.728 |
| SIGIRR | 0.623 | 0.055 | 0.027 | 0.516 | 0.731 |
| KCNAB2 | 0.623 | 0.055 | 0.027 | 0.516 | 0.731 |
| STIL | 0.623 | 0.056 | 0.027 | 0.513 | 0.734 |
| TTC9C | 0.623 | 0.054 | 0.027 | 0.517 | 0.730 |
| CD86 | 0.623 | 0.056 | 0.027 | 0.513 | 0.733 |
| CD80 | 0.623 | 0.056 | 0.027 | 0.514 | 0.732 |
| DENND4A | 0.623 | 0.052 | 0.027 | 0.520 | 0.726 |
| RBM38 | 0.623 | 0.052 | 0.028 | 0.520 | 0.726 |
| GIMAP5 | 0.623 | 0.055 | 0.028 | 0.516 | 0.730 |
| PSMD13 | 0.623 | 0.058 | 0.028 | 0.509 | 0.737 |
| ISG20 | 0.623 | 0.052 | 0.028 | 0.521 | 0.724 |
| MRPL38 | 0.623 | 0.055 | 0.028 | 0.514 | 0.732 |
| MIR744 | 0.623 | 0.055 | 0.028 | 0.514 | 0.731 |
| QTRT2 | 0.623 | 0.052 | 0.028 | 0.520 | 0.725 |
| ZNF557 | 0.623 | 0.054 | 0.028 | 0.517 | 0.728 |
| ZFP3 | 0.623 | 0.053 | 0.028 | 0.518 | 0.727 |
| METTL2A | 0.623 | 0.054 | 0.028 | 0.516 | 0.729 |
| PLA2G7 | 0.622 | 0.053 | 0.028 | 0.517 | 0.727 |
| SLC16A6 | 0.622 | 0.054 | 0.028 | 0.517 | 0.727 |
| MIR548AQ | 0.622 | 0.055 | 0.028 | 0.514 | 0.731 |
| SLC23A1 | 0.622 | 0.055 | 0.028 | 0.514 | 0.730 |
| ENGASE | 0.622 | 0.053 | 0.028 | 0.519 | 0.726 |
| ZDBF2 | 0.622 | 0.057 | 0.028 | 0.512 | 0.733 |
| PI4K2B | 0.622 | 0.051 | 0.029 | 0.521 | 0.723 |
| FNBP1 | 0.622 | 0.052 | 0.029 | 0.519 | 0.725 |
| RELB | 0.622 | 0.054 | 0.029 | 0.516 | 0.728 |
| RAC2 | 0.622 | 0.054 | 0.029 | 0.516 | 0.728 |
| FGF14.AS2 | 0.622 | 0.055 | 0.029 | 0.515 | 0.729 |
| ANP32B | 0.622 | 0.055 | 0.029 | 0.514 | 0.730 |
| FAM118A | 0.622 | 0.055 | 0.029 | 0.513 | 0.730 |
| NCF2 | 0.622 | 0.055 | 0.029 | 0.515 | 0.729 |
| TYK2 | 0.622 | 0.052 | 0.029 | 0.519 | 0.724 |
| TIMELESS | 0.622 | 0.056 | 0.029 | 0.513 | 0.731 |
| PPP1R3E | 0.622 | 0.056 | 0.029 | 0.513 | 0.731 |
| ALYREF | 0.622 | 0.054 | 0.029 | 0.516 | 0.728 |
| TMUB2 | 0.621 | 0.054 | 0.030 | 0.516 | 0.727 |
| SDR42E1 | 0.621 | 0.052 | 0.030 | 0.519 | 0.723 |
| CTLA4 | 0.621 | 0.054 | 0.030 | 0.516 | 0.727 |
| RRM2 | 0.621 | 0.055 | 0.030 | 0.513 | 0.730 |
| TOR1B | 0.621 | 0.054 | 0.030 | 0.515 | 0.727 |
| H2AFX | 0.621 | 0.055 | 0.030 | 0.515 | 0.728 |
| PSME3 | 0.621 | 0.053 | 0.030 | 0.517 | 0.726 |
| FPR3 | 0.621 | 0.056 | 0.030 | 0.511 | 0.732 |
| LRRCC1 | 0.621 | 0.057 | 0.030 | 0.510 | 0.732 |
| SLC15A2 | 0.621 | 0.053 | 0.030 | 0.518 | 0.724 |
| INCENP | 0.621 | 0.054 | 0.030 | 0.515 | 0.727 |
| NUDT5 | 0.621 | 0.055 | 0.030 | 0.514 | 0.728 |
| IKZF1 | 0.621 | 0.054 | 0.030 | 0.516 | 0.726 |
| RACGAP1 | 0.621 | 0.055 | 0.030 | 0.513 | 0.729 |
| CDKAL1 | 0.621 | 0.052 | 0.030 | 0.518 | 0.723 |
| RBPJ | 0.621 | 0.053 | 0.030 | 0.518 | 0.724 |
| SHKBP1 | 0.621 | 0.053 | 0.030 | 0.516 | 0.725 |
| TOR1A | 0.621 | 0.053 | 0.030 | 0.517 | 0.724 |
| IQCH.AS1 | 0.621 | 0.055 | 0.030 | 0.513 | 0.728 |
| AIM2 | 0.621 | 0.054 | 0.030 | 0.515 | 0.726 |
| C19orf66 | 0.621 | 0.056 | 0.030 | 0.510 | 0.732 |
| TBX21 | 0.621 | 0.052 | 0.030 | 0.518 | 0.723 |
| EIF4A3 | 0.621 | 0.055 | 0.030 | 0.514 | 0.728 |
| DMXL2 | 0.621 | 0.056 | 0.030 | 0.511 | 0.730 |
| SIGLEC7 | 0.621 | 0.055 | 0.030 | 0.512 | 0.730 |
| CCR7 | 0.621 | 0.052 | 0.030 | 0.518 | 0.723 |
| IFI30 | 0.620 | 0.056 | 0.031 | 0.511 | 0.730 |
| FGR | 0.620 | 0.054 | 0.031 | 0.514 | 0.727 |
| PARP16 | 0.620 | 0.054 | 0.031 | 0.515 | 0.726 |
| PADI2 | 0.620 | 0.054 | 0.031 | 0.515 | 0.726 |
| HIST1H2BI | 0.620 | 0.057 | 0.031 | 0.509 | 0.732 |
| ABCA5 | 0.620 | 0.054 | 0.031 | 0.514 | 0.727 |
| HGS | 0.620 | 0.054 | 0.031 | 0.514 | 0.726 |
| CIT | 0.620 | 0.058 | 0.031 | 0.507 | 0.733 |
| ZC3H12D | 0.620 | 0.054 | 0.031 | 0.514 | 0.726 |
| MIR4261 | 0.620 | 0.057 | 0.031 | 0.509 | 0.732 |
| PTRH2 | 0.620 | 0.053 | 0.031 | 0.516 | 0.724 |
| CD38 | 0.620 | 0.055 | 0.031 | 0.512 | 0.728 |
| CRYBG1 | 0.620 | 0.052 | 0.031 | 0.517 | 0.723 |
| METTL23 | 0.620 | 0.055 | 0.032 | 0.513 | 0.727 |
| UNC93B1 | 0.620 | 0.057 | 0.032 | 0.509 | 0.731 |
| PA2G4 | 0.620 | 0.053 | 0.032 | 0.515 | 0.724 |
| RHBDF2 | 0.620 | 0.056 | 0.032 | 0.510 | 0.730 |
| GAA | 0.620 | 0.053 | 0.032 | 0.516 | 0.724 |
| CCDC14 | 0.620 | 0.055 | 0.032 | 0.512 | 0.728 |
| FAAP100 | 0.620 | 0.055 | 0.032 | 0.511 | 0.728 |
| SPI1 | 0.620 | 0.055 | 0.032 | 0.512 | 0.727 |
| ITGB2 | 0.620 | 0.056 | 0.032 | 0.510 | 0.729 |
| DBF4B | 0.620 | 0.054 | 0.032 | 0.513 | 0.726 |
| POLA1 | 0.620 | 0.055 | 0.032 | 0.511 | 0.728 |
| TACO1 | 0.619 | 0.056 | 0.033 | 0.510 | 0.729 |
| GBP5 | 0.619 | 0.054 | 0.033 | 0.513 | 0.726 |
| CYBA | 0.619 | 0.056 | 0.033 | 0.509 | 0.729 |
| ETV7 | 0.619 | 0.054 | 0.033 | 0.514 | 0.725 |
| TEX2 | 0.619 | 0.053 | 0.033 | 0.515 | 0.724 |
| STK11 | 0.619 | 0.053 | 0.033 | 0.515 | 0.723 |
| PCBP2 | 0.619 | 0.055 | 0.033 | 0.512 | 0.726 |
| MIR155HG | 0.619 | 0.054 | 0.033 | 0.512 | 0.726 |
| GRK6 | 0.619 | 0.052 | 0.033 | 0.517 | 0.721 |
| IGF2BP2 | 0.619 | 0.051 | 0.033 | 0.520 | 0.718 |
| SART3 | 0.619 | 0.057 | 0.033 | 0.508 | 0.730 |
| SCAF4 | 0.619 | 0.054 | 0.033 | 0.513 | 0.725 |
| PCLAF | 0.619 | 0.056 | 0.033 | 0.509 | 0.729 |
| LSM12 | 0.619 | 0.054 | 0.033 | 0.512 | 0.726 |
| RFC5 | 0.619 | 0.056 | 0.033 | 0.509 | 0.729 |
| ASTE1 | 0.619 | 0.056 | 0.033 | 0.510 | 0.728 |
| AKAP10 | 0.619 | 0.054 | 0.033 | 0.513 | 0.725 |
| TMEM116 | 0.619 | 0.053 | 0.033 | 0.516 | 0.722 |
| TAF3 | 0.619 | 0.053 | 0.033 | 0.514 | 0.723 |
| PIK3R5 | 0.619 | 0.055 | 0.033 | 0.511 | 0.726 |
| EXOSC9 | 0.619 | 0.055 | 0.033 | 0.511 | 0.726 |
| ANAPC10 | 0.619 | 0.056 | 0.033 | 0.509 | 0.729 |
| RNF168 | 0.619 | 0.051 | 0.033 | 0.520 | 0.718 |
| ATAD5 | 0.619 | 0.055 | 0.033 | 0.511 | 0.727 |
| FBXO48 | 0.619 | 0.052 | 0.033 | 0.517 | 0.721 |
| SLF2 | 0.619 | 0.054 | 0.033 | 0.514 | 0.724 |
| SP140 | 0.619 | 0.053 | 0.033 | 0.515 | 0.723 |
| CD8B | 0.618 | 0.052 | 0.034 | 0.516 | 0.721 |
| APOBEC3F | 0.618 | 0.054 | 0.034 | 0.512 | 0.725 |
| YY1AP1 | 0.618 | 0.053 | 0.034 | 0.515 | 0.721 |
| NPL | 0.618 | 0.057 | 0.034 | 0.508 | 0.729 |
| TAF4B | 0.618 | 0.052 | 0.034 | 0.516 | 0.721 |
| MLX | 0.618 | 0.055 | 0.034 | 0.511 | 0.726 |
| VAV1 | 0.618 | 0.055 | 0.034 | 0.510 | 0.726 |
| TMIGD3 | 0.618 | 0.055 | 0.034 | 0.511 | 0.725 |
| PRIM1 | 0.618 | 0.052 | 0.034 | 0.517 | 0.720 |
| SEC61B | 0.618 | 0.053 | 0.034 | 0.514 | 0.723 |
| CELF2.AS1 | 0.618 | 0.053 | 0.034 | 0.515 | 0.722 |
| SPAAR | 0.618 | 0.055 | 0.034 | 0.510 | 0.727 |
| USF3 | 0.618 | 0.054 | 0.034 | 0.512 | 0.724 |
| SLCO2B1 | 0.618 | 0.055 | 0.034 | 0.511 | 0.726 |
| HJURP | 0.618 | 0.056 | 0.034 | 0.508 | 0.729 |
| AKNA | 0.618 | 0.053 | 0.034 | 0.515 | 0.721 |
| HCK | 0.618 | 0.054 | 0.034 | 0.511 | 0.725 |
| RFNG | 0.618 | 0.055 | 0.034 | 0.510 | 0.726 |
| CXCL10 | 0.618 | 0.053 | 0.034 | 0.515 | 0.722 |
| CCDC97 | 0.618 | 0.053 | 0.035 | 0.513 | 0.723 |
| C2CD5 | 0.618 | 0.053 | 0.035 | 0.514 | 0.722 |
| ECSIT | 0.618 | 0.053 | 0.035 | 0.514 | 0.721 |
| RPS6KB1 | 0.618 | 0.054 | 0.035 | 0.512 | 0.723 |
| WDR5B | 0.618 | 0.054 | 0.035 | 0.512 | 0.724 |
| GZMM | 0.618 | 0.052 | 0.035 | 0.515 | 0.720 |
| CD84 | 0.617 | 0.056 | 0.035 | 0.508 | 0.727 |
| ITGAL | 0.617 | 0.054 | 0.035 | 0.512 | 0.723 |
| ZBED2 | 0.617 | 0.053 | 0.035 | 0.514 | 0.721 |
| C5orf56 | 0.617 | 0.055 | 0.035 | 0.510 | 0.725 |
| PTPRO | 0.617 | 0.058 | 0.035 | 0.503 | 0.732 |
| ARAF | 0.617 | 0.054 | 0.036 | 0.511 | 0.724 |
| KLHL11 | 0.617 | 0.052 | 0.036 | 0.514 | 0.720 |
| TRA2B | 0.617 | 0.053 | 0.036 | 0.513 | 0.721 |
| SASH3 | 0.617 | 0.055 | 0.036 | 0.510 | 0.725 |
| CANT1 | 0.617 | 0.054 | 0.036 | 0.510 | 0.724 |
| C10orf128 | 0.617 | 0.057 | 0.036 | 0.505 | 0.729 |
| EBAG9 | 0.617 | 0.054 | 0.036 | 0.511 | 0.722 |
| HIST1H2BN | 0.617 | 0.056 | 0.036 | 0.507 | 0.727 |
| RETREG3 | 0.617 | 0.056 | 0.036 | 0.508 | 0.726 |
| SLFN13 | 0.617 | 0.058 | 0.036 | 0.502 | 0.731 |
| ARSG | 0.617 | 0.053 | 0.036 | 0.513 | 0.720 |
| KAT7 | 0.617 | 0.053 | 0.036 | 0.512 | 0.721 |
| TSSC4 | 0.617 | 0.060 | 0.036 | 0.499 | 0.735 |
| INTS14 | 0.617 | 0.054 | 0.036 | 0.511 | 0.722 |
| SMARCA4 | 0.617 | 0.055 | 0.036 | 0.510 | 0.724 |
| WDR88 | 0.617 | 0.052 | 0.036 | 0.515 | 0.719 |
| U2AF1L4 | 0.617 | 0.054 | 0.037 | 0.511 | 0.722 |
| PRR22 | 0.617 | 0.054 | 0.037 | 0.511 | 0.722 |
| NDUFAF6 | 0.617 | 0.053 | 0.037 | 0.512 | 0.721 |
| NXPE3 | 0.617 | 0.053 | 0.037 | 0.512 | 0.721 |
| MFNG | 0.617 | 0.055 | 0.037 | 0.508 | 0.725 |
| LRRC23 | 0.617 | 0.051 | 0.037 | 0.516 | 0.717 |
| MIR921 | 0.616 | 0.053 | 0.037 | 0.512 | 0.720 |
| WDR34 | 0.616 | 0.057 | 0.037 | 0.505 | 0.728 |
| HERC1 | 0.616 | 0.053 | 0.037 | 0.512 | 0.721 |
| ICOS | 0.616 | 0.054 | 0.037 | 0.511 | 0.722 |
| NASP | 0.616 | 0.056 | 0.037 | 0.507 | 0.725 |
| SMCR8 | 0.616 | 0.054 | 0.037 | 0.510 | 0.723 |
| GOSR2 | 0.616 | 0.056 | 0.037 | 0.507 | 0.725 |
| DPYSL2 | 0.616 | 0.054 | 0.037 | 0.511 | 0.722 |
| UPF3B | 0.616 | 0.054 | 0.037 | 0.510 | 0.722 |
| TIPIN | 0.616 | 0.053 | 0.037 | 0.512 | 0.721 |
| HIST1H4H | 0.616 | 0.054 | 0.037 | 0.511 | 0.721 |
| CD69 | 0.616 | 0.052 | 0.038 | 0.514 | 0.718 |
| MTMR14 | 0.616 | 0.050 | 0.038 | 0.518 | 0.714 |
| LY9 | 0.616 | 0.052 | 0.038 | 0.514 | 0.718 |
| MFSD13A | 0.616 | 0.055 | 0.038 | 0.507 | 0.725 |
| RELL1 | 0.616 | 0.052 | 0.038 | 0.515 | 0.717 |
| CDC45 | 0.616 | 0.056 | 0.038 | 0.507 | 0.725 |
| COIL | 0.616 | 0.053 | 0.038 | 0.511 | 0.720 |
| KCNA3 | 0.616 | 0.052 | 0.038 | 0.513 | 0.718 |
| CDCA3 | 0.616 | 0.053 | 0.038 | 0.513 | 0.719 |
| MYO1F | 0.616 | 0.054 | 0.038 | 0.509 | 0.722 |
| TNIP1 | 0.616 | 0.053 | 0.038 | 0.512 | 0.720 |
| DONSON | 0.615 | 0.054 | 0.039 | 0.510 | 0.721 |
| HTRA4 | 0.615 | 0.056 | 0.039 | 0.506 | 0.725 |
| PLEK | 0.615 | 0.054 | 0.039 | 0.510 | 0.721 |
| RAB20 | 0.615 | 0.055 | 0.039 | 0.508 | 0.723 |
| CRELD2 | 0.615 | 0.054 | 0.039 | 0.510 | 0.720 |
| SF1 | 0.615 | 0.051 | 0.039 | 0.515 | 0.715 |
| SLC38A10 | 0.615 | 0.054 | 0.039 | 0.509 | 0.721 |
| PARVG | 0.615 | 0.057 | 0.039 | 0.504 | 0.727 |
| HCFC1 | 0.615 | 0.054 | 0.039 | 0.509 | 0.721 |
| SAFB | 0.615 | 0.055 | 0.039 | 0.508 | 0.723 |
| WAS | 0.615 | 0.056 | 0.039 | 0.506 | 0.724 |
| EMILIN2 | 0.615 | 0.055 | 0.039 | 0.507 | 0.723 |
| SOCS1 | 0.615 | 0.053 | 0.039 | 0.511 | 0.719 |
| RFWD3 | 0.615 | 0.056 | 0.039 | 0.505 | 0.725 |
| GTPBP1 | 0.615 | 0.056 | 0.039 | 0.505 | 0.726 |
| PXMP2 | 0.615 | 0.057 | 0.039 | 0.503 | 0.727 |
| TMEM11 | 0.615 | 0.057 | 0.039 | 0.503 | 0.727 |
| VPS25 | 0.615 | 0.054 | 0.039 | 0.510 | 0.721 |
| AL109955.1 | 0.615 | 0.055 | 0.039 | 0.507 | 0.723 |
| SMPDL3A | 0.615 | 0.055 | 0.039 | 0.508 | 0.722 |
| HIST1H3B | 0.615 | 0.056 | 0.039 | 0.506 | 0.724 |
| MGAT1 | 0.615 | 0.054 | 0.039 | 0.509 | 0.721 |
| ZBP1 | 0.615 | 0.056 | 0.040 | 0.506 | 0.724 |
| HIST1H2AE | 0.615 | 0.054 | 0.040 | 0.508 | 0.721 |
| GNG5 | 0.615 | 0.054 | 0.040 | 0.509 | 0.720 |
| MAP4K1 | 0.615 | 0.054 | 0.040 | 0.508 | 0.722 |
| U62317.2 | 0.615 | 0.055 | 0.040 | 0.507 | 0.723 |
| ZBTB33 | 0.615 | 0.052 | 0.040 | 0.512 | 0.718 |
| PCNX3 | 0.615 | 0.053 | 0.040 | 0.511 | 0.718 |
| KLHL6 | 0.615 | 0.055 | 0.040 | 0.507 | 0.722 |
| MGAT3.AS1 | 0.615 | 0.055 | 0.040 | 0.506 | 0.723 |
| AC012615.6 | 0.614 | 0.053 | 0.040 | 0.511 | 0.718 |
| ARF6 | 0.614 | 0.054 | 0.040 | 0.508 | 0.721 |
| DEPDC1B | 0.614 | 0.058 | 0.040 | 0.501 | 0.728 |
| ZBTB39 | 0.614 | 0.052 | 0.040 | 0.512 | 0.717 |
| PLEKHO1 | 0.614 | 0.054 | 0.040 | 0.509 | 0.720 |
| RNF138 | 0.614 | 0.052 | 0.040 | 0.512 | 0.717 |
| RTKN2 | 0.614 | 0.054 | 0.040 | 0.508 | 0.720 |
| ADA2 | 0.614 | 0.055 | 0.040 | 0.507 | 0.722 |
| KLHL5 | 0.614 | 0.056 | 0.040 | 0.505 | 0.724 |
| CORO1C | 0.614 | 0.053 | 0.041 | 0.510 | 0.718 |
| SGO1 | 0.614 | 0.055 | 0.041 | 0.507 | 0.721 |
| PALB2 | 0.614 | 0.055 | 0.041 | 0.506 | 0.723 |
| CORO1A | 0.614 | 0.055 | 0.041 | 0.506 | 0.722 |
| PRSS12 | 0.614 | 0.062 | 0.041 | 0.493 | 0.735 |
| HARS | 0.614 | 0.056 | 0.041 | 0.504 | 0.724 |
| SNX10 | 0.614 | 0.054 | 0.041 | 0.508 | 0.720 |
| RORA.AS1 | 0.614 | 0.051 | 0.041 | 0.514 | 0.714 |
| CHIT1 | 0.614 | 0.054 | 0.041 | 0.508 | 0.720 |
| HMCES | 0.614 | 0.053 | 0.041 | 0.510 | 0.718 |
| LETMD1 | 0.614 | 0.055 | 0.041 | 0.507 | 0.721 |
| H1FX | 0.614 | 0.054 | 0.041 | 0.507 | 0.720 |
| IL32 | 0.614 | 0.051 | 0.041 | 0.514 | 0.713 |
| LSP1 | 0.614 | 0.055 | 0.041 | 0.506 | 0.722 |
| MRPL11 | 0.614 | 0.053 | 0.041 | 0.510 | 0.718 |
| TDRD7 | 0.614 | 0.053 | 0.041 | 0.509 | 0.718 |
| TPRA1 | 0.614 | 0.052 | 0.041 | 0.512 | 0.715 |
| ABHD15 | 0.614 | 0.054 | 0.041 | 0.509 | 0.719 |
| NUP62CL | 0.614 | 0.056 | 0.041 | 0.504 | 0.723 |
| DNASE2 | 0.614 | 0.055 | 0.042 | 0.506 | 0.721 |
| MIR135A1 | 0.614 | 0.056 | 0.042 | 0.504 | 0.723 |
| WNT10A | 0.614 | 0.054 | 0.042 | 0.508 | 0.719 |
| OXR1 | 0.614 | 0.054 | 0.042 | 0.507 | 0.720 |
| ADGRG5 | 0.614 | 0.053 | 0.042 | 0.509 | 0.718 |
| MLF2 | 0.614 | 0.053 | 0.042 | 0.510 | 0.717 |
| CLPP | 0.614 | 0.054 | 0.042 | 0.508 | 0.719 |
| LCP2 | 0.614 | 0.056 | 0.042 | 0.504 | 0.723 |
| MED11 | 0.614 | 0.056 | 0.042 | 0.504 | 0.723 |
| SLFN12L | 0.614 | 0.053 | 0.042 | 0.509 | 0.718 |
| NECAP1 | 0.614 | 0.054 | 0.042 | 0.507 | 0.720 |
| SMIM6 | 0.613 | 0.060 | 0.042 | 0.496 | 0.731 |
| NUP98 | 0.613 | 0.058 | 0.042 | 0.499 | 0.727 |
| ZNF586 | 0.613 | 0.052 | 0.042 | 0.511 | 0.716 |
| USP48 | 0.613 | 0.056 | 0.042 | 0.503 | 0.723 |
| TCF3 | 0.613 | 0.052 | 0.042 | 0.511 | 0.715 |
| DHPS | 0.613 | 0.053 | 0.042 | 0.510 | 0.716 |
| SMG8 | 0.613 | 0.053 | 0.042 | 0.510 | 0.717 |
| MAP3K14.AS1 | 0.613 | 0.054 | 0.042 | 0.508 | 0.719 |
| ATG2A | 0.613 | 0.051 | 0.042 | 0.513 | 0.713 |
| USP37 | 0.613 | 0.055 | 0.042 | 0.505 | 0.722 |
| CLUHP3 | 0.613 | 0.054 | 0.042 | 0.507 | 0.720 |
| ZNF830 | 0.613 | 0.053 | 0.043 | 0.510 | 0.716 |
| HIST1H3G | 0.613 | 0.059 | 0.043 | 0.497 | 0.729 |
| BATF2 | 0.613 | 0.054 | 0.043 | 0.507 | 0.719 |
| UNC13D | 0.613 | 0.054 | 0.043 | 0.507 | 0.719 |
| PLCG2 | 0.613 | 0.056 | 0.043 | 0.504 | 0.722 |
| PCP4L1 | 0.613 | 0.055 | 0.043 | 0.506 | 0.720 |
| NCF1C | 0.613 | 0.054 | 0.043 | 0.506 | 0.719 |
| KAT2B | 0.613 | 0.052 | 0.043 | 0.512 | 0.714 |
| ZNF367 | 0.613 | 0.056 | 0.043 | 0.502 | 0.723 |
| WDFY4 | 0.613 | 0.056 | 0.043 | 0.504 | 0.722 |
| HSPBAP1 | 0.613 | 0.054 | 0.043 | 0.506 | 0.719 |
| SBF2.AS1 | 0.613 | 0.057 | 0.043 | 0.501 | 0.724 |
| LUC7L3 | 0.613 | 0.056 | 0.043 | 0.503 | 0.722 |
| GLE1 | 0.613 | 0.053 | 0.043 | 0.510 | 0.716 |
| CDCA5 | 0.612 | 0.055 | 0.044 | 0.504 | 0.721 |
| C17orf80 | 0.612 | 0.054 | 0.044 | 0.506 | 0.719 |
| MSR1 | 0.612 | 0.054 | 0.044 | 0.506 | 0.719 |
| MRPS7 | 0.612 | 0.055 | 0.044 | 0.504 | 0.721 |
| CALHM6 | 0.612 | 0.056 | 0.044 | 0.503 | 0.722 |
| PLD4 | 0.612 | 0.053 | 0.044 | 0.508 | 0.717 |
| GPR52 | 0.612 | 0.055 | 0.045 | 0.504 | 0.720 |
| PLCD4 | 0.612 | 0.052 | 0.045 | 0.509 | 0.715 |
| MYL6B | 0.612 | 0.052 | 0.045 | 0.511 | 0.713 |
| BIRC3 | 0.612 | 0.053 | 0.045 | 0.508 | 0.716 |
| LMNB1 | 0.612 | 0.058 | 0.045 | 0.499 | 0.725 |
| TOP2A | 0.612 | 0.058 | 0.045 | 0.499 | 0.725 |
| OAS2 | 0.612 | 0.054 | 0.045 | 0.507 | 0.717 |
| ASAP1 | 0.612 | 0.052 | 0.045 | 0.510 | 0.714 |
| FCHSD1 | 0.612 | 0.055 | 0.045 | 0.505 | 0.719 |
| NCKAP1L | 0.612 | 0.056 | 0.045 | 0.503 | 0.721 |
| MS4A4E | 0.612 | 0.053 | 0.045 | 0.507 | 0.717 |
| RELA | 0.612 | 0.052 | 0.045 | 0.510 | 0.713 |
| BIRC5 | 0.612 | 0.055 | 0.045 | 0.504 | 0.720 |
| PTDSS2 | 0.612 | 0.055 | 0.045 | 0.503 | 0.720 |
| ING4 | 0.612 | 0.053 | 0.045 | 0.508 | 0.715 |
| ZNF79 | 0.612 | 0.053 | 0.045 | 0.508 | 0.715 |
| SNF8 | 0.612 | 0.054 | 0.045 | 0.506 | 0.718 |
| SLAMF7 | 0.612 | 0.053 | 0.045 | 0.507 | 0.716 |
| CMC2 | 0.611 | 0.056 | 0.046 | 0.503 | 0.720 |
| SNRPD1 | 0.611 | 0.054 | 0.046 | 0.506 | 0.717 |
| PTPN7 | 0.611 | 0.054 | 0.046 | 0.506 | 0.717 |
| IL15RA | 0.611 | 0.052 | 0.046 | 0.509 | 0.714 |
| GTF2F1 | 0.611 | 0.058 | 0.046 | 0.498 | 0.725 |
| STOML1 | 0.611 | 0.055 | 0.046 | 0.503 | 0.720 |
| CCDC94 | 0.611 | 0.053 | 0.046 | 0.508 | 0.715 |
| CD53 | 0.611 | 0.056 | 0.046 | 0.501 | 0.721 |
| FTSJ3 | 0.611 | 0.057 | 0.046 | 0.498 | 0.724 |
| PER3 | 0.611 | 0.055 | 0.046 | 0.504 | 0.718 |
| ILF3 | 0.611 | 0.055 | 0.046 | 0.503 | 0.719 |
| ZNF76 | 0.611 | 0.054 | 0.046 | 0.505 | 0.717 |
| PTTG2 | 0.611 | 0.054 | 0.046 | 0.505 | 0.717 |
| IL6R | 0.611 | 0.053 | 0.046 | 0.507 | 0.715 |
| GPATCH11 | 0.611 | 0.053 | 0.046 | 0.508 | 0.715 |
| PRF1 | 0.611 | 0.054 | 0.046 | 0.506 | 0.716 |
| PLAC8 | 0.611 | 0.052 | 0.047 | 0.509 | 0.713 |
| USP1 | 0.611 | 0.054 | 0.047 | 0.504 | 0.718 |
| CXCL16 | 0.611 | 0.055 | 0.047 | 0.504 | 0.718 |
| GUSB | 0.611 | 0.052 | 0.047 | 0.509 | 0.713 |
| CD300A | 0.611 | 0.054 | 0.047 | 0.505 | 0.717 |
| TROAP | 0.611 | 0.058 | 0.047 | 0.498 | 0.724 |
| LINC01775 | 0.611 | 0.058 | 0.047 | 0.497 | 0.725 |
| TRAF1 | 0.611 | 0.053 | 0.047 | 0.507 | 0.715 |
| ATP5H | 0.611 | 0.054 | 0.047 | 0.505 | 0.717 |
| TFEC | 0.611 | 0.056 | 0.047 | 0.502 | 0.720 |
| PIDD1 | 0.611 | 0.054 | 0.047 | 0.504 | 0.718 |
| NFATC2 | 0.611 | 0.053 | 0.047 | 0.507 | 0.715 |
| ADPGK | 0.611 | 0.053 | 0.048 | 0.508 | 0.714 |
| NDUFA11 | 0.611 | 0.055 | 0.048 | 0.503 | 0.718 |
| ORC1 | 0.611 | 0.056 | 0.048 | 0.501 | 0.720 |
| NUDT3 | 0.611 | 0.055 | 0.048 | 0.504 | 0.717 |
| IFNK | 0.611 | 0.054 | 0.048 | 0.505 | 0.716 |
| STX11 | 0.611 | 0.056 | 0.048 | 0.501 | 0.721 |
| NLGN3 | 0.611 | 0.052 | 0.048 | 0.508 | 0.713 |
| SELPLG | 0.610 | 0.055 | 0.048 | 0.503 | 0.718 |
| NFKB1 | 0.610 | 0.056 | 0.048 | 0.500 | 0.720 |
| CDC7 | 0.610 | 0.058 | 0.048 | 0.497 | 0.723 |
| UTP18 | 0.610 | 0.052 | 0.048 | 0.507 | 0.713 |
| TNNI2 | 0.610 | 0.055 | 0.048 | 0.503 | 0.718 |
| TNFRSF1A | 0.610 | 0.052 | 0.048 | 0.508 | 0.713 |
| TNFSF14 | 0.610 | 0.054 | 0.048 | 0.505 | 0.715 |
| HELZ2 | 0.610 | 0.053 | 0.048 | 0.507 | 0.714 |
| LPXN | 0.610 | 0.055 | 0.048 | 0.503 | 0.718 |
| SF3A2 | 0.610 | 0.057 | 0.049 | 0.498 | 0.722 |
| RNASE6 | 0.610 | 0.056 | 0.049 | 0.500 | 0.720 |
| HIST1H2AJ | 0.610 | 0.054 | 0.049 | 0.504 | 0.716 |
| SH3BP1 | 0.610 | 0.053 | 0.049 | 0.505 | 0.715 |
| CLPTM1L | 0.610 | 0.057 | 0.049 | 0.499 | 0.721 |
| MCRS1 | 0.610 | 0.052 | 0.049 | 0.507 | 0.713 |
| TNFSF13 | 0.610 | 0.055 | 0.049 | 0.503 | 0.717 |
| X1.Sep | 0.610 | 0.054 | 0.049 | 0.504 | 0.715 |
| TBCD | 0.610 | 0.054 | 0.049 | 0.503 | 0.716 |
| MX1 | 0.610 | 0.053 | 0.049 | 0.506 | 0.713 |
| NAT9 | 0.610 | 0.056 | 0.049 | 0.500 | 0.719 |
| THRA1.BTR | 0.610 | 0.054 | 0.049 | 0.505 | 0.715 |
| TOR3A | 0.610 | 0.055 | 0.049 | 0.503 | 0.716 |
| UCP2 | 0.610 | 0.051 | 0.049 | 0.510 | 0.709 |
| DAPK1 | 0.610 | 0.055 | 0.049 | 0.502 | 0.717 |
| HSH2D | 0.610 | 0.054 | 0.049 | 0.504 | 0.715 |

TABLE 6
Genes predictive of RD from the GSE164458_C cohort..
Area under the curve

| | | | | Asymptotic 95% | |
|---|---|---|---|---|---|
| | | | | Confidence Interval | |
| Test Result | | Std. | Asymptotic | Lower | Upper |
| Variable(s) | Area | Error^{a} | Sig.^{b} | Bound | Bound |
| PTPN21 | 0.692 | 0.049 | 0.001 | 0.596 | 0.788 |
| TACC2 | 0.688 | 0.050 | 0.001 | 0.590 | 0.786 |
| EPB41L5 | 0.685 | 0.051 | 0.001 | 0.585 | 0.785 |
| EGLN1 | 0.685 | 0.056 | 0.001 | 0.576 | 0.794 |
| PON2 | 0.682 | 0.049 | 0.001 | 0.585 | 0.778 |
| RNF217 | 0.681 | 0.049 | 0.001 | 0.584 | 0.777 |
| Z93022.1 | 0.678 | 0.050 | 0.001 | 0.579 | 0.776 |
| AKR1C2 | 0.678 | 0.050 | 0.001 | 0.580 | 0.775 |
| PCYOX1 | 0.677 | 0.049 | 0.002 | 0.581 | 0.773 |
| TMEM181 | 0.676 | 0.052 | 0.002 | 0.575 | 0.778 |
| AIG1 | 0.676 | 0.051 | 0.002 | 0.576 | 0.776 |
| FSIP2 | 0.675 | 0.049 | 0.002 | 0.579 | 0.771 |
| THSD4 | 0.675 | 0.051 | 0.002 | 0.574 | 0.775 |
| TUBB2A | 0.671 | 0.052 | 0.002 | 0.569 | 0.773 |
| PAPPA | 0.667 | 0.050 | 0.003 | 0.569 | 0.765 |
| EGFR | 0.666 | 0.053 | 0.003 | 0.561 | 0.771 |
| PLAT | 0.666 | 0.053 | 0.003 | 0.563 | 0.769 |
| PRKAR2A | 0.666 | 0.054 | 0.003 | 0.561 | 0.771 |
| HEYL | 0.664 | 0.051 | 0.003 | 0.564 | 0.764 |
| TTC8 | 0.663 | 0.049 | 0.003 | 0.567 | 0.760 |
| LINC01876 | 0.662 | 0.054 | 0.004 | 0.557 | 0.768 |
| PBOV1 | 0.662 | 0.053 | 0.004 | 0.559 | 0.765 |
| CD55 | 0.661 | 0.052 | 0.004 | 0.559 | 0.763 |
| COL7A1 | 0.661 | 0.054 | 0.004 | 0.555 | 0.766 |
| RNU4ATAC | 0.660 | 0.053 | 0.004 | 0.555 | 0.764 |
| RSPH3 | 0.658 | 0.053 | 0.005 | 0.555 | 0.762 |
| FLRT3 | 0.658 | 0.052 | 0.005 | 0.556 | 0.761 |
| ARFGEF3 | 0.657 | 0.052 | 0.005 | 0.555 | 0.759 |
| RAPGEF4 | 0.657 | 0.055 | 0.005 | 0.549 | 0.764 |
| PERP | 0.657 | 0.052 | 0.005 | 0.555 | 0.758 |
| FITM2 | 0.656 | 0.049 | 0.005 | 0.559 | 0.752 |
| INPP5A | 0.655 | 0.051 | 0.006 | 0.554 | 0.755 |
| PKP2 | 0.654 | 0.050 | 0.006 | 0.555 | 0.752 |
| PDGFA | 0.652 | 0.054 | 0.006 | 0.547 | 0.758 |
| JAG1 | 0.652 | 0.052 | 0.006 | 0.549 | 0.755 |
| AC108062.1 | 0.652 | 0.051 | 0.006 | 0.552 | 0.752 |
| MTOR.AS1 | 0.651 | 0.053 | 0.007 | 0.547 | 0.754 |
| BX322234.1 | 0.651 | 0.050 | 0.007 | 0.552 | 0.750 |
| SUN1 | 0.650 | 0.052 | 0.007 | 0.549 | 0.752 |
| CAV2 | 0.650 | 0.053 | 0.007 | 0.546 | 0.755 |
| IFT43 | 0.650 | 0.052 | 0.007 | 0.547 | 0.752 |
| HEY1 | 0.649 | 0.055 | 0.007 | 0.542 | 0.756 |
| TP53INP2 | 0.649 | 0.054 | 0.008 | 0.544 | 0.754 |
| MIR221 | 0.648 | 0.054 | 0.008 | 0.543 | 0.754 |
| X10.Sep | 0.648 | 0.052 | 0.008 | 0.546 | 0.750 |
| MAFK | 0.648 | 0.052 | 0.008 | 0.547 | 0.749 |
| PICSAR | 0.647 | 0.054 | 0.008 | 0.541 | 0.754 |
| GPC4 | 0.647 | 0.054 | 0.008 | 0.541 | 0.753 |
| MIR3936HG | 0.647 | 0.054 | 0.009 | 0.542 | 0.752 |
| ACKR2 | 0.646 | 0.052 | 0.009 | 0.544 | 0.749 |
| PYGB | 0.646 | 0.052 | 0.009 | 0.544 | 0.749 |
| ISM1 | 0.646 | 0.054 | 0.009 | 0.540 | 0.752 |
| SLIT2.IT1 | 0.645 | 0.053 | 0.009 | 0.541 | 0.750 |
| DNAJB4 | 0.645 | 0.052 | 0.009 | 0.544 | 0.747 |
| PDLIM3 | 0.645 | 0.050 | 0.009 | 0.547 | 0.744 |
| DST | 0.645 | 0.054 | 0.010 | 0.540 | 0.750 |
| ICA1 | 0.645 | 0.053 | 0.010 | 0.542 | 0.748 |
| FBLN5 | 0.644 | 0.056 | 0.010 | 0.534 | 0.754 |
| CNN3 | 0.644 | 0.053 | 0.010 | 0.539 | 0.749 |
| STARD10 | 0.643 | 0.054 | 0.010 | 0.538 | 0.748 |
| ENPP1 | 0.642 | 0.056 | 0.011 | 0.532 | 0.751 |
| ADARB1 | 0.642 | 0.052 | 0.011 | 0.540 | 0.744 |
| MAST4 | 0.642 | 0.051 | 0.011 | 0.542 | 0.741 |
| SRPX2 | 0.641 | 0.052 | 0.011 | 0.540 | 0.743 |
| XPR1 | 0.641 | 0.054 | 0.011 | 0.536 | 0.746 |
| CALD1 | 0.641 | 0.052 | 0.012 | 0.539 | 0.743 |
| CYP4Z2P | 0.641 | 0.054 | 0.012 | 0.535 | 0.747 |
| METRN | 0.641 | 0.055 | 0.012 | 0.532 | 0.749 |
| KLHL18 | 0.641 | 0.056 | 0.012 | 0.531 | 0.750 |
| CYTH3 | 0.641 | 0.052 | 0.012 | 0.539 | 0.742 |
| MAP7 | 0.640 | 0.052 | 0.012 | 0.538 | 0.743 |
| PDZD8 | 0.639 | 0.056 | 0.012 | 0.530 | 0.749 |
| RNF11 | 0.639 | 0.053 | 0.012 | 0.535 | 0.744 |
| FOXA1 | 0.639 | 0.052 | 0.013 | 0.537 | 0.741 |
| FNDC4 | 0.639 | 0.053 | 0.013 | 0.534 | 0.743 |
| GLI3 | 0.639 | 0.053 | 0.013 | 0.535 | 0.742 |
| DYNC112 | 0.638 | 0.050 | 0.013 | 0.540 | 0.736 |
| SPTAN1 | 0.637 | 0.052 | 0.014 | 0.535 | 0.740 |
| SNORD113.2 | 0.637 | 0.053 | 0.014 | 0.532 | 0.742 |
| CALM1 | 0.637 | 0.052 | 0.014 | 0.536 | 0.738 |
| AGR2 | 0.636 | 0.054 | 0.014 | 0.530 | 0.743 |
| GOPC | 0.636 | 0.052 | 0.014 | 0.534 | 0.739 |
| NHS | 0.636 | 0.052 | 0.015 | 0.534 | 0.739 |
| EPPK1 | 0.636 | 0.054 | 0.015 | 0.530 | 0.742 |
| SLC25A13 | 0.636 | 0.053 | 0.015 | 0.532 | 0.740 |
| EGFR.AS1 | 0.636 | 0.054 | 0.015 | 0.529 | 0.742 |
| NHS.AS1 | 0.635 | 0.055 | 0.015 | 0.528 | 0.743 |
| CYBRD1 | 0.635 | 0.055 | 0.016 | 0.528 | 0.742 |
| LZIC | 0.635 | 0.053 | 0.016 | 0.531 | 0.738 |
| ACVR1 | 0.635 | 0.052 | 0.016 | 0.533 | 0.737 |
| RNF24 | 0.635 | 0.055 | 0.016 | 0.527 | 0.743 |
| ACVR2A | 0.635 | 0.055 | 0.016 | 0.527 | 0.742 |
| KIAA1024 | 0.634 | 0.054 | 0.016 | 0.529 | 0.739 |
| C1orf53 | 0.634 | 0.052 | 0.016 | 0.533 | 0.735 |
| ATP2B1 | 0.634 | 0.055 | 0.016 | 0.527 | 0.742 |
| FZD1 | 0.634 | 0.054 | 0.017 | 0.528 | 0.739 |
| PRKACB | 0.634 | 0.055 | 0.017 | 0.527 | 0.741 |
| TPM2 | 0.634 | 0.055 | 0.017 | 0.525 | 0.742 |
| GOLGA8B | 0.634 | 0.053 | 0.017 | 0.530 | 0.737 |
| USP35 | 0.633 | 0.053 | 0.017 | 0.530 | 0.737 |
| ANO10 | 0.633 | 0.054 | 0.017 | 0.528 | 0.739 |
| NAMPT | 0.633 | 0.052 | 0.017 | 0.532 | 0.734 |
| PLLP | 0.633 | 0.052 | 0.017 | 0.531 | 0.735 |
| KRT18 | 0.632 | 0.053 | 0.018 | 0.528 | 0.736 |
| POF1B | 0.632 | 0.058 | 0.018 | 0.519 | 0.746 |
| CCND1 | 0.632 | 0.055 | 0.018 | 0.524 | 0.740 |
| SCGB3A2 | 0.632 | 0.053 | 0.018 | 0.527 | 0.736 |
| RNF215 | 0.632 | 0.056 | 0.018 | 0.522 | 0.741 |
| SNORA22 | 0.631 | 0.057 | 0.019 | 0.520 | 0.742 |
| C11orf96 | 0.631 | 0.053 | 0.019 | 0.527 | 0.736 |
| UBE2Q2 | 0.631 | 0.053 | 0.019 | 0.527 | 0.735 |
| SPOPL | 0.631 | 0.051 | 0.019 | 0.531 | 0.731 |
| SDCBP2 | 0.631 | 0.055 | 0.019 | 0.523 | 0.738 |
| SNORA11 | 0.631 | 0.052 | 0.019 | 0.529 | 0.733 |
| INHBB | 0.630 | 0.053 | 0.019 | 0.526 | 0.735 |
| AC105942.1 | 0.630 | 0.052 | 0.019 | 0.528 | 0.733 |
| YAP1 | 0.630 | 0.054 | 0.019 | 0.525 | 0.736 |
| TPD52L1 | 0.630 | 0.054 | 0.020 | 0.524 | 0.736 |
| VSTM4 | 0.630 | 0.052 | 0.020 | 0.528 | 0.732 |
| UACA | 0.630 | 0.052 | 0.020 | 0.527 | 0.732 |
| KIAA1549 | 0.630 | 0.058 | 0.020 | 0.516 | 0.743 |
| ITGAV | 0.630 | 0.051 | 0.020 | 0.530 | 0.729 |
| TTPAL | 0.629 | 0.052 | 0.021 | 0.527 | 0.732 |
| DOK4 | 0.629 | 0.053 | 0.021 | 0.526 | 0.732 |
| THRB | 0.629 | 0.053 | 0.021 | 0.526 | 0.732 |
| PTGR2 | 0.629 | 0.052 | 0.021 | 0.526 | 0.731 |
| BMI1 | 0.629 | 0.054 | 0.021 | 0.522 | 0.735 |
| ADCK1 | 0.628 | 0.053 | 0.021 | 0.524 | 0.733 |
| FSTL3 | 0.628 | 0.053 | 0.021 | 0.525 | 0.731 |
| MAP3K4 | 0.628 | 0.056 | 0.021 | 0.519 | 0.737 |
| CDH2 | 0.628 | 0.056 | 0.022 | 0.518 | 0.738 |
| MIR1295A | 0.628 | 0.054 | 0.022 | 0.522 | 0.734 |
| PRKAB2 | 0.628 | 0.052 | 0.022 | 0.526 | 0.730 |
| CPT1A | 0.628 | 0.054 | 0.022 | 0.521 | 0.734 |
| PRSS48 | 0.627 | 0.054 | 0.023 | 0.522 | 0.732 |
| HOXC10 | 0.627 | 0.051 | 0.023 | 0.527 | 0.727 |
| ETNK2 | 0.627 | 0.052 | 0.023 | 0.525 | 0.729 |
| DIXDC1 | 0.627 | 0.052 | 0.023 | 0.525 | 0.728 |
| ATP7B | 0.626 | 0.054 | 0.024 | 0.519 | 0.733 |
| DYRK3 | 0.626 | 0.055 | 0.024 | 0.518 | 0.734 |
| STXBP5 | 0.626 | 0.055 | 0.024 | 0.519 | 0.733 |
| MSMO1 | 0.626 | 0.053 | 0.024 | 0.521 | 0.730 |
| KITLG | 0.625 | 0.052 | 0.025 | 0.524 | 0.727 |
| TANC1 | 0.625 | 0.057 | 0.025 | 0.514 | 0.737 |
| LONP2 | 0.625 | 0.053 | 0.025 | 0.521 | 0.730 |
| ALS2CL | 0.625 | 0.054 | 0.025 | 0.519 | 0.731 |
| NR2F1.AS1 | 0.625 | 0.054 | 0.025 | 0.520 | 0.730 |
| ZNF432 | 0.625 | 0.052 | 0.025 | 0.523 | 0.727 |
| SLC7A2 | 0.625 | 0.056 | 0.025 | 0.516 | 0.734 |
| CNN1 | 0.625 | 0.054 | 0.025 | 0.518 | 0.732 |
| ENDOD1 | 0.625 | 0.053 | 0.025 | 0.521 | 0.728 |
| RGS2 | 0.624 | 0.053 | 0.026 | 0.521 | 0.728 |
| SLC44A3.AS1 | 0.624 | 0.051 | 0.026 | 0.523 | 0.725 |
| SLC44A3 | 0.624 | 0.053 | 0.026 | 0.520 | 0.728 |
| BICC1 | 0.624 | 0.056 | 0.026 | 0.515 | 0.734 |
| GARNL3 | 0.624 | 0.056 | 0.026 | 0.515 | 0.733 |
| CD46 | 0.624 | 0.054 | 0.026 | 0.518 | 0.730 |
| RABGEF1 | 0.624 | 0.055 | 0.026 | 0.516 | 0.732 |
| CLDN12 | 0.623 | 0.053 | 0.027 | 0.520 | 0.726 |
| MATN3 | 0.623 | 0.053 | 0.027 | 0.520 | 0.727 |
| LTBP2 | 0.623 | 0.053 | 0.027 | 0.519 | 0.728 |
| DNAL1 | 0.623 | 0.052 | 0.028 | 0.521 | 0.725 |
| PLAGL1 | 0.623 | 0.051 | 0.028 | 0.523 | 0.723 |
| POSTN | 0.623 | 0.052 | 0.028 | 0.521 | 0.724 |
| REXO2 | 0.623 | 0.054 | 0.028 | 0.517 | 0.728 |
| DENND2C | 0.623 | 0.055 | 0.028 | 0.515 | 0.730 |
| NEDD4L | 0.623 | 0.054 | 0.028 | 0.517 | 0.728 |
| GULP1 | 0.623 | 0.053 | 0.028 | 0.519 | 0.726 |
| TECPR2 | 0.623 | 0.054 | 0.028 | 0.516 | 0.729 |
| RBMS1 | 0.622 | 0.055 | 0.028 | 0.514 | 0.730 |
| KDM5B | 0.622 | 0.054 | 0.028 | 0.516 | 0.728 |
| SF3B5 | 0.622 | 0.055 | 0.028 | 0.514 | 0.730 |
| MAGED2 | 0.622 | 0.052 | 0.029 | 0.521 | 0.723 |
| CAB39L | 0.622 | 0.053 | 0.029 | 0.518 | 0.725 |
| SF3B1 | 0.622 | 0.053 | 0.029 | 0.517 | 0.726 |
| CHP1 | 0.622 | 0.052 | 0.029 | 0.520 | 0.723 |
| PHF20 | 0.621 | 0.057 | 0.030 | 0.511 | 0.732 |
| TMEM150C | 0.621 | 0.052 | 0.030 | 0.520 | 0.722 |
| CHST1 | 0.621 | 0.053 | 0.030 | 0.517 | 0.726 |
| PMEPA1 | 0.621 | 0.055 | 0.030 | 0.514 | 0.728 |
| MIR4324 | 0.621 | 0.057 | 0.030 | 0.509 | 0.733 |
| PCSK1 | 0.621 | 0.054 | 0.030 | 0.516 | 0.726 |
| MIR3909 | 0.621 | 0.053 | 0.030 | 0.517 | 0.724 |
| SIAH1 | 0.621 | 0.053 | 0.030 | 0.516 | 0.725 |
| SNX9 | 0.621 | 0.053 | 0.030 | 0.517 | 0.725 |
| EOGT | 0.621 | 0.052 | 0.030 | 0.519 | 0.723 |
| SUSD2 | 0.621 | 0.054 | 0.030 | 0.516 | 0.726 |
| SOWAHC | 0.621 | 0.053 | 0.030 | 0.517 | 0.724 |
| CBR3.AS1 | 0.620 | 0.055 | 0.031 | 0.513 | 0.728 |
| AKR1C1 | 0.620 | 0.054 | 0.031 | 0.514 | 0.727 |
| DALRD3 | 0.620 | 0.055 | 0.031 | 0.512 | 0.728 |
| ZNF677 | 0.620 | 0.054 | 0.031 | 0.514 | 0.726 |
| ZNF502 | 0.620 | 0.054 | 0.031 | 0.514 | 0.726 |
| MIR125B1 | 0.620 | 0.053 | 0.031 | 0.517 | 0.723 |
| GRB10 | 0.620 | 0.053 | 0.032 | 0.516 | 0.723 |
| TWSG1 | 0.620 | 0.053 | 0.032 | 0.517 | 0.723 |
| PPP3CA | 0.620 | 0.057 | 0.032 | 0.507 | 0.732 |
| FLNA | 0.620 | 0.055 | 0.032 | 0.512 | 0.727 |
| ECI2 | 0.620 | 0.053 | 0.032 | 0.515 | 0.724 |
| P2RY2 | 0.619 | 0.056 | 0.033 | 0.510 | 0.728 |
| NRDE2 | 0.619 | 0.056 | 0.033 | 0.510 | 0.728 |
| TIMP3 | 0.619 | 0.053 | 0.033 | 0.516 | 0.723 |
| PID1 | 0.619 | 0.052 | 0.033 | 0.518 | 0.721 |
| GGCX | 0.619 | 0.052 | 0.033 | 0.517 | 0.722 |
| ZNF273 | 0.619 | 0.052 | 0.033 | 0.518 | 0.721 |
| MICALL2 | 0.619 | 0.054 | 0.033 | 0.514 | 0.724 |
| PFKFB4 | 0.619 | 0.052 | 0.033 | 0.517 | 0.720 |
| TSPYL1 | 0.619 | 0.052 | 0.033 | 0.517 | 0.721 |
| DARS | 0.619 | 0.050 | 0.033 | 0.521 | 0.717 |
| ARHGEF17 | 0.619 | 0.055 | 0.033 | 0.511 | 0.726 |
| DAAM1 | 0.618 | 0.054 | 0.034 | 0.513 | 0.723 |
| SPOCK1 | 0.618 | 0.052 | 0.034 | 0.517 | 0.719 |
| AL109809.1 | 0.618 | 0.054 | 0.034 | 0.513 | 0.723 |
| FAM220A | 0.618 | 0.053 | 0.034 | 0.515 | 0.721 |
| CTNNB1 | 0.618 | 0.055 | 0.034 | 0.510 | 0.726 |
| AL137145.2 | 0.618 | 0.054 | 0.035 | 0.512 | 0.724 |
| HOXC.AS2 | 0.618 | 0.053 | 0.035 | 0.514 | 0.722 |
| PJA2 | 0.617 | 0.055 | 0.035 | 0.509 | 0.726 |
| SCGB1D2 | 0.617 | 0.053 | 0.035 | 0.514 | 0.721 |
| OXTR | 0.617 | 0.058 | 0.035 | 0.503 | 0.732 |
| CYP2J2 | 0.617 | 0.055 | 0.036 | 0.510 | 0.724 |
| TMEM106B | 0.617 | 0.051 | 0.036 | 0.517 | 0.717 |
| CPE | 0.617 | 0.054 | 0.036 | 0.512 | 0.723 |
| MIRLET7C | 0.617 | 0.055 | 0.036 | 0.510 | 0.724 |
| KREMEN1 | 0.617 | 0.054 | 0.036 | 0.511 | 0.724 |
| AKAP12 | 0.617 | 0.057 | 0.036 | 0.505 | 0.729 |
| WBP2NL | 0.617 | 0.056 | 0.036 | 0.507 | 0.727 |
| AFAP1L2 | 0.617 | 0.056 | 0.036 | 0.507 | 0.727 |
| MIR378G | 0.617 | 0.054 | 0.036 | 0.511 | 0.723 |
| MIR5787 | 0.617 | 0.054 | 0.037 | 0.511 | 0.722 |
| REEP3 | 0.617 | 0.051 | 0.037 | 0.517 | 0.716 |
| HDX | 0.617 | 0.053 | 0.037 | 0.513 | 0.720 |
| RAMP1 | 0.617 | 0.052 | 0.037 | 0.515 | 0.718 |
| SEMA4F | 0.616 | 0.052 | 0.037 | 0.514 | 0.719 |
| GPR153 | 0.616 | 0.054 | 0.037 | 0.510 | 0.722 |
| FANK1 | 0.616 | 0.054 | 0.037 | 0.511 | 0.721 |
| ARMC9 | 0.616 | 0.052 | 0.037 | 0.514 | 0.719 |
| TTLL7 | 0.616 | 0.056 | 0.037 | 0.507 | 0.726 |
| ARHGAP32 | 0.616 | 0.058 | 0.037 | 0.503 | 0.729 |
| TMEM87A | 0.616 | 0.055 | 0.037 | 0.508 | 0.724 |
| ZSCAN31 | 0.616 | 0.054 | 0.038 | 0.511 | 0.721 |
| MEIS2 | 0.616 | 0.053 | 0.038 | 0.513 | 0.719 |
| PTGS2 | 0.616 | 0.055 | 0.038 | 0.508 | 0.723 |
| AFDN | 0.616 | 0.053 | 0.038 | 0.511 | 0.720 |
| DNAJA2 | 0.616 | 0.055 | 0.038 | 0.509 | 0.723 |
| RNF152 | 0.616 | 0.055 | 0.038 | 0.507 | 0.724 |
| AR | 0.616 | 0.050 | 0.038 | 0.517 | 0.714 |
| TREH | 0.615 | 0.056 | 0.039 | 0.506 | 0.724 |
| PRKG1 | 0.615 | 0.053 | 0.039 | 0.512 | 0.718 |
| LOXL1 | 0.615 | 0.052 | 0.039 | 0.514 | 0.716 |
| ANKRD50 | 0.615 | 0.052 | 0.039 | 0.514 | 0.717 |
| CTTN | 0.615 | 0.053 | 0.039 | 0.511 | 0.719 |
| CARNMT1 | 0.615 | 0.053 | 0.039 | 0.511 | 0.719 |
| GINM1 | 0.615 | 0.053 | 0.039 | 0.512 | 0.719 |
| SIPA1L2 | 0.615 | 0.054 | 0.040 | 0.510 | 0.720 |
| FAM110B | 0.615 | 0.052 | 0.040 | 0.513 | 0.717 |
| SH3D19 | 0.614 | 0.054 | 0.040 | 0.509 | 0.720 |
| FBXO30 | 0.614 | 0.053 | 0.040 | 0.510 | 0.719 |
| SLC9A3R2 | 0.614 | 0.053 | 0.040 | 0.511 | 0.717 |
| CDH11 | 0.614 | 0.055 | 0.041 | 0.507 | 0.722 |
| VTA1 | 0.614 | 0.055 | 0.041 | 0.506 | 0.722 |
| UGDH | 0.614 | 0.054 | 0.042 | 0.508 | 0.719 |
| ID3 | 0.614 | 0.054 | 0.042 | 0.508 | 0.719 |
| ARF4 | 0.613 | 0.056 | 0.042 | 0.504 | 0.723 |
| RAB11FIP5 | 0.613 | 0.053 | 0.042 | 0.509 | 0.717 |
| FLNB | 0.613 | 0.056 | 0.042 | 0.504 | 0.722 |
| GNG10 | 0.613 | 0.054 | 0.042 | 0.507 | 0.720 |
| EZR | 0.613 | 0.052 | 0.043 | 0.511 | 0.714 |
| C2orf76 | 0.613 | 0.053 | 0.043 | 0.510 | 0.716 |
| TMEM150A | 0.613 | 0.053 | 0.043 | 0.509 | 0.717 |
| SOX12 | 0.613 | 0.055 | 0.043 | 0.505 | 0.720 |
| TMEM47 | 0.613 | 0.053 | 0.043 | 0.508 | 0.717 |
| HOXC11 | 0.613 | 0.052 | 0.043 | 0.510 | 0.715 |
| SYNJ2 | 0.613 | 0.054 | 0.043 | 0.508 | 0.718 |
| NPR3 | 0.613 | 0.057 | 0.043 | 0.502 | 0.724 |
| NIPSNAP1 | 0.612 | 0.056 | 0.044 | 0.503 | 0.722 |
| MSRB3 | 0.612 | 0.054 | 0.044 | 0.507 | 0.718 |
| FERMT2 | 0.612 | 0.055 | 0.044 | 0.504 | 0.721 |
| SLC5A4 | 0.612 | 0.055 | 0.044 | 0.504 | 0.720 |
| UFSP1 | 0.612 | 0.054 | 0.044 | 0.506 | 0.718 |
| EGLN3 | 0.612 | 0.053 | 0.044 | 0.508 | 0.716 |
| CDKL5 | 0.612 | 0.055 | 0.045 | 0.505 | 0.719 |
| CTGF | 0.612 | 0.057 | 0.045 | 0.501 | 0.723 |
| SERAC1 | 0.612 | 0.056 | 0.045 | 0.503 | 0.722 |
| EVA1A | 0.612 | 0.054 | 0.045 | 0.507 | 0.717 |
| SLC4A11 | 0.612 | 0.051 | 0.045 | 0.512 | 0.712 |
| TNFRSF19 | 0.612 | 0.056 | 0.045 | 0.501 | 0.723 |
| STX19 | 0.612 | 0.053 | 0.045 | 0.508 | 0.716 |
| MPZL3 | 0.612 | 0.055 | 0.045 | 0.504 | 0.720 |
| GALNT10 | 0.612 | 0.054 | 0.045 | 0.506 | 0.717 |
| CPD | 0.612 | 0.053 | 0.045 | 0.507 | 0.716 |
| PHACTR2 | 0.612 | 0.051 | 0.045 | 0.512 | 0.711 |
| CEACAM6 | 0.612 | 0.057 | 0.045 | 0.501 | 0.723 |
| PEX11A | 0.611 | 0.059 | 0.046 | 0.496 | 0.727 |
| PPP1R21 | 0.611 | 0.054 | 0.046 | 0.505 | 0.718 |
| PLEKHN1 | 0.611 | 0.052 | 0.046 | 0.509 | 0.713 |
| EHBP1 | 0.611 | 0.057 | 0.046 | 0.500 | 0.723 |
| ZNF615 | 0.611 | 0.053 | 0.046 | 0.506 | 0.716 |
| UBR3 | 0.611 | 0.053 | 0.046 | 0.507 | 0.715 |
| SNX21 | 0.611 | 0.053 | 0.047 | 0.507 | 0.714 |
| LCA5 | 0.611 | 0.053 | 0.047 | 0.507 | 0.715 |
| MIR3128 | 0.611 | 0.053 | 0.047 | 0.508 | 0.714 |
| SEC14L2 | 0.611 | 0.053 | 0.047 | 0.506 | 0.715 |
| SERINC3 | 0.611 | 0.053 | 0.047 | 0.507 | 0.715 |
| IFNGR 1 | 0.611 | 0.052 | 0.047 | 0.509 | 0.713 |
| BMP4 | 0.611 | 0.054 | 0.047 | 0.506 | 0.716 |
| REPS1 | 0.611 | 0.054 | 0.048 | 0.505 | 0.716 |
| F2RL3 | 0.611 | 0.053 | 0.048 | 0.507 | 0.714 |
| SLC20A2 | 0.610 | 0.055 | 0.048 | 0.502 | 0.719 |
| FER | 0.610 | 0.054 | 0.049 | 0.504 | 0.716 |
| TEX9 | 0.610 | 0.053 | 0.049 | 0.506 | 0.714 |
| STRADB | 0.610 | 0.050 | 0.049 | 0.512 | 0.708 |
| RHOB | 0.610 | 0.055 | 0.049 | 0.503 | 0.717 |
| IFT52 | 0.610 | 0.056 | 0.049 | 0.500 | 0.719 |
| NR2F1 | 0.610 | 0.057 | 0.049 | 0.498 | 0.721 |
| MIR643 | 0.610 | 0.054 | 0.049 | 0.503 | 0.716 |
| ARSI | 0.610 | 0.052 | 0.049 | 0.508 | 0.712 |

TABLE 7
Genes predictive of pCR from the GSE106977 cohort.
Area under the curve

| | | | | Asymptotic 95% | |
|---|---|---|---|---|---|
| | | | | Confidence Interval | |
| Test Result | | Std. | Asymptotic | Lower | Upper |
| Variable(s) | Area | Error^{a} | Sig.^{b} | Bound | Bound |
| TNK2 | 0.723 | 0.048 | 0.000 | 0.629 | 0.816 |
| BRIP1 | 0.721 | 0.048 | 0.000 | 0.627 | 0.816 |
| SH3BP5 | 0.717 | 0.049 | 0.000 | 0.622 | 0.813 |
| SYNE2 | 0.708 | 0.046 | 0.000 | 0.618 | 0.799 |
| TCF19 | 0.708 | 0.048 | 0.000 | 0.614 | 0.802 |
| RAD54L | 0.704 | 0.048 | 0.000 | 0.609 | 0.798 |
| KLF6 | 0.703 | 0.048 | 0.000 | 0.610 | 0.797 |
| LOC100652973 | 0.701 | 0.049 | 0.000 | 0.606 | 0.797 |
| TP53 | 0.700 | 0.050 | 0.000 | 0.601 | 0.799 |
| IQGAP3 | 0.700 | 0.050 | 0.000 | 0.602 | 0.797 |
| BTN2A1 | 0.698 | 0.048 | 0.000 | 0.603 | 0.793 |
| NUP210 | 0.694 | 0.049 | 0.000 | 0.597 | 0.790 |
| KIFC1 | 0.694 | 0.049 | 0.000 | 0.597 | 0.790 |
| GIMAP7 | 0.692 | 0.049 | 0.000 | 0.595 | 0.789 |
| MTMR14 | 0.692 | 0.049 | 0.000 | 0.596 | 0.788 |
| RSBN1 | 0.690 | 0.049 | 0.000 | 0.594 | 0.787 |
| RTTN | 0.690 | 0.050 | 0.001 | 0.591 | 0.789 |
| ACPL2 | 0.690 | 0.049 | 0.001 | 0.593 | 0.786 |
| OXNAD1 | 0.689 | 0.050 | 0.001 | 0.591 | 0.788 |
| CYP27A1 | 0.689 | 0.049 | 0.001 | 0.594 | 0.784 |
| FAM193B | 0.689 | 0.048 | 0.001 | 0.594 | 0.784 |
| LBH | 0.689 | 0.049 | 0.001 | 0.592 | 0.785 |
| PSTPIP2 | 0.688 | 0.050 | 0.001 | 0.591 | 0.786 |
| ATP11A | 0.688 | 0.050 | 0.001 | 0.589 | 0.786 |
| MTMR1 | 0.687 | 0.050 | 0.001 | 0.588 | 0.786 |
| CHAF1B | 0.687 | 0.050 | 0.001 | 0.589 | 0.785 |
| CDKAL1 | 0.686 | 0.050 | 0.001 | 0.589 | 0.784 |
| TCF7 | 0.686 | 0.050 | 0.001 | 0.589 | 0.783 |
| CARS2 | 0.686 | 0.051 | 0.001 | 0.585 | 0.786 |
| MBOAT1 | 0.685 | 0.048 | 0.001 | 0.590 | 0.779 |
| ISG20 | 0.684 | 0.049 | 0.001 | 0.589 | 0.780 |
| TTC7A | 0.683 | 0.048 | 0.001 | 0.588 | 0.778 |
| NCAPD2 | 0.681 | 0.050 | 0.001 | 0.583 | 0.779 |
| EZH2 | 0.681 | 0.051 | 0.001 | 0.581 | 0.780 |
| EPB41L2 | 0.679 | 0.050 | 0.001 | 0.581 | 0.777 |
| IGF2BP2 | 0.679 | 0.051 | 0.001 | 0.578 | 0.780 |
| WDR47 | 0.679 | 0.052 | 0.001 | 0.577 | 0.781 |
| TUBGCP3 | 0.679 | 0.051 | 0.001 | 0.579 | 0.778 |
| LOC100505519 | 0.678 | 0.051 | 0.001 | 0.579 | 0.777 |
| TFCP2L1 | 0.678 | 0.049 | 0.001 | 0.582 | 0.774 |
| SLAMF8 | 0.678 | 0.048 | 0.001 | 0.583 | 0.773 |
| SCIMP | 0.677 | 0.052 | 0.001 | 0.576 | 0.779 |
| TFDP1 | 0.677 | 0.051 | 0.001 | 0.576 | 0.778 |
| MDC1 | 0.677 | 0.051 | 0.001 | 0.577 | 0.776 |
| TLK2 | 0.676 | 0.052 | 0.001 | 0.574 | 0.778 |
| TAP2 | 0.675 | 0.050 | 0.001 | 0.577 | 0.773 |
| MCM3 | 0.675 | 0.052 | 0.001 | 0.572 | 0.777 |
| RLTPR | 0.674 | 0.050 | 0.001 | 0.576 | 0.772 |
| GBP1 | 0.673 | 0.050 | 0.002 | 0.574 | 0.772 |
| METTL2B | 0.672 | 0.049 | 0.002 | 0.575 | 0.769 |
| SELL | 0.671 | 0.051 | 0.002 | 0.572 | 0.770 |
| RASAL3 | 0.671 | 0.050 | 0.002 | 0.573 | 0.770 |
| GRK6 | 0.671 | 0.050 | 0.002 | 0.573 | 0.769 |
| HMGN4 | 0.671 | 0.053 | 0.002 | 0.568 | 0.774 |
| CNIH2 | 0.671 | 0.050 | 0.002 | 0.572 | 0.770 |
| NRAS | 0.670 | 0.052 | 0.002 | 0.568 | 0.773 |
| ARHGAP30 | 0.670 | 0.049 | 0.002 | 0.574 | 0.767 |
| MICB | 0.670 | 0.052 | 0.002 | 0.569 | 0.771 |
| TMEM98 | 0.670 | 0.050 | 0.002 | 0.571 | 0.768 |
| SP4 | 0.670 | 0.051 | 0.002 | 0.570 | 0.769 |
| CDKN1B | 0.669 | 0.052 | 0.002 | 0.568 | 0.770 |
| SMURF2 | 0.669 | 0.051 | 0.002 | 0.569 | 0.769 |
| CARKD | 0.669 | 0.051 | 0.002 | 0.570 | 0.769 |
| TAS2R50 | 0.669 | 0.049 | 0.002 | 0.572 | 0.766 |
| GTSE1 | 0.669 | 0.052 | 0.002 | 0.567 | 0.771 |
| FYN | 0.669 | 0.051 | 0.002 | 0.569 | 0.768 |
| RASA2 | 0.669 | 0.050 | 0.002 | 0.570 | 0.767 |
| MF12 | 0.668 | 0.050 | 0.002 | 0.570 | 0.766 |
| HNRNPUL1 | 0.668 | 0.052 | 0.002 | 0.566 | 0.770 |
| ULK2 | 0.668 | 0.050 | 0.002 | 0.569 | 0.766 |
| SNORD84 | 0.667 | 0.051 | 0.002 | 0.566 | 0.768 |
| TRIM26 | 0.667 | 0.050 | 0.002 | 0.568 | 0.765 |
| NLRC5 | 0.666 | 0.050 | 0.002 | 0.569 | 0.764 |
| KIAA0226L | 0.666 | 0.051 | 0.002 | 0.565 | 0.767 |
| HLA-DOB | 0.666 | 0.049 | 0.002 | 0.569 | 0.762 |
| C6orf228 | 0.666 | 0.050 | 0.002 | 0.567 | 0.765 |
| ZNF234 | 0.666 | 0.052 | 0.002 | 0.565 | 0.767 |
| TOP3A | 0.666 | 0.051 | 0.002 | 0.565 | 0.766 |
| HAPLN3 | 0.665 | 0.049 | 0.003 | 0.568 | 0.762 |
| PELP1 | 0.665 | 0.050 | 0.003 | 0.567 | 0.763 |
| SLC25A51 | 0.665 | 0.051 | 0.003 | 0.564 | 0.765 |
| ABCF1 | 0.664 | 0.052 | 0.003 | 0.563 | 0.766 |
| ZNF529 | 0.664 | 0.051 | 0.003 | 0.564 | 0.765 |
| HIST1H1E | 0.664 | 0.054 | 0.003 | 0.559 | 0.769 |
| MIR1178 | 0.663 | 0.050 | 0.003 | 0.565 | 0.762 |
| CNTROB | 0.663 | 0.050 | 0.003 | 0.566 | 0.761 |
| KIF18A | 0.663 | 0.052 | 0.003 | 0.562 | 0.765 |
| ARHGEF6 | 0.663 | 0.051 | 0.003 | 0.563 | 0.763 |
| STAG1 | 0.663 | 0.054 | 0.003 | 0.557 | 0.768 |
| SYNE2 | 0.663 | 0.053 | 0.003 | 0.559 | 0.767 |
| EN1 | 0.663 | 0.052 | 0.003 | 0.562 | 0.764 |
| DHX16 | 0.663 | 0.051 | 0.003 | 0.562 | 0.764 |
| LMO4 | 0.663 | 0.049 | 0.003 | 0.566 | 0.760 |
| HLA-F | 0.663 | 0.051 | 0.003 | 0.562 | 0.763 |
| FAM126A | 0.662 | 0.051 | 0.003 | 0.563 | 0.762 |
| TMC8 | 0.662 | 0.051 | 0.003 | 0.561 | 0.763 |
| GPSM2 | 0.662 | 0.052 | 0.003 | 0.561 | 0.763 |
| ARHGEF7 | 0.662 | 0.053 | 0.003 | 0.558 | 0.766 |
| UHRF2 | 0.662 | 0.053 | 0.003 | 0.558 | 0.766 |
| ARGLU1 | 0.662 | 0.052 | 0.003 | 0.560 | 0.764 |
| TAF4B | 0.662 | 0.049 | 0.003 | 0.565 | 0.758 |
| POGLUT1 | 0.662 | 0.051 | 0.003 | 0.561 | 0.762 |
| TRNAU1AP | 0.661 | 0.052 | 0.003 | 0.559 | 0.764 |
| SFXN1 | 0.661 | 0.051 | 0.003 | 0.562 | 0.761 |
| CXCL13 | 0.661 | 0.050 | 0.003 | 0.564 | 0.759 |
| RASSF8 | 0.661 | 0.050 | 0.003 | 0.562 | 0.760 |
| FAM102B | 0.661 | 0.051 | 0.003 | 0.561 | 0.760 |
| STK24 | 0.661 | 0.052 | 0.003 | 0.560 | 0.762 |
| KRT5 | 0.660 | 0.050 | 0.003 | 0.563 | 0.758 |
| FAM40A | 0.660 | 0.050 | 0.003 | 0.562 | 0.758 |
| CC2D1B | 0.660 | 0.051 | 0.003 | 0.560 | 0.760 |
| BCL2 | 0.660 | 0.051 | 0.003 | 0.560 | 0.759 |
| BIN2 | 0.659 | 0.051 | 0.004 | 0.558 | 0.760 |
| GABBR1 | 0.659 | 0.051 | 0.004 | 0.560 | 0.759 |
| CEP192 | 0.659 | 0.053 | 0.004 | 0.555 | 0.763 |
| APOM | 0.659 | 0.051 | 0.004 | 0.559 | 0.758 |
| C1orf162 | 0.659 | 0.050 | 0.004 | 0.560 | 0.757 |
| ATG7 | 0.658 | 0.052 | 0.004 | 0.557 | 0.759 |
| DCLRE1B | 0.658 | 0.051 | 0.004 | 0.558 | 0.759 |
| LRRC3 | 0.658 | 0.050 | 0.004 | 0.560 | 0.757 |
| CYFIP2 | 0.658 | 0.051 | 0.004 | 0.559 | 0.758 |
| NUP50 | 0.658 | 0.053 | 0.004 | 0.555 | 0.761 |
| MUTED | 0.658 | 0.051 | 0.004 | 0.558 | 0.757 |
| TTF2 | 0.658 | 0.050 | 0.004 | 0.559 | 0.757 |
| WDR53 | 0.658 | 0.050 | 0.004 | 0.560 | 0.755 |
| ANKRD10 | 0.657 | 0.053 | 0.004 | 0.553 | 0.761 |
| TMPO | 0.657 | 0.053 | 0.004 | 0.552 | 0.762 |
| LOC100505668 | 0.657 | 0.052 | 0.004 | 0.556 | 0.758 |
| ZNF185 | 0.657 | 0.050 | 0.004 | 0.560 | 0.755 |
| RAB2B | 0.657 | 0.052 | 0.004 | 0.556 | 0.759 |
| MAP4K1 | 0.657 | 0.050 | 0.004 | 0.560 | 0.755 |
| ZNF791 | 0.657 | 0.053 | 0.004 | 0.554 | 0.760 |
| STIL | 0.657 | 0.051 | 0.004 | 0.558 | 0.756 |
| C6orf47 | 0.657 | 0.050 | 0.004 | 0.560 | 0.754 |
| SH3KBP1 | 0.657 | 0.051 | 0.004 | 0.557 | 0.757 |
| BTN3A2 | 0.656 | 0.052 | 0.004 | 0.554 | 0.759 |
| NR2C2 | 0.656 | 0.054 | 0.004 | 0.551 | 0.761 |
| MTHFD1L | 0.656 | 0.050 | 0.004 | 0.558 | 0.755 |
| PRKX | 0.656 | 0.050 | 0.004 | 0.558 | 0.755 |
| TINAGL1 | 0.656 | 0.051 | 0.004 | 0.557 | 0.756 |
| CDC20 | 0.656 | 0.051 | 0.004 | 0.556 | 0.757 |
| UTP18 | 0.656 | 0.053 | 0.004 | 0.553 | 0.759 |
| CD69 | 0.656 | 0.050 | 0.004 | 0.557 | 0.755 |
| SPIN4 | 0.656 | 0.051 | 0.004 | 0.555 | 0.757 |
| CFL2 | 0.655 | 0.050 | 0.004 | 0.558 | 0.753 |
| HLA-C | 0.655 | 0.052 | 0.004 | 0.554 | 0.757 |
| FANCD2 | 0.655 | 0.050 | 0.004 | 0.557 | 0.754 |
| SLC25A19 | 0.655 | 0.051 | 0.004 | 0.556 | 0.755 |
| ZNF239 | 0.655 | 0.050 | 0.004 | 0.557 | 0.754 |
| ZNF121 | 0.655 | 0.053 | 0.004 | 0.551 | 0.760 |
| RPS19 | 0.655 | 0.053 | 0.004 | 0.552 | 0.758 |
| TPCN2 | 0.655 | 0.052 | 0.004 | 0.553 | 0.757 |
| GBP5 | 0.655 | 0.051 | 0.004 | 0.556 | 0.754 |
| CD2 | 0.655 | 0.052 | 0.005 | 0.554 | 0.756 |
| AURKB | 0.655 | 0.051 | 0.005 | 0.556 | 0.754 |
| LOC100128398 | 0.655 | 0.051 | 0.005 | 0.554 | 0.755 |
| ZNF254 | 0.655 | 0.052 | 0.005 | 0.554 | 0.756 |
| TRAF6 | 0.654 | 0.052 | 0.005 | 0.552 | 0.757 |
| CINP | 0.654 | 0.051 | 0.005 | 0.555 | 0.754 |
| PM20D2 | 0.654 | 0.051 | 0.005 | 0.554 | 0.754 |
| PRR11 | 0.654 | 0.050 | 0.005 | 0.555 | 0.753 |
| MCM5 | 0.654 | 0.052 | 0.005 | 0.552 | 0.755 |
| ISY1 | 0.654 | 0.050 | 0.005 | 0.556 | 0.752 |
| GNL1 | 0.654 | 0.051 | 0.005 | 0.554 | 0.753 |
| HLA-B | 0.653 | 0.052 | 0.005 | 0.552 | 0.755 |
| MIR4632 | 0.653 | 0.051 | 0.005 | 0.554 | 0.753 |
| TAP1 | 0.653 | 0.051 | 0.005 | 0.552 | 0.754 |
| INTS2 | 0.653 | 0.051 | 0.005 | 0.553 | 0.753 |
| ITPK1 | 0.653 | 0.053 | 0.005 | 0.549 | 0.756 |
| GXYLT1 | 0.653 | 0.051 | 0.005 | 0.553 | 0.752 |
| LOC100294341 | 0.653 | 0.051 | 0.005 | 0.553 | 0.753 |
| PPM1D | 0.652 | 0.053 | 0.005 | 0.549 | 0.755 |
| CNOT10 | 0.652 | 0.054 | 0.005 | 0.547 | 0.758 |
| UBASH3B | 0.652 | 0.050 | 0.005 | 0.554 | 0.751 |
| GTPBP1 | 0.652 | 0.050 | 0.005 | 0.554 | 0.751 |
| GTF2H4 | 0.652 | 0.052 | 0.005 | 0.551 | 0.754 |
| CSGALNACT2 | 0.652 | 0.051 | 0.005 | 0.551 | 0.753 |
| ADCY7 | 0.652 | 0.052 | 0.005 | 0.551 | 0.754 |
| MOB3B | 0.652 | 0.051 | 0.005 | 0.552 | 0.752 |
| PCNX | 0.652 | 0.052 | 0.005 | 0.551 | 0.754 |
| UPF3A | 0.652 | 0.051 | 0.005 | 0.552 | 0.752 |
| TCP11L1 | 0.652 | 0.053 | 0.005 | 0.548 | 0.756 |
| CLDND1 | 0.652 | 0.051 | 0.005 | 0.551 | 0.752 |
| ZNF426 | 0.652 | 0.050 | 0.005 | 0.554 | 0.749 |
| ITGB8 | 0.651 | 0.051 | 0.006 | 0.551 | 0.751 |
| SELRC1 | 0.651 | 0.051 | 0.006 | 0.550 | 0.752 |
| FXYD5 | 0.651 | 0.050 | 0.006 | 0.553 | 0.748 |
| TUBB | 0.651 | 0.054 | 0.006 | 0.545 | 0.756 |
| GHRLOS | 0.650 | 0.053 | 0.006 | 0.547 | 0.753 |
| RACGAP1 | 0.650 | 0.051 | 0.006 | 0.550 | 0.751 |
| CELF2 | 0.650 | 0.052 | 0.006 | 0.548 | 0.753 |
| PLA2G7 | 0.650 | 0.051 | 0.006 | 0.550 | 0.751 |
| PRPF38A | 0.650 | 0.052 | 0.006 | 0.548 | 0.753 |
| CORO1A | 0.650 | 0.052 | 0.006 | 0.548 | 0.752 |
| TOPBP1 | 0.650 | 0.051 | 0.006 | 0.551 | 0.750 |
| SLC25A24 | 0.650 | 0.052 | 0.006 | 0.548 | 0.751 |
| CHAF1A | 0.649 | 0.051 | 0.006 | 0.549 | 0.750 |
| MIR3917 | 0.649 | 0.053 | 0.006 | 0.545 | 0.754 |
| PARP15 | 0.649 | 0.053 | 0.006 | 0.545 | 0.754 |
| OPTN | 0.649 | 0.050 | 0.006 | 0.552 | 0.746 |
| FBL | 0.649 | 0.053 | 0.006 | 0.546 | 0.753 |
| USP33 | 0.649 | 0.052 | 0.006 | 0.548 | 0.750 |
| HLA-G | 0.649 | 0.051 | 0.006 | 0.549 | 0.749 |
| SLC23A2 | 0.649 | 0.051 | 0.006 | 0.550 | 0.749 |
| POLR1E | 0.649 | 0.053 | 0.006 | 0.546 | 0.753 |
| RASGRP1 | 0.649 | 0.051 | 0.006 | 0.548 | 0.750 |
| DSC2 | 0.649 | 0.050 | 0.006 | 0.551 | 0.748 |
| HIST1H2BL | 0.649 | 0.052 | 0.006 | 0.548 | 0.750 |
| LY6G5B | 0.649 | 0.052 | 0.006 | 0.547 | 0.750 |
| CKAP2L | 0.649 | 0.050 | 0.006 | 0.550 | 0.747 |
| RIMKLB | 0.649 | 0.051 | 0.006 | 0.549 | 0.749 |
| UBD | 0.649 | 0.050 | 0.006 | 0.551 | 0.747 |
| RPS18 | 0.649 | 0.050 | 0.006 | 0.550 | 0.747 |
| C1RL | 0.649 | 0.051 | 0.006 | 0.550 | 0.748 |
| CEP89 | 0.648 | 0.051 | 0.007 | 0.548 | 0.749 |
| FAM100B | 0.648 | 0.052 | 0.007 | 0.546 | 0.750 |
| DHX36 | 0.648 | 0.053 | 0.007 | 0.543 | 0.753 |
| FLJ42393 | 0.648 | 0.053 | 0.007 | 0.545 | 0.751 |
| RAF1 | 0.648 | 0.055 | 0.007 | 0.539 | 0.756 |
| ZNF326 | 0.648 | 0.053 | 0.007 | 0.544 | 0.752 |
| C5orf56 | 0.647 | 0.051 | 0.007 | 0.547 | 0.748 |
| CDCA7 | 0.647 | 0.051 | 0.007 | 0.548 | 0.747 |
| FAM78A | 0.647 | 0.054 | 0.007 | 0.542 | 0.753 |
| SMYD4 | 0.647 | 0.052 | 0.007 | 0.546 | 0.749 |
| DENND4B | 0.647 | 0.053 | 0.007 | 0.544 | 0.750 |
| RCAN3 | 0.647 | 0.053 | 0.007 | 0.544 | 0.751 |
| RAD51AP1 | 0.647 | 0.051 | 0.007 | 0.546 | 0.748 |
| MCM2 | 0.647 | 0.052 | 0.007 | 0.544 | 0.749 |
| KDM5A | 0.647 | 0.053 | 0.007 | 0.542 | 0.752 |
| ZNF440 | 0.647 | 0.053 | 0.007 | 0.544 | 0.750 |
| METTL2A | 0.647 | 0.053 | 0.007 | 0.542 | 0.751 |
| CD4 | 0.646 | 0.053 | 0.007 | 0.542 | 0.750 |
| PKN1 | 0.646 | 0.052 | 0.007 | 0.544 | 0.748 |
| POLH | 0.646 | 0.054 | 0.007 | 0.541 | 0.751 |
| HCST | 0.646 | 0.051 | 0.007 | 0.546 | 0.745 |
| ECT2 | 0.646 | 0.052 | 0.007 | 0.543 | 0.749 |
| RNF220 | 0.646 | 0.053 | 0.008 | 0.542 | 0.750 |
| MIR4444-1 | 0.646 | 0.053 | 0.008 | 0.541 | 0.750 |
| C3orf65 | 0.646 | 0.053 | 0.008 | 0.542 | 0.749 |
| C1QL3 | 0.646 | 0.053 | 0.008 | 0.542 | 0.749 |
| L3MBTL4 | 0.646 | 0.052 | 0.008 | 0.543 | 0.748 |
| NBPF8 | 0.645 | 0.051 | 0.008 | 0.546 | 0.744 |
| STX7 | 0.645 | 0.054 | 0.008 | 0.540 | 0.751 |
| MBTD1 | 0.645 | 0.052 | 0.008 | 0.544 | 0.746 |
| DEK | 0.645 | 0.052 | 0.008 | 0.544 | 0.747 |
| KDM1B | 0.645 | 0.053 | 0.008 | 0.541 | 0.750 |
| HACE1 | 0.645 | 0.051 | 0.008 | 0.545 | 0.746 |
| MRPS25 | 0.645 | 0.053 | 0.008 | 0.541 | 0.749 |
| SUN2 | 0.645 | 0.052 | 0.008 | 0.542 | 0.748 |
| NSUN4 | 0.645 | 0.050 | 0.008 | 0.546 | 0.744 |
| GMFG | 0.645 | 0.051 | 0.008 | 0.545 | 0.745 |
| KIF4A | 0.645 | 0.051 | 0.008 | 0.545 | 0.744 |
| NCOA7 | 0.645 | 0.051 | 0.008 | 0.545 | 0.744 |
| CYR61 | 0.645 | 0.051 | 0.008 | 0.545 | 0.744 |
| LRRC41 | 0.644 | 0.051 | 0.008 | 0.544 | 0.744 |
| ZCCHC11 | 0.644 | 0.054 | 0.008 | 0.538 | 0.751 |
| ARHGAP4 | 0.644 | 0.051 | 0.008 | 0.545 | 0.744 |
| KIAA0528 | 0.644 | 0.053 | 0.008 | 0.541 | 0.747 |
| DPY30 | 0.644 | 0.052 | 0.008 | 0.542 | 0.746 |
| SET | 0.644 | 0.056 | 0.008 | 0.534 | 0.754 |
| SFI1 | 0.644 | 0.052 | 0.008 | 0.542 | 0.746 |
| FANCA | 0.644 | 0.051 | 0.008 | 0.544 | 0.744 |
| TCTEX1D2 | 0.644 | 0.050 | 0.008 | 0.546 | 0.742 |
| FCGRT | 0.644 | 0.052 | 0.008 | 0.543 | 0.745 |
| HERC1 | 0.644 | 0.053 | 0.008 | 0.540 | 0.748 |
| IL12RB2 | 0.644 | 0.051 | 0.008 | 0.543 | 0.744 |
| OSBPL3 | 0.644 | 0.054 | 0.009 | 0.539 | 0.748 |
| RNMT | 0.644 | 0.053 | 0.009 | 0.539 | 0.748 |
| ZNF514 | 0.644 | 0.050 | 0.009 | 0.545 | 0.742 |
| HLA-E | 0.644 | 0.052 | 0.009 | 0.541 | 0.746 |
| PLCB2 | 0.643 | 0.051 | 0.009 | 0.543 | 0.744 |
| RAVER2 | 0.643 | 0.051 | 0.009 | 0.544 | 0.743 |
| PLSCR1 | 0.643 | 0.051 | 0.009 | 0.543 | 0.744 |
| GAS6 | 0.643 | 0.051 | 0.009 | 0.543 | 0.743 |
| ATP9B | 0.643 | 0.053 | 0.009 | 0.539 | 0.747 |
| CD52 | 0.643 | 0.051 | 0.009 | 0.543 | 0.742 |
| ACAP1 | 0.643 | 0.052 | 0.009 | 0.540 | 0.745 |
| SDR42E1 | 0.643 | 0.052 | 0.009 | 0.541 | 0.745 |
| ITGAX | 0.643 | 0.051 | 0.009 | 0.542 | 0.744 |
| DNMT1 | 0.643 | 0.052 | 0.009 | 0.541 | 0.745 |
| BTN3A1 | 0.643 | 0.053 | 0.009 | 0.539 | 0.746 |
| CMKLR1 | 0.643 | 0.055 | 0.009 | 0.535 | 0.750 |
| SKAP2 | 0.643 | 0.051 | 0.009 | 0.542 | 0.743 |
| TBC1D22A | 0.643 | 0.054 | 0.009 | 0.537 | 0.748 |
| PAK6 | 0.643 | 0.052 | 0.009 | 0.541 | 0.744 |
| MICALL 1 | 0.642 | 0.051 | 0.009 | 0.543 | 0.742 |
| TRPV2 | 0.642 | 0.051 | 0.009 | 0.543 | 0.742 |
| TNRC6A | 0.642 | 0.052 | 0.009 | 0.541 | 0.744 |
| SFT2D2 | 0.642 | 0.050 | 0.009 | 0.544 | 0.741 |
| ABHD13 | 0.642 | 0.051 | 0.009 | 0.542 | 0.743 |
| PTK7 | 0.642 | 0.052 | 0.009 | 0.541 | 0.744 |
| SLC18B1 | 0.642 | 0.053 | 0.009 | 0.537 | 0.746 |
| EXOSC3 | 0.642 | 0.052 | 0.009 | 0.541 | 0.743 |
| TPX2 | 0.642 | 0.052 | 0.009 | 0.540 | 0.743 |
| YEATS2 | 0.642 | 0.051 | 0.009 | 0.541 | 0.742 |
| POU5F1 | 0.641 | 0.052 | 0.010 | 0.540 | 0.743 |
| UBP1 | 0.641 | 0.054 | 0.010 | 0.536 | 0.747 |
| HIST1H2BN | 0.641 | 0.053 | 0.010 | 0.539 | 0.744 |
| ARNTL2 | 0.641 | 0.053 | 0.010 | 0.538 | 0.745 |
| ACOT9 | 0.641 | 0.051 | 0.010 | 0.542 | 0.741 |
| AL162431.1 | 0.641 | 0.053 | 0.010 | 0.539 | 0.744 |
| APOL3 | 0.641 | 0.051 | 0.010 | 0.541 | 0.742 |
| @44805 | 0.641 | 0.052 | 0.010 | 0.539 | 0.743 |
| API5 | 0.641 | 0.053 | 0.010 | 0.537 | 0.746 |
| APOBEC3D | 0.641 | 0.050 | 0.010 | 0.543 | 0.739 |
| KIF2C | 0.641 | 0.052 | 0.010 | 0.540 | 0.742 |
| FTSJD2 | 0.641 | 0.051 | 0.010 | 0.542 | 0.740 |
| C3orf38 | 0.641 | 0.053 | 0.010 | 0.538 | 0.744 |
| USP24 | 0.641 | 0.053 | 0.010 | 0.537 | 0.745 |
| ZFP82 | 0.641 | 0.051 | 0.010 | 0.542 | 0.740 |
| PHC3 | 0.641 | 0.053 | 0.010 | 0.537 | 0.745 |
| ANKRD28 | 0.641 | 0.054 | 0.010 | 0.536 | 0.746 |
| ACTL6A | 0.641 | 0.052 | 0.010 | 0.539 | 0.742 |
| E2F7 | 0.641 | 0.052 | 0.010 | 0.538 | 0.743 |
| IGSF3 | 0.641 | 0.051 | 0.010 | 0.540 | 0.741 |
| LOC401093 | 0.641 | 0.053 | 0.010 | 0.537 | 0.744 |
| BTN3A3 | 0.640 | 0.052 | 0.010 | 0.538 | 0.743 |
| TRA2B | 0.640 | 0.054 | 0.010 | 0.534 | 0.746 |
| BARD1 | 0.640 | 0.053 | 0.010 | 0.537 | 0.743 |
| E2F8 | 0.640 | 0.053 | 0.010 | 0.536 | 0.745 |
| HLA-A | 0.640 | 0.053 | 0.010 | 0.537 | 0.744 |
| LOC100294341 | 0.640 | 0.052 | 0.010 | 0.539 | 0.742 |
| AKR1B1 | 0.640 | 0.051 | 0.010 | 0.539 | 0.741 |
| FAM82A2 | 0.640 | 0.051 | 0.010 | 0.540 | 0.741 |
| SKA2 | 0.640 | 0.051 | 0.010 | 0.540 | 0.740 |
| RORA | 0.640 | 0.051 | 0.010 | 0.541 | 0.739 |
| ACAP2 | 0.640 | 0.056 | 0.010 | 0.531 | 0.749 |
| PRKCI | 0.640 | 0.052 | 0.010 | 0.537 | 0.742 |
| BTBD10 | 0.640 | 0.053 | 0.010 | 0.537 | 0.743 |
| ZNF43 | 0.640 | 0.052 | 0.010 | 0.538 | 0.741 |
| BAG6 | 0.640 | 0.052 | 0.010 | 0.537 | 0.742 |
| ITGAL | 0.640 | 0.053 | 0.010 | 0.537 | 0.743 |
| HECA | 0.640 | 0.053 | 0.010 | 0.535 | 0.744 |
| NINJ2 | 0.639 | 0.053 | 0.011 | 0.536 | 0.743 |
| UBA2 | 0.639 | 0.053 | 0.011 | 0.535 | 0.744 |
| LHFPL5 | 0.639 | 0.051 | 0.011 | 0.539 | 0.740 |
| ZFX | 0.639 | 0.052 | 0.011 | 0.536 | 0.742 |
| CEBPG | 0.639 | 0.051 | 0.011 | 0.539 | 0.739 |
| NNT | 0.639 | 0.051 | 0.011 | 0.539 | 0.739 |
| CEP78 | 0.639 | 0.052 | 0.011 | 0.537 | 0.741 |
| COMMD2 | 0.639 | 0.054 | 0.011 | 0.532 | 0.745 |
| AC092964.1 | 0.639 | 0.054 | 0.011 | 0.533 | 0.744 |
| C15orf42 | 0.639 | 0.052 | 0.011 | 0.536 | 0.742 |
| MIR922 | 0.639 | 0.054 | 0.011 | 0.533 | 0.744 |
| TCF20 | 0.639 | 0.054 | 0.011 | 0.533 | 0.744 |
| NFATC2 | 0.639 | 0.052 | 0.011 | 0.538 | 0.740 |
| SMC4 | 0.638 | 0.054 | 0.011 | 0.533 | 0.744 |
| RPA1 | 0.638 | 0.054 | 0.011 | 0.532 | 0.745 |
| CEP95 | 0.638 | 0.052 | 0.011 | 0.536 | 0.741 |
| LSM14A | 0.638 | 0.053 | 0.011 | 0.535 | 0.741 |
| KLRB1 | 0.638 | 0.051 | 0.011 | 0.537 | 0.739 |
| ZNF227 | 0.638 | 0.055 | 0.011 | 0.530 | 0.747 |
| CD300A | 0.638 | 0.052 | 0.011 | 0.536 | 0.740 |
| KAT6A | 0.638 | 0.054 | 0.011 | 0.532 | 0.744 |
| SLC15A3 | 0.638 | 0.052 | 0.011 | 0.536 | 0.740 |
| PLAGL1 | 0.638 | 0.052 | 0.011 | 0.536 | 0.740 |
| LUC7L3 | 0.638 | 0.053 | 0.011 | 0.535 | 0.741 |
| HTRA4 | 0.638 | 0.051 | 0.012 | 0.537 | 0.738 |
| CSNK2B | 0.638 | 0.052 | 0.012 | 0.536 | 0.739 |
| RAB12 | 0.638 | 0.052 | 0.012 | 0.536 | 0.740 |
| CD163 | 0.638 | 0.052 | 0.012 | 0.537 | 0.739 |
| MPHOSPH8 | 0.638 | 0.053 | 0.012 | 0.534 | 0.741 |
| CDCA3 | 0.638 | 0.051 | 0.012 | 0.537 | 0.738 |
| TMEM43 | 0.638 | 0.053 | 0.012 | 0.533 | 0.742 |
| RNF168 | 0.638 | 0.054 | 0.012 | 0.531 | 0.744 |
| ARAP2 | 0.638 | 0.053 | 0.012 | 0.534 | 0.741 |
| FANCI | 0.637 | 0.054 | 0.012 | 0.532 | 0.743 |
| TBPL1 | 0.637 | 0.052 | 0.012 | 0.535 | 0.740 |
| FAF1 | 0.637 | 0.053 | 0.012 | 0.534 | 0.741 |
| SRSF5 | 0.637 | 0.053 | 0.012 | 0.534 | 0.741 |
| IL17RE | 0.637 | 0.051 | 0.012 | 0.538 | 0.737 |
| IGSF9 | 0.637 | 0.051 | 0.012 | 0.537 | 0.737 |
| PREP | 0.637 | 0.053 | 0.012 | 0.534 | 0.740 |
| ASPM | 0.637 | 0.053 | 0.012 | 0.533 | 0.741 |
| BCLAF1 | 0.637 | 0.054 | 0.012 | 0.530 | 0.743 |
| RNF122 | 0.637 | 0.054 | 0.012 | 0.531 | 0.742 |
| TBC1D10C | 0.637 | 0.053 | 0.012 | 0.532 | 0.741 |
| ARHGEF18 | 0.637 | 0.052 | 0.012 | 0.535 | 0.739 |
| CDC16 | 0.637 | 0.053 | 0.012 | 0.532 | 0.741 |
| VANGL1 | 0.636 | 0.052 | 0.012 | 0.534 | 0.738 |
| ZNF418 | 0.636 | 0.053 | 0.012 | 0.533 | 0.739 |
| KAT7 | 0.636 | 0.053 | 0.012 | 0.533 | 0.740 |
| SLFN11 | 0.636 | 0.053 | 0.012 | 0.533 | 0.740 |
| XRCC1 | 0.636 | 0.052 | 0.012 | 0.535 | 0.738 |
| C11orf41 | 0.636 | 0.053 | 0.013 | 0.533 | 0.740 |
| BTN2A2 | 0.636 | 0.052 | 0.013 | 0.534 | 0.739 |
| LRFN4 | 0.636 | 0.052 | 0.013 | 0.535 | 0.737 |
| C14orf43 | 0.636 | 0.052 | 0.013 | 0.534 | 0.738 |
| CTSZ | 0.636 | 0.052 | 0.013 | 0.535 | 0.737 |
| MAP1S | 0.636 | 0.053 | 0.013 | 0.532 | 0.739 |
| FOXM1 | 0.636 | 0.052 | 0.013 | 0.533 | 0.739 |
| EHD1 | 0.636 | 0.051 | 0.013 | 0.535 | 0.736 |
| PHF5A | 0.636 | 0.052 | 0.013 | 0.533 | 0.739 |
| GNG4 | 0.635 | 0.052 | 0.013 | 0.534 | 0.737 |
| MBNL1 | 0.635 | 0.053 | 0.013 | 0.532 | 0.738 |
| BUB1 | 0.635 | 0.052 | 0.013 | 0.534 | 0.737 |
| GLS | 0.635 | 0.051 | 0.013 | 0.535 | 0.736 |
| ABCC10 | 0.635 | 0.051 | 0.013 | 0.535 | 0.736 |
| VAMP1 | 0.635 | 0.052 | 0.013 | 0.534 | 0.737 |
| PCID2 | 0.635 | 0.052 | 0.013 | 0.533 | 0.738 |
| PDE7A | 0.635 | 0.052 | 0.013 | 0.533 | 0.738 |
| U2SURP | 0.635 | 0.052 | 0.013 | 0.532 | 0.738 |
| ING4 | 0.635 | 0.051 | 0.013 | 0.535 | 0.736 |
| LAT | 0.635 | 0.053 | 0.013 | 0.531 | 0.739 |
| PYGO2 | 0.635 | 0.052 | 0.013 | 0.533 | 0.737 |
| UNG | 0.635 | 0.051 | 0.013 | 0.535 | 0.734 |
| ARHGAP11A | 0.635 | 0.051 | 0.013 | 0.535 | 0.735 |
| CCL19 | 0.635 | 0.054 | 0.013 | 0.529 | 0.741 |
| SIGLEC10 | 0.635 | 0.052 | 0.013 | 0.534 | 0.736 |
| RAB27A | 0.635 | 0.052 | 0.013 | 0.533 | 0.737 |
| ATP6VOA2 | 0.635 | 0.052 | 0.014 | 0.532 | 0.737 |
| ANLN | 0.635 | 0.052 | 0.014 | 0.533 | 0.736 |
| PCTP | 0.635 | 0.052 | 0.014 | 0.532 | 0.737 |
| GNA13 | 0.635 | 0.053 | 0.014 | 0.531 | 0.738 |
| RASGRP3 | 0.635 | 0.053 | 0.014 | 0.531 | 0.738 |
| TUBD1 | 0.634 | 0.051 | 0.014 | 0.534 | 0.735 |
| ZNF507 | 0.634 | 0.053 | 0.014 | 0.531 | 0.738 |
| CACHD1 | 0.634 | 0.053 | 0.014 | 0.531 | 0.737 |
| ZBED2 | 0.634 | 0.051 | 0.014 | 0.534 | 0.735 |
| C3orf26 | 0.634 | 0.052 | 0.014 | 0.533 | 0.735 |
| NUP62 | 0.634 | 0.052 | 0.014 | 0.532 | 0.737 |
| PRR3 | 0.634 | 0.051 | 0.014 | 0.534 | 0.734 |
| IKZF1 | 0.634 | 0.053 | 0.014 | 0.530 | 0.738 |
| PRMT3 | 0.634 | 0.052 | 0.014 | 0.532 | 0.735 |
| ILF2 | 0.634 | 0.054 | 0.014 | 0.529 | 0.739 |
| CMTM7 | 0.634 | 0.053 | 0.014 | 0.529 | 0.738 |
| DDX11 | 0.634 | 0.052 | 0.014 | 0.531 | 0.736 |
| KPNA5 | 0.633 | 0.054 | 0.014 | 0.528 | 0.739 |
| GPR52 | 0.633 | 0.055 | 0.014 | 0.526 | 0.741 |
| SLC2A3 | 0.633 | 0.053 | 0.014 | 0.530 | 0.736 |
| MED13 | 0.633 | 0.054 | 0.014 | 0.528 | 0.739 |
| FAIM3 | 0.633 | 0.054 | 0.015 | 0.528 | 0.738 |
| BAG5 | 0.633 | 0.053 | 0.015 | 0.529 | 0.737 |
| USP11 | 0.633 | 0.052 | 0.015 | 0.531 | 0.735 |
| SAFB | 0.633 | 0.054 | 0.015 | 0.528 | 0.738 |
| GIMAP1- | 0.633 | 0.053 | 0.015 | 0.528 | 0.738 |
| GIMAP5 | | | | | |
| ZNF808 | 0.633 | 0.053 | 0.015 | 0.530 | 0.736 |
| HIST1H2BJ | 0.633 | 0.053 | 0.015 | 0.528 | 0.737 |
| ACTN1 | 0.633 | 0.052 | 0.015 | 0.531 | 0.734 |
| ZNF621 | 0.633 | 0.053 | 0.015 | 0.529 | 0.736 |
| MFSD1 | 0.633 | 0.052 | 0.015 | 0.530 | 0.735 |
| CYBB | 0.633 | 0.053 | 0.015 | 0.529 | 0.736 |
| LAMP3 | 0.633 | 0.053 | 0.015 | 0.528 | 0.737 |
| GRAMD1A | 0.633 | 0.052 | 0.015 | 0.532 | 0.733 |
| SBF1 | 0.633 | 0.054 | 0.015 | 0.528 | 0.737 |
| METTL16 | 0.633 | 0.052 | 0.015 | 0.530 | 0.735 |
| MMP7 | 0.633 | 0.050 | 0.015 | 0.534 | 0.731 |
| APOL6 | 0.633 | 0.050 | 0.015 | 0.534 | 0.731 |
| GNAZ | 0.633 | 0.053 | 0.015 | 0.528 | 0.737 |
| LAIR1 | 0.633 | 0.052 | 0.015 | 0.530 | 0.735 |
| SMG9 | 0.633 | 0.052 | 0.015 | 0.531 | 0.734 |
| PDCD10 | 0.632 | 0.052 | 0.015 | 0.530 | 0.735 |
| CCDC94 | 0.632 | 0.052 | 0.015 | 0.530 | 0.735 |
| RNF219 | 0.632 | 0.051 | 0.015 | 0.532 | 0.732 |
| LOC100288637 | 0.632 | 0.052 | 0.015 | 0.531 | 0.734 |
| SYNCRIP | 0.632 | 0.054 | 0.015 | 0.526 | 0.738 |
| H2AFX | 0.632 | 0.053 | 0.015 | 0.529 | 0.735 |
| TAS2R20 | 0.632 | 0.052 | 0.015 | 0.531 | 0.734 |
| SDC3 | 0.632 | 0.051 | 0.015 | 0.532 | 0.732 |
| ZFYVE20 | 0.632 | 0.052 | 0.015 | 0.531 | 0.733 |
| ZNF674 | 0.632 | 0.054 | 0.016 | 0.526 | 0.738 |
| CYTH1 | 0.632 | 0.054 | 0.016 | 0.526 | 0.737 |
| URI1 | 0.632 | 0.054 | 0.016 | 0.527 | 0.737 |
| KCNMB3 | 0.632 | 0.053 | 0.016 | 0.529 | 0.735 |
| PAG1 | 0.632 | 0.051 | 0.016 | 0.531 | 0.732 |
| PDCD5 | 0.632 | 0.053 | 0.016 | 0.528 | 0.736 |
| ZNF302 | 0.632 | 0.052 | 0.016 | 0.530 | 0.734 |
| POLR3C | 0.632 | 0.052 | 0.016 | 0.530 | 0.734 |
| FAM49A | 0.632 | 0.052 | 0.016 | 0.531 | 0.733 |
| AP4B1 | 0.632 | 0.051 | 0.016 | 0.532 | 0.732 |
| ZNF266 | 0.632 | 0.055 | 0.016 | 0.525 | 0.739 |
| LMNB1 | 0.632 | 0.054 | 0.016 | 0.525 | 0.738 |
| KRTCAP3 | 0.632 | 0.052 | 0.016 | 0.530 | 0.733 |
| TAF3 | 0.632 | 0.052 | 0.016 | 0.529 | 0.734 |
| ZNF738 | 0.632 | 0.052 | 0.016 | 0.531 | 0.733 |
| LRP3 | 0.632 | 0.052 | 0.016 | 0.530 | 0.733 |
| MYO1G | 0.632 | 0.053 | 0.016 | 0.528 | 0.735 |
| TAS2R31 | 0.631 | 0.053 | 0.016 | 0.528 | 0.735 |
| ZNF283 | 0.631 | 0.053 | 0.016 | 0.528 | 0.735 |
| SRPK1 | 0.631 | 0.052 | 0.016 | 0.529 | 0.734 |
| RASA4B | 0.631 | 0.050 | 0.016 | 0.533 | 0.730 |
| BRPF1 | 0.631 | 0.051 | 0.016 | 0.530 | 0.732 |
| NOP2 | 0.631 | 0.056 | 0.016 | 0.522 | 0.740 |
| KIF15 | 0.631 | 0.052 | 0.016 | 0.530 | 0.732 |
| YIPF4 | 0.631 | 0.053 | 0.016 | 0.528 | 0.734 |
| NAB1 | 0.631 | 0.051 | 0.016 | 0.531 | 0.731 |
| C2CD3 | 0.631 | 0.051 | 0.016 | 0.531 | 0.731 |
| CD96 | 0.631 | 0.052 | 0.016 | 0.530 | 0.732 |
| XRN1 | 0.631 | 0.053 | 0.016 | 0.526 | 0.736 |
| PIM1 | 0.631 | 0.052 | 0.017 | 0.529 | 0.733 |
| CCL3L3 | 0.631 | 0.054 | 0.017 | 0.525 | 0.737 |
| PIK3CD | 0.631 | 0.051 | 0.017 | 0.530 | 0.731 |
| FTSJ3 | 0.631 | 0.052 | 0.017 | 0.529 | 0.732 |
| PHACTR2 | 0.631 | 0.054 | 0.017 | 0.526 | 0.736 |
| NUP85 | 0.631 | 0.054 | 0.017 | 0.525 | 0.736 |
| PTGDR | 0.631 | 0.053 | 0.017 | 0.527 | 0.735 |
| MYO5A | 0.631 | 0.053 | 0.017 | 0.526 | 0.735 |
| ABCB10 | 0.631 | 0.053 | 0.017 | 0.526 | 0.735 |
| FUBP1 | 0.631 | 0.053 | 0.017 | 0.528 | 0.734 |
| MELK | 0.631 | 0.052 | 0.017 | 0.528 | 0.733 |
| CCHCR1 | 0.630 | 0.052 | 0.017 | 0.528 | 0.732 |
| ANKRD44 | 0.630 | 0.054 | 0.017 | 0.525 | 0.735 |
| HELZ | 0.630 | 0.054 | 0.017 | 0.524 | 0.736 |
| WDR3 | 0.630 | 0.052 | 0.017 | 0.528 | 0.733 |
| LRRFIP2 | 0.630 | 0.053 | 0.017 | 0.527 | 0.733 |
| BCL11B | 0.630 | 0.052 | 0.017 | 0.529 | 0.732 |
| TLR7 | 0.630 | 0.054 | 0.017 | 0.525 | 0.736 |
| EMC3 | 0.630 | 0.053 | 0.017 | 0.526 | 0.735 |
| NFIB | 0.630 | 0.055 | 0.017 | 0.522 | 0.738 |
| CD22 | 0.630 | 0.053 | 0.017 | 0.527 | 0.733 |
| DOCK11 | 0.630 | 0.052 | 0.017 | 0.528 | 0.732 |
| NRTN | 0.630 | 0.054 | 0.017 | 0.525 | 0.735 |
| PARVB | 0.630 | 0.052 | 0.017 | 0.528 | 0.732 |
| VARS2 | 0.630 | 0.052 | 0.017 | 0.528 | 0.731 |
| DEPDC5 | 0.630 | 0.053 | 0.017 | 0.527 | 0.733 |
| ACYP1 | 0.630 | 0.053 | 0.017 | 0.525 | 0.734 |
| MICAL3 | 0.630 | 0.051 | 0.017 | 0.529 | 0.730 |
| FMR1 | 0.630 | 0.053 | 0.017 | 0.527 | 0.733 |
| TTLL4 | 0.630 | 0.052 | 0.017 | 0.529 | 0.731 |
| CLSTN3 | 0.630 | 0.053 | 0.017 | 0.527 | 0.733 |
| P2RY10 | 0.630 | 0.053 | 0.017 | 0.527 | 0.733 |
| PARVG | 0.630 | 0.053 | 0.017 | 0.526 | 0.733 |
| SPOCK2 | 0.630 | 0.051 | 0.017 | 0.530 | 0.730 |
| SEC13 | 0.630 | 0.053 | 0.017 | 0.526 | 0.734 |
| TPI1 | 0.630 | 0.053 | 0.017 | 0.527 | 0.733 |
| CUL4A | 0.630 | 0.055 | 0.018 | 0.522 | 0.737 |
| GGA2 | 0.630 | 0.053 | 0.018 | 0.525 | 0.734 |
| PHIP | 0.630 | 0.055 | 0.018 | 0.522 | 0.737 |
| PRRC2A | 0.630 | 0.054 | 0.018 | 0.524 | 0.735 |
| HAUS1 | 0.630 | 0.052 | 0.018 | 0.527 | 0.732 |
| RANBP3 | 0.629 | 0.053 | 0.018 | 0.525 | 0.734 |
| KCNN4 | 0.629 | 0.052 | 0.018 | 0.527 | 0.732 |
| KRT17 | 0.629 | 0.051 | 0.018 | 0.529 | 0.730 |
| TRAF1 | 0.629 | 0.051 | 0.018 | 0.529 | 0.729 |
| SNORA11D | 0.629 | 0.053 | 0.018 | 0.525 | 0.733 |
| DKC1 | 0.629 | 0.053 | 0.018 | 0.525 | 0.732 |
| PLCH1 | 0.629 | 0.054 | 0.018 | 0.524 | 0.734 |
| FGD3 | 0.629 | 0.052 | 0.018 | 0.528 | 0.730 |
| RANBP1 | 0.629 | 0.052 | 0.018 | 0.527 | 0.730 |
| GCNT2 | 0.629 | 0.052 | 0.018 | 0.526 | 0.731 |
| TMEM41A | 0.629 | 0.053 | 0.018 | 0.524 | 0.733 |
| GIMAP2 | 0.629 | 0.052 | 0.018 | 0.527 | 0.730 |
| ODF3B | 0.629 | 0.053 | 0.018 | 0.525 | 0.732 |
| NFAM1 | 0.628 | 0.051 | 0.019 | 0.528 | 0.728 |
| HACL1 | 0.628 | 0.053 | 0.019 | 0.524 | 0.732 |
| NDEL1 | 0.628 | 0.052 | 0.019 | 0.527 | 0.730 |
| TAS2R13 | 0.628 | 0.052 | 0.019 | 0.527 | 0.729 |
| RASSF2 | 0.628 | 0.054 | 0.019 | 0.522 | 0.735 |
| FAIM | 0.628 | 0.054 | 0.019 | 0.522 | 0.735 |
| FLI1 | 0.628 | 0.052 | 0.019 | 0.526 | 0.731 |
| POLR2F | 0.628 | 0.053 | 0.019 | 0.524 | 0.733 |
| HEATR6 | 0.628 | 0.054 | 0.019 | 0.522 | 0.734 |
| AMPD3 | 0.628 | 0.052 | 0.019 | 0.526 | 0.731 |
| IL17RD | 0.628 | 0.052 | 0.019 | 0.526 | 0.731 |
| KLRC4-KLRK1 | 0.628 | 0.054 | 0.019 | 0.523 | 0.734 |
| COTL1 | 0.628 | 0.052 | 0.019 | 0.526 | 0.730 |
| EPS15 | 0.628 | 0.053 | 0.019 | 0.524 | 0.732 |
| CCNE1 | 0.628 | 0.052 | 0.019 | 0.525 | 0.731 |
| ZNF431 | 0.628 | 0.052 | 0.019 | 0.527 | 0.730 |
| PMAIP1 | 0.628 | 0.054 | 0.019 | 0.523 | 0.733 |
| TBCB | 0.628 | 0.051 | 0.019 | 0.528 | 0.729 |
| STK10 | 0.628 | 0.052 | 0.019 | 0.526 | 0.730 |
| LBR | 0.628 | 0.052 | 0.019 | 0.525 | 0.731 |
| ANKRD27 | 0.628 | 0.052 | 0.019 | 0.526 | 0.729 |
| PSMB9 | 0.628 | 0.053 | 0.019 | 0.524 | 0.731 |
| TUBB6 | 0.628 | 0.052 | 0.019 | 0.526 | 0.730 |
| KIAA1737 | 0.628 | 0.052 | 0.019 | 0.527 | 0.729 |
| CHTOP | 0.628 | 0.053 | 0.019 | 0.524 | 0.731 |
| ZNF17 | 0.628 | 0.052 | 0.019 | 0.526 | 0.729 |
| RXRB | 0.628 | 0.053 | 0.019 | 0.523 | 0.732 |
| ANP32B | 0.628 | 0.053 | 0.019 | 0.524 | 0.732 |
| ZNF347 | 0.628 | 0.052 | 0.019 | 0.527 | 0.729 |
| ARHGEF1 | 0.628 | 0.051 | 0.019 | 0.527 | 0.729 |
| RAPGEF1 | 0.628 | 0.054 | 0.019 | 0.521 | 0.734 |
| SLC4A1AP | 0.628 | 0.053 | 0.019 | 0.524 | 0.731 |
| SH3BP1 | 0.628 | 0.052 | 0.019 | 0.526 | 0.729 |
| LARS2 | 0.628 | 0.052 | 0.019 | 0.526 | 0.729 |
| DDAH2 | 0.628 | 0.052 | 0.019 | 0.525 | 0.730 |
| ZNF267 | 0.628 | 0.053 | 0.019 | 0.524 | 0.732 |
| ZNF644 | 0.628 | 0.052 | 0.019 | 0.526 | 0.730 |
| FAM58A | 0.628 | 0.052 | 0.019 | 0.527 | 0.729 |
| CCDC97 | 0.627 | 0.053 | 0.020 | 0.525 | 0.730 |
| CKAP5 | 0.627 | 0.054 | 0.020 | 0.522 | 0.733 |
| IFI30 | 0.627 | 0.054 | 0.020 | 0.522 | 0.733 |
| UBE2E1 | 0.627 | 0.054 | 0.020 | 0.522 | 0.733 |
| TMEFF1 | 0.627 | 0.053 | 0.020 | 0.523 | 0.731 |
| BCL10 | 0.627 | 0.054 | 0.020 | 0.521 | 0.733 |
| ITGAE | 0.627 | 0.053 | 0.020 | 0.522 | 0.732 |
| PRSS16 | 0.627 | 0.051 | 0.020 | 0.528 | 0.726 |
| CDS2 | 0.627 | 0.052 | 0.020 | 0.524 | 0.730 |
| CCR7 | 0.627 | 0.052 | 0.020 | 0.524 | 0.730 |
| RNF8 | 0.627 | 0.052 | 0.020 | 0.526 | 0.729 |
| TLE4 | 0.627 | 0.052 | 0.020 | 0.525 | 0.729 |
| RHBDF2 | 0.627 | 0.052 | 0.020 | 0.526 | 0.729 |
| FAM125B | 0.627 | 0.054 | 0.020 | 0.522 | 0.732 |
| FAM65B | 0.627 | 0.053 | 0.020 | 0.522 | 0.731 |
| RCL1 | 0.627 | 0.053 | 0.020 | 0.523 | 0.730 |
| C2orf56 | 0.627 | 0.053 | 0.020 | 0.523 | 0.731 |
| STAT1 | 0.627 | 0.054 | 0.020 | 0.521 | 0.732 |
| CYLD | 0.627 | 0.053 | 0.020 | 0.523 | 0.731 |
| CLIP4 | 0.627 | 0.051 | 0.020 | 0.527 | 0.727 |
| SF3A3 | 0.627 | 0.053 | 0.020 | 0.523 | 0.731 |
| NBPF10 | 0.627 | 0.052 | 0.020 | 0.525 | 0.728 |
| FAM8A1 | 0.627 | 0.055 | 0.020 | 0.520 | 0.733 |
| FGD4 | 0.627 | 0.053 | 0.020 | 0.522 | 0.731 |
| TNFAIP3 | 0.627 | 0.052 | 0.020 | 0.525 | 0.728 |
| BRD1 | 0.627 | 0.054 | 0.020 | 0.520 | 0.733 |
| ASF1A | 0.627 | 0.052 | 0.020 | 0.524 | 0.729 |
| TRIT1 | 0.627 | 0.051 | 0.020 | 0.526 | 0.727 |
| ACTN4 | 0.627 | 0.051 | 0.020 | 0.526 | 0.727 |
| WDFY4 | 0.627 | 0.053 | 0.020 | 0.523 | 0.730 |
| MAP3K3 | 0.627 | 0.052 | 0.020 | 0.525 | 0.728 |
| LDLRAD3 | 0.627 | 0.053 | 0.020 | 0.523 | 0.730 |
| KIAA0355 | 0.627 | 0.053 | 0.020 | 0.524 | 0.730 |
| ZNF496 | 0.627 | 0.053 | 0.020 | 0.524 | 0.729 |
| BIRC3 | 0.626 | 0.053 | 0.021 | 0.522 | 0.731 |
| CCNL1 | 0.626 | 0.053 | 0.021 | 0.523 | 0.730 |
| NDE1 | 0.626 | 0.052 | 0.021 | 0.524 | 0.729 |
| PLCG2 | 0.626 | 0.052 | 0.021 | 0.525 | 0.727 |
| LRRN1 | 0.626 | 0.053 | 0.021 | 0.523 | 0.730 |
| SYNJ1 | 0.626 | 0.053 | 0.021 | 0.523 | 0.730 |
| CNTRL | 0.626 | 0.054 | 0.021 | 0.521 | 0.732 |
| RRM2 | 0.626 | 0.053 | 0.021 | 0.523 | 0.729 |
| SMC1A | 0.626 | 0.054 | 0.021 | 0.521 | 0.731 |
| DEF6 | 0.626 | 0.052 | 0.021 | 0.524 | 0.728 |
| PHB2 | 0.626 | 0.054 | 0.021 | 0.521 | 0.731 |
| DDX42 | 0.626 | 0.053 | 0.021 | 0.521 | 0.731 |
| TTC4 | 0.626 | 0.051 | 0.021 | 0.526 | 0.726 |
| MCM6 | 0.626 | 0.053 | 0.021 | 0.522 | 0.730 |
| QSER1 | 0.626 | 0.055 | 0.021 | 0.519 | 0.733 |
| LTB | 0.626 | 0.052 | 0.021 | 0.525 | 0.727 |
| RPP21 | 0.626 | 0.053 | 0.021 | 0.521 | 0.730 |
| PRC1 | 0.626 | 0.054 | 0.021 | 0.519 | 0.732 |
| DDX18 | 0.626 | 0.052 | 0.021 | 0.523 | 0.729 |
| ARID5A | 0.626 | 0.053 | 0.021 | 0.521 | 0.730 |
| DYNC1LI1 | 0.626 | 0.053 | 0.021 | 0.522 | 0.729 |
| PTPN2 | 0.626 | 0.054 | 0.021 | 0.521 | 0.731 |
| SIPA1L1 | 0.625 | 0.052 | 0.022 | 0.523 | 0.728 |
| GBP2 | 0.625 | 0.052 | 0.022 | 0.523 | 0.728 |
| EMR2 | 0.625 | 0.052 | 0.022 | 0.523 | 0.727 |
| PKN2 | 0.625 | 0.053 | 0.022 | 0.521 | 0.730 |
| ATAT1 | 0.625 | 0.052 | 0.022 | 0.523 | 0.728 |
| AMZ2 | 0.625 | 0.051 | 0.022 | 0.525 | 0.726 |
| PLEKHG2 | 0.625 | 0.052 | 0.022 | 0.523 | 0.728 |
| ANAPC7 | 0.625 | 0.053 | 0.022 | 0.520 | 0.730 |
| DR1 | 0.625 | 0.053 | 0.022 | 0.521 | 0.729 |
| EGR1 | 0.625 | 0.053 | 0.022 | 0.521 | 0.729 |
| NR1D2 | 0.625 | 0.052 | 0.022 | 0.522 | 0.728 |
| ADRBK2 | 0.625 | 0.052 | 0.022 | 0.523 | 0.727 |
| LRRC8B | 0.625 | 0.053 | 0.022 | 0.521 | 0.729 |
| MSI1 | 0.625 | 0.053 | 0.022 | 0.521 | 0.729 |
| MTHFD1 | 0.625 | 0.054 | 0.022 | 0.519 | 0.731 |
| SNRPC | 0.625 | 0.053 | 0.022 | 0.521 | 0.729 |
| HSPA13 | 0.625 | 0.053 | 0.022 | 0.522 | 0.728 |
| PSIP1 | 0.625 | 0.053 | 0.022 | 0.521 | 0.729 |
| RBFA | 0.625 | 0.053 | 0.022 | 0.521 | 0.728 |
| IL7R | 0.625 | 0.052 | 0.022 | 0.523 | 0.727 |
| AC104472.1 | 0.625 | 0.052 | 0.022 | 0.523 | 0.727 |
| CHEK1 | 0.625 | 0.052 | 0.022 | 0.523 | 0.726 |
| HMGB3 | 0.624 | 0.053 | 0.023 | 0.521 | 0.727 |
| NICN1 | 0.624 | 0.054 | 0.023 | 0.519 | 0.730 |
| DCUN1D1 | 0.624 | 0.055 | 0.023 | 0.517 | 0.731 |
| C14orf118 | 0.624 | 0.054 | 0.023 | 0.519 | 0.730 |
| ZNF655 | 0.624 | 0.052 | 0.023 | 0.522 | 0.726 |
| ETV6 | 0.624 | 0.054 | 0.023 | 0.519 | 0.729 |
| YARS2 | 0.624 | 0.055 | 0.023 | 0.517 | 0.732 |
| MCTP2 | 0.624 | 0.052 | 0.023 | 0.523 | 0.725 |
| FBXO7 | 0.624 | 0.053 | 0.023 | 0.520 | 0.728 |
| MNDA | 0.624 | 0.053 | 0.023 | 0.519 | 0.728 |
| SLC43A3 | 0.624 | 0.051 | 0.023 | 0.524 | 0.724 |
| DDB2 | 0.624 | 0.053 | 0.023 | 0.520 | 0.728 |
| USP18 | 0.624 | 0.052 | 0.023 | 0.522 | 0.726 |
| M6PR | 0.624 | 0.054 | 0.024 | 0.518 | 0.729 |
| TRIM59 | 0.624 | 0.052 | 0.024 | 0.522 | 0.725 |
| CDCA8 | 0.624 | 0.051 | 0.024 | 0.523 | 0.724 |
| LSP1 | 0.624 | 0.053 | 0.024 | 0.520 | 0.727 |
| MKI67 | 0.624 | 0.054 | 0.024 | 0.518 | 0.729 |
| SNRPA | 0.624 | 0.052 | 0.024 | 0.522 | 0.725 |
| CYTH4 | 0.624 | 0.051 | 0.024 | 0.523 | 0.724 |
| RPAIN | 0.624 | 0.051 | 0.024 | 0.524 | 0.723 |
| SELO | 0.624 | 0.053 | 0.024 | 0.519 | 0.728 |
| BRD2 | 0.623 | 0.055 | 0.024 | 0.517 | 0.730 |
| SNX20 | 0.623 | 0.053 | 0.024 | 0.519 | 0.727 |
| ASXL1 | 0.623 | 0.053 | 0.024 | 0.519 | 0.727 |
| SERBP1 | 0.623 | 0.053 | 0.024 | 0.519 | 0.728 |
| ZNF639 | 0.623 | 0.052 | 0.024 | 0.521 | 0.725 |
| TBC1D23 | 0.623 | 0.051 | 0.024 | 0.523 | 0.724 |
| DGKA | 0.623 | 0.054 | 0.024 | 0.518 | 0.729 |
| ETS1 | 0.623 | 0.053 | 0.024 | 0.520 | 0.727 |
| CD3G | 0.623 | 0.053 | 0.024 | 0.520 | 0.727 |
| ZBTB8A | 0.623 | 0.052 | 0.024 | 0.521 | 0.726 |
| ZNF142 | 0.623 | 0.052 | 0.024 | 0.520 | 0.726 |
| FAM26F | 0.623 | 0.054 | 0.024 | 0.517 | 0.729 |
| LRRC8C | 0.623 | 0.052 | 0.024 | 0.520 | 0.726 |
| MYC | 0.623 | 0.052 | 0.024 | 0.521 | 0.725 |
| CD53 | 0.623 | 0.053 | 0.024 | 0.518 | 0.728 |
| IRF8 | 0.623 | 0.053 | 0.024 | 0.520 | 0.726 |
| SAAL1 | 0.623 | 0.053 | 0.024 | 0.519 | 0.727 |
| ANKRD36 | 0.623 | 0.053 | 0.024 | 0.519 | 0.727 |
| SEMA4D | 0.623 | 0.051 | 0.024 | 0.522 | 0.724 |
| PPP6R2 | 0.623 | 0.054 | 0.025 | 0.518 | 0.728 |
| IL21R | 0.623 | 0.053 | 0.025 | 0.519 | 0.726 |
| WHSC1L1 | 0.623 | 0.055 | 0.025 | 0.515 | 0.730 |
| MTO1 | 0.623 | 0.053 | 0.025 | 0.519 | 0.726 |
| PTPN6 | 0.623 | 0.053 | 0.025 | 0.518 | 0.727 |
| MIR4517 | 0.622 | 0.054 | 0.025 | 0.516 | 0.729 |
| PDCD1LG2 | 0.622 | 0.052 | 0.025 | 0.521 | 0.724 |
| NUP153 | 0.622 | 0.054 | 0.025 | 0.517 | 0.728 |
| CD3D | 0.622 | 0.052 | 0.025 | 0.521 | 0.724 |
| SGCB | 0.622 | 0.052 | 0.025 | 0.520 | 0.725 |
| ZDHHC2 | 0.622 | 0.051 | 0.025 | 0.522 | 0.723 |
| EXT2 | 0.622 | 0.054 | 0.025 | 0.516 | 0.729 |
| RAD51D | 0.622 | 0.052 | 0.025 | 0.520 | 0.724 |
| CXCL 10 | 0.622 | 0.052 | 0.025 | 0.521 | 0.723 |
| GLT25D1 | 0.622 | 0.053 | 0.025 | 0.518 | 0.726 |
| RBM10 | 0.622 | 0.054 | 0.025 | 0.515 | 0.729 |
| BCAS2 | 0.622 | 0.054 | 0.025 | 0.516 | 0.728 |
| REL | 0.622 | 0.052 | 0.025 | 0.519 | 0.725 |
| LPHN1 | 0.622 | 0.052 | 0.025 | 0.519 | 0.725 |
| KLK5 | 0.622 | 0.052 | 0.025 | 0.519 | 0.725 |
| DENND2D | 0.622 | 0.053 | 0.025 | 0.517 | 0.727 |
| YBX1 | 0.622 | 0.053 | 0.026 | 0.518 | 0.726 |
| INPP5B | 0.622 | 0.053 | 0.026 | 0.518 | 0.726 |
| CSF3R | 0.622 | 0.052 | 0.026 | 0.519 | 0.725 |
| INSR | 0.622 | 0.052 | 0.026 | 0.519 | 0.724 |
| AHI1 | 0.622 | 0.053 | 0.026 | 0.517 | 0.727 |
| CALM1 | 0.622 | 0.053 | 0.026 | 0.518 | 0.725 |
| CSTF3 | 0.622 | 0.053 | 0.026 | 0.517 | 0.726 |
| PAK1IP1 | 0.622 | 0.052 | 0.026 | 0.519 | 0.724 |
| KRT14 | 0.622 | 0.052 | 0.026 | 0.520 | 0.723 |
| KDM4C | 0.622 | 0.055 | 0.026 | 0.514 | 0.729 |
| NUP160 | 0.622 | 0.054 | 0.026 | 0.515 | 0.728 |
| TPP2 | 0.622 | 0.054 | 0.026 | 0.515 | 0.728 |
| FUS | 0.622 | 0.054 | 0.026 | 0.516 | 0.727 |
| SMCHD1 | 0.622 | 0.055 | 0.026 | 0.513 | 0.730 |
| SUV39H2 | 0.622 | 0.052 | 0.026 | 0.520 | 0.723 |
| RARRES1 | 0.621 | 0.052 | 0.026 | 0.520 | 0.723 |
| KDM2B | 0.621 | 0.054 | 0.026 | 0.516 | 0.727 |
| DLGAP5 | 0.621 | 0.053 | 0.026 | 0.518 | 0.725 |
| MSL2 | 0.621 | 0.055 | 0.026 | 0.513 | 0.729 |
| SNORA11D | 0.621 | 0.053 | 0.026 | 0.517 | 0.725 |
| NFYA | 0.621 | 0.053 | 0.026 | 0.517 | 0.725 |
| H1FX | 0.621 | 0.056 | 0.027 | 0.512 | 0.730 |
| FAM189A2 | 0.621 | 0.053 | 0.027 | 0.518 | 0.724 |
| TTC17 | 0.621 | 0.054 | 0.027 | 0.515 | 0.727 |
| STK38 | 0.621 | 0.053 | 0.027 | 0.517 | 0.724 |
| CERK | 0.621 | 0.054 | 0.027 | 0.516 | 0.726 |
| HDGFRP2 | 0.621 | 0.054 | 0.027 | 0.515 | 0.726 |
| ZNF672 | 0.621 | 0.053 | 0.027 | 0.516 | 0.726 |
| WIPF1 | 0.621 | 0.053 | 0.027 | 0.518 | 0.724 |
| DEPDC1B | 0.621 | 0.052 | 0.027 | 0.520 | 0.722 |
| NCAPH2 | 0.621 | 0.052 | 0.027 | 0.518 | 0.724 |
| CEPT1 | 0.621 | 0.055 | 0.027 | 0.513 | 0.729 |
| SIK1 | 0.621 | 0.054 | 0.027 | 0.515 | 0.726 |
| DIRAS1 | 0.621 | 0.052 | 0.027 | 0.518 | 0.723 |
| ITGB2 | 0.621 | 0.052 | 0.027 | 0.519 | 0.723 |
| BIVM-ERCC5 | 0.621 | 0.054 | 0.027 | 0.514 | 0.727 |
| ACAD11 | 0.621 | 0.051 | 0.027 | 0.520 | 0.721 |
| MRPL37 | 0.621 | 0.053 | 0.027 | 0.518 | 0.724 |
| SRF | 0.621 | 0.053 | 0.027 | 0.517 | 0.724 |
| THADA | 0.620 | 0.054 | 0.027 | 0.514 | 0.727 |
| ZNF180 | 0.620 | 0.053 | 0.027 | 0.517 | 0.723 |
| PPP1R13L | 0.620 | 0.054 | 0.027 | 0.514 | 0.726 |
| P2RY13 | 0.620 | 0.053 | 0.027 | 0.516 | 0.724 |
| PPP5C | 0.620 | 0.055 | 0.027 | 0.512 | 0.728 |
| ANKRD18A | 0.620 | 0.054 | 0.027 | 0.515 | 0.725 |
| MLL4 | 0.620 | 0.053 | 0.027 | 0.517 | 0.723 |
| EVL | 0.620 | 0.054 | 0.027 | 0.515 | 0.726 |
| ATP11B | 0.620 | 0.053 | 0.027 | 0.517 | 0.724 |
| CNBP | 0.620 | 0.053 | 0.027 | 0.517 | 0.724 |
| NEDD1 | 0.620 | 0.054 | 0.027 | 0.515 | 0.725 |
| CDK6 | 0.620 | 0.052 | 0.027 | 0.518 | 0.723 |
| UNK | 0.620 | 0.052 | 0.027 | 0.518 | 0.723 |
| TYMP | 0.620 | 0.052 | 0.027 | 0.518 | 0.723 |
| DSP | 0.620 | 0.052 | 0.027 | 0.518 | 0.722 |
| EBP | 0.620 | 0.053 | 0.027 | 0.517 | 0.724 |
| ZNF559 | 0.620 | 0.052 | 0.028 | 0.517 | 0.723 |
| PTPLA | 0.620 | 0.052 | 0.028 | 0.519 | 0.721 |
| LRRC42 | 0.620 | 0.053 | 0.028 | 0.516 | 0.724 |
| DCLRE1C | 0.620 | 0.053 | 0.028 | 0.515 | 0.725 |
| DCAF7 | 0.620 | 0.054 | 0.028 | 0.514 | 0.726 |
| PDCD2L | 0.620 | 0.053 | 0.028 | 0.516 | 0.724 |
| ARHGAP33 | 0.620 | 0.053 | 0.028 | 0.516 | 0.724 |
| CR1 | 0.620 | 0.053 | 0.028 | 0.517 | 0.723 |
| BCL11A | 0.620 | 0.051 | 0.028 | 0.520 | 0.720 |
| PAFAH1B1 | 0.620 | 0.053 | 0.028 | 0.516 | 0.724 |
| ZNF146 | 0.620 | 0.054 | 0.028 | 0.515 | 0.725 |
| SENP6 | 0.620 | 0.053 | 0.028 | 0.516 | 0.724 |
| CD3E | 0.620 | 0.053 | 0.028 | 0.516 | 0.723 |
| MRPS23 | 0.620 | 0.053 | 0.028 | 0.516 | 0.724 |
| LOC389906 | 0.620 | 0.053 | 0.028 | 0.516 | 0.724 |
| RBM12B | 0.619 | 0.053 | 0.029 | 0.516 | 0.722 |
| SUMO2 | 0.619 | 0.055 | 0.029 | 0.512 | 0.727 |
| SERPINB8 | 0.619 | 0.053 | 0.029 | 0.516 | 0.723 |
| USP13 | 0.619 | 0.054 | 0.029 | 0.514 | 0.725 |
| CD58 | 0.619 | 0.052 | 0.029 | 0.517 | 0.722 |
| AKAP8 | 0.619 | 0.052 | 0.029 | 0.517 | 0.722 |
| MICAL1 | 0.619 | 0.052 | 0.029 | 0.517 | 0.722 |
| EIF2C4 | 0.619 | 0.053 | 0.029 | 0.516 | 0.723 |
| SCML2 | 0.619 | 0.052 | 0.029 | 0.517 | 0.722 |
| RRP7A | 0.619 | 0.052 | 0.029 | 0.517 | 0.722 |
| CCDC9 | 0.619 | 0.051 | 0.029 | 0.519 | 0.719 |
| DSG2 | 0.619 | 0.052 | 0.029 | 0.517 | 0.721 |
| CD6 | 0.619 | 0.055 | 0.029 | 0.512 | 0.726 |
| CCL18 | 0.619 | 0.052 | 0.029 | 0.518 | 0.720 |
| TCEAL2 | 0.619 | 0.051 | 0.029 | 0.519 | 0.720 |
| OIP5 | 0.619 | 0.053 | 0.029 | 0.516 | 0.722 |
| IRF1 | 0.619 | 0.053 | 0.029 | 0.515 | 0.723 |
| MAP3K1 | 0.619 | 0.054 | 0.029 | 0.513 | 0.725 |
| TLN1 | 0.619 | 0.053 | 0.029 | 0.516 | 0.723 |
| USP37 | 0.619 | 0.053 | 0.029 | 0.516 | 0.722 |
| FGFR1OP | 0.619 | 0.052 | 0.029 | 0.517 | 0.721 |
| ZBED4 | 0.619 | 0.056 | 0.029 | 0.510 | 0.728 |
| TRMT10B | 0.619 | 0.053 | 0.029 | 0.514 | 0.724 |
| IL32 | 0.619 | 0.052 | 0.029 | 0.517 | 0.721 |
| MPP1 | 0.619 | 0.054 | 0.029 | 0.513 | 0.725 |
| NDC80 | 0.619 | 0.052 | 0.029 | 0.517 | 0.720 |
| FAR1 | 0.619 | 0.054 | 0.029 | 0.513 | 0.724 |
| LRRC37B | 0.619 | 0.053 | 0.029 | 0.514 | 0.723 |
| C5orf46 | 0.619 | 0.052 | 0.029 | 0.517 | 0.721 |
| TRIM22 | 0.619 | 0.054 | 0.030 | 0.513 | 0.724 |
| BAK1 | 0.619 | 0.053 | 0.030 | 0.515 | 0.722 |
| FAM64A | 0.619 | 0.053 | 0.030 | 0.515 | 0.722 |
| AGTPBP1 | 0.619 | 0.055 | 0.030 | 0.512 | 0.725 |
| SLC2A14 | 0.619 | 0.054 | 0.030 | 0.514 | 0.724 |
| ZNF253 | 0.619 | 0.054 | 0.030 | 0.513 | 0.724 |
| B3GALNT1 | 0.619 | 0.053 | 0.030 | 0.515 | 0.722 |
| ELF3 | 0.619 | 0.052 | 0.030 | 0.516 | 0.721 |
| AMICA1 | 0.619 | 0.053 | 0.030 | 0.514 | 0.723 |
| IKBKB | 0.619 | 0.052 | 0.030 | 0.516 | 0.721 |
| VHL | 0.618 | 0.054 | 0.030 | 0.512 | 0.725 |
| COL4A2 | 0.618 | 0.054 | 0.030 | 0.513 | 0.723 |
| MDN1 | 0.618 | 0.053 | 0.030 | 0.514 | 0.723 |
| AVEN | 0.618 | 0.052 | 0.030 | 0.517 | 0.720 |
| KLHDC7B | 0.618 | 0.053 | 0.030 | 0.514 | 0.723 |
| TMEM14B | 0.618 | 0.054 | 0.030 | 0.512 | 0.725 |
| LOC100507424 | 0.618 | 0.053 | 0.030 | 0.515 | 0.722 |
| C12orf32 | 0.618 | 0.053 | 0.030 | 0.514 | 0.722 |
| C5orf25 | 0.618 | 0.051 | 0.030 | 0.518 | 0.719 |
| SDCCAG3 | 0.618 | 0.053 | 0.030 | 0.514 | 0.722 |
| TRAF3IP3 | 0.618 | 0.052 | 0.030 | 0.515 | 0.721 |
| TTC19 | 0.618 | 0.053 | 0.030 | 0.514 | 0.722 |
| CRTC2 | 0.618 | 0.053 | 0.031 | 0.515 | 0.721 |
| GMPS | 0.618 | 0.054 | 0.031 | 0.512 | 0.724 |
| UQCRHL | 0.618 | 0.053 | 0.031 | 0.514 | 0.722 |
| KPNA2 | 0.618 | 0.053 | 0.031 | 0.514 | 0.722 |
| CLEC2D | 0.618 | 0.053 | 0.031 | 0.515 | 0.721 |
| CLEC10A | 0.618 | 0.053 | 0.031 | 0.515 | 0.721 |
| CIITA | 0.618 | 0.053 | 0.031 | 0.514 | 0.721 |
| ALG14 | 0.618 | 0.054 | 0.031 | 0.513 | 0.723 |
| HNRPLL | 0.618 | 0.053 | 0.031 | 0.515 | 0.721 |
| KCTD9 | 0.618 | 0.052 | 0.031 | 0.517 | 0.719 |
| STAC | 0.618 | 0.054 | 0.031 | 0.513 | 0.723 |
| SNX10 | 0.618 | 0.051 | 0.031 | 0.517 | 0.718 |
| CXCL9 | 0.618 | 0.052 | 0.031 | 0.516 | 0.720 |
| GPRIN1 | 0.618 | 0.052 | 0.031 | 0.517 | 0.719 |
| C18orf25 | 0.618 | 0.053 | 0.031 | 0.513 | 0.722 |
| VAV1 | 0.618 | 0.052 | 0.031 | 0.515 | 0.720 |
| TCN2 | 0.618 | 0.052 | 0.031 | 0.516 | 0.720 |
| KCNJ8 | 0.618 | 0.053 | 0.031 | 0.514 | 0.721 |
| GPR137B | 0.618 | 0.052 | 0.031 | 0.515 | 0.720 |
| LOC100506032 | 0.618 | 0.052 | 0.031 | 0.516 | 0.719 |
| IL2RG | 0.618 | 0.052 | 0.031 | 0.516 | 0.719 |
| HDAC5 | 0.618 | 0.052 | 0.031 | 0.515 | 0.720 |
| RELT | 0.617 | 0.054 | 0.032 | 0.512 | 0.723 |
| EPB41 | 0.617 | 0.053 | 0.032 | 0.514 | 0.721 |
| HNRNPUL2 | 0.617 | 0.053 | 0.032 | 0.513 | 0.722 |
| SKIL | 0.617 | 0.053 | 0.032 | 0.514 | 0.721 |
| TRAPPC10 | 0.617 | 0.053 | 0.032 | 0.514 | 0.721 |
| SAMD9L | 0.617 | 0.054 | 0.032 | 0.512 | 0.722 |
| OGFR | 0.617 | 0.052 | 0.032 | 0.515 | 0.720 |
| PARL | 0.617 | 0.054 | 0.032 | 0.512 | 0.722 |
| ZNF384 | 0.617 | 0.053 | 0.032 | 0.513 | 0.722 |
| TYMS | 0.617 | 0.053 | 0.032 | 0.513 | 0.721 |
| LIG1 | 0.617 | 0.051 | 0.032 | 0.517 | 0.718 |
| PCSK7 | 0.617 | 0.052 | 0.032 | 0.516 | 0.718 |
| CD3EAP | 0.617 | 0.051 | 0.032 | 0.517 | 0.717 |
| HMGA1P8 | 0.617 | 0.052 | 0.032 | 0.515 | 0.719 |
| POLA1 | 0.617 | 0.054 | 0.032 | 0.510 | 0.724 |
| HNRNPA1L2 | 0.617 | 0.054 | 0.032 | 0.511 | 0.723 |
| VARS | 0.617 | 0.052 | 0.032 | 0.515 | 0.719 |
| AFG3L2 | 0.617 | 0.054 | 0.032 | 0.511 | 0.723 |
| USF2 | 0.617 | 0.052 | 0.032 | 0.514 | 0.720 |
| SIAH2 | 0.617 | 0.053 | 0.032 | 0.514 | 0.720 |
| HIST1H2BO | 0.617 | 0.055 | 0.032 | 0.509 | 0.724 |
| GFOD1 | 0.617 | 0.053 | 0.032 | 0.513 | 0.720 |
| PLEKHA2 | 0.617 | 0.054 | 0.032 | 0.511 | 0.723 |
| PAN3 | 0.617 | 0.054 | 0.032 | 0.511 | 0.723 |
| RIF1 | 0.617 | 0.054 | 0.032 | 0.511 | 0.723 |
| ZXDC | 0.617 | 0.054 | 0.032 | 0.511 | 0.722 |
| CNPY3 | 0.617 | 0.053 | 0.032 | 0.513 | 0.721 |
| HN1 | 0.617 | 0.052 | 0.032 | 0.514 | 0.719 |
| LRRC37A | 0.617 | 0.053 | 0.032 | 0.513 | 0.721 |
| CBL | 0.617 | 0.054 | 0.032 | 0.511 | 0.722 |
| C19orf12 | 0.617 | 0.051 | 0.033 | 0.516 | 0.717 |
| CENPV | 0.616 | 0.053 | 0.033 | 0.512 | 0.720 |
| ZNF592 | 0.616 | 0.053 | 0.033 | 0.513 | 0.720 |
| IL17RA | 0.616 | 0.055 | 0.033 | 0.509 | 0.723 |
| RC3H2 | 0.616 | 0.055 | 0.033 | 0.509 | 0.724 |
| C2 | 0.616 | 0.052 | 0.033 | 0.515 | 0.718 |
| NAT10 | 0.616 | 0.053 | 0.033 | 0.512 | 0.721 |
| CD27 | 0.616 | 0.052 | 0.033 | 0.514 | 0.718 |
| GBP4 | 0.616 | 0.053 | 0.033 | 0.512 | 0.721 |
| CHIC2 | 0.616 | 0.052 | 0.033 | 0.514 | 0.719 |
| ANKRD16 | 0.616 | 0.053 | 0.033 | 0.513 | 0.720 |
| IPO5 | 0.616 | 0.053 | 0.033 | 0.512 | 0.721 |
| WDTC1 | 0.616 | 0.052 | 0.033 | 0.514 | 0.718 |
| ZNF549 | 0.616 | 0.053 | 0.033 | 0.512 | 0.720 |
| CCDC82 | 0.616 | 0.055 | 0.033 | 0.508 | 0.724 |
| SF3A2 | 0.616 | 0.055 | 0.033 | 0.509 | 0.724 |
| WAS | 0.616 | 0.054 | 0.033 | 0.511 | 0.721 |
| CD226 | 0.616 | 0.054 | 0.033 | 0.511 | 0.721 |
| GYG1 | 0.616 | 0.054 | 0.033 | 0.510 | 0.722 |
| HOMER3 | 0.616 | 0.052 | 0.033 | 0.515 | 0.717 |
| OSGEP | 0.616 | 0.053 | 0.033 | 0.513 | 0.719 |
| MYD88 | 0.616 | 0.054 | 0.033 | 0.510 | 0.723 |
| APOBEC3F | 0.616 | 0.052 | 0.033 | 0.515 | 0.718 |
| FMNL1 | 0.616 | 0.054 | 0.033 | 0.511 | 0.721 |
| HIPK3 | 0.616 | 0.056 | 0.033 | 0.507 | 0.725 |
| PFKFB3 | 0.616 | 0.051 | 0.033 | 0.516 | 0.716 |
| DCUN1D2 | 0.616 | 0.053 | 0.034 | 0.512 | 0.720 |
| HMGN2 | 0.616 | 0.053 | 0.034 | 0.512 | 0.720 |
| JUND | 0.616 | 0.053 | 0.034 | 0.512 | 0.719 |
| PLP2 | 0.616 | 0.056 | 0.034 | 0.507 | 0.725 |
| PTDSS1 | 0.616 | 0.054 | 0.034 | 0.509 | 0.722 |
| COIL | 0.616 | 0.053 | 0.034 | 0.511 | 0.720 |
| WDR33 | 0.616 | 0.055 | 0.034 | 0.508 | 0.724 |
| TMTC4 | 0.616 | 0.054 | 0.034 | 0.510 | 0.721 |
| NT5C3 | 0.616 | 0.053 | 0.034 | 0.511 | 0.720 |
| SEMA4A | 0.616 | 0.052 | 0.034 | 0.513 | 0.718 |
| CAMKMT | 0.616 | 0.052 | 0.034 | 0.514 | 0.718 |
| MGAT1 | 0.616 | 0.053 | 0.034 | 0.512 | 0.719 |
| MAU2 | 0.616 | 0.053 | 0.034 | 0.512 | 0.719 |
| ZC3H7B | 0.616 | 0.054 | 0.034 | 0.510 | 0.721 |
| LRCH3 | 0.616 | 0.053 | 0.034 | 0.511 | 0.720 |
| PLEKHO1 | 0.615 | 0.052 | 0.035 | 0.512 | 0.718 |
| MGAT2 | 0.615 | 0.054 | 0.035 | 0.509 | 0.721 |
| UBA5 | 0.615 | 0.053 | 0.035 | 0.511 | 0.719 |
| ABTB1 | 0.615 | 0.054 | 0.035 | 0.510 | 0.721 |
| FERMT3 | 0.615 | 0.052 | 0.035 | 0.513 | 0.717 |
| CTPS2 | 0.615 | 0.052 | 0.035 | 0.513 | 0.718 |
| ZNF594 | 0.615 | 0.053 | 0.035 | 0.511 | 0.719 |
| SENP5 | 0.615 | 0.055 | 0.035 | 0.508 | 0.723 |
| PTPRC | 0.615 | 0.054 | 0.035 | 0.509 | 0.721 |
| SETD5 | 0.615 | 0.053 | 0.035 | 0.512 | 0.719 |
| CDC42SE2 | 0.615 | 0.055 | 0.035 | 0.507 | 0.723 |
| TRAPPC6A | 0.615 | 0.053 | 0.035 | 0.511 | 0.719 |
| IFNAR2 | 0.615 | 0.053 | 0.035 | 0.510 | 0.720 |
| RASA4B | 0.615 | 0.051 | 0.035 | 0.515 | 0.715 |
| LRRC37A2 | 0.615 | 0.053 | 0.035 | 0.510 | 0.719 |
| THUMPD2 | 0.615 | 0.053 | 0.035 | 0.510 | 0.719 |
| E2F3 | 0.615 | 0.052 | 0.035 | 0.513 | 0.717 |
| AGPAT6 | 0.615 | 0.052 | 0.035 | 0.513 | 0.717 |
| LOC100653018 | 0.615 | 0.053 | 0.035 | 0.511 | 0.719 |
| MUC16 | 0.615 | 0.053 | 0.035 | 0.512 | 0.718 |
| DAXX | 0.615 | 0.053 | 0.035 | 0.511 | 0.719 |
| FNBP1 | 0.615 | 0.055 | 0.035 | 0.507 | 0.723 |
| SERPING1 | 0.615 | 0.052 | 0.036 | 0.513 | 0.717 |
| CMC1 | 0.615 | 0.052 | 0.036 | 0.513 | 0.716 |
| SRSF10 | 0.615 | 0.052 | 0.036 | 0.513 | 0.716 |
| ELOVL1 | 0.615 | 0.051 | 0.036 | 0.514 | 0.716 |
| ASH1L | 0.615 | 0.054 | 0.036 | 0.509 | 0.720 |
| CAPN7 | 0.614 | 0.056 | 0.036 | 0.505 | 0.724 |
| PLEKHG4B | 0.614 | 0.054 | 0.036 | 0.509 | 0.720 |
| NUF2 | 0.614 | 0.053 | 0.036 | 0.511 | 0.718 |
| AIF1 | 0.614 | 0.054 | 0.036 | 0.509 | 0.720 |
| MRS2 | 0.614 | 0.054 | 0.036 | 0.509 | 0.720 |
| POLG | 0.614 | 0.055 | 0.036 | 0.506 | 0.722 |
| CDCA7L | 0.614 | 0.053 | 0.036 | 0.511 | 0.718 |
| CEP85 | 0.614 | 0.052 | 0.036 | 0.513 | 0.716 |
| WDR48 | 0.614 | 0.055 | 0.036 | 0.506 | 0.722 |
| NMT2 | 0.614 | 0.054 | 0.036 | 0.508 | 0.720 |
| CNTNAP3 | 0.614 | 0.053 | 0.037 | 0.510 | 0.719 |
| PDGFA | 0.614 | 0.054 | 0.037 | 0.507 | 0.721 |
| METTL4 | 0.614 | 0.053 | 0.037 | 0.510 | 0.718 |
| ICOSLG | 0.614 | 0.053 | 0.037 | 0.510 | 0.718 |
| ZNF673 | 0.614 | 0.052 | 0.037 | 0.512 | 0.716 |
| GIMAP4 | 0.614 | 0.054 | 0.037 | 0.508 | 0.720 |
| SUPT4H1 | 0.614 | 0.054 | 0.037 | 0.507 | 0.721 |
| KLHL24 | 0.614 | 0.054 | 0.037 | 0.508 | 0.720 |
| ALPL | 0.614 | 0.053 | 0.037 | 0.510 | 0.717 |
| NPC1 | 0.614 | 0.052 | 0.037 | 0.511 | 0.716 |
| ESD | 0.614 | 0.053 | 0.037 | 0.511 | 0.717 |
| RPS12 | 0.614 | 0.054 | 0.037 | 0.508 | 0.719 |
| CTLA4 | 0.614 | 0.054 | 0.037 | 0.509 | 0.719 |
| LCORL | 0.614 | 0.053 | 0.037 | 0.509 | 0.718 |
| ZNF766 | 0.614 | 0.053 | 0.037 | 0.509 | 0.719 |
| LOC100507424 | 0.614 | 0.055 | 0.037 | 0.506 | 0.721 |
| DOCK2 | 0.614 | 0.053 | 0.037 | 0.509 | 0.718 |
| EIF2S1 | 0.614 | 0.052 | 0.037 | 0.511 | 0.717 |
| ZDBF2 | 0.614 | 0.052 | 0.037 | 0.512 | 0.716 |
| DAPK2 | 0.614 | 0.053 | 0.037 | 0.510 | 0.718 |
| SATB1 | 0.614 | 0.055 | 0.037 | 0.506 | 0.721 |
| NOL11 | 0.613 | 0.054 | 0.038 | 0.508 | 0.718 |
| SH2D1A | 0.613 | 0.051 | 0.038 | 0.513 | 0.714 |
| KRT16 | 0.613 | 0.051 | 0.038 | 0.513 | 0.714 |
| ARHGAP25 | 0.613 | 0.054 | 0.038 | 0.509 | 0.718 |
| CXCR4 | 0.613 | 0.053 | 0.038 | 0.509 | 0.718 |
| UBXN7 | 0.613 | 0.057 | 0.038 | 0.503 | 0.724 |
| TAGAP | 0.613 | 0.053 | 0.038 | 0.510 | 0.717 |
| MAGOHB | 0.613 | 0.052 | 0.038 | 0.512 | 0.715 |
| TNFAIP2 | 0.613 | 0.052 | 0.038 | 0.511 | 0.716 |
| CCL2 | 0.613 | 0.052 | 0.038 | 0.511 | 0.716 |
| SCAI | 0.613 | 0.054 | 0.038 | 0.508 | 0.718 |
| NIN | 0.613 | 0.054 | 0.038 | 0.508 | 0.718 |
| TMEM14E | 0.613 | 0.053 | 0.038 | 0.509 | 0.717 |
| SRSF1 | 0.613 | 0.053 | 0.038 | 0.509 | 0.717 |
| ZZEF1 | 0.613 | 0.054 | 0.038 | 0.507 | 0.719 |
| RP11-408E5.4 | 0.613 | 0.052 | 0.038 | 0.511 | 0.716 |
| SPOP | 0.613 | 0.053 | 0.038 | 0.509 | 0.717 |
| MSL3 | 0.613 | 0.052 | 0.038 | 0.511 | 0.716 |
| LCP2 | 0.613 | 0.053 | 0.038 | 0.509 | 0.717 |
| ZNF45 | 0.613 | 0.054 | 0.038 | 0.507 | 0.720 |
| CNOT6 | 0.613 | 0.054 | 0.038 | 0.508 | 0.719 |
| MIR3614 | 0.613 | 0.053 | 0.038 | 0.509 | 0.717 |
| C11orf91 | 0.613 | 0.052 | 0.038 | 0.511 | 0.715 |
| CHAMP1 | 0.613 | 0.052 | 0.038 | 0.511 | 0.716 |
| INIP | 0.613 | 0.052 | 0.039 | 0.511 | 0.715 |
| C6orf141 | 0.613 | 0.054 | 0.039 | 0.507 | 0.719 |
| DCK | 0.613 | 0.052 | 0.039 | 0.511 | 0.715 |
| H2AFY | 0.613 | 0.055 | 0.039 | 0.506 | 0.720 |
| DGKH | 0.613 | 0.053 | 0.039 | 0.509 | 0.717 |
| MYO9B | 0.613 | 0.053 | 0.039 | 0.509 | 0.717 |
| CARD11 | 0.613 | 0.052 | 0.039 | 0.510 | 0.716 |
| SLC35A1 | 0.613 | 0.053 | 0.039 | 0.509 | 0.716 |
| FAAH2 | 0.613 | 0.053 | 0.039 | 0.508 | 0.718 |
| NFATC1 | 0.613 | 0.053 | 0.039 | 0.510 | 0.716 |
| PQLC1 | 0.613 | 0.053 | 0.039 | 0.508 | 0.718 |
| CD97 | 0.613 | 0.052 | 0.039 | 0.510 | 0.715 |
| KIF23 | 0.613 | 0.052 | 0.039 | 0.511 | 0.715 |
| ZNF236 | 0.613 | 0.054 | 0.039 | 0.506 | 0.719 |
| SPTAN1 | 0.613 | 0.054 | 0.039 | 0.507 | 0.718 |
| LDHB | 0.613 | 0.053 | 0.039 | 0.510 | 0.716 |
| LCP1 | 0.613 | 0.052 | 0.039 | 0.510 | 0.715 |
| ITGB4 | 0.613 | 0.053 | 0.039 | 0.510 | 0.716 |
| CARD8 | 0.613 | 0.054 | 0.039 | 0.506 | 0.719 |
| LIMD1 | 0.613 | 0.054 | 0.039 | 0.506 | 0.719 |
| DOCK9 | 0.613 | 0.052 | 0.039 | 0.510 | 0.715 |
| WDR1 | 0.612 | 0.053 | 0.039 | 0.508 | 0.717 |
| RNF138 | 0.612 | 0.054 | 0.040 | 0.506 | 0.719 |
| DDX47 | 0.612 | 0.054 | 0.040 | 0.507 | 0.718 |
| KIAA1524 | 0.612 | 0.051 | 0.040 | 0.512 | 0.713 |
| LPAR1 | 0.612 | 0.053 | 0.040 | 0.508 | 0.717 |
| PCED1B | 0.612 | 0.053 | 0.040 | 0.509 | 0.716 |
| CIR1 | 0.612 | 0.054 | 0.040 | 0.506 | 0.719 |
| KDM3B | 0.612 | 0.056 | 0.040 | 0.503 | 0.722 |
| IER2 | 0.612 | 0.054 | 0.040 | 0.506 | 0.718 |
| TMED5 | 0.612 | 0.052 | 0.040 | 0.510 | 0.715 |
| ZBTB11 | 0.612 | 0.053 | 0.040 | 0.509 | 0.715 |
| TMCO3 | 0.612 | 0.053 | 0.040 | 0.508 | 0.717 |
| ADAM19 | 0.612 | 0.051 | 0.040 | 0.511 | 0.713 |
| METTL8 | 0.612 | 0.052 | 0.040 | 0.510 | 0.714 |
| GNL2 | 0.612 | 0.053 | 0.040 | 0.508 | 0.715 |
| CTSL1 | 0.612 | 0.053 | 0.040 | 0.508 | 0.716 |
| PPP2R5D | 0.612 | 0.053 | 0.040 | 0.508 | 0.716 |
| KIF11 | 0.612 | 0.053 | 0.040 | 0.508 | 0.716 |
| RFC4 | 0.612 | 0.055 | 0.040 | 0.505 | 0.719 |
| WDR43 | 0.612 | 0.053 | 0.040 | 0.508 | 0.716 |
| IMMP1L | 0.612 | 0.053 | 0.040 | 0.508 | 0.716 |
| PTPN22 | 0.612 | 0.052 | 0.040 | 0.510 | 0.714 |
| MAP2K5 | 0.612 | 0.053 | 0.040 | 0.507 | 0.717 |
| TLE2 | 0.612 | 0.054 | 0.040 | 0.507 | 0.717 |
| FXR1 | 0.612 | 0.054 | 0.040 | 0.506 | 0.718 |
| SLC15A4 | 0.612 | 0.052 | 0.040 | 0.509 | 0.715 |
| ZGPAT | 0.612 | 0.052 | 0.040 | 0.511 | 0.713 |
| GRB2 | 0.612 | 0.055 | 0.041 | 0.505 | 0.719 |
| LMNB2 | 0.612 | 0.056 | 0.041 | 0.502 | 0.721 |
| YY1AP1 | 0.612 | 0.054 | 0.041 | 0.507 | 0.717 |
| LOC283070 | 0.612 | 0.052 | 0.041 | 0.509 | 0.714 |
| CTPS1 | 0.612 | 0.052 | 0.041 | 0.509 | 0.714 |
| CLCC1 | 0.612 | 0.053 | 0.041 | 0.508 | 0.715 |
| ARL4C | 0.612 | 0.053 | 0.041 | 0.508 | 0.715 |
| CLNS1A | 0.612 | 0.053 | 0.041 | 0.508 | 0.715 |
| RASA3 | 0.612 | 0.053 | 0.041 | 0.507 | 0.716 |
| LAP3 | 0.612 | 0.054 | 0.041 | 0.506 | 0.717 |
| @44807 | 0.611 | 0.053 | 0.041 | 0.507 | 0.716 |
| CHFR | 0.611 | 0.053 | 0.041 | 0.507 | 0.716 |
| PASK | 0.611 | 0.054 | 0.041 | 0.506 | 0.717 |
| USP41 | 0.611 | 0.053 | 0.041 | 0.507 | 0.716 |
| VDAC3 | 0.611 | 0.053 | 0.041 | 0.507 | 0.716 |
| MMP1 | 0.611 | 0.053 | 0.041 | 0.508 | 0.715 |
| COQ2 | 0.611 | 0.052 | 0.041 | 0.510 | 0.713 |
| SLC9A9 | 0.611 | 0.053 | 0.041 | 0.507 | 0.716 |
| CCL4 | 0.611 | 0.052 | 0.042 | 0.510 | 0.712 |
| C2orf44 | 0.611 | 0.056 | 0.042 | 0.501 | 0.721 |
| GNAI3 | 0.611 | 0.055 | 0.042 | 0.503 | 0.719 |
| DCTN3 | 0.611 | 0.053 | 0.042 | 0.508 | 0.714 |
| RAVER1 | 0.611 | 0.055 | 0.042 | 0.503 | 0.719 |
| RP11-166B2.1 | 0.611 | 0.055 | 0.042 | 0.504 | 0.719 |
| RPL35A | 0.611 | 0.052 | 0.042 | 0.508 | 0.714 |
| GPATCH1 | 0.611 | 0.052 | 0.042 | 0.510 | 0.712 |
| HIPK1 | 0.611 | 0.053 | 0.042 | 0.507 | 0.715 |
| ZNF318 | 0.611 | 0.053 | 0.042 | 0.508 | 0.714 |
| NASP | 0.611 | 0.052 | 0.042 | 0.508 | 0.713 |
| CASC5 | 0.611 | 0.053 | 0.042 | 0.507 | 0.714 |
| SP2 | 0.611 | 0.054 | 0.042 | 0.505 | 0.716 |
| DOCK8 | 0.611 | 0.053 | 0.042 | 0.506 | 0.715 |
| MAGEH1 | 0.611 | 0.054 | 0.042 | 0.505 | 0.717 |
| CLASP2 | 0.611 | 0.056 | 0.042 | 0.500 | 0.721 |
| ETV3 | 0.611 | 0.052 | 0.042 | 0.509 | 0.712 |
| LMF2 | 0.611 | 0.055 | 0.042 | 0.502 | 0.719 |
| MALT1 | 0.611 | 0.053 | 0.043 | 0.507 | 0.715 |
| B2M | 0.610 | 0.055 | 0.043 | 0.503 | 0.718 |
| MIR548N | 0.610 | 0.053 | 0.043 | 0.507 | 0.714 |
| HAUS2 | 0.610 | 0.054 | 0.043 | 0.505 | 0.716 |
| SREBF2 | 0.610 | 0.053 | 0.043 | 0.506 | 0.715 |
| ZFYVE9 | 0.610 | 0.055 | 0.043 | 0.502 | 0.719 |
| SAMD4A | 0.610 | 0.052 | 0.043 | 0.508 | 0.713 |
| EFTUD1 | 0.610 | 0.055 | 0.043 | 0.503 | 0.718 |
| RDH14 | 0.610 | 0.053 | 0.043 | 0.507 | 0.714 |
| DAPK1 | 0.610 | 0.052 | 0.043 | 0.509 | 0.712 |
| GADD45B | 0.610 | 0.052 | 0.043 | 0.509 | 0.712 |
| LYZ | 0.610 | 0.054 | 0.043 | 0.505 | 0.716 |
| NT5C3L | 0.610 | 0.053 | 0.043 | 0.508 | 0.713 |
| PRPF8 | 0.610 | 0.055 | 0.043 | 0.504 | 0.717 |
| TTLL3 | 0.610 | 0.053 | 0.043 | 0.506 | 0.715 |
| TRMT11 | 0.610 | 0.054 | 0.043 | 0.505 | 0.716 |
| RBP5 | 0.610 | 0.053 | 0.043 | 0.505 | 0.715 |
| ZNF607 | 0.610 | 0.053 | 0.043 | 0.506 | 0.714 |
| PTGES | 0.610 | 0.053 | 0.043 | 0.507 | 0.714 |
| KDM2A | 0.610 | 0.053 | 0.043 | 0.507 | 0.714 |
| MAPK14 | 0.610 | 0.053 | 0.043 | 0.506 | 0.715 |
| FAM189B | 0.610 | 0.053 | 0.043 | 0.507 | 0.714 |
| FAM110A | 0.610 | 0.052 | 0.043 | 0.507 | 0.713 |
| HBS1L | 0.610 | 0.055 | 0.043 | 0.503 | 0.717 |
| DSC3 | 0.610 | 0.052 | 0.043 | 0.509 | 0.712 |
| SPAG9 | 0.610 | 0.053 | 0.043 | 0.507 | 0.713 |
| ATP1B3 | 0.610 | 0.053 | 0.043 | 0.506 | 0.715 |
| XRCC6 | 0.610 | 0.054 | 0.043 | 0.504 | 0.717 |
| POLDIP3 | 0.610 | 0.055 | 0.043 | 0.503 | 0.717 |
| TBC1D22B | 0.610 | 0.053 | 0.044 | 0.506 | 0.714 |
| PIK3CA | 0.610 | 0.053 | 0.044 | 0.506 | 0.714 |
| ZNF749 | 0.610 | 0.051 | 0.044 | 0.509 | 0.710 |
| QKI | 0.610 | 0.053 | 0.045 | 0.506 | 0.713 |
| UQCRFS1 | 0.610 | 0.053 | 0.045 | 0.505 | 0.714 |
| MCOLN2 | 0.610 | 0.053 | 0.045 | 0.506 | 0.713 |
| CSRNP1 | 0.610 | 0.052 | 0.045 | 0.508 | 0.712 |
| P2RX5 | 0.610 | 0.053 | 0.045 | 0.505 | 0.714 |
| RPS6KA3 | 0.610 | 0.053 | 0.045 | 0.505 | 0.714 |
| CCDC134 | 0.610 | 0.054 | 0.045 | 0.504 | 0.715 |
| TCTN2 | 0.610 | 0.054 | 0.045 | 0.505 | 0.714 |
| TMEM91 | 0.610 | 0.052 | 0.045 | 0.507 | 0.712 |
| DEPDC1 | 0.609 | 0.053 | 0.045 | 0.505 | 0.713 |
| NFKBIL1 | 0.609 | 0.053 | 0.045 | 0.505 | 0.714 |
| FLNA | 0.609 | 0.053 | 0.045 | 0.506 | 0.712 |
| MECP2 | 0.609 | 0.053 | 0.045 | 0.506 | 0.713 |
| DLST | 0.609 | 0.053 | 0.045 | 0.505 | 0.714 |
| TCF7L1 | 0.609 | 0.052 | 0.045 | 0.507 | 0.712 |
| GIMAP6 | 0.609 | 0.054 | 0.045 | 0.504 | 0.715 |
| C1R | 0.609 | 0.054 | 0.045 | 0.504 | 0.714 |
| ARPC2 | 0.609 | 0.053 | 0.045 | 0.505 | 0.714 |
| LDHAL6A | 0.609 | 0.054 | 0.045 | 0.504 | 0.715 |
| ADNP2 | 0.609 | 0.053 | 0.045 | 0.505 | 0.713 |
| KIAA 1804 | 0.609 | 0.053 | 0.045 | 0.506 | 0.713 |
| C9orf72 | 0.609 | 0.053 | 0.045 | 0.506 | 0.712 |
| CD40 | 0.609 | 0.053 | 0.045 | 0.506 | 0.713 |
| UBAC2 | 0.609 | 0.054 | 0.045 | 0.504 | 0.715 |
| EMB | 0.609 | 0.053 | 0.045 | 0.506 | 0.713 |
| DDHD2 | 0.609 | 0.052 | 0.045 | 0.506 | 0.712 |
| FOXJ2 | 0.609 | 0.053 | 0.046 | 0.506 | 0.712 |
| SAMD4B | 0.609 | 0.053 | 0.046 | 0.504 | 0.714 |
| KCNAB2 | 0.609 | 0.055 | 0.046 | 0.502 | 0.716 |
| CNTNAP3B | 0.609 | 0.054 | 0.046 | 0.504 | 0.714 |
| TMEM241 | 0.609 | 0.053 | 0.046 | 0.506 | 0.712 |
| ADPRHL2 | 0.609 | 0.053 | 0.046 | 0.504 | 0.714 |
| ZBTB43 | 0.609 | 0.054 | 0.046 | 0.504 | 0.714 |
| PPP1R18 | 0.609 | 0.052 | 0.046 | 0.507 | 0.711 |
| WBP11 | 0.609 | 0.053 | 0.046 | 0.505 | 0.713 |
| ZNF708 | 0.609 | 0.052 | 0.046 | 0.506 | 0.712 |
| APPL1 | 0.609 | 0.053 | 0.046 | 0.505 | 0.713 |
| RPL14 | 0.609 | 0.054 | 0.046 | 0.502 | 0.716 |
| KRT15 | 0.609 | 0.052 | 0.046 | 0.506 | 0.712 |
| KLC1 | 0.609 | 0.057 | 0.046 | 0.498 | 0.720 |
| ZNF317 | 0.609 | 0.054 | 0.046 | 0.503 | 0.715 |
| FAM96A | 0.609 | 0.053 | 0.046 | 0.504 | 0.713 |
| ANKRD13A | 0.609 | 0.054 | 0.046 | 0.503 | 0.714 |
| ABCC1 | 0.609 | 0.053 | 0.046 | 0.504 | 0.713 |
| RBM26 | 0.609 | 0.054 | 0.046 | 0.503 | 0.714 |
| ZMYM5 | 0.609 | 0.054 | 0.046 | 0.503 | 0.714 |
| ZNF66 | 0.609 | 0.052 | 0.046 | 0.506 | 0.711 |
| DESI1 | 0.609 | 0.054 | 0.046 | 0.502 | 0.715 |
| UNC13D | 0.609 | 0.054 | 0.046 | 0.503 | 0.714 |
| MAST2 | 0.609 | 0.053 | 0.046 | 0.505 | 0.712 |
| ZNF525 | 0.609 | 0.053 | 0.046 | 0.505 | 0.712 |
| MKNK1 | 0.609 | 0.052 | 0.047 | 0.506 | 0.711 |
| NME2 | 0.608 | 0.054 | 0.047 | 0.503 | 0.714 |
| GFM1 | 0.608 | 0.053 | 0.047 | 0.504 | 0.713 |
| HNRNPH1 | 0.608 | 0.054 | 0.047 | 0.504 | 0.713 |
| CLIC1 | 0.608 | 0.054 | 0.047 | 0.503 | 0.713 |
| WDR76 | 0.608 | 0.054 | 0.047 | 0.503 | 0.714 |
| RNF115 | 0.608 | 0.054 | 0.047 | 0.503 | 0.714 |
| PTMS | 0.608 | 0.052 | 0.047 | 0.506 | 0.711 |
| PRKCB | 0.608 | 0.053 | 0.047 | 0.504 | 0.712 |
| GK5 | 0.608 | 0.053 | 0.047 | 0.505 | 0.711 |
| GAPDH | 0.608 | 0.054 | 0.048 | 0.503 | 0.714 |
| LOC146880 | 0.608 | 0.055 | 0.048 | 0.501 | 0.715 |
| DUSP11 | 0.608 | 0.053 | 0.048 | 0.504 | 0.712 |
| ABL2 | 0.608 | 0.054 | 0.048 | 0.502 | 0.714 |
| SRSF4 | 0.608 | 0.053 | 0.048 | 0.504 | 0.712 |
| GDI1 | 0.608 | 0.052 | 0.048 | 0.507 | 0.710 |
| MFHAS1 | 0.608 | 0.054 | 0.048 | 0.503 | 0.714 |
| DCBLD1 | 0.608 | 0.054 | 0.048 | 0.503 | 0.713 |
| @44810 | 0.608 | 0.055 | 0.048 | 0.501 | 0.716 |
| GNB4 | 0.608 | 0.053 | 0.048 | 0.505 | 0.712 |
| ANKLE2 | 0.608 | 0.052 | 0.048 | 0.506 | 0.710 |
| KANSL2 | 0.608 | 0.054 | 0.048 | 0.502 | 0.713 |
| ANKRD40 | 0.608 | 0.053 | 0.048 | 0.504 | 0.711 |
| FPR3 | 0.608 | 0.053 | 0.048 | 0.504 | 0.712 |
| ST6GALNAC5 | 0.608 | 0.054 | 0.048 | 0.503 | 0.713 |
| CD84 | 0.608 | 0.054 | 0.048 | 0.503 | 0.713 |
| DAZAP1 | 0.608 | 0.056 | 0.048 | 0.498 | 0.717 |
| AP4E1 | 0.608 | 0.056 | 0.048 | 0.498 | 0.718 |
| DICER1 | 0.608 | 0.054 | 0.048 | 0.502 | 0.714 |
| MT2A | 0.608 | 0.052 | 0.048 | 0.505 | 0.711 |
| MAGOH | 0.608 | 0.053 | 0.048 | 0.504 | 0.712 |
| RAB40B | 0.608 | 0.052 | 0.048 | 0.506 | 0.710 |
| TDP2 | 0.608 | 0.056 | 0.049 | 0.498 | 0.717 |
| POLD3 | 0.608 | 0.055 | 0.049 | 0.500 | 0.715 |
| LRRC25 | 0.608 | 0.053 | 0.049 | 0.503 | 0.712 |
| EAF1 | 0.608 | 0.055 | 0.049 | 0.499 | 0.716 |
| NLRP2 | 0.608 | 0.053 | 0.049 | 0.504 | 0.711 |
| NIPA1 | 0.608 | 0.052 | 0.049 | 0.506 | 0.709 |
| PROSC | 0.608 | 0.054 | 0.049 | 0.501 | 0.714 |
| RRAS2 | 0.608 | 0.053 | 0.049 | 0.504 | 0.711 |
| ME2 | 0.608 | 0.053 | 0.049 | 0.503 | 0.712 |
| MIR4442 | 0.608 | 0.056 | 0.049 | 0.498 | 0.718 |
| IL6R | 0.608 | 0.054 | 0.049 | 0.502 | 0.713 |
| LCK | 0.608 | 0.053 | 0.049 | 0.504 | 0.711 |
| PHTF2 | 0.607 | 0.056 | 0.049 | 0.498 | 0.717 |
| UVRAG | 0.607 | 0.054 | 0.049 | 0.501 | 0.713 |
| PLA2G2D | 0.607 | 0.054 | 0.049 | 0.501 | 0.713 |
| SDF2 | 0.607 | 0.052 | 0.049 | 0.506 | 0.709 |
| NCBP2 | 0.607 | 0.055 | 0.049 | 0.500 | 0.715 |
| ILF3 | 0.607 | 0.053 | 0.049 | 0.502 | 0.712 |
| TIGIT | 0.607 | 0.053 | 0.049 | 0.503 | 0.711 |
| CAPG | 0.607 | 0.052 | 0.049 | 0.505 | 0.709 |
| SYMPK | 0.607 | 0.052 | 0.049 | 0.505 | 0.710 |
| ELAC1 | 0.607 | 0.055 | 0.049 | 0.500 | 0.714 |
| RBM7 | 0.607 | 0.052 | 0.049 | 0.505 | 0.709 |
| STRN4 | 0.607 | 0.054 | 0.049 | 0.501 | 0.714 |
| DKFZp686K1684 | 0.607 | 0.053 | 0.049 | 0.504 | 0.710 |
| MIR4745 | 0.607 | 0.056 | 0.049 | 0.498 | 0.717 |
| ABCA5 | 0.607 | 0.053 | 0.049 | 0.503 | 0.711 |

TABLE 8
Genes predictive of RD from the GSE106977 cohort.
Area under the curve

| | | | | Asymptotic 95% | |
|---|---|---|---|---|---|
| Test | | | | Confidence Interval | |
| Result | | Std. | Asymptotic | Lower | Upper |
| Variable(s) | Area | Error^{a} | Sig.^{b} | Bound | Bound |
| FMO4 | 0.691 | 0.049 | 0.000 | 0.595 | 0.787 |
| OR2AE1 | 0.688 | 0.048 | 0.001 | 0.593 | 0.782 |
| ZNF467 | 0.686 | 0.049 | 0.001 | 0.589 | 0.782 |
| MTRNR2L10 | 0.683 | 0.050 | 0.001 | 0.585 | 0.781 |
| SLC13A3 | 0.683 | 0.052 | 0.001 | 0.580 | 0.786 |
| GDF15 | 0.681 | 0.049 | 0.001 | 0.584 | 0.778 |
| PRDM13 | 0.680 | 0.049 | 0.001 | 0.584 | 0.777 |
| TMPRSS4 | 0.674 | 0.052 | 0.001 | 0.573 | 0.775 |
| MMP23B | 0.673 | 0.050 | 0.002 | 0.575 | 0.771 |
| PRTG | 0.672 | 0.051 | 0.002 | 0.571 | 0.773 |
| CYP2E1 | 0.672 | 0.051 | 0.002 | 0.572 | 0.772 |
| TMEM150A | 0.671 | 0.049 | 0.002 | 0.574 | 0.767 |
| XKR6 | 0.670 | 0.051 | 0.002 | 0.570 | 0.770 |
| CLCA2 | 0.670 | 0.050 | 0.002 | 0.572 | 0.768 |
| POF1B | 0.669 | 0.049 | 0.002 | 0.572 | 0.766 |
| NQO1 | 0.668 | 0.051 | 0.002 | 0.569 | 0.768 |
| PRKACG | 0.668 | 0.051 | 0.002 | 0.568 | 0.767 |
| KNDC1 | 0.668 | 0.050 | 0.002 | 0.569 | 0.766 |
| HSF4 | 0.666 | 0.054 | 0.002 | 0.561 | 0.771 |
| HOMER2 | 0.666 | 0.050 | 0.002 | 0.568 | 0.765 |
| KRTAP4-4 | 0.663 | 0.053 | 0.003 | 0.559 | 0.767 |
| PAFAH2 | 0.663 | 0.051 | 0.003 | 0.563 | 0.763 |
| MTHFR | 0.663 | 0.052 | 0.003 | 0.560 | 0.766 |
| CCDC85C | 0.663 | 0.051 | 0.003 | 0.563 | 0.763 |
| RASL10B | 0.662 | 0.051 | 0.003 | 0.562 | 0.761 |
| SEMA3E | 0.661 | 0.051 | 0.003 | 0.561 | 0.762 |
| RAP1GAP | 0.661 | 0.051 | 0.003 | 0.560 | 0.762 |
| ZKSCAN5 | 0.660 | 0.053 | 0.003 | 0.556 | 0.764 |
| HS6ST2 | 0.660 | 0.050 | 0.003 | 0.561 | 0.759 |
| PGC | 0.658 | 0.052 | 0.004 | 0.557 | 0.760 |
| FOXN1 | 0.658 | 0.053 | 0.004 | 0.554 | 0.762 |
| LOC100652860 | 0.658 | 0.050 | 0.004 | 0.559 | 0.756 |
| C11orf94 | 0.658 | 0.053 | 0.004 | 0.554 | 0.761 |
| FAM5C | 0.657 | 0.050 | 0.004 | 0.558 | 0.756 |
| TRIM62 | 0.657 | 0.051 | 0.004 | 0.557 | 0.756 |
| SPATA24 | 0.657 | 0.051 | 0.004 | 0.556 | 0.757 |
| DHRS7B | 0.656 | 0.052 | 0.004 | 0.554 | 0.758 |
| PCDHB14 | 0.656 | 0.053 | 0.004 | 0.553 | 0.759 |
| C1orf129 | 0.656 | 0.051 | 0.004 | 0.556 | 0.756 |
| DIO2 | 0.655 | 0.052 | 0.004 | 0.553 | 0.757 |
| CMBL | 0.655 | 0.052 | 0.005 | 0.553 | 0.756 |
| AFAP1L2 | 0.654 | 0.051 | 0.005 | 0.553 | 0.755 |
| NKX2-3 | 0.654 | 0.053 | 0.005 | 0.550 | 0.757 |
| ZBTB42 | 0.653 | 0.051 | 0.005 | 0.553 | 0.753 |
| FAM131B | 0.653 | 0.054 | 0.005 | 0.547 | 0.759 |
| NRXN2 | 0.653 | 0.054 | 0.005 | 0.548 | 0.758 |
| PLA2G12B | 0.653 | 0.051 | 0.005 | 0.553 | 0.753 |
| BPIFB1 | 0.653 | 0.051 | 0.005 | 0.552 | 0.753 |
| PWWP2B | 0.652 | 0.052 | 0.005 | 0.551 | 0.754 |
| PNLDC1 | 0.652 | 0.051 | 0.005 | 0.552 | 0.752 |
| SCRG1 | 0.652 | 0.051 | 0.005 | 0.551 | 0.753 |
| GALNT7 | 0.652 | 0.050 | 0.005 | 0.553 | 0.750 |
| C20orf197 | 0.652 | 0.055 | 0.005 | 0.544 | 0.759 |
| AKR7L | 0.651 | 0.052 | 0.006 | 0.550 | 0.752 |
| MUC2 | 0.651 | 0.053 | 0.006 | 0.547 | 0.755 |
| FLJ46300 | 0.650 | 0.051 | 0.006 | 0.549 | 0.751 |
| ARHGEF16 | 0.650 | 0.052 | 0.006 | 0.549 | 0.751 |
| REEP6 | 0.650 | 0.052 | 0.006 | 0.548 | 0.751 |
| PGBD2 | 0.649 | 0.052 | 0.006 | 0.547 | 0.752 |
| ABCC2 | 0.649 | 0.051 | 0.006 | 0.550 | 0.749 |
| PON3 | 0.649 | 0.052 | 0.006 | 0.547 | 0.751 |
| CCNO | 0.649 | 0.054 | 0.006 | 0.544 | 0.754 |
| VSTM2L | 0.648 | 0.050 | 0.007 | 0.551 | 0.745 |
| HSPB3 | 0.648 | 0.051 | 0.007 | 0.547 | 0.748 |
| RPGRIP1L | 0.648 | 0.052 | 0.007 | 0.546 | 0.750 |
| C2CD4B | 0.647 | 0.051 | 0.007 | 0.547 | 0.748 |
| ETNK2 | 0.647 | 0.051 | 0.007 | 0.547 | 0.748 |
| SPA17 | 0.647 | 0.051 | 0.007 | 0.547 | 0.747 |
| AGR2 | 0.647 | 0.050 | 0.007 | 0.550 | 0.745 |
| GKAP1 | 0.647 | 0.054 | 0.007 | 0.542 | 0.753 |
| LOC646471 | 0.647 | 0.052 | 0.007 | 0.545 | 0.749 |
| ZNF596 | 0.647 | 0.052 | 0.007 | 0.545 | 0.749 |
| RET | 0.646 | 0.050 | 0.007 | 0.548 | 0.744 |
| ABCC11 | 0.646 | 0.051 | 0.007 | 0.546 | 0.746 |
| IL17F | 0.646 | 0.052 | 0.007 | 0.545 | 0.747 |
| KRT20 | 0.646 | 0.054 | 0.008 | 0.540 | 0.751 |
| CSNK1A1L | 0.646 | 0.054 | 0.008 | 0.540 | 0.751 |
| EGFLAM | 0.646 | 0.054 | 0.008 | 0.541 | 0.751 |
| CES4A | 0.645 | 0.053 | 0.008 | 0.541 | 0.750 |
| DIO2 | 0.645 | 0.052 | 0.008 | 0.544 | 0.746 |
| MTRNR2L1 | 0.645 | 0.050 | 0.008 | 0.547 | 0.743 |
| HOXC5 | 0.645 | 0.053 | 0.008 | 0.542 | 0.748 |
| CLVS1 | 0.644 | 0.052 | 0.008 | 0.542 | 0.746 |
| ANKRD33 | 0.644 | 0.052 | 0.008 | 0.543 | 0.745 |
| OR10Q1 | 0.644 | 0.053 | 0.008 | 0.540 | 0.748 |
| VENTX | 0.644 | 0.051 | 0.009 | 0.544 | 0.743 |
| MIEN1 | 0.643 | 0.052 | 0.009 | 0.541 | 0.745 |
| MAPK10 | 0.643 | 0.051 | 0.009 | 0.542 | 0.744 |
| AL359878.1 | 0.642 | 0.055 | 0.009 | 0.534 | 0.749 |
| COL4A5 | 0.641 | 0.052 | 0.010 | 0.539 | 0.744 |
| GPR98 | 0.641 | 0.051 | 0.010 | 0.542 | 0.741 |
| C11orf70 | 0.641 | 0.052 | 0.010 | 0.539 | 0.743 |
| MTERFD3 | 0.641 | 0.054 | 0.010 | 0.535 | 0.746 |
| PRPS1L1 | 0.641 | 0.052 | 0.010 | 0.540 | 0.742 |
| IGFL1 | 0.641 | 0.054 | 0.010 | 0.535 | 0.746 |
| TDRD9 | 0.640 | 0.052 | 0.010 | 0.538 | 0.741 |
| RND1 | 0.640 | 0.051 | 0.010 | 0.539 | 0.740 |
| NTS | 0.640 | 0.050 | 0.010 | 0.542 | 0.738 |
| PPFIA3 | 0.640 | 0.053 | 0.010 | 0.536 | 0.744 |
| GSTA3 | 0.640 | 0.052 | 0.011 | 0.538 | 0.741 |
| ARFIP2 | 0.639 | 0.053 | 0.011 | 0.536 | 0.743 |
| KIAA2022 | 0.639 | 0.051 | 0.011 | 0.539 | 0.740 |
| PIGM | 0.639 | 0.050 | 0.011 | 0.540 | 0.738 |
| CLDN12 | 0.639 | 0.055 | 0.011 | 0.531 | 0.747 |
| SCAND1 | 0.639 | 0.052 | 0.011 | 0.537 | 0.740 |
| FAM110C | 0.639 | 0.051 | 0.011 | 0.539 | 0.739 |
| AKR7A3 | 0.638 | 0.051 | 0.011 | 0.538 | 0.739 |
| CCNJL | 0.638 | 0.054 | 0.011 | 0.532 | 0.745 |
| CDHR1 | 0.638 | 0.052 | 0.011 | 0.536 | 0.741 |
| DNAJC18 | 0.638 | 0.051 | 0.011 | 0.538 | 0.739 |
| DEFB132 | 0.638 | 0.052 | 0.011 | 0.536 | 0.741 |
| MTNR1B | 0.638 | 0.055 | 0.012 | 0.530 | 0.745 |
| NUDT6 | 0.638 | 0.051 | 0.012 | 0.537 | 0.738 |
| FOLR3 | 0.638 | 0.051 | 0.012 | 0.537 | 0.738 |
| CAPN8 | 0.638 | 0.053 | 0.012 | 0.534 | 0.742 |
| GNMT | 0.638 | 0.051 | 0.012 | 0.537 | 0.739 |
| CAPN13 | 0.638 | 0.051 | 0.012 | 0.537 | 0.739 |
| SMUG1 | 0.638 | 0.051 | 0.012 | 0.537 | 0.739 |
| FAM166A | 0.638 | 0.053 | 0.012 | 0.535 | 0.740 |
| AR | 0.638 | 0.051 | 0.012 | 0.537 | 0.738 |
| PROC | 0.637 | 0.052 | 0.012 | 0.536 | 0.739 |
| ARHGEF38 | 0.637 | 0.051 | 0.012 | 0.538 | 0.736 |
| C20orf96 | 0.637 | 0.052 | 0.012 | 0.535 | 0.739 |
| VWA2 | 0.637 | 0.052 | 0.012 | 0.535 | 0.738 |
| MAFA | 0.637 | 0.052 | 0.012 | 0.534 | 0.739 |
| OR51I1 | 0.637 | 0.052 | 0.012 | 0.536 | 0.738 |
| FANK1 | 0.636 | 0.051 | 0.012 | 0.536 | 0.737 |
| KCND2 | 0.636 | 0.052 | 0.012 | 0.534 | 0.739 |
| LRRC30 | 0.636 | 0.052 | 0.012 | 0.534 | 0.739 |
| TMEM151A | 0.636 | 0.053 | 0.013 | 0.532 | 0.740 |
| KLLN | 0.636 | 0.055 | 0.013 | 0.529 | 0.743 |
| ENOX1 | 0.636 | 0.053 | 0.013 | 0.533 | 0.739 |
| SLC25A34 | 0.636 | 0.052 | 0.013 | 0.535 | 0.737 |
| AC010336.1 | 0.636 | 0.054 | 0.013 | 0.530 | 0.741 |
| KLHDC7A | 0.635 | 0.051 | 0.013 | 0.535 | 0.736 |
| ZSCAN4 | 0.635 | 0.054 | 0.013 | 0.529 | 0.742 |
| WFDC6 | 0.635 | 0.053 | 0.013 | 0.533 | 0.738 |
| CR392000.1 | 0.635 | 0.051 | 0.013 | 0.535 | 0.736 |
| SULT1B1 | 0.635 | 0.053 | 0.013 | 0.531 | 0.740 |
| LOC730102 | 0.635 | 0.054 | 0.013 | 0.530 | 0.741 |
| ONECUT3 | 0.635 | 0.054 | 0.013 | 0.530 | 0.740 |
| C9orf171 | 0.635 | 0.053 | 0.013 | 0.531 | 0.739 |
| DCD | 0.635 | 0.054 | 0.013 | 0.530 | 0.740 |
| NHLH1 | 0.635 | 0.053 | 0.013 | 0.530 | 0.739 |
| CTXN2 | 0.635 | 0.053 | 0.013 | 0.531 | 0.739 |
| CTD-2260A17.2 | 0.635 | 0.053 | 0.014 | 0.530 | 0.739 |
| GPR139 | 0.635 | 0.054 | 0.014 | 0.529 | 0.741 |
| TSC22D3 | 0.634 | 0.053 | 0.014 | 0.531 | 0.738 |
| PRR23A | 0.634 | 0.053 | 0.014 | 0.531 | 0.738 |
| CROT | 0.634 | 0.053 | 0.014 | 0.531 | 0.737 |
| FEZF1 | 0.634 | 0.052 | 0.014 | 0.531 | 0.737 |
| MUSK | 0.634 | 0.054 | 0.014 | 0.528 | 0.740 |
| FAM216B | 0.634 | 0.053 | 0.014 | 0.531 | 0.737 |
| SWI5 | 0.634 | 0.052 | 0.014 | 0.532 | 0.736 |
| ORM2 | 0.634 | 0.053 | 0.014 | 0.530 | 0.738 |
| SLX4 | 0.634 | 0.052 | 0.014 | 0.532 | 0.736 |
| APOBEC4 | 0.634 | 0.052 | 0.014 | 0.531 | 0.737 |
| C1QTNF9B-AS1 | 0.634 | 0.052 | 0.014 | 0.532 | 0.736 |
| C9orf170 | 0.634 | 0.053 | 0.014 | 0.530 | 0.737 |
| NAT1 | 0.634 | 0.053 | 0.014 | 0.530 | 0.738 |
| CYP8B1 | 0.634 | 0.053 | 0.014 | 0.531 | 0.737 |
| RTL1 | 0.634 | 0.052 | 0.014 | 0.532 | 0.735 |
| ZG16B | 0.634 | 0.053 | 0.014 | 0.530 | 0.737 |
| PCDHB11 | 0.633 | 0.054 | 0.014 | 0.528 | 0.738 |
| OR6N2 | 0.633 | 0.054 | 0.014 | 0.528 | 0.739 |
| NEUROG1 | 0.633 | 0.052 | 0.014 | 0.531 | 0.736 |
| FOXA1 | 0.633 | 0.051 | 0.014 | 0.534 | 0.733 |
| HPGD | 0.633 | 0.052 | 0.014 | 0.532 | 0.735 |
| TMEM63C | 0.633 | 0.052 | 0.015 | 0.531 | 0.735 |
| C5orf30 | 0.633 | 0.053 | 0.015 | 0.530 | 0.736 |
| C20orf85 | 0.633 | 0.053 | 0.015 | 0.529 | 0.737 |
| @44816 | 0.633 | 0.055 | 0.015 | 0.525 | 0.740 |
| ADSSL1 | 0.632 | 0.052 | 0.015 | 0.531 | 0.733 |
| OR2T4 | 0.632 | 0.052 | 0.015 | 0.530 | 0.734 |
| SLC6A1 | 0.632 | 0.053 | 0.015 | 0.528 | 0.736 |
| RAB3IL1 | 0.632 | 0.052 | 0.015 | 0.530 | 0.735 |
| GJC3 | 0.632 | 0.053 | 0.015 | 0.529 | 0.736 |
| GPR115 | 0.632 | 0.054 | 0.016 | 0.526 | 0.737 |
| OR51M1 | 0.632 | 0.052 | 0.016 | 0.530 | 0.734 |
| OR5M1 | 0.632 | 0.053 | 0.016 | 0.529 | 0.735 |
| LRG1 | 0.632 | 0.053 | 0.016 | 0.529 | 0.735 |
| TMEM53 | 0.632 | 0.053 | 0.016 | 0.529 | 0.735 |
| RPH3A | 0.632 | 0.052 | 0.016 | 0.530 | 0.734 |
| ENO4 | 0.632 | 0.052 | 0.016 | 0.529 | 0.734 |
| WDR78 | 0.632 | 0.056 | 0.016 | 0.523 | 0.741 |
| KATNAL2 | 0.632 | 0.052 | 0.016 | 0.529 | 0.734 |
| FA2H | 0.632 | 0.052 | 0.016 | 0.530 | 0.733 |
| PKIB | 0.632 | 0.051 | 0.016 | 0.532 | 0.732 |
| TMEM78 | 0.632 | 0.056 | 0.016 | 0.522 | 0.741 |
| SPRED3 | 0.631 | 0.053 | 0.016 | 0.528 | 0.735 |
| KBTBD12 | 0.631 | 0.053 | 0.016 | 0.528 | 0.735 |
| GPR135 | 0.631 | 0.051 | 0.016 | 0.532 | 0.731 |
| STRA8 | 0.631 | 0.053 | 0.016 | 0.527 | 0.736 |
| NFIA | 0.631 | 0.054 | 0.016 | 0.526 | 0.736 |
| NXPH1 | 0.631 | 0.052 | 0.016 | 0.529 | 0.733 |
| C2CD4C | 0.631 | 0.051 | 0.016 | 0.531 | 0.731 |
| C9orf9 | 0.631 | 0.053 | 0.016 | 0.528 | 0.734 |
| MINPP1 | 0.631 | 0.052 | 0.016 | 0.529 | 0.733 |
| TMEM133 | 0.631 | 0.052 | 0.016 | 0.529 | 0.733 |
| ORM1 | 0.631 | 0.053 | 0.016 | 0.527 | 0.735 |
| H2AFB2 | 0.631 | 0.052 | 0.017 | 0.529 | 0.732 |
| OR2T1 | 0.631 | 0.053 | 0.017 | 0.526 | 0.735 |
| SLC22A18 | 0.631 | 0.053 | 0.017 | 0.526 | 0.735 |
| SPATA25 | 0.630 | 0.052 | 0.017 | 0.529 | 0.732 |
| ATP8B3 | 0.630 | 0.055 | 0.017 | 0.522 | 0.739 |
| ARMCX5 | 0.630 | 0.056 | 0.017 | 0.521 | 0.740 |
| PLEKHA6 | 0.630 | 0.052 | 0.017 | 0.528 | 0.733 |
| NFS1 | 0.630 | 0.054 | 0.017 | 0.524 | 0.737 |
| FHAD1 | 0.630 | 0.052 | 0.017 | 0.528 | 0.733 |
| EFHC2 | 0.630 | 0.055 | 0.017 | 0.523 | 0.738 |
| CXXC4 | 0.630 | 0.054 | 0.017 | 0.525 | 0.735 |
| EPS8L3 | 0.630 | 0.053 | 0.017 | 0.526 | 0.734 |
| GUCY1A2 | 0.630 | 0.053 | 0.017 | 0.526 | 0.734 |
| DKFZP667F0711 | 0.630 | 0.053 | 0.017 | 0.527 | 0.734 |
| INSM1 | 0.630 | 0.053 | 0.017 | 0.526 | 0.733 |
| IGFBP1 | 0.630 | 0.052 | 0.017 | 0.528 | 0.732 |
| C1QTNF2 | 0.630 | 0.051 | 0.017 | 0.529 | 0.731 |
| ABCC12 | 0.630 | 0.052 | 0.017 | 0.527 | 0.732 |
| GNA14 | 0.630 | 0.053 | 0.018 | 0.526 | 0.733 |
| FGFBP3 | 0.630 | 0.052 | 0.018 | 0.528 | 0.731 |
| CCDC87 | 0.630 | 0.051 | 0.018 | 0.529 | 0.730 |
| SLC25A33 | 0.630 | 0.053 | 0.018 | 0.525 | 0.734 |
| AC099552.4 | 0.630 | 0.053 | 0.018 | 0.525 | 0.734 |
| PIP | 0.630 | 0.052 | 0.018 | 0.528 | 0.731 |
| GRAMD2 | 0.630 | 0.054 | 0.018 | 0.525 | 0.734 |
| PAQR6 | 0.629 | 0.052 | 0.018 | 0.527 | 0.732 |
| NBEA | 0.629 | 0.051 | 0.018 | 0.529 | 0.730 |
| TMBIM6 | 0.629 | 0.051 | 0.018 | 0.529 | 0.730 |
| SOX1 | 0.629 | 0.051 | 0.018 | 0.529 | 0.730 |
| FGF6 | 0.629 | 0.054 | 0.018 | 0.523 | 0.736 |
| FAM83A | 0.629 | 0.055 | 0.018 | 0.522 | 0.737 |
| AKR1B10 | 0.629 | 0.053 | 0.018 | 0.525 | 0.734 |
| CAV2 | 0.629 | 0.055 | 0.018 | 0.522 | 0.737 |
| ZRANB3 | 0.629 | 0.055 | 0.018 | 0.522 | 0.736 |
| MMP15 | 0.629 | 0.053 | 0.018 | 0.525 | 0.733 |
| C1QTNF4 | 0.629 | 0.053 | 0.018 | 0.525 | 0.733 |
| CHSY3 | 0.629 | 0.052 | 0.018 | 0.528 | 0.730 |
| RP11-204N11.1 | 0.629 | 0.055 | 0.018 | 0.520 | 0.738 |
| RFPL3 | 0.629 | 0.053 | 0.018 | 0.525 | 0.733 |
| ERVMER34-1 | 0.629 | 0.054 | 0.018 | 0.523 | 0.735 |
| NXF3 | 0.629 | 0.054 | 0.018 | 0.523 | 0.735 |
| HNF1B | 0.629 | 0.054 | 0.018 | 0.524 | 0.734 |
| ARHGAP40 | 0.629 | 0.052 | 0.018 | 0.527 | 0.730 |
| CRYGC | 0.629 | 0.051 | 0.018 | 0.528 | 0.730 |
| RNF32 | 0.628 | 0.055 | 0.019 | 0.520 | 0.737 |
| CXorf30 | 0.628 | 0.051 | 0.019 | 0.528 | 0.729 |
| LOC150935 | 0.628 | 0.054 | 0.019 | 0.523 | 0.734 |
| FMO5 | 0.628 | 0.052 | 0.019 | 0.527 | 0.730 |
| LOC440910 | 0.628 | 0.054 | 0.019 | 0.523 | 0.734 |
| BAAT | 0.628 | 0.052 | 0.019 | 0.526 | 0.730 |
| DHX35 | 0.628 | 0.052 | 0.019 | 0.526 | 0.731 |
| INSRR | 0.628 | 0.054 | 0.019 | 0.522 | 0.733 |
| GK2 | 0.628 | 0.054 | 0.019 | 0.521 | 0.734 |
| TPPP3 | 0.628 | 0.054 | 0.019 | 0.522 | 0.733 |
| MAOA | 0.628 | 0.053 | 0.019 | 0.525 | 0.731 |
| ATP13A4 | 0.628 | 0.052 | 0.019 | 0.525 | 0.730 |
| KCNK15 | 0.627 | 0.053 | 0.020 | 0.524 | 0.731 |
| XKR9 | 0.627 | 0.054 | 0.020 | 0.522 | 0.733 |
| TP53INP2 | 0.627 | 0.051 | 0.020 | 0.527 | 0.727 |
| MLIP | 0.627 | 0.053 | 0.020 | 0.524 | 0.731 |
| GATM | 0.627 | 0.052 | 0.020 | 0.525 | 0.730 |
| SRPK3 | 0.627 | 0.052 | 0.020 | 0.524 | 0.730 |
| CACNA1D | 0.627 | 0.055 | 0.020 | 0.520 | 0.734 |
| ABCG8 | 0.627 | 0.053 | 0.020 | 0.523 | 0.730 |
| DNASE1L2 | 0.627 | 0.053 | 0.020 | 0.523 | 0.730 |
| PPP2R2B | 0.627 | 0.054 | 0.020 | 0.522 | 0.732 |
| CCDC149 | 0.627 | 0.053 | 0.020 | 0.522 | 0.731 |
| INTS12 | 0.627 | 0.053 | 0.020 | 0.524 | 0.730 |
| UGT2B4 | 0.627 | 0.052 | 0.020 | 0.525 | 0.728 |
| FOXN4 | 0.626 | 0.053 | 0.021 | 0.522 | 0.731 |
| CAB39L | 0.626 | 0.054 | 0.021 | 0.521 | 0.732 |
| OR13H1 | 0.626 | 0.053 | 0.021 | 0.523 | 0.730 |
| MOGAT1 | 0.626 | 0.054 | 0.021 | 0.520 | 0.732 |
| MIR4695 | 0.626 | 0.053 | 0.021 | 0.523 | 0.729 |
| SHISA6 | 0.626 | 0.054 | 0.021 | 0.520 | 0.732 |
| IFT43 | 0.626 | 0.053 | 0.021 | 0.523 | 0.729 |
| MCC | 0.626 | 0.052 | 0.021 | 0.524 | 0.728 |
| FEZ1 | 0.626 | 0.053 | 0.021 | 0.523 | 0.729 |
| SERPINA5 | 0.626 | 0.054 | 0.021 | 0.520 | 0.732 |
| HMX2 | 0.626 | 0.053 | 0.021 | 0.522 | 0.729 |
| AGR3 | 0.626 | 0.052 | 0.021 | 0.523 | 0.728 |
| DHDH | 0.626 | 0.054 | 0.021 | 0.520 | 0.731 |
| IL1RN | 0.626 | 0.052 | 0.021 | 0.524 | 0.727 |
| SLC17A8 | 0.626 | 0.053 | 0.021 | 0.523 | 0.729 |
| SEBOX | 0.626 | 0.054 | 0.021 | 0.520 | 0.731 |
| CA4 | 0.625 | 0.053 | 0.022 | 0.522 | 0.728 |
| SLC2A10 | 0.625 | 0.051 | 0.022 | 0.525 | 0.726 |
| NKX2-5 | 0.625 | 0.052 | 0.022 | 0.523 | 0.727 |
| RBMXL2 | 0.625 | 0.054 | 0.022 | 0.520 | 0.730 |
| NR0B2 | 0.625 | 0.054 | 0.022 | 0.520 | 0.730 |
| CCL23 | 0.625 | 0.052 | 0.022 | 0.522 | 0.728 |
| IFNA13 | 0.625 | 0.052 | 0.022 | 0.522 | 0.727 |
| IQCH | 0.625 | 0.054 | 0.022 | 0.520 | 0.730 |
| ZNF23 | 0.625 | 0.052 | 0.022 | 0.522 | 0.727 |
| P2RY1 | 0.625 | 0.052 | 0.022 | 0.523 | 0.726 |
| C9orf169 | 0.625 | 0.052 | 0.022 | 0.523 | 0.727 |
| PCDHB16 | 0.625 | 0.053 | 0.022 | 0.520 | 0.730 |
| MSTN | 0.624 | 0.052 | 0.023 | 0.522 | 0.727 |
| CDH6 | 0.624 | 0.055 | 0.023 | 0.518 | 0.731 |
| MED11 | 0.624 | 0.054 | 0.023 | 0.519 | 0.730 |
| CFC1 | 0.624 | 0.053 | 0.023 | 0.520 | 0.729 |
| CCNG2 | 0.624 | 0.051 | 0.023 | 0.524 | 0.724 |
| B3GNT9 | 0.624 | 0.052 | 0.023 | 0.523 | 0.725 |
| HGD | 0.624 | 0.052 | 0.023 | 0.523 | 0.725 |
| DNASE1 | 0.624 | 0.052 | 0.023 | 0.522 | 0.726 |
| OR8D4 | 0.624 | 0.051 | 0.023 | 0.524 | 0.724 |
| C1orf227 | 0.624 | 0.054 | 0.023 | 0.519 | 0.729 |
| PP14571 | 0.624 | 0.053 | 0.023 | 0.520 | 0.728 |
| INPP5J | 0.624 | 0.053 | 0.023 | 0.520 | 0.728 |
| DLEU7 | 0.624 | 0.055 | 0.023 | 0.516 | 0.731 |
| PBLD | 0.624 | 0.052 | 0.023 | 0.521 | 0.727 |
| MORN1 | 0.624 | 0.052 | 0.023 | 0.521 | 0.726 |
| ERCC4 | 0.624 | 0.053 | 0.023 | 0.521 | 0.727 |
| ZNF530 | 0.624 | 0.054 | 0.024 | 0.518 | 0.729 |
| TBATA | 0.624 | 0.053 | 0.024 | 0.520 | 0.727 |
| SLC12A1 | 0.624 | 0.052 | 0.024 | 0.521 | 0.726 |
| C7orf61 | 0.624 | 0.052 | 0.024 | 0.521 | 0.726 |
| CARD14 | 0.623 | 0.051 | 0.024 | 0.523 | 0.724 |
| KCNMB2 | 0.623 | 0.053 | 0.024 | 0.519 | 0.727 |
| AC016251.1 | 0.623 | 0.052 | 0.024 | 0.521 | 0.725 |
| LOC641515 | 0.623 | 0.053 | 0.024 | 0.519 | 0.728 |
| KRTAP10-12 | 0.623 | 0.056 | 0.024 | 0.514 | 0.732 |
| RAB3C | 0.623 | 0.053 | 0.024 | 0.520 | 0.726 |
| C17orf50 | 0.623 | 0.052 | 0.024 | 0.521 | 0.725 |
| THSD4 | 0.623 | 0.052 | 0.024 | 0.521 | 0.725 |
| ASTN2 | 0.623 | 0.052 | 0.024 | 0.521 | 0.725 |
| CYP4Z1 | 0.623 | 0.052 | 0.024 | 0.522 | 0.724 |
| KIF2B | 0.623 | 0.055 | 0.024 | 0.516 | 0.730 |
| RP11-67H2.1 | 0.623 | 0.054 | 0.024 | 0.517 | 0.729 |
| TMEM202 | 0.623 | 0.052 | 0.024 | 0.520 | 0.726 |
| PGAP3 | 0.623 | 0.054 | 0.024 | 0.517 | 0.729 |
| EFCAB1 | 0.623 | 0.056 | 0.024 | 0.513 | 0.733 |
| PCDH8 | 0.623 | 0.052 | 0.025 | 0.521 | 0.724 |
| CYP26C1 | 0.623 | 0.052 | 0.025 | 0.521 | 0.724 |
| RAD21L1 | 0.623 | 0.052 | 0.025 | 0.520 | 0.726 |
| GAB4 | 0.622 | 0.054 | 0.025 | 0.516 | 0.729 |
| GRIN2A | 0.622 | 0.053 | 0.025 | 0.519 | 0.725 |
| DDI1 | 0.622 | 0.053 | 0.025 | 0.519 | 0.726 |
| PNLIPRP3 | 0.622 | 0.052 | 0.025 | 0.521 | 0.724 |
| ZNF608 | 0.622 | 0.053 | 0.025 | 0.519 | 0.726 |
| SCG2 | 0.622 | 0.052 | 0.025 | 0.521 | 0.723 |
| PPP2R2C | 0.622 | 0.052 | 0.025 | 0.520 | 0.725 |
| CNTNAP5 | 0.622 | 0.053 | 0.025 | 0.518 | 0.726 |
| OR5B12 | 0.622 | 0.054 | 0.025 | 0.517 | 0.727 |
| SLC44A5 | 0.622 | 0.051 | 0.025 | 0.521 | 0.723 |
| SPAG16 | 0.622 | 0.053 | 0.025 | 0.518 | 0.726 |
| OR51E2 | 0.622 | 0.053 | 0.026 | 0.518 | 0.725 |
| ASB17 | 0.622 | 0.054 | 0.026 | 0.516 | 0.728 |
| FAM47A | 0.622 | 0.052 | 0.026 | 0.520 | 0.724 |
| KRTAP24-1 | 0.622 | 0.052 | 0.026 | 0.519 | 0.724 |
| C16orf73 | 0.622 | 0.053 | 0.026 | 0.517 | 0.726 |
| ABCB9 | 0.622 | 0.054 | 0.026 | 0.515 | 0.728 |
| CYP4F11 | 0.622 | 0.052 | 0.026 | 0.519 | 0.724 |
| CYP46A1 | 0.622 | 0.054 | 0.026 | 0.516 | 0.727 |
| GCNT6 | 0.621 | 0.051 | 0.026 | 0.521 | 0.721 |
| ZNF200 | 0.621 | 0.054 | 0.026 | 0.516 | 0.727 |
| NR1D1 | 0.621 | 0.053 | 0.026 | 0.517 | 0.726 |
| CTNND2 | 0.621 | 0.054 | 0.026 | 0.516 | 0.727 |
| C12orf61 | 0.621 | 0.052 | 0.026 | 0.520 | 0.722 |
| SDCBP2 | 0.621 | 0.053 | 0.026 | 0.517 | 0.725 |
| IZUMO4 | 0.621 | 0.053 | 0.027 | 0.518 | 0.725 |
| TLL2 | 0.621 | 0.054 | 0.027 | 0.515 | 0.727 |
| KRTAP10-5 | 0.621 | 0.052 | 0.027 | 0.519 | 0.723 |
| OPTC | 0.621 | 0.054 | 0.027 | 0.515 | 0.727 |
| TMPRSS2 | 0.621 | 0.052 | 0.027 | 0.518 | 0.723 |
| DUSP5 | 0.621 | 0.052 | 0.027 | 0.519 | 0.723 |
| RXRG | 0.621 | 0.052 | 0.027 | 0.518 | 0.724 |
| C3orf78 | 0.621 | 0.053 | 0.027 | 0.516 | 0.726 |
| LBX1 | 0.621 | 0.052 | 0.027 | 0.518 | 0.724 |
| LOC100128071 | 0.621 | 0.053 | 0.027 | 0.518 | 0.724 |
| KRT10 | 0.621 | 0.051 | 0.027 | 0.520 | 0.722 |
| ZBTB42 | 0.621 | 0.053 | 0.027 | 0.518 | 0.724 |
| PLA2G4A | 0.621 | 0.053 | 0.027 | 0.516 | 0.726 |
| ABCA12 | 0.621 | 0.052 | 0.027 | 0.519 | 0.722 |
| ELFN1 | 0.621 | 0.053 | 0.027 | 0.517 | 0.724 |
| KCNC1 | 0.620 | 0.053 | 0.027 | 0.517 | 0.724 |
| LDHD | 0.620 | 0.053 | 0.027 | 0.516 | 0.724 |
| ARHGAP6 | 0.620 | 0.052 | 0.027 | 0.518 | 0.723 |
| CST4 | 0.620 | 0.052 | 0.028 | 0.518 | 0.723 |
| C11orf35 | 0.620 | 0.052 | 0.028 | 0.518 | 0.722 |
| C7orf53 | 0.620 | 0.054 | 0.028 | 0.513 | 0.727 |
| GJD3 | 0.620 | 0.053 | 0.028 | 0.516 | 0.724 |
| KRT79 | 0.620 | 0.052 | 0.028 | 0.517 | 0.723 |
| C12orf74 | 0.620 | 0.054 | 0.028 | 0.514 | 0.726 |
| LPPR3 | 0.620 | 0.053 | 0.028 | 0.517 | 0.723 |
| TFF3 | 0.620 | 0.052 | 0.028 | 0.519 | 0.721 |
| TMEM246 | 0.620 | 0.053 | 0.028 | 0.517 | 0.723 |
| NUDT10 | 0.620 | 0.052 | 0.028 | 0.519 | 0.721 |
| KCNS3 | 0.620 | 0.052 | 0.028 | 0.518 | 0.722 |
| PDYN | 0.620 | 0.053 | 0.028 | 0.516 | 0.723 |
| VN1R2 | 0.619 | 0.054 | 0.029 | 0.514 | 0.725 |
| PDE8B | 0.619 | 0.054 | 0.029 | 0.513 | 0.725 |
| CYP27B1 | 0.619 | 0.056 | 0.029 | 0.509 | 0.730 |
| ASB18 | 0.619 | 0.053 | 0.029 | 0.516 | 0.723 |
| SEMG1 | 0.619 | 0.053 | 0.029 | 0.515 | 0.724 |
| SH2D7 | 0.619 | 0.055 | 0.029 | 0.512 | 0.726 |
| FUT6 | 0.619 | 0.052 | 0.029 | 0.518 | 0.721 |
| EYS | 0.619 | 0.053 | 0.029 | 0.516 | 0.723 |
| TMEM239 | 0.619 | 0.054 | 0.029 | 0.514 | 0.725 |
| AC102948.2 | 0.619 | 0.052 | 0.029 | 0.517 | 0.722 |
| NKX3-1 | 0.619 | 0.052 | 0.029 | 0.518 | 0.721 |
| FCAMR | 0.619 | 0.054 | 0.029 | 0.514 | 0.725 |
| AGPHD1 | 0.619 | 0.054 | 0.029 | 0.513 | 0.725 |
| HTR1A | 0.619 | 0.055 | 0.029 | 0.511 | 0.727 |
| AWAT2 | 0.619 | 0.053 | 0.029 | 0.516 | 0.722 |
| UNC5A | 0.619 | 0.053 | 0.029 | 0.515 | 0.724 |
| LIN7A | 0.619 | 0.053 | 0.029 | 0.515 | 0.723 |
| VRTN | 0.619 | 0.053 | 0.029 | 0.514 | 0.723 |
| DUSP28 | 0.619 | 0.052 | 0.029 | 0.517 | 0.721 |
| CAPNS2 | 0.619 | 0.052 | 0.030 | 0.516 | 0.721 |
| HTR2C | 0.619 | 0.053 | 0.030 | 0.515 | 0.722 |
| TTLL10 | 0.619 | 0.056 | 0.030 | 0.508 | 0.729 |
| AKR1B15 | 0.619 | 0.053 | 0.030 | 0.515 | 0.722 |
| KCNK3 | 0.619 | 0.053 | 0.030 | 0.514 | 0.723 |
| TRPC6 | 0.619 | 0.053 | 0.030 | 0.515 | 0.722 |
| CNTN1 | 0.619 | 0.051 | 0.030 | 0.518 | 0.719 |
| IL22RA1 | 0.618 | 0.054 | 0.030 | 0.512 | 0.724 |
| AC011294.3 | 0.618 | 0.053 | 0.030 | 0.514 | 0.723 |
| SLIT1 | 0.618 | 0.053 | 0.030 | 0.513 | 0.723 |
| H2BFWT | 0.618 | 0.055 | 0.030 | 0.511 | 0.726 |
| RP11-794P6.2 | 0.618 | 0.054 | 0.030 | 0.513 | 0.723 |
| FZD5 | 0.618 | 0.052 | 0.030 | 0.516 | 0.721 |
| CFHR3 | 0.618 | 0.052 | 0.030 | 0.517 | 0.720 |
| PROCR | 0.618 | 0.053 | 0.030 | 0.515 | 0.721 |
| ROPN1L | 0.618 | 0.052 | 0.030 | 0.516 | 0.721 |
| KISS1 | 0.618 | 0.052 | 0.030 | 0.516 | 0.721 |
| TTC39A | 0.618 | 0.052 | 0.030 | 0.516 | 0.721 |
| C3orf30 | 0.618 | 0.053 | 0.031 | 0.515 | 0.721 |
| ZFP42 | 0.618 | 0.053 | 0.031 | 0.515 | 0.721 |
| LOC100652903 | 0.618 | 0.054 | 0.031 | 0.512 | 0.723 |
| SRCIN1 | 0.618 | 0.051 | 0.031 | 0.517 | 0.719 |
| TCN1 | 0.618 | 0.052 | 0.031 | 0.515 | 0.721 |
| ZBTB48 | 0.618 | 0.054 | 0.031 | 0.511 | 0.725 |
| CCDC40 | 0.618 | 0.053 | 0.031 | 0.513 | 0.722 |
| GOLT1A | 0.618 | 0.056 | 0.031 | 0.507 | 0.728 |
| GABRG1 | 0.618 | 0.053 | 0.031 | 0.514 | 0.722 |
| CXorf22 | 0.618 | 0.052 | 0.031 | 0.516 | 0.720 |
| CNTNAP2 | 0.618 | 0.053 | 0.031 | 0.514 | 0.722 |
| MORN4 | 0.618 | 0.052 | 0.031 | 0.516 | 0.719 |
| GGTLC2 | 0.618 | 0.053 | 0.031 | 0.513 | 0.722 |
| OR52A1 | 0.618 | 0.054 | 0.031 | 0.512 | 0.724 |
| TARBP2 | 0.618 | 0.052 | 0.031 | 0.516 | 0.719 |
| LACRT | 0.618 | 0.054 | 0.031 | 0.512 | 0.723 |
| REG4 | 0.617 | 0.053 | 0.032 | 0.514 | 0.721 |
| CGA | 0.617 | 0.054 | 0.032 | 0.511 | 0.723 |
| MIR1287 | 0.617 | 0.054 | 0.032 | 0.512 | 0.723 |
| MIR29B2 | 0.617 | 0.053 | 0.032 | 0.514 | 0.720 |
| ROGDI | 0.617 | 0.052 | 0.032 | 0.516 | 0.719 |
| CAMK1G | 0.617 | 0.055 | 0.032 | 0.510 | 0.725 |
| SYT2 | 0.617 | 0.053 | 0.032 | 0.514 | 0.720 |
| POU3F2 | 0.617 | 0.054 | 0.032 | 0.512 | 0.723 |
| LOC100507547 | 0.617 | 0.053 | 0.032 | 0.514 | 0.720 |
| MLPH | 0.617 | 0.051 | 0.032 | 0.517 | 0.717 |
| CYP4X1 | 0.617 | 0.054 | 0.032 | 0.512 | 0.722 |
| RAX | 0.617 | 0.055 | 0.032 | 0.510 | 0.724 |
| CYP2F1 | 0.617 | 0.057 | 0.032 | 0.506 | 0.728 |
| RILP | 0.617 | 0.053 | 0.032 | 0.512 | 0.722 |
| CYP8B1 | 0.617 | 0.052 | 0.032 | 0.515 | 0.719 |
| C15orf37 | 0.617 | 0.053 | 0.032 | 0.514 | 0.720 |
| DNAH7 | 0.617 | 0.053 | 0.032 | 0.513 | 0.721 |
| SLC9A5 | 0.617 | 0.056 | 0.032 | 0.508 | 0.726 |
| TMEM249 | 0.617 | 0.052 | 0.032 | 0.516 | 0.718 |
| LOC148824 | 0.617 | 0.054 | 0.032 | 0.512 | 0.722 |
| SMC1B | 0.617 | 0.057 | 0.032 | 0.505 | 0.728 |
| CAPN9 | 0.617 | 0.057 | 0.032 | 0.505 | 0.728 |
| SPRR2F | 0.617 | 0.054 | 0.032 | 0.510 | 0.723 |
| MIR4321 | 0.617 | 0.056 | 0.032 | 0.508 | 0.726 |
| LONRF3 | 0.617 | 0.053 | 0.032 | 0.512 | 0.721 |
| REN | 0.617 | 0.054 | 0.032 | 0.511 | 0.722 |
| SIX1 | 0.616 | 0.054 | 0.033 | 0.511 | 0.722 |
| SPR | 0.616 | 0.053 | 0.033 | 0.513 | 0.720 |
| C15orf56 | 0.616 | 0.054 | 0.033 | 0.511 | 0.722 |
| WRAP73 | 0.616 | 0.055 | 0.033 | 0.509 | 0.724 |
| P2RX3 | 0.616 | 0.055 | 0.033 | 0.510 | 0.723 |
| PPFIA2 | 0.616 | 0.052 | 0.033 | 0.515 | 0.718 |
| C16orf89 | 0.616 | 0.053 | 0.033 | 0.512 | 0.720 |
| PPP1R32 | 0.616 | 0.054 | 0.033 | 0.510 | 0.723 |
| GPRC5C | 0.616 | 0.053 | 0.033 | 0.512 | 0.720 |
| OR51A2 | 0.616 | 0.054 | 0.034 | 0.509 | 0.723 |
| CBX8 | 0.616 | 0.053 | 0.034 | 0.511 | 0.720 |
| P2RY4 | 0.616 | 0.054 | 0.034 | 0.509 | 0.722 |
| REM2 | 0.616 | 0.055 | 0.034 | 0.508 | 0.724 |
| C12orf39 | 0.616 | 0.053 | 0.034 | 0.512 | 0.720 |
| NME5 | 0.616 | 0.052 | 0.034 | 0.514 | 0.718 |
| PKDCC | 0.616 | 0.053 | 0.034 | 0.512 | 0.720 |
| DNAH5 | 0.616 | 0.054 | 0.034 | 0.510 | 0.721 |
| A2LD1 | 0.616 | 0.053 | 0.034 | 0.513 | 0.719 |
| GLTPD2 | 0.616 | 0.052 | 0.034 | 0.513 | 0.718 |
| HKDC1 | 0.616 | 0.053 | 0.034 | 0.513 | 0.719 |
| GALR2 | 0.616 | 0.056 | 0.034 | 0.505 | 0.726 |
| DLX4 | 0.616 | 0.052 | 0.034 | 0.514 | 0.717 |
| FABP1 | 0.616 | 0.054 | 0.034 | 0.510 | 0.721 |
| UBXN10 | 0.616 | 0.052 | 0.034 | 0.513 | 0.718 |
| WFDC10B | 0.616 | 0.057 | 0.034 | 0.504 | 0.727 |
| ANKRD37 | 0.616 | 0.054 | 0.034 | 0.510 | 0.721 |
| RELL2 | 0.615 | 0.052 | 0.035 | 0.514 | 0.717 |
| ATP1B2 | 0.615 | 0.053 | 0.035 | 0.512 | 0.718 |
| GBX2 | 0.615 | 0.052 | 0.035 | 0.513 | 0.717 |
| OR6T1 | 0.615 | 0.056 | 0.035 | 0.506 | 0.724 |
| PTGES3L | 0.615 | 0.054 | 0.035 | 0.509 | 0.722 |
| FOXA3 | 0.615 | 0.052 | 0.035 | 0.513 | 0.718 |
| ZNF174 | 0.615 | 0.051 | 0.035 | 0.515 | 0.716 |
| TM4SF18 | 0.615 | 0.052 | 0.035 | 0.514 | 0.717 |
| CXorf66 | 0.615 | 0.053 | 0.035 | 0.510 | 0.720 |
| CCDC48 | 0.615 | 0.053 | 0.035 | 0.511 | 0.719 |
| GJE1 | 0.615 | 0.054 | 0.035 | 0.508 | 0.722 |
| TRIM15 | 0.615 | 0.053 | 0.035 | 0.512 | 0.718 |
| CDHR5 | 0.615 | 0.054 | 0.035 | 0.509 | 0.721 |
| FRMPD3 | 0.615 | 0.053 | 0.035 | 0.511 | 0.718 |
| CGNL1 | 0.615 | 0.053 | 0.035 | 0.512 | 0.718 |
| ANKRD34B | 0.615 | 0.053 | 0.036 | 0.510 | 0.719 |
| MEP1B | 0.615 | 0.053 | 0.036 | 0.511 | 0.718 |
| C4orf6 | 0.615 | 0.055 | 0.036 | 0.507 | 0.722 |
| TP53AIP1 | 0.615 | 0.055 | 0.036 | 0.508 | 0.722 |
| LHFPL4 | 0.615 | 0.053 | 0.036 | 0.511 | 0.718 |
| OR8H1 | 0.615 | 0.054 | 0.036 | 0.509 | 0.720 |
| DNAJA4 | 0.615 | 0.056 | 0.036 | 0.506 | 0.724 |
| AC096644.1 | 0.615 | 0.054 | 0.036 | 0.509 | 0.720 |
| RIMKLA | 0.615 | 0.053 | 0.036 | 0.510 | 0.719 |
| DMBT1 | 0.615 | 0.055 | 0.036 | 0.507 | 0.722 |
| ENPP6 | 0.614 | 0.053 | 0.036 | 0.510 | 0.719 |
| LOC283731 | 0.614 | 0.053 | 0.036 | 0.510 | 0.718 |
| GSC | 0.614 | 0.054 | 0.036 | 0.509 | 0.720 |
| ILDR1 | 0.614 | 0.054 | 0.036 | 0.508 | 0.721 |
| NHS | 0.614 | 0.052 | 0.036 | 0.513 | 0.716 |
| HCN4 | 0.614 | 0.052 | 0.036 | 0.513 | 0.716 |
| RNF133 | 0.614 | 0.052 | 0.036 | 0.512 | 0.717 |
| S100A12 | 0.614 | 0.056 | 0.036 | 0.505 | 0.724 |
| PTK6 | 0.614 | 0.051 | 0.036 | 0.514 | 0.715 |
| MAGEA1 | 0.614 | 0.055 | 0.036 | 0.507 | 0.722 |
| FAM47E | 0.614 | 0.055 | 0.036 | 0.506 | 0.722 |
| LHCGR | 0.614 | 0.054 | 0.036 | 0.508 | 0.720 |
| FCRLB | 0.614 | 0.052 | 0.037 | 0.513 | 0.716 |
| ABAT | 0.614 | 0.053 | 0.037 | 0.510 | 0.718 |
| OR10H3 | 0.614 | 0.053 | 0.037 | 0.511 | 0.717 |
| CA14 | 0.614 | 0.054 | 0.037 | 0.508 | 0.720 |
| KAZN | 0.614 | 0.054 | 0.037 | 0.508 | 0.720 |
| WDR66 | 0.614 | 0.052 | 0.037 | 0.511 | 0.717 |
| C1orf159 | 0.614 | 0.056 | 0.037 | 0.505 | 0.723 |
| THAP8 | 0.614 | 0.055 | 0.037 | 0.507 | 0.721 |
| LMX1A | 0.614 | 0.054 | 0.037 | 0.508 | 0.719 |
| PYY | 0.614 | 0.054 | 0.037 | 0.508 | 0.720 |
| TNNT2 | 0.613 | 0.054 | 0.038 | 0.508 | 0.719 |
| LOC339760 | 0.613 | 0.054 | 0.038 | 0.507 | 0.720 |
| MSRB2 | 0.613 | 0.053 | 0.038 | 0.510 | 0.717 |
| FTMT | 0.613 | 0.052 | 0.038 | 0.511 | 0.716 |
| THRB | 0.613 | 0.055 | 0.038 | 0.506 | 0.721 |
| MAML3 | 0.613 | 0.052 | 0.038 | 0.512 | 0.715 |
| SRRM4 | 0.613 | 0.055 | 0.038 | 0.506 | 0.721 |
| TSPEAR | 0.613 | 0.052 | 0.038 | 0.510 | 0.716 |
| PHYHIP | 0.613 | 0.053 | 0.038 | 0.509 | 0.717 |
| USP50 | 0.613 | 0.053 | 0.038 | 0.509 | 0.717 |
| CDX1 | 0.613 | 0.052 | 0.038 | 0.511 | 0.715 |
| CTSE | 0.613 | 0.055 | 0.038 | 0.505 | 0.722 |
| GRB14 | 0.613 | 0.052 | 0.038 | 0.510 | 0.716 |
| MUC19 | 0.613 | 0.053 | 0.038 | 0.508 | 0.718 |
| DEFB116 | 0.613 | 0.056 | 0.038 | 0.504 | 0.723 |
| PCDHB6 | 0.613 | 0.052 | 0.038 | 0.512 | 0.714 |
| CCDC140 | 0.613 | 0.055 | 0.039 | 0.506 | 0.720 |
| FBXL16 | 0.613 | 0.053 | 0.039 | 0.508 | 0.717 |
| KCND3 | 0.613 | 0.052 | 0.039 | 0.510 | 0.716 |
| CFC1 | 0.613 | 0.053 | 0.039 | 0.508 | 0.717 |
| PF4 | 0.613 | 0.055 | 0.039 | 0.505 | 0.720 |
| ARFIP1 | 0.613 | 0.055 | 0.039 | 0.506 | 0.720 |
| RGS7 | 0.613 | 0.053 | 0.039 | 0.509 | 0.717 |
| HTR3C | 0.613 | 0.055 | 0.039 | 0.504 | 0.721 |
| ACSM1 | 0.613 | 0.053 | 0.039 | 0.510 | 0.716 |
| DNAJB5 | 0.613 | 0.054 | 0.039 | 0.508 | 0.718 |
| GNRH2 | 0.613 | 0.055 | 0.039 | 0.504 | 0.721 |
| POU2AF1 | 0.613 | 0.052 | 0.039 | 0.510 | 0.715 |
| GDF9 | 0.613 | 0.052 | 0.039 | 0.510 | 0.715 |
| PRM2 | 0.613 | 0.054 | 0.039 | 0.506 | 0.719 |
| FAM151A | 0.613 | 0.053 | 0.039 | 0.509 | 0.716 |
| HYDIN | 0.613 | 0.053 | 0.039 | 0.509 | 0.716 |
| KIR3DX1 | 0.613 | 0.053 | 0.039 | 0.509 | 0.717 |
| MRPL43 | 0.612 | 0.052 | 0.040 | 0.511 | 0.714 |
| AGXT | 0.612 | 0.055 | 0.040 | 0.504 | 0.720 |
| IL29 | 0.612 | 0.056 | 0.040 | 0.502 | 0.722 |
| ASCL4 | 0.612 | 0.053 | 0.040 | 0.508 | 0.716 |
| SMPX | 0.612 | 0.053 | 0.040 | 0.509 | 0.715 |
| CPEB3 | 0.612 | 0.052 | 0.040 | 0.509 | 0.715 |
| WNK4 | 0.612 | 0.056 | 0.040 | 0.502 | 0.723 |
| NPHS2 | 0.612 | 0.052 | 0.040 | 0.510 | 0.714 |
| LNX1 | 0.612 | 0.053 | 0.040 | 0.508 | 0.716 |
| GSG1L | 0.612 | 0.051 | 0.040 | 0.512 | 0.712 |
| RFPL2 | 0.612 | 0.055 | 0.040 | 0.504 | 0.719 |
| AC129492.6 | 0.612 | 0.053 | 0.040 | 0.509 | 0.715 |
| OR51A4 | 0.612 | 0.055 | 0.040 | 0.505 | 0.719 |
| HABP2 | 0.612 | 0.054 | 0.040 | 0.506 | 0.718 |
| NR0B1 | 0.612 | 0.053 | 0.040 | 0.509 | 0.715 |
| C10orf82 | 0.612 | 0.055 | 0.040 | 0.505 | 0.719 |
| LY6K | 0.612 | 0.054 | 0.040 | 0.505 | 0.719 |
| RPA3 | 0.612 | 0.053 | 0.040 | 0.508 | 0.716 |
| ISX | 0.612 | 0.054 | 0.040 | 0.507 | 0.717 |
| LRRC4B | 0.612 | 0.053 | 0.040 | 0.507 | 0.717 |
| KRTAP9-9 | 0.612 | 0.053 | 0.040 | 0.508 | 0.716 |
| DPP6 | 0.612 | 0.053 | 0.040 | 0.508 | 0.715 |
| NPTX2 | 0.612 | 0.051 | 0.041 | 0.511 | 0.712 |
| ABCG2 | 0.612 | 0.053 | 0.041 | 0.508 | 0.716 |
| OR5J2 | 0.612 | 0.053 | 0.041 | 0.507 | 0.717 |
| TSPAN1 | 0.612 | 0.052 | 0.041 | 0.510 | 0.713 |
| CCDC135 | 0.612 | 0.053 | 0.041 | 0.507 | 0.716 |
| DEAF1 | 0.612 | 0.053 | 0.041 | 0.508 | 0.716 |
| FOXJ1 | 0.612 | 0.054 | 0.041 | 0.506 | 0.718 |
| IL19 | 0.612 | 0.054 | 0.041 | 0.507 | 0.717 |
| NKX2-1 | 0.612 | 0.052 | 0.041 | 0.509 | 0.714 |
| C9orf152 | 0.612 | 0.052 | 0.041 | 0.509 | 0.714 |
| STK36 | 0.612 | 0.054 | 0.041 | 0.506 | 0.717 |
| FAM186B | 0.611 | 0.053 | 0.041 | 0.507 | 0.716 |
| MS4A15 | 0.611 | 0.054 | 0.041 | 0.505 | 0.717 |
| SRCRB4D | 0.611 | 0.055 | 0.041 | 0.504 | 0.719 |
| AC023590.1 | 0.611 | 0.053 | 0.041 | 0.508 | 0.715 |
| TTC18 | 0.611 | 0.052 | 0.041 | 0.510 | 0.713 |
| SRY | 0.611 | 0.053 | 0.042 | 0.507 | 0.715 |
| LRRC3B | 0.611 | 0.054 | 0.042 | 0.506 | 0.717 |
| ASTN1 | 0.611 | 0.053 | 0.042 | 0.507 | 0.716 |
| C5orf63 | 0.611 | 0.053 | 0.042 | 0.508 | 0.714 |
| DDC | 0.611 | 0.053 | 0.042 | 0.506 | 0.716 |
| C19orf73 | 0.611 | 0.052 | 0.042 | 0.508 | 0.714 |
| AE000662.92 | 0.611 | 0.053 | 0.042 | 0.506 | 0.716 |
| CDRT1 | 0.611 | 0.054 | 0.042 | 0.505 | 0.717 |
| CCDC157 | 0.611 | 0.053 | 0.042 | 0.507 | 0.715 |
| SH2D4A | 0.611 | 0.053 | 0.042 | 0.508 | 0.714 |
| VIL1 | 0.611 | 0.054 | 0.042 | 0.506 | 0.716 |
| OR6V1 | 0.611 | 0.053 | 0.042 | 0.507 | 0.715 |
| GLT25D2 | 0.611 | 0.054 | 0.042 | 0.505 | 0.717 |
| CYP17A1 | 0.611 | 0.054 | 0.042 | 0.505 | 0.717 |
| C2orf62 | 0.611 | 0.055 | 0.042 | 0.504 | 0.718 |
| OSGIN1 | 0.611 | 0.055 | 0.042 | 0.503 | 0.719 |
| MTRNR2L4 | 0.611 | 0.052 | 0.042 | 0.509 | 0.713 |
| CILP2 | 0.611 | 0.053 | 0.042 | 0.507 | 0.714 |
| SYCN | 0.611 | 0.054 | 0.042 | 0.506 | 0.716 |
| MPP7 | 0.611 | 0.052 | 0.043 | 0.508 | 0.713 |
| TSGA13 | 0.610 | 0.054 | 0.043 | 0.506 | 0.715 |
| CLC | 0.610 | 0.054 | 0.043 | 0.504 | 0.717 |
| MTRNR2L5 | 0.610 | 0.051 | 0.043 | 0.510 | 0.710 |
| GRXCR2 | 0.610 | 0.054 | 0.043 | 0.505 | 0.716 |
| PIPOX | 0.610 | 0.055 | 0.043 | 0.503 | 0.718 |
| NPPA | 0.610 | 0.054 | 0.043 | 0.504 | 0.716 |
| FAM46D | 0.610 | 0.053 | 0.043 | 0.506 | 0.715 |
| DLL4 | 0.610 | 0.053 | 0.043 | 0.506 | 0.715 |
| C10orf71 | 0.610 | 0.053 | 0.043 | 0.507 | 0.714 |
| TDRD15 | 0.610 | 0.053 | 0.043 | 0.506 | 0.715 |
| UNC5CL | 0.610 | 0.054 | 0.043 | 0.504 | 0.717 |
| ABCC6 | 0.610 | 0.053 | 0.043 | 0.506 | 0.715 |
| SLC45A1 | 0.610 | 0.053 | 0.043 | 0.507 | 0.714 |
| KLF13 | 0.610 | 0.053 | 0.043 | 0.506 | 0.714 |
| KCNJ1 | 0.610 | 0.053 | 0.043 | 0.506 | 0.714 |
| IFNA4 | 0.610 | 0.052 | 0.043 | 0.508 | 0.713 |
| CCDC144A | 0.610 | 0.054 | 0.044 | 0.504 | 0.716 |
| SLC9A2 | 0.610 | 0.053 | 0.044 | 0.506 | 0.714 |
| CCDC159 | 0.610 | 0.053 | 0.044 | 0.507 | 0.713 |
| PFN3 | 0.610 | 0.053 | 0.044 | 0.506 | 0.714 |
| DMRTB1 | 0.610 | 0.056 | 0.044 | 0.500 | 0.720 |
| DEFB129 | 0.610 | 0.055 | 0.044 | 0.502 | 0.718 |
| STX1A | 0.610 | 0.053 | 0.044 | 0.506 | 0.714 |
| GKN1 | 0.610 | 0.053 | 0.044 | 0.506 | 0.713 |
| OIT3 | 0.610 | 0.054 | 0.044 | 0.505 | 0.715 |
| PGAP2 | 0.610 | 0.053 | 0.044 | 0.506 | 0.714 |
| ZNF668 | 0.610 | 0.057 | 0.044 | 0.499 | 0.721 |
| FAM40B | 0.610 | 0.052 | 0.044 | 0.508 | 0.712 |
| HTR1B | 0.610 | 0.055 | 0.044 | 0.502 | 0.718 |
| C1orf100 | 0.610 | 0.053 | 0.044 | 0.505 | 0.714 |
| SCGB2A1 | 0.610 | 0.054 | 0.044 | 0.504 | 0.716 |
| LOC100653137 | 0.610 | 0.053 | 0.044 | 0.506 | 0.714 |
| DYRK3 | 0.610 | 0.053 | 0.045 | 0.505 | 0.714 |
| OSBPL7 | 0.610 | 0.055 | 0.045 | 0.502 | 0.717 |
| DSCAML1 | 0.610 | 0.054 | 0.045 | 0.504 | 0.715 |
| TAS2R40 | 0.609 | 0.054 | 0.045 | 0.503 | 0.716 |
| PSCA | 0.609 | 0.053 | 0.045 | 0.505 | 0.714 |
| SLC4A8 | 0.609 | 0.053 | 0.045 | 0.505 | 0.714 |
| LRRC39 | 0.609 | 0.052 | 0.045 | 0.508 | 0.711 |
| SLC1A7 | 0.609 | 0.054 | 0.045 | 0.503 | 0.716 |
| IFNA2 | 0.609 | 0.053 | 0.045 | 0.505 | 0.714 |
| SLC24A2 | 0.609 | 0.054 | 0.045 | 0.503 | 0.716 |
| FGF18 | 0.609 | 0.052 | 0.045 | 0.507 | 0.712 |
| ANKRD61 | 0.609 | 0.053 | 0.045 | 0.505 | 0.714 |
| NRAP | 0.609 | 0.054 | 0.045 | 0.504 | 0.715 |
| GPX6 | 0.609 | 0.054 | 0.045 | 0.504 | 0.714 |
| RNF148 | 0.609 | 0.053 | 0.046 | 0.505 | 0.713 |
| ARL4A | 0.609 | 0.054 | 0.046 | 0.504 | 0.714 |
| HOXB1 | 0.609 | 0.055 | 0.046 | 0.501 | 0.717 |
| GRIN1 | 0.609 | 0.053 | 0.046 | 0.505 | 0.713 |
| SPINT3 | 0.609 | 0.054 | 0.046 | 0.503 | 0.715 |
| ASPA | 0.609 | 0.052 | 0.046 | 0.508 | 0.710 |
| SHISA9 | 0.609 | 0.052 | 0.046 | 0.506 | 0.712 |
| LGALS13 | 0.609 | 0.056 | 0.046 | 0.500 | 0.718 |
| METTL24 | 0.609 | 0.052 | 0.046 | 0.508 | 0.710 |
| ENTHD1 | 0.609 | 0.054 | 0.046 | 0.503 | 0.714 |
| CBX4 | 0.609 | 0.053 | 0.046 | 0.505 | 0.713 |
| ETV1 | 0.609 | 0.052 | 0.046 | 0.506 | 0.712 |
| GABRG2 | 0.609 | 0.054 | 0.046 | 0.504 | 0.714 |
| UMOD | 0.609 | 0.054 | 0.046 | 0.503 | 0.715 |
| CCDC62 | 0.609 | 0.055 | 0.046 | 0.501 | 0.716 |
| PKNOX2 | 0.609 | 0.055 | 0.046 | 0.501 | 0.717 |
| MMP21 | 0.609 | 0.053 | 0.046 | 0.504 | 0.713 |
| NDNF | 0.609 | 0.052 | 0.047 | 0.507 | 0.710 |
| CIB3 | 0.608 | 0.056 | 0.047 | 0.498 | 0.718 |
| TEX264 | 0.608 | 0.052 | 0.047 | 0.506 | 0.711 |
| C1orf98 | 0.608 | 0.053 | 0.047 | 0.505 | 0.712 |
| ZSWIM1 | 0.608 | 0.054 | 0.047 | 0.503 | 0.714 |
| LHX9 | 0.608 | 0.054 | 0.048 | 0.502 | 0.714 |
| SEPHS2 | 0.608 | 0.052 | 0.048 | 0.506 | 0.710 |
| KRTAP29-1 | 0.608 | 0.054 | 0.048 | 0.503 | 0.713 |
| BAMBI | 0.608 | 0.052 | 0.048 | 0.506 | 0.710 |
| NPC1L1 | 0.608 | 0.054 | 0.048 | 0.501 | 0.715 |
| SPATC1 | 0.608 | 0.052 | 0.048 | 0.505 | 0.711 |
| C11orf9 | 0.608 | 0.054 | 0.048 | 0.502 | 0.714 |
| CELA3A | 0.608 | 0.055 | 0.048 | 0.501 | 0.715 |
| TACC2 | 0.608 | 0.052 | 0.048 | 0.506 | 0.709 |
| TCTE1 | 0.608 | 0.053 | 0.048 | 0.504 | 0.711 |
| TGIF2LX | 0.608 | 0.052 | 0.048 | 0.505 | 0.711 |
| RPS4Y1 | 0.608 | 0.053 | 0.048 | 0.504 | 0.711 |
| SPRR4 | 0.608 | 0.054 | 0.048 | 0.502 | 0.714 |
| SPACA7 | 0.608 | 0.053 | 0.048 | 0.504 | 0.712 |
| FIGLA | 0.608 | 0.054 | 0.048 | 0.502 | 0.714 |
| NRK | 0.608 | 0.054 | 0.048 | 0.502 | 0.714 |
| GPR22 | 0.608 | 0.052 | 0.048 | 0.505 | 0.710 |
| RP11-3B7.1 | 0.608 | 0.053 | 0.048 | 0.503 | 0.712 |
| HOPX | 0.608 | 0.052 | 0.049 | 0.506 | 0.709 |
| BFSP1 | 0.608 | 0.054 | 0.049 | 0.502 | 0.713 |
| TMC5 | 0.608 | 0.052 | 0.049 | 0.506 | 0.709 |
| TFAP2B | 0.608 | 0.052 | 0.049 | 0.505 | 0.710 |
| ARMC12 | 0.608 | 0.054 | 0.049 | 0.501 | 0.714 |
| RAB19 | 0.608 | 0.053 | 0.049 | 0.504 | 0.711 |
| IQCA1 | 0.608 | 0.053 | 0.049 | 0.504 | 0.711 |
| SAMD11 | 0.608 | 0.053 | 0.049 | 0.503 | 0.712 |
| KCNS1 | 0.607 | 0.053 | 0.049 | 0.504 | 0.710 |
| BHLHE22 | 0.607 | 0.053 | 0.049 | 0.504 | 0.711 |
| LRRC19 | 0.607 | 0.053 | 0.049 | 0.503 | 0.711 |
| SAMD12 | 0.607 | 0.053 | 0.049 | 0.503 | 0.711 |
| MMEL1 | 0.607 | 0.054 | 0.049 | 0.501 | 0.713 |
| DDX3Y | 0.607 | 0.053 | 0.049 | 0.504 | 0.710 |
| KLK15 | 0.607 | 0.056 | 0.049 | 0.497 | 0.717 |
| GNRHR | 0.607 | 0.055 | 0.049 | 0.500 | 0.715 |
| LRRC71 | 0.607 | 0.054 | 0.049 | 0.501 | 0.714 |

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

Furthermore, references to patents and printed publications may have been made in this specification. Each of the above-cited references and printed publications are individually incorporated herein by reference in their entirety.

In closing, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

We claim:

1. A method for predicting pathological complete response (pCR) in triple negative cancer patients (TNBC) based on measuring transcript per million expression values of genes in a gene panel, wherein the gene panel comprises one or more of the following genes: LMNB1, TFDP1, SMC4, NASP, RAD54L, NUP210, SEMA4D, BRIP1, GBP5, STIL, FERMT3, LAMP3, SLAMF8, SNX20, HIST1H2BO, SLC25A19, IL32, KIF18A, LAP3, PLEKHO1, TUBGCP3, CORO1A, ZBED2, TCN2, MSL2, CHAF1A, UNC13D, SKA2, IFI30, ZNF672, CTLA4, MAGOH, CYTH1, LCP2, TRA2B, CD300A, IRF8, IKZF1, BIRC3, FAM78A, CXCL10, HCST, POLA1, KCNAB2, PDCD1LG2, NCAPH2, RRM2, FOXM1, C5orf56, RAD51AP1, ANKRD10, CD53, ILF3, CARS2, CD52, and DAPK2.

2. A method for predicting residual disease (RD) in triple negative cancer patients (TNBC) based on measuring transcript per million expression values of genes in a gene panel, wherein the gene panel comprises one or more of the following genes: AKR1C2, ALS2CL, BAMBI, CAB39L, CAV2, CBR3.AS1, CCDC149, CD46, CHST1, CLDN12, EPB41L5, FBXL16, FITM2, FSIP2, FSTL3, HSF4, INHBB, LONP2, MATN3, NQO1, PLLP, PTGR2, PTK6, SERINC3, SLC2A10, SNX21, TACC2, THSD4, TMEM150A, TP53INP2, and UNC5CL.

3. A method for predicting disease free survival (DFS), distant metastasis free survival (DMFS) or overall survival (OS) in basal breast cancer patients based on measuring transcript per million expression values of genes in a gene panel of claim 1.

4. A method for predicting disease free survival (DFS), distant metastasis free survival (DMFS) or overall survival (OS) in basal breast cancer patients based on measuring transcript per million expression values of genes in a gene panel of claim 2.

5. The method of claim 1, wherein the area under the curve (AUC) calculated using ROC curve analysis is at least about 0.6 for each gene.

6. The method of claim 2, wherein the area under the curve (AUC) calculated using ROC curve analysis is at least about 0.6 for each gene.

7. The method of claim 3, wherein the area under the curve (AUC) calculated using ROC curve analysis is at least about 0.6 for each gene.

8. A method for selecting patients suffering from test triple negative breast cancer patients for neoadjuvant chemotherapy, the method comprising measuring transcript per million expression values of genes in the gene panel of claim 1 and selecting patients for neoadjuvant chemotherapy if the AUC for each gene in the gene panel is at least about 0.6.

9. A method for selecting patients suffering from test triple negative breast cancer patients for neoadjuvant chemotherapy, the method comprising measuring transcript per million expression values of genes in the gene panel of claim 2 and selecting patients for neoadjuvant chemotherapy if the AUC for each gene in the gene panel is at least about 0.6.

10. A kit comprising the gene panel of claim 1.

11. A kit comprising the gene panel of claim 2.

12. A kit comprising a gene panel comprising genes LMNB1, TFDP1, SMC4, NASP, RAD54L, NUP210, SEMA4D, BRIP1, GBP5, STIL, FERMT3, LAMP3, SLAMF8, SNX20, HIST1H2BO, SLC25A19, IL32, KIF18A, LAP3, PLEKHO1, TUBGCP3, CORO1A, ZBED2, TCN2, MSL2, CHAF1A, UNC13D, SKA2, IFI30, ZNF672, CTLA4, MAGOH, CYTH1, LCP2, TRA2B, CD300A, IRF8, IKZF1, BIRC3, FAM78A, CXCL10, HCST, POLA1, KCNAB2, PDCD1LG2, NCAPH2, RRM2, FOXM1, C5orf56, RAD51AP1, ANKRD10, CD53, ILF3, CARS2, CD52, and DAPK2; and
a gene panel comprising genes AKR1C2, ALS2CL, BAMBI, CAB39L, CAV2, CBR3.AS1, CCDC149, CD46, CHST1, CLDN12, EPB41L5, FBXL16, FITM2, FSIP2, FSTL3, HSF4, INHBB, LONP2, MATN3, NQO1, PLLP, PTGR2, PTK6, SERINC3, SLC2A10, SNX21, TACC2, THSD4, TMEM150A, TP53INP2, and UNC5CL.
